(19) United States
(12) Patent Application Publication
(10) Pub. No.: US 2019/0031665 A1
(43) Pub. Date: Jan. 31, 2019

(54) DERIVATIVES OF PYRROLOIMIDAZOLE OR ANALOGUES THEREOF WHICH ARE USEFUL FOR THE TREATMENT OF INTER ALIA CANCER

(71) Applicant: EMCURE PHARMACEUTICALS LIMITED, Bhosari, Pune (IN)

(72) Inventors: Mukund Keshav GURJAR, Bhosari, Pune (IN); Abhijit ROYCHOWDHURY, Bhosari, Pune (IN); Tushar Pandurang KHALADKAR, Bhosari, Pune (IN); Sangmeshwar Prabhakar SAWARGAVE, Bhosari, Pune (IN); Ravindra Ashok JANRAO, Bhosari, Pune (IN); Vijay Keshav KALHAPURE, Bhosari, Pune (IN); Ganesh Devidas URUNKAR, Bhosari, Pune (IN); Srinivas GULLAPALLI, Bhosari, Pune (IN); Jayanarayan KULATHINGAL, Bhosari, Pune (IN); Rammohan Reddy LEKKALA, Bhosari, Pune (IN)

(73) Assignee: Emcure Pharmaceuticals Limited, Bhosari, Pune (IN)

(21) Appl. No.: 16/071,712

(22) PCT Filed: Jan. 31, 2017
(86) PCT No.: PCT/IB2017/050507

§ 371 Date: Jul. 20, 2018

(30) Foreign Application Priority Data
Feb. 2, 2016 (IN) 201621003596
Jul. 14, 2016 (IN) 201621024110

(51) Int. Cl. C07D 487/04; C07D 471/14

(52) U.S. Cl. CPC C07D 487/04; C07D 471/14

(57) ABSTRACT

Present invention relates to novel heterocyclic compounds as indoleamine 2,3-dioxygenase (IDO) and/or tryptophan 2,3-dioxygenase (TDO) modulators. Compounds of the present invention inhibit tryptophan degradation by modulating IDO and/or TDO.

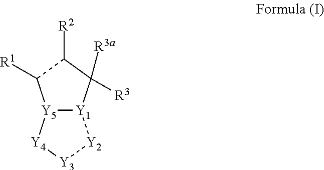

The invention further relates to the process of their preparation, pharmaceutical composition and their use in modulating the activity of indoleamine 2,3-dioxygenase (IDO) and/or tryptophan 2,3-dioxygenase (TDO). The compounds of the invention can be used alone or in combination for the treatment of conditions that benefits from the inhibition of tryptophan degradation.

This application claims the benefit of Indian provisional applications IN 201621003596 filed on 2 Feb. 2016 and IN 201621024110 filed on 14 Jul. 2016 which are hereby incorporated in their entirety.

### FIELD OF THE INVENTION

Present invention relates to novel pharmaceutical compounds that are inhibitors of indoleamine 2,3-dioxygenase (IDO) and/or tryptophan 2,3-dioxygenase (TDO). The invention further relates to preparation of these novel compounds and method of treatment for conditions related to tryptophan degradation using the compounds of the invention.

### BACKGROUND OF THE INVENTION

Indoleamine 2,3-dioxygenase (IDO) and tryptophan 2,3-dioxygenase (TDO) are tryptophan degrading enzymes that catalyze the first step in tryptophan catabolism independently in the kynurenine pathway (KP). The catabolism in turn results in depletion of tryptophan levels and formation of KP metabolites which modulates the activity of the mammalian immune, reproductive, and central nervous systems. Tryptophan (Trp) is an essential amino acid in humans as it has to be obtained through diet as body do not biosynthesize it and most of the dietary Trp being metabolized through the kynurenine pathway. Trp is also required for bio-synthesis of proteins, neurotransmitters like serotonin, melatonin and Vitamin B3 (Niacin). Excessive activation of the kynurenine pathway not only causes depletion of Trp levels but also give rise to production Kynurenine based metabolites and thereby causing suppression of T cell proliferation. In addition, the production of metabolites can provide a source of nicotinamide dinucleotide (NAD⁺) and have other biological effects, particularly in the immune, reproductive, and central nervous systems. (Ball H J et al., Front Immunol. 2014; 5: Article 485)

Though both IDO & TDO catalyze oxidative cleavage of tryptophan to N′-formylkynurenine, they differ from each other in many aspects. IDO is a monomer, which is distributed ubiquitously in extrahepatic tissues particularly in lung, small intestine & placenta. There are two major subtypes of IDO (IDO1 & IDO2). Sequence analysis indicates that for humans and mice, IDO1 and IDO2 proteins possess 43% homology and that the residues required for tryptophan catalytic activity are highly conserved (Ball H J, et al. Gene 2007; 396(1):203-213). It's important to note, however, that IDO1 possesses a higher affinity for L-tryptophan, when compared to IDO2 (Yuasa H J, et al. Comp Biochem Physiol B. 2009; 153(2):137-144). On the other hand TDO is a tetramer, located extensively in liver & placenta. Structural studies of IDO versus TDO show that conserved Arg117 and Tyr113 are found both in TDO and IDO which presents active site environments, however, His55 in TDO is replaced by Ser167b in IDO. (Thackray, S. et al., Biochem Society Transaction. 2008; pp. 36, 1120-1123). Till date, KP appears to be implicated in a variety of diseases and disorders, including immune system disorders, Cancer, acquired immune deficiency syndrome (AIDS), dementia complex, alzheimer's disease (AD), huntington's disease, amyotrophic lateral sclerosis (ALS), schizophrenia, psychiatric disorders, depressive disorders and neoplasias. Numerous studies have measured the levels of tryptophan and kynurenines under those conditions. Significant imbalances in Trp and its metabolites were frequently observed, which when brought back within normal ranges, often resulted in alleviation of symptoms.

The Trp catabolism is a central pathway maintaining the immunosuppressive microenvironment in many types of cancers. A relationship between cancer and elevated Trp catabolism was recognized in the early 1950s by analyzing the urine of bladder cancer patients (Boyland E. Biochem J. 1995; 60:v. Annual General Meeting). The classic concept proposes that tumor cells or myeloid cells in the tumor microenvironment or draining lymph nodes express high levels of indoleamine 2,3-dioxygenase 1 (IDO1) which leads to tumour escape from immunologically mediated rejection. Recently, it has been found that tumor cells and possibly specialized myeloid cells may express and catabolize Trp via TDO instead of or in addition to IDO1. A survey of cancer cell lines indicates that 16% of tumor cell lines are IDO1 positive, while 19% are TDO positive and 15% express both TDO and IDO1 (Pilotte L et al. Proc Natl Acad Sci USA., 2012; 109:2497-2502). These observations suggest that targeting TDO may complement IDO1 inhibition. Thus, TDO may represent an additional target for cancer immunotherapy. Remarkably, IDO1 inhibitors available to date do not cross-inhibit TDO and vice-versa, probably due to low sequence homology of these two enzymes despite similar enzymatic properties (Platten M et al., Front Immunol, 2015; 5: Article 673).

Many small molecules like 1-methyl-tryptophan & its derivatives, natural product derivatives like epigallocatechin gallate, brassilexin, coumaric acid are used in medicament as IDO inhibitor. WO 2007/054348 describes ‘Novel Medicaments’ and WO 2010/008427 describes ‘Tryptophan Catabolism in Cancer Treatment and Diagnosis’. Both these references talks about use of natural product derivatives in the treatment of disorders related to tryptophan catabolism pathway.

Application number WO 2006/122150 describes ‘Modulators Of Indoleamine 2,3-Dioxygenase And Methods Of Using The Same’, WO 2014/150677 describes ‘Inhibitors Of Indoleamine 2,3-Dioxygenase (IDO)’, WO 2014/186035 describes ‘Inhibitors Of The Kynurenine Pathway’, WO 2014/159248 describes ‘Tricyclic Compounds As Inhibitors Of Immunosuppression Mediated By Tryptophan Metabolization’, WO 2012/142237 describes ‘Fused Imidazole Derivatives Useful As IDO Inhibitors’, WO 2011/056652 describes ‘Imidazole Derivatives As IDO Inhibitors’, US 2016/0075711 describes ‘Compounds For The Inhibition Of Indoleamine-2,3-Dioxygenase’, U.S. Pat. No. 5,428,160 describes ‘Substituted imidazo[5-a]pyridine derivatives and other substituted bicyclic derivatives’, U.S. Pat. No. 6,420,057 describes ‘Organic electroluminescent element’ and JPH 0971586 describes ‘New Bicyclic Condensed Imidazole Derivative’.

Some other additional references which have disclosed Imidazo-isoindole derivatives. For example WO 2016/037026 discloses compounds for the inhibition of Indoleamine-2,3-Dioxygenase; WO 2012/142237 discloses fused imidazole derivatives useful as IDO inhibitors; WO 2016/059412 describes 6,7-heterocyclic fused 5H-Pyrrolo[1,2-C]Imidazole derivatives and their use as Indoleamine 2,3-Dioxygenase (IDO) and/or Tryptophan 2,3-Dioxygenase (TDO2) Modulators; WO 2016/051181 describe 4H-Imidazo[1,5-A]Indole derivatives and their use as Indoleamine 2,3-Dioxygenase (IDO) and/or Tryptophan 2,3-Dioxygenase (TDO2) Modulators.

In view of the world wide epidemic level of cancer, there is a strong continued need for new effective drugs for treatment of cancer such as by discovering new IDO and/or TDO inhibitor compounds with novel structures.

The present invention includes novel compounds that are inhibitors of IDO and/or TDO, methods for preparing the novel compounds, pharmaceutical compositions comprising the novel compounds, methods for using the novel compounds and a novel approach to identify promising compounds that can be potential IDO and/or TDO inhibitors. The compounds of the invention herein will help to meet the need to develop potential inhibitors of IDO and/or TDO. Considering the role of IDO & TDO in many clinical manifestations like cancer, acquired immune deficiency syndrome (AIDS), dementia, Alzheimer's disease (AD), schizophrenia, Huntington's disease, amyotrophic lateral sclerosis (ALS), autoimmune disorders like rheumatoid arthritis etc. compounds of the present invention will prove beneficial for the treatment of these diseases.

### SUMMARY OF THE INVENTION

Present invention provides indoleamine 2,3-dioxygenase (IDO) and/or tryptophan 2,3-dioxygenase (TDO) inhibitor compounds of the general Formula (I):

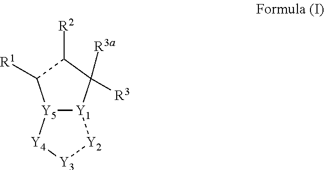

wherein,
each R¹, R² & R³ can be selected independently from a radical

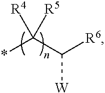

hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
and R¹ and R² can be combined together with their adjacent carbon atom to form 5-8 membered substituted or unsubstituted monocyclic or 10-12 membered substituted or unsubstituted bicyclic cycloalkyl or heterocycloalkyl ring;
R^{3a} can be selected from hydrogen, halogen, substituted or unsubstituted alkyl, substituted or unsubstituted cycloalkyl, substituted or unsubstituted aryl or substituted or unsubstituted heteroaryl;
R^{A}, R^{B} and R^{C} can be independently selected from hydrogen, substituted or unsubstituted alkyl, substituted or unsubstituted arylalkyl, substituted or unsubstituted aryl, substituted or unsubstituted cycloalkyl, substituted or unsubstituted cycloalkylalkyl, substituted or unsubstituted heterocycloalkyl, substituted or unsubstituted heteroaryl, substituted or unsubstituted heteroarylalkyl;
R⁴ and R⁵ can be independently selected from hydrogen, substituted or unsubstituted alkyl or substituted or unsubstituted aryl;
R⁶ can be selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
W can be selected from oxo (C═O), thio (C═S), OR^{A}, SR^{A}, NR^{A}R^{B} or halogen;
n can be an integer 1-6;
Y₁, Y₂, Y₃, Y₄ and Y₅ can be independently selected from CR^{D}R^{E}, N or NR^{D};
each R^{D} & R^{E} can be independently selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
   --- bond can be a single or double bond.

Pharmaceutically acceptable salts of the compounds of the Formula (I) are also contemplated. Likewise, pharmaceutically acceptable solvates, including hydrates, of the compounds of the Formula (I) are also contemplated.

It should be understood that Formula (I) structurally encompasses all stereoisomers, including enantiomers, diastereomers, racemates, and combinations thereof, which may be contemplated from the chemical structure of the genus described herein.

Also contemplated are prodrugs of the compounds of the Formula (I), including ester prodrugs.

According to one embodiment, there is provided a compound of Formula (I), wherein R¹ is hydrogen, substituted or unsubstituted alkyl or substituted or unsubstituted aryl and most preferably hydrogen, substituted or unsubstituted C₁-C₄ alkyl or substituted or unsubstituted phenyl.

According to one embodiment, there is provided a compound of Formula (I), wherein R² is hydrogen, substituted or unsubstituted alkyl, substituted or unsubstituted alkoxy or substituted or unsubstituted aryl, preferably hydrogen, C₁-C₁₂ alkyl or a substituted or unsubstituted phenyl and most preferably hydrogen or substituted or unsubstituted phenyl.

According to one embodiment, there is provided a compound of Formula (I), wherein R¹ and R² can be combined together with their adjacent carbon atom to form 5-8 membered substituted or unsubstituted monocyclic or 10-12 membered substituted or unsubstituted bicyclic cycloalkyl or heterocycloalkyl ring and most preferably 6 membered monocyclic cycloalkyl or heterocycloalkyl ring.

According to one embodiment, there is provided a compound of Formula (I), wherein R3 is a radical

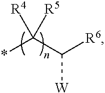

substituted or unsubstituted alkyl, substituted or unsubstituted aryloxy, substituted or unsubstituted heteroaryloxy, —R^{A}C(O)OR^{B}, and most preferably

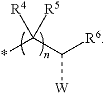

According to one embodiment, there is provided a compound of Formula (I), wherein R^{3a} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably hydrogen.

Another embodiment of the present invention provides indoleamine 2,3-dioxygenase (IDO) and/or tryptophan 2,3-dioxygenase (TDO) inhibitor compounds of the general Formula (IA):

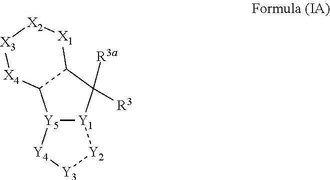

wherein,
X₁, X₂, X₃ and X₄ can be independently selected from (CR^{D}R^{E})ₚ, O, S, NR^{D}, SO or SO₂ p can be an integer 0-3;
each R^{D} & R^{E} can be independently selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
R³ can be selected independently from a radical

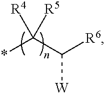

hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
R^{3a} can be selected from hydrogen, halogen, substituted or unsubstituted alkyl, substituted or unsubstituted cycloalkyl, substituted or unsubstituted aryl or substituted or unsubstituted heteroaryl;
R⁴ & R⁵ can be independently selected from hydrogen, substituted or unsubstituted alkyl or substituted or unsubstituted aryl;
R⁶ can be selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
W can be selected from oxo (C═O), thio (C═S), OR^{A}, SR^{A}, NR^{A}R^{B} or halogen;
Y₁, Y₂, Y₃, Y₄ and Y₅ can be independently selected from CR^{D}R^{E}, N or NR^{D};
R^{A}, R^{B} and R^{C} can be independently selected from hydrogen, substituted or unsubstituted alkyl, substituted or unsubstituted arylalkyl, substituted or unsubstituted aryl, substituted or unsubstituted cycloalkyl, substituted or unsubstituted cycloalkylalkyl, substituted or unsubstituted heterocycloalkyl, substituted or unsubstituted heteroaryl, substituted or unsubstituted heteroarylalkyl;
n can be an integer 1-6;
--- bond can be single or double bond.

Pharmaceutically acceptable salts of the compounds of the Formula (IA) are also contemplated. Likewise, pharmaceutically acceptable solvates, including hydrates, of the compounds of the Formula (IA) are contemplated.

It should be understood that Formula (IA) structurally encompasses all stereoisomers, including enantiomers, diastereomers, racemates, and combinations thereof, which may be contemplated from the chemical structure of the genus described herein.

Also contemplated are prodrugs of the compounds of the Formula (IA), including ester prodrugs.

According to one embodiment, there is provided a compound of Formula (IA), wherein X₁ is —CHR^{D} and preferably wherein R^{D} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably —CH₂—.

According to one embodiment, there is provided a compound of Formula (IA), wherein X₂ is —CHR^{D} or NR^{D} and preferably wherein R^{D} is hydrogen, halogen, S(O)₂R^{A}, —S(O)OR^{A}, substituted or unsubstituted C₁-C₆ alkyl, substituted or unsubstituted C₁-C₆ alkoxy, substituted or unsubstituted C₃-C₈ cycloalkyl, and most preferably —CH₂— or N (toluene-4-sulfonyl).

According to one embodiment, there is provided a compound of Formula (IA), wherein X₃ is —CHR^{D} and preferably wherein R^{D} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably —CH₂—.

According to one embodiment, there is provided a compound of Formula (IA), wherein X₄ is —CHR^{D} and preferably wherein R^{D} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably —CH₂—.

According to one embodiment, there is provided a compound of Formula (IA), wherein R³ is a radical

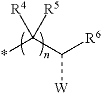

substituted or unsubstituted alkyl, substituted or unsubstituted aryloxy, substituted or unsubstituted heteroaryloxy, —R^{A}C(O)OR^{B}, and most preferably

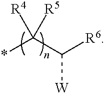

According to one embodiment, there is provided a compound of Formula (IA), wherein R^{3a} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably hydrogen.

Another embodiment of the present invention provides indoleamine 2,3-dioxygenase (IDO) and/or tryptophan 2,3-dioxygenase (TDO) inhibitor compounds of the general Formula (IB):

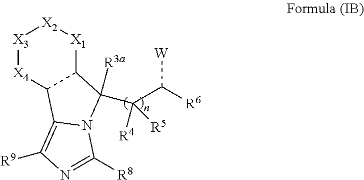

wherein,
X₁, X₂, X₃ and X₄ can be independently selected from (CR^{D}R^{E})ₚ, O, S, NR^{D}, SO or SO₂ p can be an integer 0-3;
each R^{D} & R^{E} can be independently selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
R⁸ and R⁹ can be independently selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
R^{3a} can be selected from hydrogen, halogen, substituted or unsubstituted alkyl, substituted or unsubstituted cycloalkyl, substituted or unsubstituted aryl or substituted or unsubstituted heteroaryl;
R⁴ & R⁵ can be independently selected from hydrogen, substituted or unsubstituted alkyl or substituted or unsubstituted aryl;
R⁶ can be selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
W can be selected from oxo (C═O), thio (C═S), OR^{A}, SR^{A}, NR^{A}R^{B} or halogen;
R^{A}, R^{B} and R^{C} can be independently selected from hydrogen, substituted or unsubstituted alkyl, substituted or unsubstituted arylalkyl, substituted or unsubstituted aryl, substituted or unsubstituted cycloalkyl, substituted or unsubstituted cycloalkylalkyl, substituted or unsubstituted heterocycloalkyl, substituted or unsubstituted heteroaryl, substituted or unsubstituted heteroarylalkyl;
n can be an integer 1-6;
--- bond can be a single or double bond.

Pharmaceutically acceptable salts of the compounds of the Formula (IB) are also contemplated. Likewise, pharmaceutically acceptable solvates, including hydrates, of the compounds of the Formula (IB) are contemplated.

It should be understood that Formula (IB) structurally encompasses all stereoisomers, including enantiomers, diastereomers, racemates, and combinations thereof, which may be contemplated from the chemical structure of the genus described herein.

Also contemplated are prodrugs of the compounds of the Formula (IB), including ester prodrugs.

According to one embodiment, there is provided a compound of Formula (IB), wherein X₁ is —CHR^{D} and preferably wherein R^{D} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably —CH₂—.

According to one embodiment, there is provided a compound of Formula (IB), wherein X₂ is —CHR^{D} and preferably wherein R^{D} is hydrogen, halogen, S(O)₂R^{A}, —S(O)OR^{A}, substituted or unsubstituted C₁-C₆ alkyl, substituted or unsubstituted C₁-C₆ alkoxy, substituted or unsubstituted C₃-C₈ cycloalkyl, and most preferably —CH₂— or N (toluene-4-sulfonyl).

According to one embodiment, there is provided a compound of Formula (IB), wherein X₃ is —CHR^{D} and preferably wherein R^{D} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably —CH₂—.

According to one embodiment, there is provided a compound of Formula (IB), wherein X₄ is —CHR^{D} and preferably wherein R^{D} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably —CH₂—.

According to one embodiment, there is provided a compound of Formula (IB), wherein R^{3a} is hydrogen, halogen, substituted or unsubstituted C₁-C₆ alkyl, substituted or unsubstituted C₁-C₆ alkoxy, substituted or unsubstituted C₃-C₈ cycloalkyl, and most preferably hydrogen.

According to one embodiment, there is provided a compound of Formula (IB), wherein R⁴ & R⁵ are hydrogen, halogen, substituted or unsubstituted C₁-C₆ alkyl, substituted or unsubstituted C₁-C₆ alkoxy, substituted or unsubstituted C₃-C₈ cycloalkyl, and most preferably hydrogen.

According to one embodiment, there is provided a compound of Formula (IB), wherein n is 1-3, preferably 1-2 and most preferably 1.

According to one embodiment, there is provided a compound of Formula (IB), wherein W is oxo (C═O) and OH.

According to one embodiment, there is provided a compound of Formula (IB), wherein R⁶ is substituted or unsubstituted aryl and most preferably substituted or unsubstituted phenyl.

According to one embodiment, there is provided a compound of Formula (IB), wherein R⁸ & R⁹ is hydrogen or substituted or unsubstituted alkyl and most preferably hydrogen.

Another embodiment of the present invention provides indoleamine 2,3-dioxygenase (IDO) and/or tryptophan 2,3-dioxygenase (TDO) inhibitor compounds with general Formula (IC):

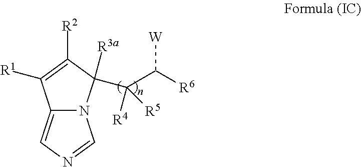

wherein,
each R¹ and R² can be selected independently from a radical

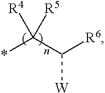

hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
each R^{A}, R^{B} and R^{C} can be independently selected from hydrogen, substituted or unsubstituted alkyl, haloalkyl, substituted or unsubstituted arylalkyl, substituted or unsubstituted aryl, substituted or unsubstituted cycloalkyl, substituted or unsubstituted cycloalkylalkyl, substituted or unsubstituted heterocycloalkyl, substituted or unsubstituted heteroaryl, substituted or unsubstituted heteroarylalkyl;
   R^{3a} can be selected from hydrogen, halogen, substituted or unsubstituted alkyl, substituted or unsubstituted cycloalkyl, substituted or unsubstituted aryl or substituted or unsubstituted heteroaryl;
   R⁴ & R⁵ can be independently selected from hydrogen, substituted or unsubstituted alkyl or substituted or unsubstituted aryl;
   R⁶ can be selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
   W can be selected from oxo (C═O), thio (C═S), OR^{A}, SR^{A}, NR^{A}R^{B} or halogen;
   n can be an integer 1-6;
   --- bond can be a single or double bond.

Pharmaceutically acceptable salts of the compounds of the Formula (IC) are also contemplated. Likewise, pharmaceutically acceptable solvates, including hydrates, of the compounds of the Formula (IC) are contemplated.

It should be understood that Formula (IC) structurally encompasses all stereoisomers, including enantiomers, diastereomers, racemates, and combinations thereof, which may be contemplated from the chemical structure of the genus described herein.

Also contemplated are prodrugs of the compounds of the Formula (IC), including ester prodrugs.

According to one embodiment, there is provided a compound of Formula (IC), wherein R¹ is hydrogen, hydrogen, substituted or unsubstituted alkyl or substituted or unsubstituted aryl and most preferably hydrogen, C₁-C₁₂ alkyl or substituted or unsubstituted phenyl.

According to one embodiment, there is provided a compound of Formula (IC), wherein R² is hydrogen, substituted or unsubstituted alkyl, substituted or unsubstituted alkoxy or substituted or unsubstituted aryl, preferably hydrogen, C₁-C₁₂ alkyl or a substituted or unsubstituted phenyl and most preferably hydrogen or substituted or unsubstituted phenyl.

According to one embodiment, there is provided a compound of Formula (IC), wherein R^{3a} is hydrogen, halogen, substituted or unsubstituted alkyl, substituted alkoxy and most preferably hydrogen.

According to one embodiment, there is provided a compound of Formula (IC), wherein R⁴ & R⁵ are hydrogen, halogen, substituted or unsubstituted alkyl, substituted alkoxy and most preferably hydrogen.

According to one embodiment, there is provided a compound of Formula (IC), wherein n is 1-3, preferably 1-2 and most preferably 1.

According to one embodiment, there is provided a compound of Formula (IC), wherein W is oxo (C═O), halogen and OH.

According to one embodiment, there is provided a compound of Formula (IC), wherein R⁶ is substituted or unsubstituted alkyl, substituted or unsubstituted aryl, —NR^{A}R^{B}, substituted or unsubstituted heterocycloalkyl, substituted or unsubstituted heteroaryl, substituted or unsubstituted cycloalkyl or OR^{A}.

In another embodiment, the compounds of the present invention have human IDO IC₅₀ values >500 nM.

In another embodiment, the compounds of the invention have human IDO IC₅₀ values <500 nM.

Below are the representative compounds, which are illustrative in nature only and are not intended to limit to the scope of the invention (Nomenclature has been generated from either ChemDraw Professional 15.0 version or ISISDraw 2.4 version):
1-Phenyl-2-(6,7,8,9-tetrahydro-5H-imidazo[5,1-a]isoindol-5-yl)-ethanone (Compound 1);
1-Phenyl-2-(6,7,8,9-tetrahydro-5H-imidazo[5,1-a]isoindol-5-yl)-ethanol (Compound 2);
1-(3-Chloro-phenyl)-2-(6,7,8,9-tetrahydro-5H-imidazo[5,1-a]isoindol-5-yl)-ethanone (Compound 3);
1-(3-Chloro-phenyl)-2-(6,7,8,9-tetrahydro-5H-imidazo[5,1-a]isoindol-5-yl)-ethanol (Compound 4);
1-Phenyl-2-[6-(toluene-4-sulfonyl)-4,6,7,8-tetrahydro-5H-2,6,8a-triaza-cyclopenta [a] inden-8-yl]-ethanone (Compound 5);
1-Phenyl-2-[6-(toluene-4-sulfonyl)-4,6,7,8-tetrahydro-5H-2,6,8a-triaza-cyclopenta [a] inden-8-yl]-ethanol (Compound 6);
1-(3-Benzyloxy-phenyl)-2-(6,7,8,9-tetrahydro-5H-imidazo[5,1-a]isoindol-5-yl)-ethanone (Compound 7);
1-(3-Benzyloxy-phenyl)-2-(6,7,8,9-tetrahydro-5H-imidazo[5,1-a]isoindol-5-yl)-ethanol (Compound 8);
1-(2,5-Difluoro-phenyl)-2-(6,7,8,9-tetrahydro-5H-imidazo[5,1-a]isoindol-5-yl)-ethanone (Compound 9);
1-(2,5-Difluoro-phenyl)-2-(6,7,8,9-tetrahydro-5H-imidazo[5,1-a]isoindol-5-yl)-ethanol (Compound 10);
2-(7-Methyl-6-phenyl-5H-pyrrolo[1,2-c]imidazol-5-yl)-1-phenyl-ethanone (Compound 11);
2-(7-Methyl-6-phenyl-5H-pyrrolo[1,2-c]imidazol-5-yl)-1-phenyl-ethanol (Compound 12);
1-Phenyl-2-(7-phenyl-5H-pyrrolo[1,2-c]imidazol-5-yl)-ethanone (Compound 13);
1-Phenyl-2-(7-phenyl-5H-pyrrolo[1,2-c]imidazol-5-yl)-ethanol (Compound 14);
2-[6-(3-Fluoro-phenyl)-7-methyl-5H-pyrrolo[1,2-c]imidazol-5-yl]-1-phenyl-ethanone (Compound 15);
2-[6-(3-Fluoro-phenyl)-7-methyl-5H-pyrrolo[1,2-c]imidazol-5-yl]-1-phenyl-ethanol (Compound 16);
1-(3-Chloro-phenyl)-2-(7-methyl-6-phenyl-5H-pyrrolo[1,2-c]imidazol-5-yl)-ethanone (Compound 17);
4-[2-(7-Methyl-6-phenyl-5H-pyrrolo[1,2-c]imidazol-5-yl)-acetyl]-benzoic acid methyl ester (Compound 18);
1-(3-Chloro-phenyl)-2-(7-methyl-6-phenyl-5H-pyrrolo[1,2-c]imidazol-5-yl)-ethanol (Compound 19);
1-(3-Fluoro-phenyl)-2-(7-methyl-6-phenyl-5H-pyrrolo[1,2-c]imidazol-5-yl)-ethanone (Compound 20);
2-(6-Methyl-7-phenyl-5H-pyrrolo[1,2-c]imidazol-5-yl)-1-phenyl-ethanone (Compound 21);
(7-Methyl-6-phenyl-5H-pyrrolo[1,2-c]imidazol-5-yl)-acetic acid ethyl ester (Compound 22);
3-[2-(7-Methyl-6-phenyl-5H-pyrrolo[1,2-c]imidazol-5-yl)-acetyl]-benzoic acid methyl ester (Compound 23);
4-[2-(7-Methyl-6-phenyl-5H-pyrrolo[1,2-c]imidazol-5-yl)-acetyl]-benzoic acid (Compound 24);
2-(7-Methyl-6-phenyl-5H-pyrrolo[1,2-c]imidazol-5-yl)-1-naphthalen-2-yl-ethanone (Compound 25);
1-Cyclopropyl-2-(7-methyl-6-phenyl-5H-pyrrolo[1,2-c]imidazol-5-yl)-ethanone (Compound 26);
2-(7-Methyl-6-phenyl-5H-pyrrolo[1,2-c]imidazol-5-yl)-1-p-tolyl-ethanone (Compound 27);
1-Benzo[1,3]dioxol-5-yl-2-(7-methyl-6-phenyl-5H-pyrrolo[1,2-c]imidazol-5-yl)-ethanone (Compound 28);
2-(7-Methyl-6-phenyl-5H-pyrrolo[1,2-c]imidazol-5-yl)-1-(3-trifluoromethyl-phenyl)-ethanone (Compound 29);
2-(7-Methyl-6-phenyl-5H-pyrrolo[1,2-c]imidazol-5-yl)-1-phenyl-ethanone (Isomer-I of Compound 11, Compound 30);
2-(7-Methyl-6-phenyl-5H-pyrrolo[1,2-c]imidazol-5-yl)-1-phenyl-ethanone (Isomer-II of Compound 11, Compound 31);
1-(2-methoxyphenyl)-2-(7-methyl-6-phenyl-5H-pyrrolo[1,2-c]imidazol-5-yl)ethan-1-one (Compound 32);
1-(3-Methoxy-phenyl)-2-(7-methyl-6-phenyl-5H-pyrrolo[1,2-c]imidazol-5-yl)-ethanone (Compound 33);
1-(4-fluorophenyl)-2-(7-methyl-6-phenyl-5H-pyrrolo[1,2-c]imidazol-5-yl)ethan-1-one (Compound 34);
1-(4-fluorophenyl)-2-(7-methyl-6-phenyl-5H-pyrrolo[1,2-c]imidazol-5-yl)ethan-1-ol (Compound 35);
1-(2,5-difluorophenyl)-2-(7-methyl-6-phenyl-5H-pyrrolo[1,2-c]imidazol-5-yl)ethan-1-one (Compound 36);
1-(4-methoxyphenyl)-2-(7-methyl-6-phenyl-5H-pyrrolo[1,2-c]imidazol-5-yl)ethan-1-one (Compound 37);
1-(4-chlorophenyl)-2-(7-methyl-6-phenyl-5H-pyrrolo[1,2-c]imidazol-5-yl)ethan-1-one (Compound 38);
1-(3-chloro-4-fluorophenyl)-2-(7-methyl-6-phenyl-5H-pyrrolo[1,2-c]imidazol-5-yl)ethan-1-one (Compound 39);
2-(7-methyl-6-phenyl-5H-pyrrolo[1,2-c]imidazol-5-yl)-1-(naphthalen-1-yl)ethan-1-one (Compound 40);
1-([1,1′-biphenyl]-4-yl)-2-(7-methyl-6-phenyl-5H-pyrrolo[1,2-c]imidazol-5-yl)ethan-1-one (Isomer-I of compound 40, Compound 41);
1-([1,1′-biphenyl]-4-yl)-2-(7-methyl-6-phenyl-5H-pyrrolo[1,2-c]imidazol-5-yl)ethan-1-one (Isomer-I of compound 40, Compound 42);
1-([1,1′-biphenyl]-4-yl)-2-(7-methyl-6-phenyl-5H-pyrrolo[1,2-c]imidazol-5-yl)ethan-1-one (Compound 43);
1-([1,1′-biphenyl]-4-yl)-2-(7-methyl-6-phenyl-5H-pyrrolo[1,2-c]imidazol-5-yl)ethan-1-ol (Compound 44);
2-(7-methyl-6-phenyl-5H-pyrrolo[1,2-c]imidazol-5-yl)-1-(thiophen-3-yl)ethan-1-one (Compound 45);
1-(4-(dimethylamino)phenyl)-2-(7-methyl-6-phenyl-5H-pyrrolo[1,2-c]imidazol-5-yl)ethan-1-one (Compound 46);
2-(7-methyl-6-phenyl-5H-pyrrolo[1,2-c]imidazol-5-yl)-1-(5,6,7,8-tetrahydronaphthalen-2-yl)ethan-1-one (Compound 47);
2-(7-methyl-6-phenyl-5H-pyrrolo[1,2-c]imidazol-5-yl)-1-(4-phenoxyphenyl)ethan-1-one (Compound 48);
2-(7-methyl-6-phenyl-5H-pyrrolo[1,2-c]imidazol-5-yl)-1-(4-morpholinophenyl)ethan-1-one (Compound 49);
2-(7-methyl-6-phenyl-5H-pyrrolo[1,2-c]imidazol-5-yl)-1-(4-(piperidin-1-yl)phenyl)ethan-1-one (Compound 50);
1-(4-bromophenyl)-2-(7-methyl-6-phenyl-5H-pyrrolo[1,2-c]imidazol-5-yl)ethan-1-one (Compound 51);
1-(4-bromophenyl)-2-(7-methyl-6-phenyl-5H-pyrrolo[1,2-c]imidazol-5-yl)ethan-1-ol (Compound 52);
2-(7-methyl-6-phenyl-5H-pyrrolo[1,2-c]imidazol-5-yl)-1-(4-(pyridin-4-yl)phenyl)ethan-1-one (Compound 53);
2-(7-methyl-6-phenyl-5H-pyrrolo[1,2-c]imidazol-5-yl)-1-(4-(pyrimidin-5-yl)phenyl)ethan-1-one (Compound 54);
2-(7-methyl-6-phenyl-5H-pyrrolo[1,2-c]imidazol-5-yl)-1-(4-(pyridin-3-yl)phenyl)ethan-1-one (Compound 55);
2-(7-methyl-6-phenyl-5H-pyrrolo[1,2-c]imidazol-5-yl)-1-(4-(thiophen-3-yl)phenyl)ethan-1-one (Compound 56);
1-(4′-hydroxy-[1,1′-biphenyl]-4-yl)-2-(7-methyl-6-phenyl-5H-pyrrolo[1,2-c]imidazol-5-yl)ethan-1-one (Compound 57);
1-(2′-fluoro-[1,1′-biphenyl]-4-yl)-2-(7-methyl-6-phenyl-5H-pyrrolo[1,2-c]imidazol-5-yl)ethan-1-one (Compound 58);
2-(7-methyl-6-phenyl-5H-pyrrolo[1,2-c]imidazol-5-yl)-1-(4-(pyridin-4-yl)phenyl)ethan-1-ol (Compound 59);
2-(7-methyl-6-phenyl-5H-pyrrolo[1,2-c]imidazol-5-yl)-1-(4-(thiophen-3-yl)phenyl)ethan-1-ol (Compound 60);
2-(7-methyl-6-phenyl-5H-pyrrolo[1,2-c]imidazol-5-yl)-1-(4′-(trifluoromethyl)-[1,1′-biphenyl]-4-yl)ethan-1-one (Compound 61);
2-(7-methyl-6-phenyl-5H-pyrrolo[1,2-c]imidazol-5-yl)-1-(4′-methyl-[1,1′-biphenyl]-4-yl)ethan-1-one (Compound 62);
1-(4′-fluoro-[1,1′-biphenyl]-4-yl)-2-(7-methyl-6-phenyl-5H-pyrrolo[1,2-c]imidazol-5-yl)ethan-1-one (Compound 63);
2-(7-methyl-6-phenyl-5H-pyrrolo[1,2-c]imidazol-5-yl)-1-(4-(5-methylthiophen-2-yl)phenyl)ethan-1-one (Compound 64);
1-([1,1′:4′,1″-terphenyl]-4-yl)-2-(7-methyl-6-phenyl-5H-pyrrolo[1,2-c]imidazol-5-yl)ethan-1-one (Compound 65);
2-(7-methyl-6-phenyl-5H-pyrrolo[1,2-c]imidazol-5-yl)-1-(4′-(methylsulfonyl)-[1,1′-biphenyl]-4-yl)ethan-1-one (Compound 66);
1-(4-(1H-imidazol-5-yl)phenyl)-2-(7-methyl-6-phenyl-5H-pyrrolo[1,2-c]imidazol-5-yl)ethan-1-one (Compound 67);
1-(3-Bromo-phenyl)-2-(7-methyl-6-phenyl-5H-pyrrolo[1,2-c]imidazol-5-yl)-ethanone (Compound 68);
1-Biphenyl-3-yl-2-(7-methyl-6-phenyl-5H-pyrrolo[1,2-c]imidazol-5-yl)-ethanone (Compound 69);
1-(2′-Fluoro-biphenyl-3-yl)-2-(7-methyl-6-phenyl-5H-pyrrolo[1,2-c]imidazol-5-yl)-ethanone (Compound 70);
4-[2-(7-Methyl-6-phenyl-5H-pyrrolo[1,2-c]imidazol-5-yl)-acetyl]-benzoic acid methyl ester (Isomer-I of compound 18, Compound 71);
4-[2-(7-Methyl-6-phenyl-5H-pyrrolo[1,2-c]imidazol-5-yl)-acetyl]-benzoic acid methyl ester (Isomer-II of compound 18, Compound 72);
1-(4-Cyclohexylphenyl)-2-(7-methyl-6-phenyl-5H-pyrrolo[1,2-c]imidazol-5-yl)ethan-1-one (Compound 73);
1-(4-Cyclohexylphenyl)-2-(7-methyl-6-phenyl-5H-pyrrolo[1,2-c]imidazol-5-yl)ethan-1-ol (Compound 74);
1-(4-(benzyloxy)phenyl)-2-(7-methyl-6-phenyl-5H-pyrrolo[1,2-c]imidazol-5-yl)ethan-1-one (Compound 75);
1-(4-(2-fluorophenoxy) phenyl)-2-(7-methyl-6-phenyl-5H-pyrrolo[1,2-c]imidazol-5-yl)ethan-1-one (Compound 76);
N-methyl-4-(2-(7-methyl-6-phenyl-5H-pyrrolo[1,2-c]imidazol-5-yl)acetyl)benzamide (Compound 77);
N-methyl-4-(2-(7-methyl-6-phenyl-5H-pyrrolo[1,2-c]imidazol-5-yl)acetyl)benzamide (Compound 78);
4-(2-(7-methyl-6-phenyl-5H-pyrrolo[1,2-c]imidazol-5-yl)acetyl)-N-(pyridin-3-yl)benzamide (Compound 79);
N-(4-chlorophenyl)-4-(2-(7-methyl-6-phenyl-5H-pyrrolo[1,2-c]imidazol-5-yl)acetyl)benzamide (Compound 80);
N-(2,4-difluorophenyl)-4-(2-(7-methyl-6-phenyl-5H-pyrrolo[1,2-c]imidazol-5-yl)acetyl)benzamide (Compound 81);
N-(4-chlorophenyl)-4-(1-hydroxy-2-(7-methyl-6-phenyl-5H-pyrrolo[1,2-c]imidazol-5-yl)ethyl)benzamide (Compound 82);
N-isobutyl-4-(2-(7-methyl-6-phenyl-5H-pyrrolo[1,2-c]imidazol-5-yl)acetyl)benzamide (Compound 83);
N-(2,4-dimethylphenyl)-4-(2-(7-methyl-6-phenyl-5H-pyrrolo[1,2-c]imidazol-5-yl)acetyl)benzamide (Compound 84);
4-(2-(7-methyl-6-phenyl-5H-pyrrolo[1,2-c]imidazol-5-yl)acetyl)-N-phenylbenzamide (Compound 85);
1-Cyclohexyl-2-(7-methyl-6-(p-tolyl)-5H-pyrrolo[1,2-c]imidazol-5-yl)ethan-1-one (Compound 86);
1-Cyclohexyl-2-(7-methyl-6-(p-tolyl)-5H-pyrrolo[1,2-c]imidazol-5-yl)ethan-1-ol (Compound 87);
1-Cyclohexyl-2-(7-methyl-6-phenyl-5H-pyrrolo[1,2-c]imidazol-5-yl)-ethanone (Compound 88);
1-Cyclohexyl-2-(7-methyl-6-phenyl-5H-pyrrolo[1,2-c]imidazol-5-yl)-ethanol (Compound 89);
1-Cyclohexyl-2-(7-methyl-6-phenyl-5H-pyrrolo[1,2-c]imidazol-5-yl)ethan-1-ol (Isomer-I of compound 89, Compound 90);
1-Cyclohexyl-2-(7-methyl-6-phenyl-5H-pyrrolo[1,2-c]imidazol-5-yl)ethan-1-ol (Isomer-II of compound 89, Compound 91);
1-Cyclohexyl-2-(7-methyl-6-phenyl-5H-pyrrolo[1,2-c]imidazol-5-yl)ethan-1-ol (Isomer-III of compound 89, Compound 92);
1-Cyclohexyl-2-(7-methyl-6-phenyl-5H-pyrrolo[1,2-c]imidazol-5-yl)ethan-1-ol (Isomer-IV of compound 89, Compound 93);
Methyl 4-(2-(7-methyl-6-phenyl-5H-pyrrolo[1,2-c]imidazol-5-yl)acetyl)cyclohexane-1-carboxylate (Compound 94);
2-(6-(3-Chlorophenyl)-7-methyl-5H-pyrrolo[1,2-c]imidazol-5-yl)-1-cyclohexylethan-1-one (Compound 95);
2-(6-(3-Chlorophenyl)-7-methyl-5H-pyrrolo[1,2-c]imidazol-5-yl)-1-cyclohexylethan-1-ol (Compound 96);
2-(6-(2-fluorophenyl)-7-methyl-5H-pyrrolo[1,2-c]imidazol-5-yl)-1-(4-hydroxycyclohexyl)ethan-1-one (Isomer-I, Compound 97);
2-(6-(2-fluorophenyl)-7-methyl-5H-pyrrolo[1,2-c]imidazol-5-yl)-1-(4-hydroxycyclohexyl)ethan-1-one (Isomer-II, Compound 98);
1-(4-Hydroxycyclohexyl)-2-(7-methyl-6-phenyl-5H-pyrrolo[1,2-c]imidazol-5-yl)ethan-1-one (Compound 99);
2-(7-methyl-6-phenyl-5H-pyrrolo[1,2-c]imidazol-5-yl)-1-(tetrahydro-2H-pyran-4-yl)ethan-1-one (Compound 100);
1-Cyclohexyl-2-(6-(2-fluorophenyl)-7-methyl-5H-pyrrolo[1,2-c]imidazol-5-yl)ethan-1-one (Compound 101);
1-Cyclohexyl-2-(6,7-diphenyl-5H-pyrrolo[1,2-c]imidazol-5-yl)ethan-1-one (Compound 102);
1-Cyclohexyl-2-(6,7-diphenyl-5H-pyrrolo[1,2-c]imidazol-5-yl)ethan-1-ol (Compound 103);
1-cyclohexyl-2-(6-(4-methoxyphenyl)-7-methyl-5H-pyrrolo[1,2-c]imidazol-5-yl)ethan-1-one (Compound 104);
1-Cyclohexyl-2-(7-methyl-6-(o-tolyl)-5H-pyrrolo[1,2-c]imidazol-5-yl)ethan-1-ol (Compound 105);
1-Cyclohexyl-2-(7-isopropyl-6-phenyl-5H-pyrrolo[1,2-c]imidazol-5-yl)ethan-1-one (Compound 106);
1-(Adamantan-1-yl)-2-(7-methyl-6-phenyl-5H-pyrrolo[1,2-c]imidazol-5-yl)ethan-1-one (Compound 107);
1-(Adamantan-1-yl)-2-(7-methyl-6-phenyl-5H-pyrrolo[1,2-c]imidazol-5-yl)ethan-1-ol (Compound 108);
4-(2-(7-methyl-6-phenyl-5H-pyrrolo[1,2-c]imidazol-5-yl)acetyl)-N-phenylcyclohexane-1-carboxamide (Compound 109);
N-(2,4-difluorophenyl)-4-(2-(7-methyl-6-phenyl-5H-pyrrolo[1,2-c]imidazol-5-yl)acetyl)cyclohexane-1-carboxamide (Compound 110);
4-(1-hydroxy-2-(7-methyl-6-phenyl-5H-pyrrolo[1,2-c]imidazol-5-yl)ethyl)cyclohexan-1-ol (Compound 111);
4-(1-hydroxy-2-(7-methyl-6-phenyl-5H-pyrrolo[1,2-c]imidazol-5-yl)ethyl)-N-phenylcyclohexane-1-carboxamide (Compound 112);
2-(7-Methyl-6-phenyl-5H-pyrrolo[1,2-c]imidazol-5-yl)-1-((S)-1-tosylpyrrolidin-2-yl)ethan-1-one (Compound 113);
(1R)-2-(7-Methyl-6-phenyl-5H-pyrrolo[1,2-c]imidazol-5-yl)-1-((R)-1-tosylpyrrolidin-2-yl)ethan-1-ol (Compound 114);
1-((S)-1-benzoylpyrrolidin-2-yl)-2-(7-methyl-6-phenyl-5H-pyrrolo[1,2-c]imidazol-5-yl)ethan-1-one (Compound 115);
((2R)-2-((1R)-1-hydroxy-2-(7-methyl-6-phenyl-5H-pyrrolo[1,2-c]imidazol-5-yl)ethyl)pyrrolidin-1-yl)(phenyl)methanone (Compound 116);
4-(2-(7-methyl-6-phenyl-5H-pyrrolo[1,2-c]imidazol-5-yl)acetyl)-N-(pyridin-4-yl)cyclohexane-1-carboxamide (Compound 117);
N-(4-chloro-2-methylphenyl)-4-(2-(7-methyl-6-phenyl-5H-pyrrolo[1,2-c]imidazol-5-yl)acetyl)cyclohexane-1-carboxamide (Compound 118);
4-(1-hydroxy-2-(7-methyl-6-phenyl-5H-pyrrolo[1,2-c]imidazol-5-yl)ethyl)-N-(pyridin-4-yl)cyclohexane-1-carboxamide (Compound 119);
N-(4-chloro-2-methylphenyl)-4-(1-hydroxy-2-(7-methyl-6-phenyl-5H-pyrrolo[1,2-c]imidazol-5-yl)ethyl)cyclohexane-1-carboxamide (Compound 120);
4-(1-hydroxy-2-(7-methyl-6-phenyl-5H-pyrrolo[1,2-c]imidazol-5-yl)ethyl)cyclohexan-1-ol (Isomer-I of Compound 111, Compound 121);
4-(1-hydroxy-2-(7-methyl-6-phenyl-5H-pyrrolo[1,2-c]imidazol-5-yl)ethyl)cyclohexan-1-ol (Isomer-II of Compound 111, Compound 122);
N-cyclohexyl-4-(2-(7-methyl-6-phenyl-5H-pyrrolo[1,2-c]imidazol-5-yl)acetyl)cyclohexane-1-carboxamide (Compound 123);
1-(4-(2-(7-methyl-6-phenyl-5H-pyrrolo[1,2-c]imidazol-5-yl)acetyl)cyclohexane-1-carbonyl)piperidin-4-one (Compound 124);
(4-(1-hydroxy-2-(7-methyl-6-phenyl-5H-pyrrolo[1,2-c]imidazol-5-yl)ethyl)cyclohexyl)(4-hydroxypiperidin-1-yl)methanone (Compound 125);
N-cyclohexyl-4-(1-hydroxy-2-(7-methyl-6-phenyl-5H-pyrrolo[1,2-c]imidazol-5-yl)ethyl)cyclohexane-1-carboxamide (Compound 126);
4-(1-Hydroxy-2-(7-methyl-6-phenyl-5H-pyrrolo[1,2-c]imidazol-5-yl)ethyl)cyclohexan-1-ol (Compound 127);
4-(1-hydroxy-2-(7-methyl-6-phenyl-5H-pyrrolo[1,2-c]imidazol-5-yl)ethyl)-N-(pyridin-3-yl)cyclohexane-1-carboxamide (Compound 128);
4-(1-hydroxy-2-(7-methyl-6-phenyl-5H-pyrrolo[1,2-c]imidazol-5-yl)ethyl)-N-(4-hydroxycyclohexyl)cyclohexane-1-carboxamide (Compound 129);
(1s,4s)-4-(2-(7-methyl-6-phenyl-5H-pyrrolo[1,2-c]imidazol-5-yl)acetyl)cyclohexane-1-carboxylic acid (Compound 130);
methyl (1R,4s)-4-((1S)-1-hydroxy-2-(7-methyl-6-phenyl-5H-pyrrolo[1,2-c]imidazol-5-yl)ethyl)cyclohexane-1-carboxylate (Compound 131);
4-(1-hydroxy-2-(7-methyl-6-phenyl-5H-pyrrolo[1,2-c]imidazol-5-yl)ethyl)-N-(pyridazin-3-yl)cyclohexane-1-carboxamide (Compound 132);
4-(1-hydroxy-2-(7-methyl-6-phenyl-5H-pyrrolo[1,2-c]imidazol-5-yl)ethyl)cyclohexane-1-carboxylic acid (Compound 133);
4-(1-hydroxy-2-(7-methyl-6-phenyl-5H-pyrrolo[1,2-c]imidazol-5-yl)ethyl)cyclohexan-1-ol (mixture of cis or trans of Compound 111, Compound 134);
N-methyl-4-(2-(7-methyl-6-phenyl-5H-pyrrolo[1,2-c]imidazol-5-yl)acetyl)cyclohexane-1-carboxamide (Compound 135);
2-(7-methyl-6-phenyl-5H-pyrrolo[1,2-c]imidazol-5-yl)-1-(4-phenylcyclohexyl)ethan-1-one (Compound 136);
4-[1-Hydroxy-2-(7-methyl-6-phenyl-5H-pyrrolo[1,2-c]imidazol-5-yl)-ethyl]-piperidine-1-carboxylic acid benzyl ester (Compound 137);
2-(2-Fluorophenyl)-1-(4-(2-(7-methyl-6-phenyl-5H-pyrrolo[1,2-c]imidazol-5-yl)acetyl)piperidin-1-yl)ethan-1-one (Compound 138);
2-(2-Fluorophenyl)-1-(4-(1-hydroxy-2-(7-methyl-6-phenyl-5H-pyrrolo[1,2-c]imidazol-5-yl)ethyl)piperidin-1-yl)ethan-1-one (Compound 139);
1-(1-Benzylpiperidin-4-yl)-2-(7-methyl-6-phenyl-5H-pyrrolo[1,2-c]imidazol-5-yl)ethan-1-one (Compound 140);
1-(1-Benzylpiperidin-4-yl)-2-(7-methyl-6-phenyl-5H-pyrrolo[1,2-c]imidazol-5-yl)ethan-1-ol (Compound 141);
1-(1-Benzoylpiperidin-4-yl)-2-(7-methyl-6-phenyl-5H-pyrrolo[1,2-c]imidazol-5-yl)ethan-1-one (Compound 142);
2-(7-Methyl-6-phenyl-5H-pyrrolo[1,2-c]imidazol-5-yl)-1-(1-(phenylsulfonyl)piperidin-4-yl)ethan-1-one (Compound 143);
(4-(1-Hydroxy-2-(7-methyl-6-phenyl-5H-pyrrolo[1,2-c]imidazol-5-yl)ethyl)piperidin-1-yl)(phenyl)methanone (Compound 144);
2-(7-Methyl-6-phenyl-5H-pyrrolo[1,2-c]imidazol-5-yl)-1-(1-(phenylsulfonyl)piperidin-4-yl)ethan-1-ol (Compound 145);
Ethyl 4-(2-(7-methyl-6-phenyl-5H-pyrrolo[1,2-c]imidazol-5-yl)acetyl)piperidine-1-carboxylate (Compound 146);
1-(1-(Cyclohexanecarbonyl)piperidin-4-yl)-2-(7-methyl-6-phenyl-H-pyrrolo[1,2-c]imidazol-5-yl)ethan-1-one (Compound 147);
Ethyl 4-(1-hydroxy-2-(7-methyl-6-phenyl-5H-pyrrolo[1,2-c]imidazol-5-yl)ethyl)piperidine-1-carboxylate (Compound 148);
Cyclohexyl(4-(1-hydroxy-2-(7-methyl-6-phenyl-5H-pyrrolo[1,2-c]imidazol-5-yl)ethyl)piperidin-1-yl)methanone (Compound 149);
(4-(1-hydroxy-2-(7-methyl-6-phenyl-5H-pyrrolo[1,2-c]imidazol-5-yl)ethyl)piperidin-1-yl)(phenyl)methanone (Compound 150);
(4-(1-hydroxy-2-(7-methyl-6-phenyl-5H-pyrrolo[1,2-c]imidazol-5-yl)ethyl)piperidin-1-yl)(phenyl)methanone (Compound 151);
{4-[1-Hydroxy-2-(7-methyl-6-phenyl-5H-pyrrolo[1,2-c]imidazol-5-yl)-ethyl]-piperidin-1-yl}-pyridin-2-yl-methanone (Compound 152);
Ethyl 4-(1-hydroxy-2-(7-methyl-6-phenyl-5H-pyrrolo[1,2-c]imidazol-5-yl)ethyl)piperidine-1-carboxylate (Compound 153);
Ethyl 4-(1-hydroxy-2-(7-methyl-6-phenyl-5H-pyrrolo[1,2-c]imidazol-5-yl)ethyl)piperidine-1-carboxylate (Compound 154);
Cyclohexyl(4-(1-hydroxy-2-(7-methyl-6-phenyl-5H-pyrrolo[1,2-c]imidazol-5-yl)ethyl)piperidin-1-yl)methanone (Compound 155);
Cyclohexyl(4-(1-hydroxy-2-(7-methyl-6-phenyl-5H-pyrrolo[1,2-c]imidazol-5-yl)ethyl)piperidin-1-yl)methanone (Compound 156);
{4-[1-Hydroxy-2-(7-methyl-6-phenyl-5H-pyrrolo[1,2-c]imidazol-5-yl)-ethyl]-piperidin-1-yl}-pyridin-3-yl-methanone (Compound 157);
4-[1-Hydroxy-2-(7-methyl-6-phenyl-5H-pyrrolo[1,2-c]imidazol-5-yl)-ethyl]-piperidine-1-carboxylic acid phenyl amide (Compound 158);
1-(1-Benzoyl-azetidin-3-yl)-2-(7-methyl-6-phenyl-5H-pyrrolo[1,2-c]imidazol-5-yl)-ethanone (Compound 159);
{4-[1-Hydroxy-2-(7-methyl-6-phenyl-5H-pyrrolo[1,2-c]imidazol-5-yl)-ethyl]-piperidin-1-yl}-pyridin-2-yl-methanone (Isomer-I of Compound 152, Compound 160);
{4-[1-Hydroxy-2-(7-methyl-6-phenyl-5H-pyrrolo[1,2-c]imidazol-5-yl)-ethyl]-piperidin-1-yl}-pyridin-2-yl-methanone (Isomer-II of Compound 152, Compound 161);
{4-[1-Hydroxy-2-(7-methyl-6-phenyl-5H-pyrrolo[1,2-c]imidazol-5-yl)-ethyl]-piperidin-1-yl}-pyridin-4-yl-methanone (Compound 162);
1-(1-Cyclohexanesulfonyl-piperidin-4-yl)-2-(7-methyl-6-phenyl-5H-pyrrolo[1,2-c]imidazol-5-yl)-ethanol (Compound 163);
{4-[1-Hydroxy-2-(7-methyl-6-phenyl-5H-pyrrolo[1,2-c]imidazol-5-yl)-ethyl]-piperidin-1-yl}-(2-methyl-pyridin-4-yl)-methanone (Compound 164);
1-(1-Methanesulfonyl-piperidin-4-yl)-2-(7-methyl-6-phenyl-5H-pyrrolo[1,2-c]imidazol-5-yl)-ethanol (Compound 165);
2-{4-[1-Hydroxy-2-(7-methyl-6-phenyl-5H-pyrrolo[1,2-c]imidazol-5-yl)-ethyl]-piperidine-1-carbonyl}-benzoic acid (Compound 166);
4-[1-Hydroxy-2-(7-methyl-6-phenyl-5H-pyrrolo[1,2-c]imidazol-5-yl)-ethyl]-piperidine-1-carboxylic acid pyridin-2-ylamide (Compound 167);
5-(2-Cyclohexyl-2-fluoro-ethyl)-7-methyl-6-phenyl-5H-pyrrolo[1,2-c]imidazole (Compound 168);
1-Phenyl-2-(6-phenyl-5H-pyrrolo[1,2-c]imidazol-5-yl)-ethanone (Compound 169);
1-(7-Methyl-6-phenyl-5H-pyrrolo[1,2-c]imidazole-5-yl)-hexan-2-ol (Compound 170);
1-(7-Methyl-6-phenyl-5H-pyrrolo[1,2-c]imidazol-5-yl)-decan-2-ol (Compound 171);
2-(7-Methyl-6-phenyl-5H-pyrrolo[1,2-c]imidazol-5-yl)-N-propylacetamide (Compound 172);
Ethyl 5-(7-methyl-6-phenyl-5H-pyrrolo[1,2-c]imidazol-5-yl)-4-oxopentanoate (Compound 173);
4-Hydroxy-1-(4-hydroxypiperidin-1-yl)-5-(7-methyl-6-phenyl-5H-pyrrolo[1,2-c]imidazol-5-yl)pentan-1-one (Compound 174);
1-(4-Hydroxypiperidin-1-yl)-2-(7-methyl-6-phenyl-5H-pyrrolo[1,2-c]imidazol-5-yl)ethan-1-one (Compound 175);
2-(2-fluorophenyl)-N-(4-(2-(7-methyl-6-phenyl-5H-pyrrolo[1,2-c]imidazol-5-yl)acetyl)phenyl)acetamide (Compound 176);
4-[2-(7-Methyl-6-phenyl-5H-pyrrolo[1,2-c]imidazol-5-yl)-acetyl]-cyclohexanone oxime (Compound 177);
4-[1-Hydroxy-2-(7-methyl-6-phenyl-5H-pyrrolo[1,2-c]imidazol-5-yl)-ethyl]-cyclohexanone oxime (Compound 178);
N-cyclohexyl-4-(1-hydroxy-2-(7-methyl-6-phenyl-5H-pyrrolo[1,2-c]imidazol-5-yl)ethyl)piperidine-1-carboxamide (Compound 179);
1-(4-(1-hydroxy-2-(7-methyl-6-phenyl-5H-pyrrolo[1,2-c]imidazol-5-yl)ethyl)piperidin-1-yl)-2-phenylethan-1-one (Compound 180);
N-(3-chlorophenyl)-4-(1-hydroxy-2-(7-methyl-6-phenyl-5H-pyrrolo[1,2-c]imidazol-5-yl)ethyl)piperidine-1-carboxamide (Compound 181);
N-(3-chloro-4-fluorophenyl)-4-(1-hydroxy-2-(7-methyl-6-phenyl-5H-pyrrolo[1,2-c]imidazol-5-yl)ethyl)piperidine-1-carboxamide (Compound 182);
4-(1-hydroxy-2-(7-methyl-6-phenyl-5H-pyrrolo[1,2-c]imidazol-5-yl)ethyl)-N-(6-methylpyridin-2-yl)piperidine-1-carboxamide (Compound 183);
1-(4,4-difluorocyclohexyl)-2-(7-methyl-6-phenyl-5H-pyrrolo[1,2-c]imidazol-5-yl)ethan-1-ol (Compound 184);
tert-butyl (1R,4s)-4-((1S)-1-hydroxy-2-(7-methyl-6-phenyl-5H-pyrrolo[1,2-c]imidazol-5-yl)ethyl)cyclohexane-1-carboxylate (Compound 185);
2-(7-Methyl-6-phenyl-5H-pyrrolo[1,2-c]imidazol-5-yl)-1-(4-phenyl-cyclohexyl)-ethanol (Compound 186);
1-(4-(1-hydroxy-2-(7-methyl-6-phenyl-5H-pyrrolo[1,2-c]imidazol-5-yl)ethyl)piperidin-1-yl)-2-methylpropan-1-one (Compound 187);
2-(7-methyl-6-phenyl-5H-pyrrolo[1,2-c]imidazol-5-yl)-1-(1-(pyridin-2-yl)piperidin-4-yl)ethan-1-ol (Compound 188);
Ethyl (1R,4s)-4-((1S)-1-hydroxy-2-(7-methyl-6-phenyl-5H-pyrrolo[1,2-c]imidazol-5-yl)ethyl)cyclohexane-1-carboxylate (Compound 189);
N-(2-(4-(1-hydroxy-2-(7-methyl-6-phenyl-5H-pyrrolo[1,2-c]imidazol-5-yl)ethyl)piperidin-1-yl)ethyl)methanesulfonamide;
2-(7-methyl-6-phenyl-5H-pyrrolo[1,2-c]imidazol-5-yl)-1-(1-phenylpiperidin-4-yl)ethan-1-ol;
1-(1-isobutylpiperidin-4-yl)-2-(7-methyl-6-phenyl-5H-pyrrolo[1,2-c]imidazol-5-yl)ethan-1-ol;
(4-(1-hydroxy-2-(7-methyl-6-phenyl-5H-pyrrolo[1,2-c]imidazol-5-yl)ethyl)piperidin-1-yl)(4-hydroxyphenyl)methanone;
(2-fluorophenyl)(4-(1-hydroxy-2-(7-methyl-6-phenyl-5H-pyrrolo[1,2-c]imidazol-5-yl)ethyl)piperidin-1-yl)methanone;
(4-(1-hydroxy-2-(7-methyl-6-phenyl-5H-pyrrolo[1,2-c]imidazol-5-yl)ethyl)piperidin-1-yl)(piperidin-4-yl)methanone;
azetidin-3-yl(4-(1-hydroxy-2-(7-methyl-6-phenyl-5H-pyrrolo[1,2-c]imidazol-5-yl)ethyl)piperidin-1-yl)methanone;
(4-(1-hydroxy-2-(7-methyl-6-phenyl-5H-pyrrolo[1,2-c]imidazol-5-yl)ethyl)piperidin-1-yl)(2-hydroxyphenyl)methanone;
2-(7-methyl-6-phenyl-5H-pyrrolo[1,2-c]imidazol-5-yl)-1-(1-(oxetan-3-yl)piperidin-4-yl)ethan-1-ol;
1-(1-(azetidin-3-yl)piperidin-4-yl)-2-(7-methyl-6-phenyl-5H-pyrrolo[1,2-c]imidazol-5-yl)ethan-1-ol;
2-(7-methyl-6-phenyl-5H-pyrrolo[1,2-c]imidazol-5-yl)-1-(1-(pyrimidin-5-yl)piperidin-4-yl)ethan-1-ol;
2-(7-methyl-6-phenyl-5H-pyrrolo[1,2-c]imidazol-5-yl)-1-(1-(pyridin-4-yl)piperidin-4-yl)ethan-1-ol;
2-(7-methyl-6-phenyl-5H-pyrrolo[1,2-c]imidazol-5-yl)-1-(1-(oxazol-4-yl)piperidin-4-yl)ethan-1-ol;
1-(1-(1H-imidazol-4-yl)piperidin-4-yl)-2-(7-methyl-6-phenyl-5H-pyrrolo[1,2-c]imidazol-5-yl)ethan-1-ol;
1-(1-(1H-pyrazol-3-yl)piperidin-4-yl)-2-(7-methyl-6-phenyl-5H-pyrrolo[1,2-c]imidazol-5-yl)ethan-1-ol;
1-(1-(2-aminophenyl)piperidin-4-yl)-2-(7-methyl-6-phenyl-5H-pyrrolo[1,2-c]imidazol-5-yl)ethan-1-ol;
methyl 4-(4-(1-hydroxy-2-(7-methyl-6-phenyl-5H-pyrrolo[1,2-c]imidazol-5-yl)ethyl)piperidin-1-yl)benzoate;
methyl 3-(4-(1-hydroxy-2-(7-methyl-6-phenyl-5H-pyrrolo[1,2-c]imidazol-5-yl)ethyl)piperidin-1-yl)benzoate;
4-(1-amino-2-(7-methyl-6-phenyl-5H-pyrrolo[1,2-c]imidazol-5-yl)ethyl)cyclohexan-1-ol;
4-(1-hydroxy-2-(7-methyl-6-phenyl-5H-pyrrolo[1,2-c]imidazol-5-yl)ethyl)cyclohexane-1-sulfonamide;
4-(1-hydroxy-2-(7-methyl-6-phenyl-5H-pyrrolo[1,2-c]imidazol-5-yl)ethyl)-1-(hydroxymethyl)cyclohexan-1-ol;
4-(1-hydroxy-2-(7-methyl-6-phenyl-5H-pyrrolo[1,2-c]imidazol-5-yl)ethyl)cyclohexyl acetate;
4-(1-hydroxy-2-(7-methyl-6-phenyl-5H-pyrrolo[1,2-c]imidazol-5-yl)ethyl)cyclohexyl benzoate;
4-(1-hydroxy-2-(7-methyl-6-phenyl-5H-pyrrolo[1,2-c]imidazol-5-yl)ethyl)cyclohexyl dihydrogen phosphate;
2-(7-methyl-6-phenyl-5H-pyrrolo[1,2-c]imidazol-5-yl)-1-(1-oxaspiro[3.5]nonan-7-yl)ethan-1-ol;
2-(7-methyl-6-phenyl-5H-pyrrolo[1,2-c]imidazol-5-yl)-1-(1-oxaspiro[4.5]decan-8-yl)ethan-1-ol;
N-(4-(1-hydroxy-2-(7-methyl-6-phenyl-5H-pyrrolo[1,2-c]imidazol-5-yl)ethyl)cyclohexyl)benzamide;
N-(4-(1-hydroxy-2-(7-methyl-6-phenyl-5H-pyrrolo[1,2-c]imidazol-5-yl)ethyl)cyclohexyl)benzamide;
Benzyl 4-(1-hydroxy-2-(7-methyl-6-phenyl-5H-pyrrolo[1,2-c]imidazol-5-yl)ethyl)cyclohexane-1-carboxylate;
pyridin-4-ylmethyl 4-(1-hydroxy-2-(7-methyl-6-phenyl-5H-pyrrolo[1,2-c]imidazol-5-yl)ethyl)cyclohexane-1-carboxylate;
1-(4-(1-hydroxy-2-(7-methyl-6-phenyl-5H-pyrrolo[1,2-c]imidazol-5-yl)ethyl)cyclohexyl)cyclopropan-1-ol;
3-(1-hydroxy-2-(7-methyl-6-phenyl-5H-pyrrolo[1,2-c]imidazol-5-yl)ethyl)cyclobutan-1-ol;
1-(3-methoxycyclobutyl)-2-(7-methyl-6-phenyl-5H-pyrrolo[1,2-c]imidazol-5-yl)ethan-1-ol;
3-(1-hydroxy-2-(7-methyl-6-phenyl-5H-pyrrolo[1,2-c]imidazol-5-yl)ethyl)cyclobutane-1-carboxylic acid;
methyl 3-(1-hydroxy-2-(7-methyl-6-phenyl-5H-pyrrolo[1,2-c]imidazol-5-yl)ethyl)cyclobutane-1-carboxylate;
3-(1-hydroxy-2-(7-methyl-6-phenyl-5H-pyrrolo[1,2-c]imidazol-5-yl)ethyl)cyclobutane-1-carboxamide;
3-(1-hydroxy-2-(7-methyl-6-phenyl-5H-pyrrolo[1,2-c]imidazol-5-yl)ethyl)-N-methylcyclobutane-1-carboxamide; or pharmaceutically acceptable salts, solvates, tautomers, stereoisomers, including hydrates and prodrugs of compounds are also contemplated.

The present invention also provides a pharmaceutical composition that includes at least one compound of described herein and at least one pharmaceutically acceptable excipient (such as a pharmaceutically acceptable carrier or diluent). Preferably, the pharmaceutical composition comprises a therapeutically effective amount of at least one compound described herein. The compound(s) present in the composition may be associated with a pharmaceutically acceptable excipient (such as a carrier or a diluent) or may be diluted by a carrier, or enclosed within a carrier which may be in the form of a capsule, sachet, paper, or other container.

The compounds and pharmaceutical compositions described herein are useful in the treatment of diseases, conditions and/or disorders mediated by an indoleamine 2,3-dioxygenase (IDO) and/or tryptophan 2,3-dioxygenase (TDO).

The present invention further provides a method of treating a disease, condition and/or disorder mediated by an indoleamine 2,3-dioxygenase (IDO) and/or tryptophan 2,3-dioxygenase (TDO) in a subject in need thereof by administering to the subject one or more compounds described herein in the amount effective to treat that condition.

Also provided herein are processes for preparing compounds described herein.

The invention provides a method for preventing, ameliorating or treating a cancer mediated disease, disorder or syndrome in a subject in need thereof comprising administering to the subject a therapeutically effective amount of a compound of the invention. The invention further provides a method, wherein the indoleamine 2,3-dioxygenase (IDO) and/or tryptophan 2,3-dioxygenase (TDO) mediated disease, disorder or syndrome is cancer for example but are not limited to a solid or liquid tumour including cancer of the eye, brain (such as gliomas, glioblastomas, medullablastomas, craniopharyngioma, ependymoma, and astrocytoma), colon, parathyroid gland, gall bladder, head and neck, breast, bone, hypopharyngeal gland, lung, bronchus, liver, skin (melanomas), ureter, urethra, urothelium, testicles, vaginal, anus, mouth, lip, throat, oral cavity, nasal cavity, Gastro-intestinal, Gastric stomach, Gastro-intestinal stromal cells, small intestine, laryngeal gland, ovary, thyroid, bile duct, cervix, heart, spinal cord, kidney, oesophagus, nasopharyngeal gland, pituitary gland, salivary gland, prostate, penile tissue, pancreas, adrenal glands; an epithelial and squamous cell cancers of various tissue types, an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukemia (T-PLL), large granular lymphocytic leukemia, adult T-cell leukemia/lymphoma (ATLL), juvenile myelomonocytic leukaemia, Hodgkin's lymphoma, classical Hodgkin's lymphoma, non-Hodgkin's lymphoma, mantle cell lymphoma, follicular lymphoma, primary effusion lymphoma, AIDS-related lymphoma, diffuse B cell lymphoma, Burkitt lymphoma, and cutaneous T-cell lymphoma), Barret's adenocarcinoma, cervical cancer, esophageal cancer, ovarian cancer, colo-rectal cancer, prostate cancer, hematologic cancers, cancer of Billary Tract, blood cancer, large in testinal colon carcinoma, histiocytic lymphoma, lung adenocarcinoma, astrocytoma, meningioma, medulloblastoma and peripheral neuroectodermal tumors, diffuse large B-cell lymphoma (DLBCL), gall bladder carcinoma, bronchial carcinoma, small cell lung carcinoma, non-small cell lung carcinoma (NSCLC), multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma, metastatic carcinomas and plasmocytoma, an inflammatory condition, an infectious disease, Chagas disease, a central nervous system disease or disorder, depression, psychosis, psychiatric disorders, bipolar disorders, anxiety, insomnia, a neurodegenerative disorder, Parkinson's disease (PD), Alzheimer's disease, Huntington's disease, stroke, amyotrophic lateral sclerosis, dementia, cognitive disorders, psychotic disorders/cognitive disorder/dementia associated with various neurodegenerative diseases, trauma, age-related cataracts, organ transplant rejection, viral infection, anti-retroviral therapy, treating or preventing HIV/AIDS, chronic HBV, malaria, schizophrenia, HCV, inflammation-associated arthritis or autoimmune arthritis, allergic airways disease, joint inflammation, multiple sclerosis, allergic encephalomyelitis, atherosclerosis, coronary artery disease, kidney disease, sepsis-induced hypotension, pain, chronic pain of inflammatory and neuropathic nature, chemotherapy induced neuropathies, musculo-skeletal pain, General anaesthesia, Cataracts, Endometriosis, Contraception and abortion, coronary heart disease, chronic renal failure, or post anaesthesia cognitive dysfunction, and the like.

An indoleamine 2,3-dioxygenase (IDO) and/or tryptophan 2,3-dioxygenase (TDO) inhibitory potential of the compounds of present invention may be demonstrated by any one or more methodologies known in the art, such as by using the assays described in BPS Bioscience, San Diego, Calif., USA and some other literature.

### DETAILED DESCRIPTION OF THE INVENTION

The present invention provides novel pharmaceutical compounds and related derivatives, which may be used as indoleamine 2,3-dioxygenase (IDO) and/or tryptophan 2,3-dioxygenase (TDO) as anti-cancer compounds and processes for the synthesis of these compounds. Pharmaceutically acceptable salts, pharmaceutically acceptable solvates, enantiomers, diastereomers, together with pharmaceutically acceptable carriers, excipients or diluents, which can be used for the treatment of diseases, condition and/or disorders mediated by indoleamine 2,3-dioxygenase (IDO) and/or tryptophan 2,3-dioxygenase (TDO), are also provided.

The following definitions apply to the terms as used herein:

The term “alkyl” refers to a straight or branched hydrocarbon chain radical having from one to eight carbon atoms, and which is attached to the rest of the molecule by a single bond, examples include but are not limited to methyl, ethyl, n-propyl, isopropyl, n-butyl, n-pentyl, 1,1-dimethylethyl and the like.

The term “alkenyl” refers to aliphatic hydrocarbon group containing a carbon-carbon double bond and which may be a straight or branched chain radical having 2 to 10 carbon atoms which is attached to the rest of the molecule by a single bond. Examples include but are not limited to ethenyl, 1-propenyl, 2-propenyl, iso-propenyl, 2-methyl-1-propenyl, 1-butenyl and 2-butenyl and the like.

The term “alkynyl” refers to straight or branched chain hydrocarbon radicals having at least one carbon-carbon triple bond, having 2 to 12 carbon which is attached to the rest of the molecule by a single bond. Examples include but are not limited to ethynyl, propynyl and butnyl.

The term “alkoxy” as used herein denotes alkyl group as defined above attached through an oxygen linkage with the main molecule. Examples of alkoxy substituents include but not limited to methoxy, ethoxy, propoxy and the like

The term “aryl” refers to aromatic radicals having 6 to 14 carbon atoms. Examples include but are not limited to phenyl, naphthyl, tetrahydronapthyl, indanyl and biphenyl.

The term “arylalkyl” refers to an aryl ring as defined above directly bonded to an alkyl group as defined above. Examples include but are not limited to —CH₂C₆H₅, and —C₂H₅C₆H₅.

The term “aryloxy” as used herein denotes aryl group as defined above attached through an oxygen linkage with the main molecule. Examples of aryloxy substituents include but not limited to phenoxy, biphenyloxy, naphthyloxy and the like.

The term “heteroaryl” as used herein refers to a stable 3 to 15 membered aromatic ring which consists of carbon atoms and from one to five heteroatoms selected from the group consisting of nitrogen, phosphorus, oxygen and sulfur. For purpose of this invention, the heteroaryl ring radical may be a monocyclic, bicyclic or tricyclic ring system, which may include fused, bridged or spiro ring systems, and the nitrogen, phosphorus, carbon, oxygen or sulfur atoms in the heteroaryl ring may be optionally oxidized to various oxidation states. Non-limiting examples include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups and the like.

The term “heteroarylalkyl” refers to heteroaryl ring radical as defined above directly bonded to alkyl group. The heteroarylalkyl radical may be attached to the main structure at any carbon atom from alkyl group that results in the creation of a stable structure.

The term “heteroaryloxy” as used herein, means a heteroaryl group, as defined herein, attached to the main molecule through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy, pyrimidin-5-yloxy, pyrimidin-2-yloxy, thien-2-yloxy, and thien-3-yloxy.

The term “cycloalkyl” denotes a non-aromatic mono or multicyclic ring system of 3 to about 14 carbon atoms attached via a single bond to the rest of the molecule. Examples of monocyclic ring system include but are not limited to cyclopropyl, cyclobutyl, cyclopentyl and, cyclohexyl. Examples of multicyclic ring system include but are not limited to perhydronapthtliyl, adamantyl and norbornyl groups bridged cyclic group or spirobicyclic groups e.g. spiro (4,4) non-2-yl.

The term “cycloalkenyl” refers to cyclic ring-containing radicals containing in the range of about 3 up to 8 carbon atoms with at least one carbon-carbon double bond. Examples include but are not limited to cyclopropenyl, cyclobutenyl and cyclopentenyl.

The term “cycloalkylalkyl” refers to cyclic ring-containing radical containing 3 to about 8 carbon atoms directly attached to alkyl group which is then attached to the main structure at any carbon from alkyl group that results in the creation of a stable structure. Examples include but are not limited to cyclopropylmethyl, cyclobutylethyl, cyclopentylethyl.

The term “heterocycloalkyl” as used herein refers to a stable 3- to 15 membered saturated non-aromatic ring which consists of carbon atoms and from one to five heteroatoms selected from the group consisting of nitrogen, phosphorus, oxygen and sulfur. For purpose of this invention, the heterocycloalkyl ring radical may be a monocyclic, bicyclic or tricyclic ring system, which may include fused, bridged or spiro ring systems, and the nitrogen, phosphorus, carbon, oxygen or sulfur atoms in the heterocycloalkyl ring may be optionally oxidized to various oxidation states. In addition, the nitrogen atom may be optionally quaternized. Examples of heterocycloalkyl ring systems include but not limited to oxetan, tetrahydrofuran, tetrahydropyran or oxepane, dioxane, azetidine, pyrrolidine, piperidine, hexahydroazepine, hexahydrodiazepine, tetrahydrothiophene, thietan, tetrahydrothiopyran, thiepan, morpholine as well as bridged heterocycloalkyl systems such as oxabicyclo[4.4.0]decane and azabicyclo[2,2,1]undecane.

The term “heterocyclolalkylalkyl” as used herein refers to a heterocyloalkyl ring as defined above attached to alkyl group. The heterocyclolalkylalkyl radical may be bonded to the main structure at any carbon atom in the alkyl group.

The term “arylamino” refers to an aryl ring as defined above attached via amino group to the rest of the molecule. Examples include but are not limited to —NHC₆H₅. For the purpose of this invention only one or both the hydrogen atoms of amino group can be substituted by aryl group.

The term “heteroarylamino” refers to heteroaryl ring as defined above attached via amino group to the rest of the molecule. Examples include but are not limited to —NH-furan, —NH-1H-imidazole, —NH-pyridine. For the purpose of this invention only one or both the hydrogen atoms of amino group can be substituted by heteroaryl group.

The term “halogen” as used herein refers to fluorine, chlorine, bromine, iodine.

The term “haloalkyl” as used herein refers to alkyl radical having one or more hydrogen atoms replaced by a halogen atom. Non-limiting examples include chloromethyl, fluoroethyl, chloroethyl, difluoromethyl, dichloromethyl, trifluoromethyl and the like.

The substituents in the ‘substituted alkyl’, ‘substituted alkenyl’, ‘substituted alkynyl’, ‘substituted cycloalkyl’, ‘substituted cycloalkylalkyl’, ‘substituted cyclocalkenyl’, ‘substituted arylalkyl’, ‘substituted aryl’, ‘substituted aryloxy’, ‘substituted heteroaryl’, ‘substituted heteroaryloxy’, ‘substituted heteroarylalkyl’, ‘substituted heterocycloalkyl’, ‘substituted heterocycloalkylalkyl’, ‘substituted spiro’ may be the same or different which one or more selected from the groups such as hydrogen, hydroxy, halogen, carboxyl, cyano, amino, nitro, oxo (═O), thio (═S), or optionally substituted groups selected from alkyl, alkoxy, alkenyl, alkynyl, aryl, arylalkyl, cycloalkyl, aryl, heteroaryl, heteroarylalkyl, heterocyclic ring, —COOR^{A}, —C(O)R^{A}, —C(S)R^{A}, —C(O)NR^{A}R^{B}, —NR^{A}CONR^{B}R^{C}, —N(R^{A})SOR^{B}, —N(R^{A})SO₂R^{B}, —(═N—N(R^{A})R^{B}), —NR^{A}C(O)OR^{B}, —NR^{A}R^{B}, —NR^{A}C(O)R^{B}, —NR^{A}C(S)R^{B}, —NR^{A}C(S)NR^{B}R^{C}, —SONR^{A}R^{B}—, —SO₂NR^{A}R^{B}, —OR^{A}, —OR^{A}C(O)NR^{B}R^{C}, —OR^{A}C(O)OR^{B}—, —OC(O)R^{A}, —OC(O)NR^{A}R^{B}, —R^{A}NR^{B}R^{C}, —R^{A}R^{B}R^{C}, —R^{A}CF₃, —R^{A}NR^{B}C(O)R^{C}, —R^{A}OR^{B}, —R^{A}C(O)OR^{B}, —R^{A}C(O)NR^{B}R^{C}, —R^{A}C(O)R^{A}, —R^{A}OC(O)R^{B}, —SR^{A}, —SOR^{A}, —SO₂R^{A}, —ONO₂, (wherein R^{A}, R^{B} and R^{C} in each of the above groups can be hydrogen atom, substituted or unsubstituted alkyl, haloalkyl, substituted or unsubstituted arylalkyl, substituted or unsubstituted aryl, substituted or unsubstituted cycloalkyl, substituted or unsubstituted cycloalkylalkyl substituted or unsubstituted heterocyclic ring, substituted or unsubstituted heterocyclylalkyl, substituted or unsubstituted heteroaryl or substituted or unsubstituted heteroarylalkyl).

“Pharmaceutically acceptable salts” as used herein refers to acid addition salts and salts derived from inorganic or organic bases. Non-limiting examples of acid addition salts include acetates, ascorbates, benzenesulfonates, benzoates, borates, citrates, glycerophosphates, hydrohalides, ketoglutarates, maleates, methanesulphonates, nitrates, palmoates, perchlorates, phosphates, salicylates, succinates, sulphates, tartrates, trifluroacetate and the like. Examples of inorganic base salt include salts derived from Li, Na, K, Ca, Mg, Fe, Cu, Zn, Mn etc. Examples of organic base salt includes salts derived from benzyl amine, choline, choline hydroxide, dicyclohexyl amine, glucamine, metformin, N,N′-diacetylethylenediamine, spermidine, thiamine, trialkyl amine, triethyl amine and the like; chiral bases like alkylphenyl amine, glycinol, phenyl glycinol and the like; alkyl halides such as methyl halide, ethyl halide and the like. Aryl alkyl halide such as benzyl halide and the like; salts of natural amino acids such as glycine, alanine, valine, leucine, isoleucine, norleucine, tyrosine, cystine, cysteine, methionine, proline, histidine, lysine, arginine, serine and the like; unnatural amino acids such as D-isomers or substituted amino acids; guanidine, substituted guanidine wherein the substituents are selected from nitro, amino, alkyl, alkenyl, alkynyl, ammonium or substituted ammonium salts and aluminum salts.

The term “prodrug” means a compound that is transformed in vivo to yield a compound of Formula (I), (IA), (IB), (IC) or a pharmaceutically acceptable salt, hydrate or solvate, or metabolite of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The term “treating” or “treatment” of a state, disease, disorder or condition includes:
(1) preventing or delaying the appearance of clinical symptoms of the state, disease, disorder or condition developing in a subject that may be afflicted with or predisposed to the state, disease, disorder or condition but does not yet experience or display clinical or subclinical symptoms of the state, disease, disorder or condition;
(2) inhibiting the state, disease, disorder or condition, i.e., arresting or reducing the development of the state, disease, disorder or condition or at least one clinical or subclinical symptom thereof; or
(3) relieving the state, disease, disorder or condition, i.e., causing regression of the state, disease, disorder or condition or at least one of its clinical or subclinical symptoms.

The benefit to a subject receiving treatment is either statistically significant or at least perceptible to the subject or to the physician.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disease, disorder or condition, is sufficient to effect such treatment. The “therapeutically effective amount” will vary depending on the compound, the state, disease, disorder or condition and its severity and the age, weight, physical condition and responsiveness of the subject receiving treatment.

The compounds of the present invention may form salts. Non-limiting examples of pharmaceutically acceptable salts forming part of this invention include salts derived from inorganic bases salts of organic bases salts of chiral bases, salts of natural amino acids and salts of non-natural amino acids. Certain compounds of the present invention are capable of existing in stereoisomeric forms (e.g., diastereomers, enantiomers, racemates, and combinations thereof). With respect to the overall compounds described by the Formula (I), (IA), (IB), (IC), the present invention extends to these stereoisomeric forms and to mixtures thereof. To the extent prior art teaches synthesis or separation of particular stereoisomers, the different stereoisomeric forms of the present invention may be separated from one another by the methods known in the art, or a given isomer may be obtained by stereospecific or asymmetric synthesis. Tautomeric forms and mixtures of compounds described herein are also contemplated.

Pharmaceutically acceptable solvates includes hydrates and other solvents of crystallization (such as alcohols). The compounds of the present invention may form solvates with low molecular weight solvents by methods known in the art.

### Pharmaceutical Compositions

The present invention provides pharmaceutical compositions which includes at least one compound described herein and at least one pharmaceutically acceptable excipient. The pharmaceutically acceptable excipient for the purpose of this invention includes but not limited to diluents or carrier, binder, bulking agent. Preferably, the contemplated pharmaceutical compositions include a compound(s) described herein in therapeutically effective amount sufficient to treat conditions related to an indoleamine 2,3-dioxygenase (IDO) and/or tryptophan 2,3-dioxygenase (TDO) in a subject. The subjects contemplated include, for example, a living cell and a mammal, including human.

Examples of suitable carriers include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone.

The carrier or diluent may include a sustained release material, such as, for example, glyceryl monostearate or glyceryl distearate, alone or mixed with a wax.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing procedures known in the art.

The pharmaceutical compositions described herein may be prepared, e.g., as described in Remington: The Science and Practice of Pharmacy, 20th Ed., 2003 (Lippincott Williams & Wilkins). For example, the active compound can be mixed with a carrier, or diluted by a carrier, or enclosed within a carrier, which may be in the form of an ampule, capsule, or sachet. When the carrier serves as a diluent, it may be a solid, semisolid, or liquid material that acts as a vehicle, excipient, or medium for the active compound.

The pharmaceutical compositions may be, for example, capsules, tablets, aerosols, solutions, suspensions, liquids, gels, or products for topical application.

The route of administration may be any route which effectively transports the active compound to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment). The oral route is preferred.

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir can be used in cases where a sweetened vehicle can be employed.

A typical tablet that may be prepared by conventional tableting techniques.

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

### Methods of Treatment

The present invention provides compounds and pharmaceutical formulations thereof that are useful in the treatment of diseases, conditions and/or disorders mediated by an indoleamine 2,3-dioxygenase (IDO) and/or tryptophan 2,3-dioxygenase (TDO).

The present invention further provides a method of treating a disease, condition and/or disorder mediated by an indoleamine 2,3-dioxygenase (IDO) and/or tryptophan 2,3-dioxygenase (TDO) in a subject in need thereof by administering to the subject a therapeutically effective amount of a compound or a pharmaceutical composition of the present invention.

Diseases, conditions, and/or disorders that are mediated by an indoleamine 2,3-dioxygenase (IDO) and/or tryptophan 2,3-dioxygenase (TDO) are believed to include, but are not limited to a solid or liquid tumour including cancer of the eye, brain (such as gliomas, glioblastomas, medullablastomas, craniopharyngioma, ependymoma, and astrocytoma), colon, parathyroid gland, gall bladder, head and neck, breast, bone, hypopharyngeal gland, lung, bronchus, liver, skin (melanomas), ureter, urethra, urothelium, testicles, vaginal, anus, mouth, lip, throat, oral cavity, nasal cavity, Gastro-intestinal, Gastric stomach, Gastro-intestinal stromal cells, small intestine, laryngeal gland, ovary, thyroid, bile duct, cervix, heart, spinal cord, kidney, oesophagus, nasopharyngeal gland, pituitary gland, salivary gland, prostate, penile tissue, pancreas, adrenal glands; an epithelial and squamous cell cancers of various tissue types, an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukemia (T-PLL), large granular lymphocytic leukemia, adult T-cell leukemia/lymphoma (ATLL), juvenile myelomonocytic leukaemia, Hodgkin's lymphoma, classical Hodgkin's lymphoma, non-Hodgkin's lymphoma, mantle cell lymphoma, follicular lymphoma, primary effusion lymphoma, AIDS-related lymphoma, diffuse B cell lymphoma, Burkitt lymphoma, and cutaneous T-cell lymphoma), Barret's adenocarcinoma, cervical cancer, esophageal cancer, ovarian cancer, colo-rectal cancer, prostate cancer, hematologic cancers, cancer of Billary Tract, blood cancer, large in testinal colon carcinoma, histiocytic lymphoma, lung adenocarcinoma, astrocytoma, meningioma, medulloblastoma and peripheral neuroectodermal tumors, diffuse large B-cell lymphoma (DLBCL), gall bladder carcinoma, bronchial carcinoma, small cell lung carcinoma, non-small cell lung carcinoma (NSCLC), multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma, metastatic carcinomas and plasmocytoma, an inflammatory condition, an infectious disease, Chagas disease, a central nervous system disease or disorder, depression, psychosis, psychiatric disorders, bipolar disorders, a neurodegenerative disorder, trauma, age-related cataracts, organ transplant rejection, viral infection, anti-retroviral therapy, treating or preventing HIV/AIDS, chronic HBV, malaria, schizophrenia, HCV, inflammation-associated arthritis or autoimmune arthritis, allergic airways disease, joint inflammation, multiple sclerosis, Parkinson's disease (PD), Alzheimer's disease, stroke, amyotrophic lateral sclerosis, dementia, cognitive disorders, psychotic disorders/cognitive disorder/dementia associated with various neurodegenerative diseases, allergic encephalomyelitis, Huntington's disease, anxiety, insomnia, atherosclerosis, coronary artery disease, kidney disease, sepsis-induced hypotension, Psychiatric disorders and pain, chronic pain, General anaesthesia, Cataracts, Endometriosis, Contraception and abortion, coronary heart disease, chronic renal failure, or post anaesthesia cognitive dysfunction, and the like.

The compounds of the present invention can obtain more advantageous effects than additive effects in the prevention or treatment of the above diseases when used suitably in combination with the available further agent/drugs. The further agent/drugs for treating cancer is not especially limited, provided that it affords some utility for cancer treatment. However, typically the further agent for treating cancer is selected from anti-hyperproliferative, anti-cancer, chemotherapeutic agents, radiation therapy, anti-microtubule agents, cell-cycle check-point inhibitors, platinum coordination complexes, alkylating agents, antibiotic agents, topoisomerase II inhibitors, antimetabolites, topoisomerase I inhibitors, hormones and hormone analogues, signal transduction pathway inhibitors, non-receptor tyrosine kinase inhibitors, receptor tyrosine kinase inhibitors, angiogenesis inhibitors or anti-angiogenic agents (VEGF (R), PDGF (R), FGF (R), TGF-beta 1), immunotherapeutic agents, immune check-point inhibitors, proapoptotic agents and cell cycle signaling inhibitors. An immunotherapeutic agent may consist of but is not limited to an anti-tumor vaccine, an oncolytic virus, an immune stimulatory agonist antibodies such as anti-OX40, anti-41BB, anti-CD27, anti-CD28, anti-CD137, anti-GITR (or TNFRSF18), anti-HVEM (or TNFRSF14) and immune inhibitory antagonist antibodies such as anti-CTLA4, anti-PD1, anti-PDL-1, anti-CD40, anti-LAG3, anti-TIM3, anti-BTLA and anti-VISTA, a peptide, a dinucleotide, a cyclic dinucleotide, STING (stimulator of interferon genes) activators/modulators, a novel adjuvant, a cancer vaccine, a cytokine, a chimeric antigen receptor T cell therapy (CAR-T), a small molecule immune modulator, tumor microenvironment modulators, a tumor immunosuppression inhibitor/modulator. Also, any novel combination (synergistic/antagonistic), orthosteric and allosteric modulators wherein the administration dose can be decreased in comparison with administration of either drug alone to improve/synergize therapeutic efficacy or minimize/reduce adverse effects/events of co-administrated anti-cancer drugs.

### Methods of Preparation

The compounds described herein may be prepared by techniques known in the art. In addition, the compounds described herein may be prepared by following the reaction sequence as depicted in Scheme-1 to 4. Further, in the following schemes, where specific bases, acids, reagents, solvents, coupling agents, etc., are mentioned, it is understood that other bases, acids, reagents, solvents, coupling agents etc., known in the art may also be used and are therefore included within the present invention. Variations in reaction conditions, for example, temperature and/or duration of the reaction, which may be used as known in the art, are also within the scope of the present invention. All the stereoisomers of the compounds in these schemes, unless otherwise specified, are also encompassed within the scope of this invention. Compounds of the present invention can be synthesized from naturally occurring sources too. Key intermediates required for synthesizing analogues are either commercially available or can be prepared by the methods published in the literature.

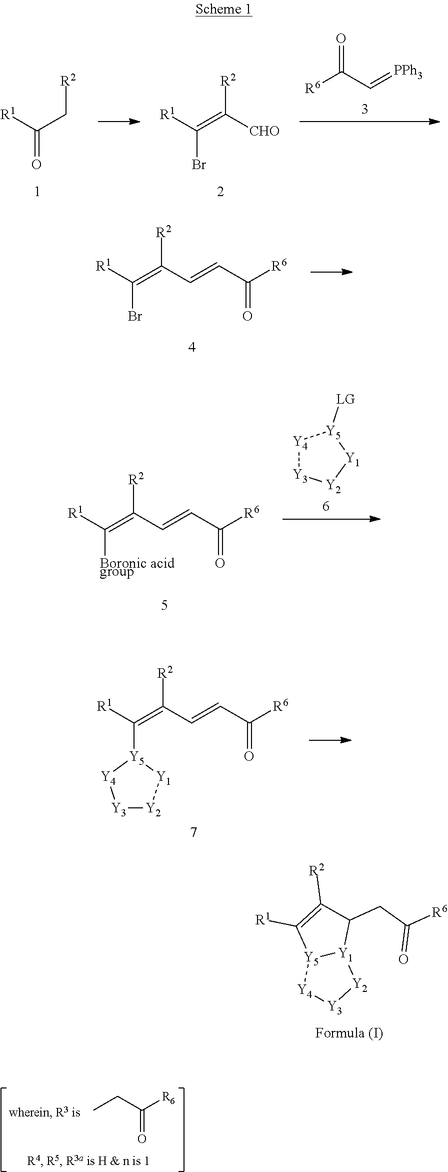

The compounds of Formula (I) (Y₁, Y₂, Y₃, Y₄, Y₅, R¹, R² & R⁶ can be same as defined above) can be synthesized as described in the above scheme 1. The keto compounds of the formula (1) can be converted to aldehyde compounds of the formula (2) in the presence of suitable formylation reagents such as Formic acid, N-Formylalkylamines, DMF, N-methylformanilide or the like and brominating reagent for example PBr₃, Br₂ or the like using suitable halogenated solvents. Commonly used halogenated solvents known in the art can be used which include but are not limited to solvents like chloroform, CCl₄, DCM or the like. Compounds of the formula (2) can be coupled with compounds of the formula (3) to give diene compounds of the formula (4) using suitable halogenated solvent. Commonly used halogenated solvents known in the art can be used which include but are not limited to solvents like chloroform, CCl₄, DCM or the like. The bromo group of compounds of the formula (4) can be replaced with boronic acid group such as bispinacolatodiborane, boronic acid or the like to get boronic compounds of the formula (5) in the presence of suitable base, transition metal catalyst and suitable solvent. The base used herein can be organic or inorganic bases known in the art. Examples of organic bases include but are not limited to Organolithiums such as n-BuLi, tert-BuLi etc, Amines such as DABCO, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. The compounds of the formula (7) can be obtained by replacement of boronic acid group of the formula (5) with compounds of the formula (6) in the presence of suitable base, transition metal catalyst and suitable solvent. The base used herein can be organic or inorganic bases known in the art. Examples of organic bases include but are not limited to Organolithiums such as n-BuLi, tert-BuLi etc, Amines such as DABCO, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. Compounds of the formula (7) can be cyclized in the presence of reagents such as AcOH:MeOH, AcOH:EtOH or the like to get compounds of Formula (I). The keto or thioketo group of the compounds of Formula (I) can be reduced using suitable reducing agent to get the corresponding alcohol or thioalcohol derivatives of Formula (I). Examples of suitable reducing agent includes but are not limited to NaBH₄, Lithium Aluminium Hydride (LAH), DIBAL-H or the like. The hydroxyl group of alcohol derivatives can be replaced with halogen atom in presence of corresponding halogenating reagent such as Diethylaminosulfur trifluoride, Cl₂, Br₂ or the like in suitable solvent to get corresponding halo derivatives of Formula (I). Solvent used herein include solvents of several categories like non-polar or polar aprotic. Examples include but are not limited to 1,4-dioxane, chloroform, diethyl ether, acetone, acetonitrile, THF or the like.

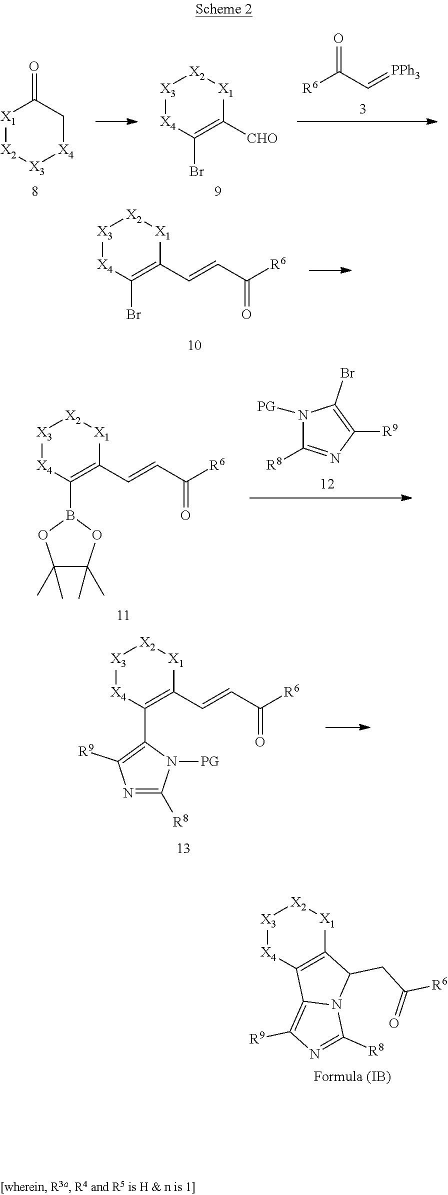

The compounds of Formula (IB) (X₁, X₂, X₃, X₄, R⁸, R⁹ & R⁶ can be same as defined above) can be synthesized as described in the above scheme 2. The keto compounds of the formula (8) can be converted to aldehyde compounds of the formula (9) in the presence reagents such as DMF, N-methylformanilide or the like and suitable brominating reagent for example PBr₃, Br₂ or the like using suitable halogenated solvents. Commonly used halogenated solvents known in the art can be used which include but are not limited to solvents like chloroform, CCl₄, DCM or the like. Aldehyde compounds of the formula (9) can be coupled with compounds of the formula (3) to give diene compounds of the formula (10) using suitable halogenated solvent. Commonly used halogenated solvents known in the art can be used which include but are not limited to solvents like chloroform, CCl₄, DCM or the like. Bromo group of compounds of the formula (10) can be replaced with boronic group using reagent such as bispinacolatodiborane, boronic acid or the like to get boronic compounds of the formula (11) in the presence of suitable base and transition metal catalyst in suitable solvent. The base used herein includes both organic and inorganic bases known in the art. Examples of organic bases include but are not limited to Organolithiums such as n-BuLi, tert-BuLi etc, Amines such as DABCO, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. The compounds of the formula (13) can be obtained by replacement of boronic group of compound (11) with compound (12) in the presence of suitable base and transition metal catalyst in suitable solvent. The base used herein can be organic or inorganic bases known in the art. Examples of organic bases include but are not limited to Organolithiums such as n-BuLi, tert-BuLi etc, Amines such as DABCO, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. Compounds of the formula (13) can be cyclized in the presence of reagents such as AcOH:MeOH, AcOH:EtOH or the like to get compound of formula Formula (IB). The keto or thioketo group of the compounds of Formula (IB) can be reduced using suitable reducing agent to get the corresponding alcohol or thioalcohol derivatives of Formula (IB). Examples of suitable reducing agent include but are not limited to NaBH₄, Lithium Aluminium Hydride (LAH), DIBAL-H or the like. The hydroxyl group of alcohol derivatives can be replaced with halogen atom in presence of corresponding halogenating reagent such as Diethylaminosulfur trifluoride, Cl₂, Br₂ or the like in suitable to get corresponding halo derivatives of the Formula (IB) Solvent used herein include solvents of several categories like non-polar or polar aprotic. Examples include but are not limited to 1,4-dioxane, chloroform, diethyl ether, acetone, acetonitrile, THF or the like.

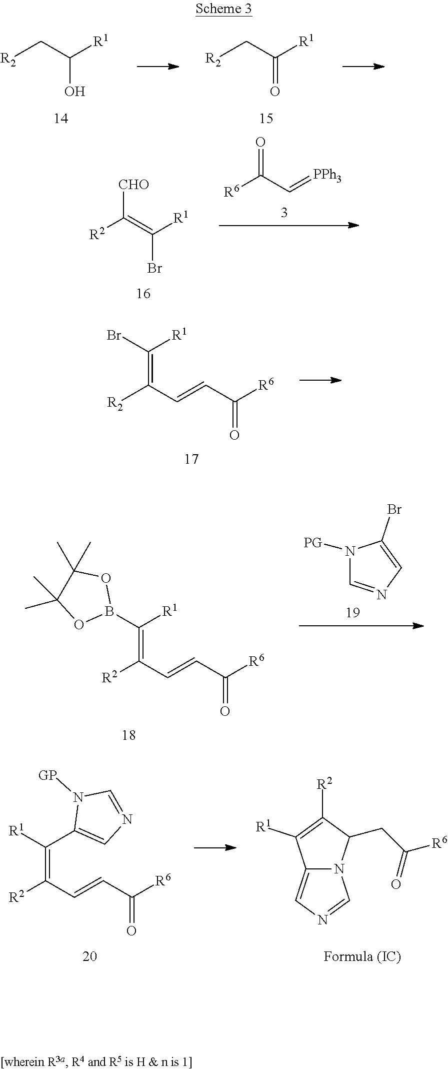

The compounds of Formula (IC) (R1, R² & R⁶ can be same as defined above) can be synthesized as described in the above scheme 3. Compounds of the formula (14) can be oxidized to compound (15) in the presence of suitable oxidizing agent. The oxidizing agent can be selected from but are not limited to Chromates such as Potassium dichromate, Pyridinium chlorochromate, Jone's reagent or the like, Hypervalent iodine reagents like Dess-Mmartin periodinane, 2-Iodoxybenzoic acid (IBX), Periodic acid or the like, Metal containing agents like KMnO₄, Selenium dioxide or the like. The keto compounds of the formula (15) can be converted to aldehyde compounds of the formula (16) in the presence of DMF and suitable brominating reagent in suitable halogenated solvent. Brominating agent can be selected from but are not limited to PBr₃, Br₂ or the like. Commonly used halogenated solvents known in the art can be used which include but are not limited to solvents like chloroform, CCl₄, DCM or the like. Aldehyde compounds of the formula (16) can be coupled with compounds of the formula (3) to give diene compounds of the formula (17) using suitable halogenated solvent such as DCM, CHCl₃, CCl₄ or the like. Bromo group of compounds of the formula (17) can be replaced with boronic group using reagent such as bispinacolatodiborane, boronic acid or the like to get compounds of the formula (18) in the presence of suitable base and transition metal catalyst in suitable solvent. The base used herein includes both organic and inorganic bases known in the art. Examples of organic bases include but are not limited to Organolithiums such as n-BuLi, tert-BuLi etc, Amines such as DABCO, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. Imidazole derivatives of the formula (20) can be obtained by replacement of boronic group of compound (18) with imidazole compounds of formula (19) in the presence of suitable base and transition metal catalyst in suitable solvent. The base used herein includes both organic and inorganic bases known in the art. Examples of organic bases include but are not limited to Organolithiums such as n-BuLi, tert-BuLi etc, Amines such as Dabco, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. Alternatively, Imidazole derivatives of the formula (20) can be derived from diene compounds of the formula (17) with their corresponding N-trityl imidazole-4-boronic acid in the presence of suitable base and transition metal catalyst in suitable solvent. The base used herein can be organic or inorganic bases known in the art. Examples of organic bases include but are not limited to Organolithiums such as n-BuLi, tert-BuLi etc, Amines such as DABCO, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. Imidazole derivatives of the formula (20) can be cyclized in the presence of reagents such as AcOH:MeOH, AcOH:EtOH or the like to get compounds of the Formula (IC). The keto or thioketo group of the compounds of Formula (IC) can be reduced using suitable reducing agent to get the corresponding alcohol or thioalcohol derivatives of Formula (IC). Examples of suitable reducing agent include but are not limited to NaBH₄, Lithium Aluminium Hydride (LAH), DIBAL-H or the like. The hydroxyl group of alcohol derivatives can be replaced with halogen atom in presence of corresponding halogenating reagent such as Diethylaminosulfur trifluoride, Br₂ or the like in suitable halogenated solvent to get corresponding halo derivatives of Formula (IC). Commonly used halogenated solvents known in the art include but are not limited to chloroform, CCl₄, DCM. Formula (IC), R⁶ itself can go for further substituted/functionalized with the corresponding functional elements/substitutions to yield the certain derivatised compounds by the available literature schematic paths.

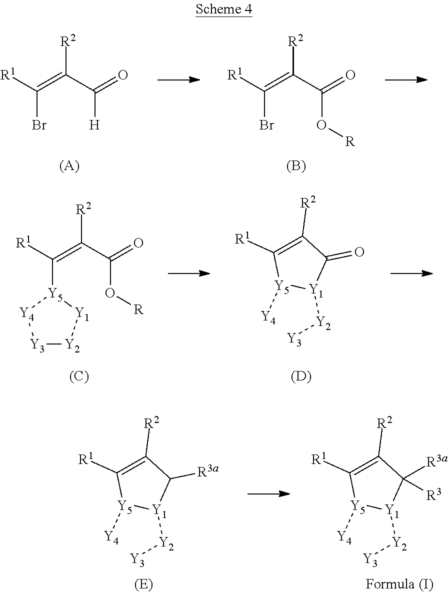

The compounds of Formula (I) (Y₁, Y₂, Y₃, Y₄, Y₅, R¹, R², R³ & R^{3a} can be same as defined above) can be synthesized as described in the above scheme 4. The aldehyde compounds of the formula (A) can be converted to ester compounds of the formula (B) in the presence of suitable oxidizing reagents and their corresponding alcohol (R—OH). The oxidizing agent used herein can be selected from but are not limited to oxygen, hydrogen peroxide, MnO₂ or the like. Compounds of formula (B) can be coupled with compound of formula 6 in presence of suitable base, transition metal catalyst and suitable solvent to get compounds of formula (C). The base used herein can be organic or inorganic bases known in the art. Examples of organic bases include but are not limited to Organolithiums such as n-BuLi, tert-BuLi or the like, Amines such as DABCO, Triethylamine, DIPEA or the like, Metal alkoxides such as Sodium tert-butoxide, Lithium tert-butoxide or the like. Examples of inorganic bases include but are not limited to Potassium hydroxide, Sodium hydroxide, Calcium carbonate, Cesium hydroxide or the like. Transition metal catalysts that can be used herein include but are not limited to Pd(dppf)Cl₂.DCM complex, Pd₂(dba)₃, Pd(PPh₃)₄, Ni(dppf)Cl₂ or the like. Solvent used herein include solvents of several categories like non-polar or polar aprotic. Examples include but are not limited to 1,4-dioxane, chloroform, diethyl ether, acetone, acetonitrile, THF or the like. Mixture of two or more solvents can also be used which includes but are not limited to mixture of dioxane:water, THF:water or the like. Compounds of formula (C) can be subjected cyclization using reagents such as AcOH:MeOH, AcOH:EtOH or the like to get cyclized compounds of formula (D). The hydrogenised compounds of formula (E) can be obtained by reduction of cyclized compounds of formula (D) in presence of suitable reducing agent. Examples of suitable reducing agent include but are not limited to NaBH₄, Lithium Aluminium Hydride (LAH), DIBAL-H or the like. The final compounds of Formula (I) can be obtained by alkylation & further functionalization of compounds of formula (E). The alkylation can be done in presence of suitable base & corresponding alkylating reagent. Examples of suitable reducing agent include but are not limited to NaBH₄, Lithium Aluminium Hydride (LAH), DIBAL-H or the like. Examples of alkylating reagent include but are not limited to alkyl halide, organometalic compounds or the like.

The compounds of Formula (IA) (X₁, X₂, X₃, X₄, Y₁, Y₂, Y₃, Y₄, Y₅, R³ & R^{3a} can be same as defined above) can also be synthesized as described in the above scheme 4 when compounds of Formula (I), R¹ and R² are combined together with their adjacent carbon atoms to form 5-8 membered cyclic ring.

The invention is explained in detail in the examples given below which are provided by way of illustration only and therefore should not be construed to limit the scope of the invention.

Abbreviations as used herein, are defined as follows:
AcOH: Acetic Acid
AD: Alzheimer's Disease
AIDS: Acquired Immune Deficiency Syndrome
ALL: Acute Lymphatic Leukemia
ALS: Amyotrophic Lateral Sclerosis
AML: Acute Myeloid Leukemia
CCl₄: Carbon Tetrachloride
CHCl₃: Trichloromethane or Chloroform
CLL: Chronic Lymphatic Leukemia
CML: Chronic Myeloid Leukemia
DABCO: 1,4-diazabicyclo[2.2.2]octane
DCM: Dichloromethane
DIBAL-H: Diisobutylaluminium Hydride
DIPEA: N,N-Diisopropylethylamine
DLBCL: Diffuse Large B-Cell Lymphoma
DM: Demineralised
DMF: Dimethylformamide
DMP: Dess-Martin Periodinane
EtOAc: Ethyl Acetate
EtOH: Ethanol
HATU: 1-[Bis(dimethylamino)methylene]-1H-1,2,3-triazolo[4,5-b]pyridinium 3-oxid hexafluorophosphate
HCl: Hydrochloric Acid
HPLC: High Performance Liquid Chromatography
IDO: Indoleamine 2,3-dioxygenase
K₂CO₃: Potassium Carbonate
KMnO₄: Potassium Permanganate
KOAc: Potassium Acetate
KP: Kynurenine Pathway
LAH: Lithium Aluminium Hydride
LCMS: Liquid Chromatography-Mass Spectrometry
MeOH: Methanol
Na₂CO₃: Sodium Carbonate
Na₂SO₄: Sodium Sulphate
NaBH₄: Sodium Borohydride
NADC: Nicotinamide Dinucleotide
NaH: Sodium Hydride
NaHCO₃: Sodium Bicarbonate
n-BuLi: n-Butyllithium
NFK: N-formylkynurenine
NMR: Nuclear Magnetic Resonance
NSCLC: Non-Samll-Cell-Lung Cancer
PBr₃: Phosphorus Tribromide
PIP: Piperidine
TBDMSCl: tert-Butyldimethylsilyl Chloride
TDO: Tryptophan 2,3-dioxygenase
THF: Tetrahydrofuran
TLC: Thin-Layer Chromatography

### EXPERIMENTAL

The present invention is further illustrated by the following examples, which are not to be construed in any way as imposing limitations upon the scope of this disclosure, but rather are intended to be illustrative only. On the contrary, it is to be clearly understood that resort may be had to various other embodiments, modifications, and equivalents thereof which, after reading the description herein, may suggest themselves to one of ordinary skill in the art without departing from the spirit of the present invention. Thus, the skilled artisan will appreciate how the experiments and examples may be further implemented as disclosed by variously altering the following examples, substituents, reagents, or conditions.

### EXAMPLES

### Example 1: Preparation of 1-Phenyl-2-(6,7,8,9-tetrahydro-5H-imidazo[5,1-a]isoindol-5-yl)-ethanone

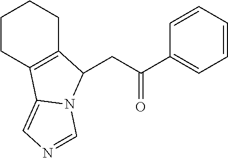 [SMILES: O=C(CC1C2=C(CCCC2)c2cncn21)c1ccccc1]

### Step 1: Preparation of 2-Bromo-cyclohex-1-enecarbaldehyde

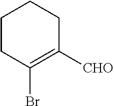

To a mixture of DMF (8 ml) and CHCl₃ (20 ml) was added PBr₃ (3.8 ml, 40.81 mmol) drop wise at about 0° C. Cyclohexanone (2.0 g, 20.4 mmol) in CHCl₃ (5 ml) was added at about 0° C. and stirred at room temperature for about 16 hours. The reaction was diluted with water, neutralized with sodium bicarbonate and extracted with chloroform (3×35 ml). The combined organic layer was dried over anhydrous sodium sulphate and concentrated under reduced pressure to give the crude product which was purified by column chromatography to give said compound (2.3 g, 59.74% yield) as colorless liquid.

¹HNMR (CDCl₃) δ: 10.02 (s, 1H), 2.77-2.72 (m, 2H), 2.30-2.26 (m, 2H), 1.78-1.73 (m, 2H), 1.71-1.66 (m, 2H).

### Step 2: Preparation of 3-(2-Bromo-cyclohex-1-enyl)-1-phenyl-propenone

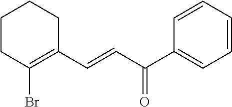 [SMILES: O=C(/C=C/C1=C(Br)CCCC1)c1ccccc1]

To a stirred solution of 2-Bromo-cyclohex-1-enecarbaldehyde (Step 1, 250 mg, 1.32 mmol) in DCM (10 ml) was added (Phenylcarbonylmethylene)triphenylphosphorane (753 mg, 1.98 mmol) at about 25-35° C. and the reaction mixture was stirred at about 25-35° C. for about 24 hours. After completion of the reaction, solvent was evaporated under reduced pressure and crude product was purified by column chromatography to give 3-(2-Bromo-cyclohex-1-enyl)-1-phenyl-propenone (375 mg, 97% yield) as pale yellow solid.

¹HNMR (CDCl₃) δ: 7.99-7.92 (m, 3H), 7.58-7.54 (m, 1H), 7.49-7.45 (m, 2H), 6.94 (d, 1H, J=15.6 Hz), 2.74-2.72 (m, 2H), 2.41-2.39 (m, 2H), 1.82-1.73 (m, 4H); Mass (LCMS): 292.1 (M+1).

### Step 3: Preparation of 1-Phenyl-3-[2-(4,4,5,5-tetramethyl-[1,3,2]dioxaborolan-2-yl)-cyclohex-1-enyl]-propenone

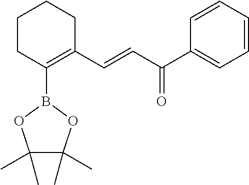 [SMILES: CC1(C)OB(C2=C(/C=C/C(=O)c3ccccc3)CCCC2)OC1(C)C]

To a mixture of 3-(2-Bromo-cyclohex-1-enyl)-1-phenyl-propenone (Step 2, 500 mg, 1.71 mmol) and bispinacolatodiborane (565 mg, 2.23 mmol) in 1,4-dioxane (10 ml) was added potassium acetate (505 mg, 5.15 mmol) at room temperature. The reaction mixture is degassed for about 10 minutes using nitrogen gas. To this suspension Pd(dppf)Cl₂-DCM complex (140 mg, 0.171 mmol) was added and reaction mixture was degassed for another five minutes. Reaction mixture was heated at about 90° C. for about 2 hours. After completion of reaction, solvent was evaporated under reduced pressure and residue was diluted with water (25 ml) and extracted with ethyl acetate (3×25 ml). Combined organic layer was dried over anhydrous sodium sulphate and concentrated to give crude product which was purified by column chromatography to give 1-Phenyl-3-[2-(4,4,5,5-tetramethyl-[1,3,2]dioxaborolan-2-yl)-cyclohex-1-enyl]-propenone (300 mg, 52% yield) as pale brown solid.

¹HNMR (CDCl₃) δ: 8.14 (d, 1H, J=15.5 Hz), 7.89-7.86 (m, 2H), 7.55-7.50 (m, 1H), 7.47-7.43 (m, 2H), 6.80 (d, 1H, J=15.5 Hz), 2.37-2.33 (m, 4H), 1.73-1.70 (m, 2H), 1.62-1.60 (m, 2H), 1.25 (s, 12H); Mass (LCMS): 339.2 (M+1).

### Step 4: Preparation of 1-Phenyl-3-[2-(3-trityl-3H-imidazol-4-yl)-cyclohex-1-enyl]-propenone

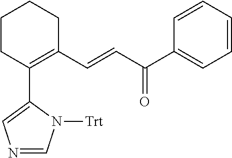

To a mixture of 1-Phenyl-3-[2-(4,4,5,5-tetramethyl-[1,3,2]dioxaborolan-2-yl)-cyclohex-1-enyl]-propenone (Step 3, 100 mg, 0.295 mmol) and 5-bromo-1-(triphenylmethyl)-imidazole (115 mg, 0.295 mmol) in 1,4-dioxane (4 ml) and water (1 ml) was added K₂CO₃ (102 mg, 0.74 mmol) at room temperature. The reaction mixture was degassed for about 10 minutes using nitrogen gas. To this suspension Pd(dppf)Cl₂-DCM complex (25 mg, 0.0295 mmol) was added and reaction mixture was degassed for another five minutes. Reaction mixture was heated at about 90° C. for about 3 hours. After completion of reaction, solvent was evaporated under reduced pressure and residue was diluted with water (10 ml) and extracted with ethyl acetate (3×20 ml). Combined organic layer was dried over anhydrous sodium sulphate and concentrated to give crude product which was purified by column chromatography to give 1-Phenyl-3-[2-(3-trityl-3H-imidazol-4-yl)-cyclohex-1-enyl]-propenone (70 mg, 81% yield) as pale brown solid.

¹HNMR (CDCl₃) δ: 8.33 (d, 1H, J=15.5 Hz), 7.93-7.91 (m, 2H), 7.51-7.49 (m, 4H), 7.37-7.31 (m, 9H) 7.17-7.14 (m, 6H), 6.89 (d, 1H, J=15.48 Hz), 6.80 (s, 1H), 2.63-2.60 (m, 2H), 2.42-2.38 (m, 2H), 1.77-1.71 (m, 4H); Mass (LCMS): 521.1 (M+1).

### Step 5: Preparation of 1-Phenyl-2-(6,7,8,9-tetrahydro-5H-imidazo[5,1-a]isoindol-5-yl)-ethanone

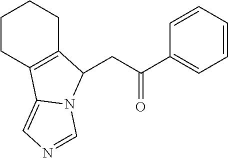 [SMILES: O=C(CC1C2=C(CCCC2)c2cncn21)c1ccccc1]

To a mixture of methanol (4 ml) and acetic acid (1 ml) was added 1-Phenyl-3-[2-(3-trityl-3H-imidazol-4-yl)-cyclohex-1-enyl]-propenone (Step 4, 70 mg, 0.134 mmol) and the reaction mixture was heated at about 90° C. for about 5 hours. After completion of the reaction, solvent was evaporated and reaction mixture was quenched with aqueous sodium bicarbonate (10 ml) and extracted with ethyl acetate (3×10 ml). Combined organic layer was dried over anhydrous sodium sulphate and concentrated to give crude product which was purified by column chromatography to give 1-Phenyl-2-(6,7,8,9-tetrahydro-5H-imidazo[5,1-a]isoindol-5-yl)-ethanone (40 mg, 60% yield) as pale yellow solid.

¹HNMR (CDCl₃) δ: 7.96-7.94 (m, 2H), 7.71 (br. s, 1H), 7.62-7.58 (m, 1H) 7.48 (t, 2H, J=7.5 Hz), 6.76 (s, 1H), 5.13 (d, 1H, J=9.2 Hz), 3.51 (dd, 1H, J=3.2 Hz, 18 Hz), 3.13 (dd, 1H, J=10.0 Hz, 18 Hz), 2.40-2.34 (m, 2H), 2.26-2.21 (m, 2H), 1.85-1.72 (m, 4H); Mass (LCMS): 279.2 (M+1). Purity: 90.54%.

Following intermediates have been synthesized as described in the above process of Example 1-Step 2 & 3:

| Intermediate No. | Analytical Data |
|---|---|
| 1-(3-Benzyloxy-phenyl)-3-(2-bromo- cyclohex-1-enyl)-propenone 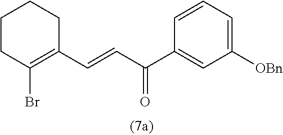 [SMILES: O=C(/C=C/C1=C(Br)CCCC1)c1cccc(OCc2ccccc2)c1] | ¹HNMR (CDCl₃, 400 MHz) δ: 7.97 (d, 1H, J = 15.6 Hz) 7.56-7.53 (m, 2H), 7.46-7.44 (m, 2H), 7.41-7.31 (m, 4H), 7.91-7.16 (m, 1H), 6.90 (d, 1H, J = 15.6 Hz), 5.13 (s, 2H), 2.74-2.73 (m, 2H), 2.39-2.37 (m, 2H), 1.82-1.76 (m, 4H); Mass (LCMS): 397.1 (M⁺) 399.1 (M + 2) |
| 1-(3-Benzyloxy-phenyl)-3-[2-(4,4,5,5- tetramethyl-[1,3,2]dioxaborolan-2-yl)- cyclohex-1-enyl]-propenone 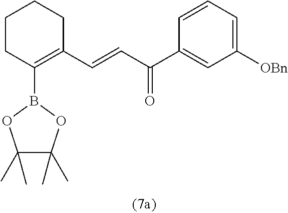 [SMILES: CC1(C)OB(C2=C(/C=C/C(=O)c3cccc(OCc4ccccc4)c3)CCCC2)OC1(C)C] | ¹HNMR (CDCl₃, 400 MHz) δ: 8.15 (d, 1H, J = 15.6 Hz), 7.51-7.43 (m, 4H), 7.40-7.32 (m, 5H), 6.79 (d, 1H, J = 15.6 Hz), 5.13 (s, 2H), 2.37-2.32 (m, 2H), 1.74-1.68 (m, 2H), 1.63-1.57 (m, 4H), 1.27 (br. s, 12H); Mass (LCMS): 445.1 (M + 1). |
| 3-(2-Bromo-cyclohex-1-enyl)-1-(2,5- difluoro-phenyl)-propenone 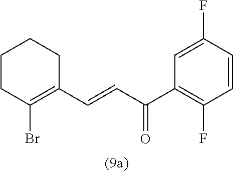 [SMILES: O=C(/C=C/C1=C(Br)CCCC1)c1cc(F)ccc1F] | ¹HNMR (CDCl₃, 400 MHz) δ: 7.93 (d, 1H, J = 15.6 Hz) 7.47-7.43 (m, 2H), 7.22-7.16 (m, 1H), 6.80 (d, 1H, J = 15.6 Hz), 2.37 (br. s, 2H), 2.36 (br. s, 2H), 1.80-1.76 (m, 4H); Mass (LCMS): 327.1 (M⁺) and 329.1 (M + 2). |
| 1-(2,5-Difluoro-phenyl)-3-[2-(4,4,5,5- tetramethyl-[1,3,2]dioxaborolan-2-yl)- cyclohex-1-enyl]-propenone 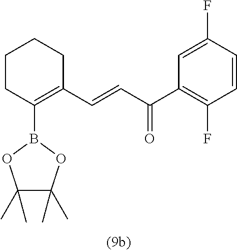 [SMILES: CC1(C)OB(C2=C(/C=C/C(=O)c3cc(F)ccc3F)CCCC2)OC1(C)C] | ¹HNMR (CDCl₃, 400 MHz) δ: 8.10 (d, 1H, J = 16 Hz), 7.34-7.29 (m, 1H), 7.17-7.06 (m, 2H), 6.63 (d, 1H, J = 16 Hz), 2.35-2.30 (m, 4H), 1.71-1.67 (m, 2H), 1.62-1.58 (m, 2H), 1.22 (br. s, 12H); Mass (LCMS): 375.1 (M + 1). |

### Example 2: Preparation of 1-Phenyl-2-(6,7,8,9-tetrahydro-5H-imidazo[5,1-a]isoindol-5-yl)-ethanol

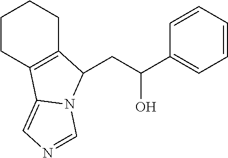 [SMILES: OC(CC1C2=C(CCCC2)c2cncn21)c1ccccc1]

To a stirred solution of 1-Phenyl-2-(6,7,8,9-tetrahydro-5H-imidazo[5,1-a]isoindol-5-yl)-ethanone (Example 1, 25 mg, 0.09 mmol) in methanol (2 ml) was added sodium borohydride (17 mg, 0.44 mmol) at about 0° C. and the reaction mixture was allowed to stir at room temperature for about 1 hour. After completion, volatiles were removed under reduced pressure to give the crude product. Water (10 ml) was added to the crude product and extracted with ethyl acetate (3×10 ml). Combined organic layer was evaporated under reduced pressure and crude product was purified by trituration to give 1-Phenyl-2-(6,7,8,9-tetrahydro-5H-imidazo[5,1-a]isoindol-5-yl)-ethanol (17 mg, 68% yield) as off white solid.

¹HNMR (CDCl₃) δ: 7.38-7.18 (m, 6H), 6.86-6.73 (m, 1H), 4.91-4.88 (m, 1H) 4.60 (br. s, 1H), 2.32-1.97 (m, 6H), 1.76-1.63 (m, 5H); Mass (LCMS): 281.1 (M+1). Purity: 90.46%.

Following examples have been synthesized by the above procedures described in Example 1 and 2 with their corresponding intermediates in similar reaction conditions:

| Example | | |
|---|---|---|
| No. | IUPAC Name/Structure | Analytical Data |
| 3. | 1-(3-Chloro-phenyl)-2- (6,7,8,9-tetrahydro-5H- imidazo[5,1-a]isoindol-5- yl)-ethanone 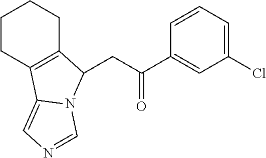 [SMILES: O=C(CC1C2=C(CCCC2)c2cncn21)c1cccc(Cl)c1] | ¹HNMR (CDCl₃, 400 MHz) δ: 7.93 (s, 1H), 7.84 (d, 1H, J = 8 Hz), 7.59-7.57 (m, 2H) 7.44 (t, 1H, J = 8 Hz), 6.71 (s, 1H), 5.10 (d, 1H, J = 9.6 Hz), 3.47 (dd, 1H, J = 2.8, 18 Hz), 3.09 (dd, 1H, J = 9.6, 18 Hz), 2.39-2.36 (m, 2H), 2.23 (br. s, 2H), 1.84-1.76 (m, 4H); Mass (LCMS): 313.1 (M + 1). Purity: 94.72% |
| 4. | 1-(3-Chloro-phenyl)-2- (6,7,8,9-tetrahydro-5H- imidazo[5,1-a]isoindol-5- yl)-ethanol 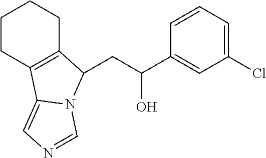 [SMILES: OC(CC1C2=C(CCCC2)c2cncn21)c1cccc(Cl)c1] | ¹HNMR (CDCl₃, 400 MHz) δ: 7.27-7.16 (m, 5H), 6.92-6.80 (m, 1H), 4.90 (s, 1H), 4.68 (br. s, 1H), 2.32-2.18 (m, 6H), 1.76-1.69 (m, 5H); Mass (LCMS): 315.1 (M + 1). Purity: 93.15% |
| 5. | 1-Phenyl-2-[6-(toluene-4- sulfonyl)-4,6,7,8-tetrahydro- 5H-2,6,8a-triaza- cyclopenta[a]inden-8-yl]- ethanone 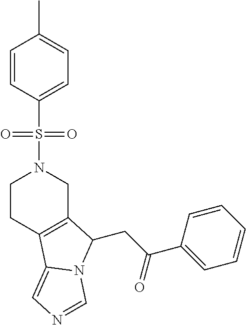 [SMILES: Cc1ccc(S(=O)(=O)N2CCC3=C(C2)C(CC(=O)c2ccccc2)n2cncc23)cc1] | ¹HNMR (DMSOd₆, 400 MHz) δ: 8.02 (d, 2H, J = 8 Hz), 7.70-7.66 (m, 3H), 7.56-7.53 (m, 3H), 7.43 (d, 2H, J = 8 Hz), 6.64 (s, 1H), 5.23-5.20 (m, 1H), 3.95-3.77 (m, 3H), 3.40- 3.33 (m, 3H), 3.22-3.16 (m, 2H), 2.32 (s, 3H); Mass (LCMS): 434.2 (M + 1); Purity: 97.36% |
| 6. | 1-Phenyl-2-[6-(toluene-4- sulfonyl)-4,6,7,8- tetrahydro-5H-2,6,8a-triaza- cyclopenta [a] inden-8-yl]- ethanol 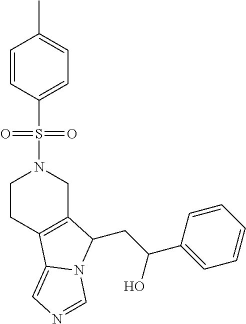 [SMILES: Cc1ccc(S(=O)(=O)N2CCC3=C(C2)C(CC(O)c2ccccc2)n2cncc23)cc1] | ¹HNMR (CDCl₃, 400 MHz) δ: 7.92-7.82 (m, 1H), 7.72 (d, 1H, J = 8 Hz), 7.67(d, 1H, J = 8 Hz), 7.43-7.40 (m, 2H), 7.31-7.30 (m, 3H) 7.28-7.15 (m, 2H), 6.72-6.66 (m, 1H), 5.63- 5.51 (m, 1H), 4.98-4.87 (m, 1H), 4.69-4.54 (m, 1H), 4.01-3.84 (m, 2H), 3.50-3.35 (m, 2H), 3.33-3.28 (m, 2H), 3.18-3.10 (m, 2H), 2.30 (s, 3H). Mass (LCMS): 436.2 (M +1). Purity: 97.71% |
| 7. | 1-(3-Benzyloxy-phenyl)-2- (6,7,8,9-tetrahydro-5H- imidazo[5,1-a]isoindol-5- yl)-ethanone 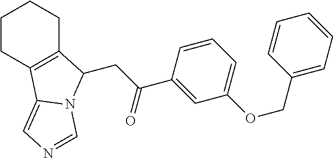 [SMILES: O=C(CC1C2=C(CCCC2)c2cncn21)c1cccc(OCc2ccccc2)c1] | ¹HNMR (CDCl₃, 400 MHz) δ: 7.64 (s, 1H), 7.58 (t, 1H, J = 1.7 Hz), 7.52 (d, 1H, J = 7.7 Hz) 7.45-7.34 (m, 6H), 7.22-7.20 (m, 1H), 6.72 (s, 1H), 5.12 (s, 2H), 5.10-5.08 (m, 1H), 3.47 (dd, 1H, J = 3, 18 Hz), 3.09 (dd, 1H, J = 10, 18 Hz), 2.40-2.36 (m, 2H), 2.25-2.20 (m, 2H), 1.85-1.72 (m, 4H); Mass (LCMS): 385.2 (M + 1); Purity: 95.97% |
| 8. | 1-(3-Benzyloxy-phenyl)-2- (6,7,8,9-tetrahydro-5H- imidazo[5,1-a]isoindol-5- yl)-ethanol 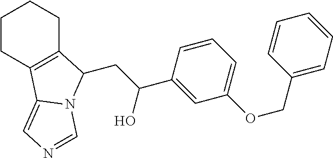 [SMILES: OC(CC1C2=C(CCCC2)c2cncn21)c1cccc(OCc2ccccc2)c1] | ¹HNMR (CDCl₃, 400 MHz) δ: 7.42-7.21 (m, 7H), 7.00-6.97 (m, 1H), 6.91-6.86 (m, 2H) 6.69 (s, 1H), 5.04-5.02 (m, 2H), 4.94-4.94 (m, 0.3H), 4.84-4.81 (m, 1H), 4.55-4.50 (m, 0.7H), 2.33-2.24 (m, 3H), 2.11-2.10 (m, 3H), 1.79-1.66 (m, 4H). Mass (LCMS): 387.2 (M + 1); Purity: 97.18% |
| 9. | 1-(2,5-Difluoro-phenyl)-2- (6,7,8,9-tetrahydro-5H- imidazo[5,1-a]isoindol-5- yl)-ethanone 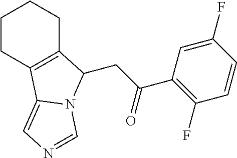 [SMILES: O=C(CC1C2=C(CCCC2)c2cncn21)c1cc(F)ccc1F] | ¹HNMR (CDCl₃, 400 MHz) δ: 7.68-7.64 (m, 1H), 7.60 (br. s, 1H), 7.29-7.23 (m, 1H), 7.17-7.11 (m, 1H), 6.70 (br. s, 1H), 5.08-5.06 (m, 1H), 3.56-3.48 (m, 1H), 3011-303 (m, 1H), 2.38-2.36 (m, 2H), 2.24-2.20 (m, 2H), 1.85-1.72 (m, 4H); Mass (LCMS): 315.2 (M + 1); Purity: 90.46% |
| 10. | 1-(2,5-Difluoro-phenyl)-2- (6,7,8,9-tetrahydro-5H- imidazo[5,1-a]isoindol-5- yl)-ethanol 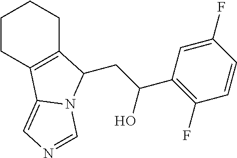 [SMILES: OC(CC1C2=C(CCCC2)c2cncn21)c1cc(F)ccc1F] | ¹HNMR (CDCl₃, 400 MHz) δ: 7.81 (br. s, 1H), 7.24-7.23 (m, 1H), 6.98-6.87 (m, 2H), 6.64 (br. s, 1H), 5.26-5.24 (m, 0.3H), 5.15- 5.13 (m, 0.7H), 4.84 (d, 0.3H, J = 9.6 Hz), 4.57 (br. s, 0.7H), 2.30 (br. s, 2H), 2.27-2.09 (m, 2H), 1.76-1.63 (m, 6H); Mass (LCMS): 315.2 (M + 1); Purity: 93.13% |

### Example 11: Preparation of 2-(7-Methyl-6-phenyl-5H-pyrrolo[1,2-c]imidazol-5-yl)-1-phenyl-ethanone

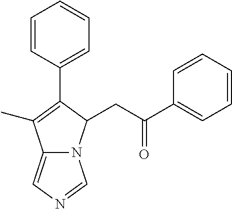 [SMILES: CC1=C(c2ccccc2)C(CC(=O)c2ccccc2)n2cncc21]

### Step 1: Preparation of 1-Phenyl-propan-2-one

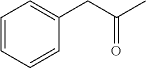

To a stirred solution of 3-Phenyl-2-propanol (5 g, 36.76 mmol) in DCM (70 ml) was added Dess-Martin periodinane (18.7 g, 44.11 mmol) portion wise at about 0° C. The reaction mixture was allowed to stir at room temperature for about 2 hours. After completion of reaction, solvent was evaporated under reduced pressure and 10% EtOAc/n-hexanes was added to the reaction mass. The precipitate obtained was filtered off and washed with 10% EtOAc/n-hexane (3×25 mL). The filtrate was concentrated; water (50 mL) was added and extracted with ethyl acetate (3×50 ml). The combined organic layer was dried over anhydrous sodium sulphate and concentrated under reduced pressure to give the pure product desired compound (4.5 g, 91.46% yield) as pale yellow liquid which was used in the next step without further purification.

### Step 2: Preparation of 3-Bromo-2-phenyl-but-2-enal

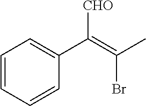

To a mixture of DMF (13 mL, 167.91 mmol) and CHCl₃ (50 mL) was added PBr₃ (6.3 mL, 67.16 mmol) drop wise at about 0° C. Then 1-Phenyl-propan-2-one (Step 1, 4.5 g, 33.58 mmol) in CHCl₃ (10 mL) was added at about 0° C., and the mixture was stirred at room temperature for 12 hours. The reaction mass was diluted with water (50 mL), neutralized with sodium bicarbonate and extracted with chloroform (3×50 mL). The combined organic layer was dried over anhydrous sodium sulphate and concentrated under reduced pressure to give the crude product which was purified by column chromatography to give the desired compound (4.0 g, 52.98% yield) as pale yellow liquid and the same is used to further step.

### Step 3: Preparation of 5-Bromo-1,4-diphenyl-hexa-2,4-dien-1-one

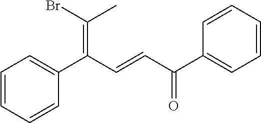 [SMILES: C/C(Br)=C(\C=C\C(=O)c1ccccc1)c1ccccc1]

To a stirred solution of 3-Bromo-2-phenyl-but-2-enal (Step 2, 2.5 g, 11.11 mmol) in acetonitrile (30 ml) was added (Phenylcarbonylmethylene)triphenylphosphorane (4.64 g, 12.22 mmol) at room temperature and the reaction mixture was stirred at room temperature for about 17 hours. After completion of reaction, solvent was evaporated under reduced pressure and crude product was purified by column chromatography to give desired compound (2.7 g, 74.38% yield) as off-white solid.

¹HNMR (CDCl₃, 400 MHz) δ: 8.16 (d, 1H, J=15.31 Hz), 7.77-7.74 (m, 2H), 7.51-7.38 (m, 6H), 7.17-7.15 (m, 2H), 6.38 (d, 1H, J=15.31 Hz), 2.30 (s, 3H); Mass (LCMS): 327.2 (M+1).

### Step 4: Preparation of 1,4-Diphenyl-5-(4,4,5,5-tetramethyl-[1,3,2]dioxaborolan-2-yl)-hexa-2,4-dien-1-one

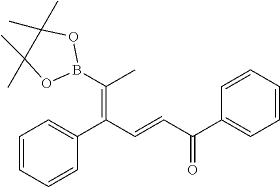 [SMILES: C/C(B1OC(C)(C)C(C)(C)O1)=C(\C=C\C(=O)c1ccccc1)c1ccccc1]

To a mixture of 5-Bromo-1,4-diphenyl-hexa-2,4-dien-1-one (Step 3, 300 mg, 0.917 mmol) and bispinacolatodiborane (348 mg, 1.37 mmol) in 1,4-dioxane (10 mL) was added KOAc (270 mg, 2.75 mmol) at room temperature. The reaction mixture was degassed for about 10 minutes using nitrogen gas. To this suspension Pd(dppf)Cl₂-DCM complex (75 mg, 0.0917 mmol) was added and reaction mixture was degassed for another five minutes. Reaction mixture was heated at about 80° C. for about 2 hours. After completion of reaction, solvent was evaporated under reduced pressure and residue was diluted with water (25 mL) and extracted with ethyl acetate (3×25 mL). Combined organic layer was dried over anhydrous sodium sulphate and concentrated to give crude product which was purified by column chromatography to give desired compound (250 mg, 72.02% yield) as pale yellow solid.

¹HNMR (CDCl₃, 400 MHz) δ: 8.36 (d, 1H, J=15.28 Hz), 7.75-7.73 (m, 2H), 7.50-7.32 (m, 6H), 7.11-7.09 (m, 2H), 6.27 (d, 1H, J=15.35 Hz), 1.71 (s, 3H), 1.32 (s, 12H); Mass (LCMS): 375.3 (M+1).

### Step 5: Preparation of 1,4-Diphenyl-5-(3-trityl-3H-imidazol-4-yl)-hexa-2,4-dien-1-one

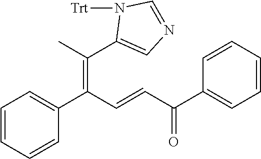

To a mixture of 1,4-Diphenyl-5-(4,4,5,5-tetramethyl-[1,3,2]dioxaborolan-2-yl)-hexa-2,4-dien-1-one (Step 4, 250 mg, 0.668 mmol) and trityl protected 2-bromoimidazole (320 mg, 0.735 mmol) in 1,4-dioxane (8 ml) and water (2 ml) was added K₂CO₃ (230 mg, 1.67 mmol) at room temperature. The reaction mixture was degassed for about 10 minutes using nitrogen gas. To this suspension Pd(dppf)Cl₂-DCM complex (55 mg, 0.0735 mmol) was added and reaction mixture was degassed for another five minutes. Reaction mixture was heated at about 90° C. for about 3 hours. After completion of reaction, solvent was evaporated under reduced pressure and residue was diluted with water (20 ml) and extracted with ethyl acetate (3×25 ml). Combined organic layer was dried over anhydrous sodium sulphate and concentrated to give crude product which was purified by column chromatography to give desired compound (100 mg, 67.26%) as pale brown solid.

¹HNMR (CDCl₃, 400 MHz) δ: 8.65 (d, 1H, J=15.15 Hz), 7.76-7.74 (m, 2H), 7.51 (s, 1H), 7.43-7.33 (m, 15H) 7.21-7.18 (m, 8H), 6.99 (s, 1H), 6.35 (d, 1H, J=15.15 Hz), 2.00 (s, 3H); Mass (LCMS): 315.1 (M+1).

### Step 6: Preparation of 2-(7-Methyl-6-phenyl-5H-pyrrolo[1,2-c]imidazol-5-yl)-1-phenyl-ethanone

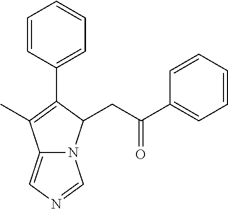 [SMILES: CC1=C(c2ccccc2)C(CC(=O)c2ccccc2)n2cncc21]

To a mixture of methanol:acetic acid (3:1, 30 mL) was added 1,4-Diphenyl-5-(3-trityl-3H-imidazol-4-yl)-hexa-2,4-dien-1-one (Step 5, 100 mg, 0.134 mmol) and the reaction mixture was heated at about 90° C. for about 12 hours. After completion of the reaction, solvents were evaporated and reaction mixture was quenched with aqueous sodium bicarbonate (20 ml) and extracted with ethyl acetate (3×20 ml). Combined organic layer was dried over anhydrous sodium sulphate and concentrated to give crude product which was purified by column chromatography to give desired compound (30 mg, 53.12% yield) as pale yellow solid.

¹HNMR (CDCl₃, 400 MHz) δ: 7.87-7.85 (m, 2H), 7.70 (s, 1H), 7.57-7.52 (m, 1H) 7.46-7.40 (m, 4H), 7.35-7.31 (m, 3H), 6.89 (s, 1H), 5.77 (d, 1H, J=10.8 Hz), 3.34 (dd, 1H, J=2 Hz, 18 Hz), 3.09 (dd, 1H, J=10.8 Hz, 18 Hz), 2.22 (s, 3H); Mass (LCMS): 315.0 (M+1). Purity: 94.79%.

Following intermediates have been synthesized as described in the above process of Example 11-Step 3 & 5:

| Intermediate No. | Analytical Data |
|---|---|
| 5-Bromo-1,5-diphenyl-penta-2,4-dien-1- one 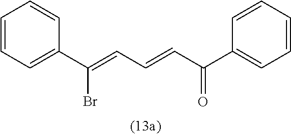 [SMILES: O=C(/C=C/C=C(\Br)c1ccccc1)c1ccccc1] | ¹HNMR (CDCl₃, 400 MHz) δ: 7.98-7.96 (m, 2H), 7.90 (dd, 1H, J = 10.8, 15.2 Hz), 7.69-7.66 (m, 2H), 7.60-7.56 (m, 1H), 7.51-7.46 (m, 2H), 7.42-7.36 (m, 3H) 7.21 (dd, 1H, J = 0.8, 15.2 Hz), 7.10 (dd, 1H, J = 0.8, 10.8 Hz); Mass (LCMS): 313.1 (M⁺) and 315.1 (M + 2) |
| 1,5-Diphenyl-5-(1-trityl-1H-imidazol-4- yl)-penta-2,4-dien-1-one 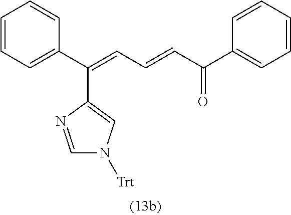 | ¹HNMR (CDCl₃, 400 MHz) δ: 8.43 (dd, 1H, J = 11.6, 15.2 Hz), 7.97-7.95 (m, 2H), 7.57 (d, 1H, J = 1.2 Hz), 7.52-7.50 (m, 1H), 7.47-7.43 (m, 2H), 7.39- 7.33 (m, 11H), 7.32-7.28 (m, 3H), 7.20-7.16 (m, 6H), 7.06 (d, 1H, J = 15.2 Hz), 6.82 (d, 1H, J = 1.6 Hz), 6.66 (d, 1H, J = 11.6 Hz); Mass (LCMS): 301.2 (M + 1), (LCMS showed detritylated mass of product). |
| 5-Bromo-4-phenyl-hexa-2,4-dienoic acid ethyl ester 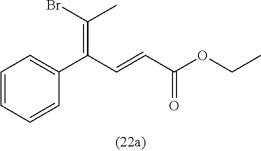 [SMILES: CCOC(=O)/C=C/C(=C(\C)Br)c1ccccc1] | ¹HNMR (CDCl₃, 400 MHz) δ: 8.05 (d, 0.6H, J = 15.6 Hz), 7.82 (d, 0.4H, J = 15.6 Hz), 7.41-7.34 (m, 3H), 7.09-7.07 (m, 2H), 5.38-5.32 (m, 1H), 4.20-4.13 (m, 2H), 2.73 (s, 1H), 2.26 (s, 2H), 1.28-1.23 (m, 3H); (LCMS): 296 (M + 1). |
| 4-Phenyl-5-(4,4,5,5-tetramethyl- [1,3,2]dioxaborolan-2-yl)-hexa-2,4- dienoic acid ethyl ester 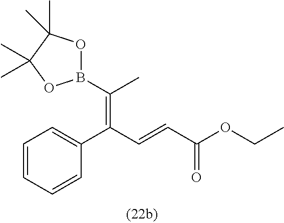 [SMILES: CCOC(=O)/C=C/C(=C(\C)B1OC(C)(C)C(C)(C)O1)c1ccccc1] | ¹HNMR (CDCl₃, 400 MHz) δ: 8.31 (d, 1H, 15.2 Hz), 7.36 (t, 2H, J = 7.6 Hz), 7.28 (d, 1H, J = 7.6 Hz), 7.03 (t, 2H, J = 7.6 Hz), 5.30 (d, 1H, J = 15.2 Hz), 4.14, q, 2H, J = 7.2 Hz), 1.67 (s, 3H), 1.37 (s, 12H), 1.26 (t, 3H, J = 7.2 Hz). |
| 4-Phenyl-5-(3-trityl-3H-imidazol-4-yl)- hexa-2,4-dienoic acid ethyl ester 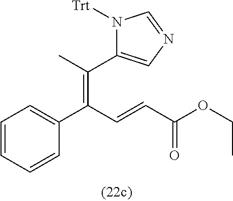 | ¹HNMR (CDCl₃, 400 MHz) δ: 8.55 (d, 1H, J = 15.6 Hz), 7.51 (d, 1H, J = 1.6 Hz), 7.39-7.30 (m, 12H), 7.22-7.19 (m, 6H), 7.13-7.11 (m, 2H), 6.98 (d, 1H, J = 1.6 Hz), 5.28 (d, 1H, J = 15.6 Hz), 4.11 (q, 2H, J = 7.2 Hz), 1.94 (s, 3h), 1.18 (t, 3H, J = 7.2 Hz). |

### Example 12: Preparation of 2-(7-Methyl-6-phenyl-5H-pyrrolo[1,2-c]imidazol-5-yl)-1-phenyl-ethanol

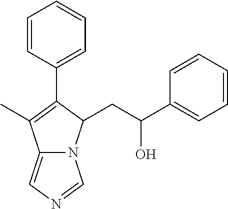 [SMILES: CC1=C(c2ccccc2)C(CC(O)c2ccccc2)n2cncc21]

To a stirred solution of 2-(7-Methyl-6-phenyl-5H-pyrrolo[1,2-c]imidazol-5-yl)-1-phenyl-ethanone (Example 11, 10 mg, 0.0318 mmol) in methanol (2 ml) was added sodium borohydride (1.2 mg, 0.0318 mmol) at about 0° C. and the reaction mixture was stirred at room temperature for about 0.5 hours. After completion, solvent was evaporated under reduced pressure to give the crude product. Water 10 ml was added to the crude product and was extracted with ethyl acetate (3×10 ml). Combined organic layer was evaporated under reduced pressure and crude product was purified by trituration with n-hexanes give desired compound (8 mg, 79.28% yield) as off white solid.

¹HNMR (CDCl₃, 400 MHz) δ: 7.44-7.17 (m, 11H), 6.86 (s, 1H), 5.46-5.44 (br. m, 0.4H) 5.60 (br. s, 0.6H), 5.06-5.03 (m, 0.4H), 4.81-4.77 (m, 0.6H), 2.17 (s, 3H), 2.16-2.15 (m, 1H), 2.10-2.04 (m, 1H); Mass (LCMS): 317.2 (M+1). Purity: 94.65%

Following examples have been synthesized by the above procedure described in Example 11 and 12 with their corresponding intermediates in similar reaction conditions:

| Example | IUPAC | |
|---|---|---|
| No. | Name/Structure | Analytical Data |
| 13. | 1-Phenyl-2-(7-phenyl- 5H-pyrrolo[1,2- c]imidazol-5-yl)- ethanone 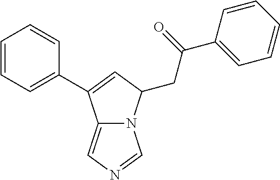 [SMILES: O=C(CC1C=C(c2ccccc2)c2cncn21)c1ccccc1] | ¹HNMR (CDCl₃, 400 MHz) δ: 8.85 (br. s, 1H), 7.97 (d, 2H, J = 7.6 Hz), 7.66-7.60 (m, 3H) 7.52- 7.28 (m, 6H), 5.72-5.70 (m, 1H), 3.81 (dd, 1H, J = 4.0, 18.4 Hz), 3.50 (dd, 1H, J = 9.6, 18.4 Hz); Mass (LCMS): 301.2 (M + 1); Purity: 92.4% |
| 14. | 1-Phenyl-2-(7-phenyl- 5H-pyrrolo[1,2- c]imidazol-5-yl)-ethanol 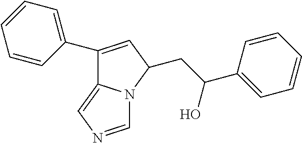 [SMILES: OC(CC1C=C(c2ccccc2)c2cncn21)c1ccccc1] | ¹HNMR (CDCl₃, 400 MHz) δ: 7.71-7.62 (m, 2H), 7.46-7.30 (m, 8H), 7.14-7.06 (m, 1H) 6.61 (d, 0.5H, J = 2.4 Hz), 6.45 (d, 0.5H, J = 2.0 Hz), 5.13- 4.95 (m, 1H), 2.49-2.31 (m, 1H), 2.17-1.98 (m, 1H); Mass (LCMS): 303.2 (M + 1); Purity: 91.26% |
| 21. | 2-(6-Methyl-7-phenyl- 5H-pyrrolo[1,2- c]imidazol-5-yl)-1- phenyl-ethanone 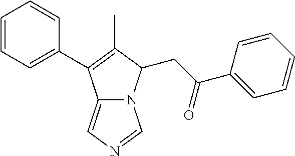 [SMILES: CC1=C(c2ccccc2)c2cncn2C1CC(=O)c1ccccc1] | ¹HNMR (CDCl₃, 400 MHz) δ: 8.00 (d, 2H, J = 7.03 Hz), 7.64-7.38 (m, 9H), 6.82 (s, 1H), 5.30- 5.28 (m, 1H), 3.66-3.64 (m, 1H), 3.26 (dd, 1H, J = 9.7, 17.6 Hz), 2.14 (s, 3H); Mass (LCMS): 315.2 (M + 1); Purity = 96.90%. |
| 22. | (7-Methyl-6-phenyl-5H- pyrrolo[1,2-c]imidazol- 5-yl)-acetic acid ethyl ester 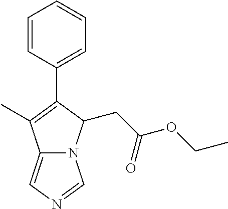 [SMILES: CCOC(=O)CC1C(c2ccccc2)=C(C)c2cncn21] | ¹HNMR (CDCl₃, 400 MHz) δ: 7.73 (s, 1H), 7.42 (d, 2H, J = 7.6 Hz), 7.34-7.26 (m, 3H), 6.89 (s, 2H), 5.47 (d, 1H, J = 10.4 Hz), 4.21-4.14 (m, 2H), 2.79 (dd, 1H, J = 2.8, 17.2 Hz), 2.31 (dd, 1H, J = 10.4, 17.2 Hz), 2.17 (d, 3H, 2 Hz), 1.24 (t, 3H, J = 3.6 Hz); (LCMS): 283.2 (M + 1), Purity = 91.10%. |
| 30. | 2-(7-Methyl-6-phenyl- 5H-pyrrolo[1,2- c]imidazol-5-yl)-1- phenyl-ethanone, Isomer-I 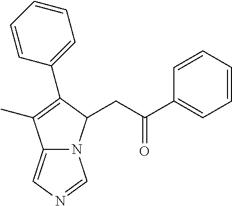 [SMILES: CC1=C(c2ccccc2)C(CC(=O)c2ccccc2)n2cncc21] | ¹HNMR (CDCl₃, 400 MHz) δ: 7.87-7.85 (m, 2H), 7.76 (br. s, 1H), 7.57-7.54 (m, 1H), 7.46-7.40 (m, 4H), 7.35-7.31 (m, 3H), 6.94 (br. s, 1H), 5-79- 5.76 (m, 1H), 3.35 (dd, 1H, J = 2, 18.8 Hz), 3.09 (dd, 1H, J = 10.4, 18.8 Hz), 2.23 (d, 3H, J = 2 Hz); Mass (LCMS): 315.2 (M + 1); Purity = 94.08%. |
| 31. | 2-(7-Methyl-6-phenyl- 5H-pyrrolo[1,2- c]imidazol-5-yl)-1- phenyl-ethanone, Isomer-II 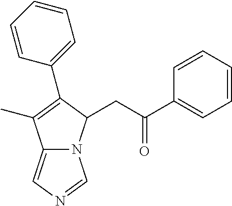 [SMILES: CC1=C(c2ccccc2)C(CC(=O)c2ccccc2)n2cncc21] | ¹HNMR (CDCl₃, 400 MHz) δ: 7.87-7.85 (m, 2H), 7.72 (br. s, 1H), 7.58-7.53 (m, 1H), 7.46-7.40 (m, 4H), 7.35-7.31 (m, 3H), 6.94 (br. s, 1H), 5-78- 5.75 (m, 1H), 3.35 (dd, 1H, J = 2, 18.4 Hz), 3.09 (dd, 1H, J = 10.4, 18.4 Hz), 2.22 (d, 3H, J = 2 Hz); Mass (LCMS): 315.2 (M + 1); Purity = 95.97%. |

### Example 17: 1-(3-Chloro-phenyl)-2-(7-methyl-6-phenyl-5H-pyrrolo[1,2-c]imidazol-5-yl)-ethanone

### Step 1: Preparation of 1-(3-chlorophenyl)-2-(triphenyl-λ⁵-phosphanylidene)ethan-1-one

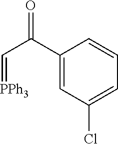

To a stirred solution of compound m-chlorobenzoyl bromide (5 g, 21.45 mmol) in toluene (60 mL) was added triphenyl phosphine (6.3 g, 23.6 mmol) at room temperature and the reaction mixture was heated at about 80° C. for about 3-5 hours. After completion of reaction, reaction mass was cooled to room temperature, solid obtained was filtered and washed with excess toluene (100 mL). The solid obtained was dissolved in DCM (10 mL) and treated with 10% aq Na₂CO₃ (50 mL) solution at room temperature for about 12 hours. The reaction mass was extracted with DCM, combined organic layer was dried over sodium sulfate and concentrated to give 1-(3-chlorophenyl)-2-(triphenyl-λ⁵-phosphanylidene)ethan-1-one (5.7 g, 64%) as white solid.

¹HNMR (CDCl₃, 400 MHz) δ: 7.94 (t, 1H, J=1.57 Hz), 7.84-7.81 (m, 1H), 7.73-7.67 (m, 6H), 7.59-7.54 (m, 3H), 7.50-7.45 (m, 6H), 7.32-7.24 (m, 2H), 4.40 (br. s, 1H); Mass (LCMS): 415.2 (M+1)

### Step 2: Preparation of 5-Bromo-1-(3-chloro-phenyl)-4-phenyl-hexa-2,4-dien-1-one

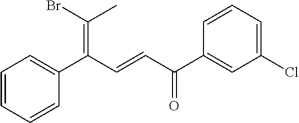 [SMILES: C/C(Br)=C(\C=C\C(=O)c1cccc(Cl)c1)c1ccccc1]

To a stirred solution of compound Benzeneacetaldehyde, α-(1-bromoethylidene) (700 mg, 3.11 mmol) in acetonitrile (15 mL) was added 1-(3-chlorophenyl)-2-(triphenyl-λ⁵-phosphanylidene)ethan-1-one (Step 1, 1.55 g, 3.73 mmol) at room temperature and the reaction mixture was refluxed at about 80° C. for about 12 hours. After completion of reaction, reaction mass was cooled to room temperature, solvent was evaporated under reduced pressure and crude product was purified by column chromatography to give compound 5-Bromo-1-(3-chloro-phenyl)-4-phenyl-hexa-2,4-dien-1-one (340 mg, 30%) as pale yellow solid.

¹HNMR (CDCl₃, 400 MHz) δ: 8.17 (d, 1H, J=14.8 Hz), 7.74 (t, 1H, J=1.6 Hz), 7.60-7.58 (m, 1H), 7.49-7.32 (m, 7H), 6.30 (d, 1H, J=14.8 Hz), 2.30 (s, 3H); Mass (LCMS): 361.1, 363.2 (M⁺M+2).

### Step 3: Preparation of 1-(3-Chloro-phenyl)-4-phenyl-5-(3-trityl-3H-imidazol-4-yl)-hexa-2,4-dien-1-one

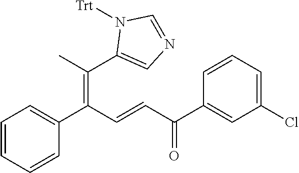

To a mixture of compound 5-Bromo-1-(3-chloro-phenyl)-4-phenyl-hexa-2,4-dien-1-one (Step 2, 330 mg, 0.914 mmol) and N-trityl imidazole-4-boronic acid (485 mg, 1.37 mmol) in 1,4-dioxane:water (4:1; 15 mL) was added K₂CO₃ (315 mg, 2.28 mmol) at room temperature. The reaction mixture was degassed for about 10 minutes using nitrogen gas. To this suspension Pd(dppf)Cl₂-DCM complex (75 mg, 0.0914 mmol) was added and reaction mixture was degassed for another five minutes. Reaction mixture was heated at about 100° C. for about 3-5 hours. After completion of reaction, solvent was evaporated under reduced pressure and residue was diluted with water (25 mL) and extracted with ethyl acetate (3×25 mL). Combined organic layer was dried over anhydrous sodium sulfate and concentrated to give crude product which was purified by column chromatography to yield compound 1-(3-Chloro-phenyl)-4-phenyl-5-(3-trityl-3H-imidazol-4-yl)-hexa-2,4-dien-1-one (120 mg, 22%) as pale yellow fluffy solid. Product formation was confirmed by LCMS and used in the next step.

LCMS: 349.2 (M+1, de-tritylated product)

### Step 4: Preparation of 1-(3-Chloro-phenyl)-2-(7-methyl-6-phenyl-5H-pyrrolo[1,2-c]imidazol-5-yl)-ethanone

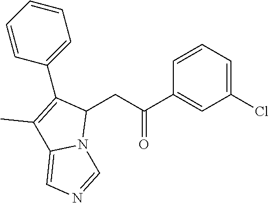 [SMILES: CC1=C(c2ccccc2)C(CC(=O)c2cccc(Cl)c2)n2cncc21]

To a mixture of methanol and acetic acid (3:1; 10 mL) was added compound 1-(3-Chloro-phenyl)-4-phenyl-5-(3-trityl-3H-imidazol-4-yl)-hexa-2,4-dien-1-one (Step 3, 120 mg, 0.203 mmol) and the reaction mixture was heated at about 90° C. for about 12 hours. After completion of the reaction, solvents were evaporated; reaction mixture was quenched with aqueous sodium bicarbonate (10 mL) and extracted with ethyl acetate (3×10 mL). Combined organic layer was dried over anhydrous sodium sulfate and concentrated to give crude product which was purified by column chromatography to yield compound 1-(3-Chloro-phenyl)-2-(7-methyl-6-phenyl-5H-pyrrolo[1,2-c]imidazol-5-yl)-ethanone (15 mg, 21%) as pale yellow solid.

¹HNMR (CDCl₃, 400 MHz) δ: 7.83 (t, 1H, J=1.6 Hz), 7.74-7.71 (m, 1H), 7.68 (s, 1H), 7.54-7.51 (m, 1H), 7.46-7.43 (m, 2H), 7.39-7.31 (m, 4H), 6.90 (s, 1H), 5.75-5.73 (m, 1H), 3.31 (dd, 1H, J=2.0, 18.4 Hz), 3.05 (dd, 1H, J=10.8, 18.6 Hz), 2.22 (d, 3H, J=1.6 Hz); Mass (LCMS): 349.1 (M+1). Purity=93.82%.

Following intermediates have been synthesized as described in the above process of Example 17-Step 3 & 5:

| Intermediate No. | Analytical Data |
|---|---|
| 1-(benzo[d][1,3]dioxol-5-yl)-2- (triphenyl-λ⁵-phosphanylidene)ethan-1- one 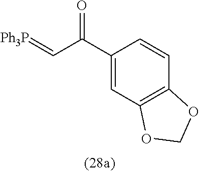 | ¹HNMR (CDCl₃, 400 MHz) δ: 7.73-7.64 (m, 6H), 7.57-7.51 (m, 4H), 7.49-7.44 (m, 7H), 6.77 (d, 1H, J = 8.0 Hz), 5.95 (s, 2H); 4.30 (br.d, 1H, J = 22.8 Hz); Mass (LCMS): 425.2 (M + 1) |
| 1-Benzo[1,3]dioxol-5-yl-5-bromo-4- methyl-5-phenyl-penta-2,4-dien-1-one 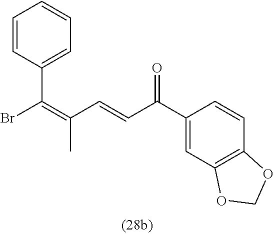 [SMILES: CC(/C=C/C(=O)c1ccc2c(c1)OCO2)=C(\Br)c1ccccc1] | ¹HNMR (CDCl₃, 400 MHz) δ: 8.14 (d, 1H, J = 15.2 Hz), 7.47-7.39 (m, 3H), 7.31-7.26 (m, 2H), 7.16- 7.14 (m, 2H), 6.77 (d, 1H, J = 8.0 Hz), 6.34 (d, 1H, J = 15.2 Hz), 6.02 (s, 2H), 2.29 (s, 3H); Mass (LCMS): 371.1 . |
| 1-(3-(trifluoromethyl)phenyl)-2- (triphenyl-λ⁵-phosphanylidene)ethan-1- one 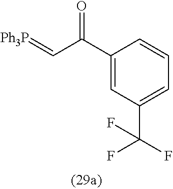 | ¹HNMR (CDCl₃, 400 MHz) δ: 8.23 (s, 1H), 8.13 (d, 1H, J = 7.6 Hz), 7.74-7.64 (m, 6H), 7.60-7.54 (m, 4H), 7.52-7.43 (m, 7H), 4.45 (d, 1H, J = 23.2 Hz). |
| 5-Bromo-4-methyl-5-phenyl-1-(3- trifluoromethyl-phenyl)-penta-2,4-dien- 1-one 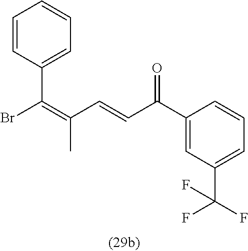 [SMILES: CC(/C=C/C(=O)c1cccc(C(F)(F)F)c1)=C(\Br)c1ccccc1] | ¹HNMR (CDCl₃, 400 MHz) δ: 8.18 (d, 1H, J = 15.2 Hz), 8.02 (s, 1H), 7.89 (d, 1H, J = 7.6 Hz), 7.76 (d, 1H, J = 8.0 Hz), 7.54 (t, 1H, J = 8.0 Hz), 7.48-7.40 (m, 4H), 7.16 (dd, 1H, J = 1.6 Hz & 8.4 Hz), 6.32 (d, 1H, J = 15.6 Hz), 2.31 (s, 3H). |
| 1-(naphthalen-2-yl)-2-(triphenyl-λ⁵- phosphanylidene)ethan-1-one 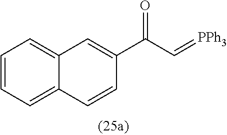 | ¹HNMR (CDCl₃, 400 MHz) δ: 8.48 (s, 1H), 8.07 (dd, 1H, J = 1.2, 8.4 Hz), 7.90-7.88 (m, 1H), 7.84-7.73 (m, 8H), 7.59-7.55 (m, 3H), 7.51-7.43 (m, 8H), 4.59 (br.s, 1H); Mass (LCMS): 431.1 (M + 1). |
| 5-Bromo-1-naphthalen-2-yl-4-phenyl- hexa-2,4-dien-1-one 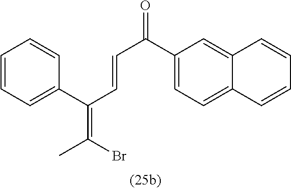 | ¹HNMR (CDCl₃, 400 MHz) δ: 8.24-8.19 (m, 2H), 7.90-7.83 (m, 4H), 7.59-7.40 (m, 5H), 7.21-7.19 (m, 2H), 6.52 (d, 1H, J = 15.6 Hz), 2.32 (s, 3H); Mass (LCMS): 478.1 (M + 1). |
| 1-cyclopropyl-2-(triphenyl-λ⁵- phosphanylidene)ethan-1-one 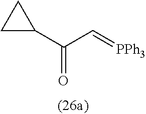 | ¹HNMR (CDCl₃, 400 MHz) δ: 7.66-7.61 (m, 6H), 7.54-7.50 (m, 3H), 7.46-7.41 (m, 6H), 3.78 (br.s, 1H), 1.82-1.76 (m, 1H), 0.89-0.86 (m, 2H), 0.61-0.58 (m, 2H); Mass (LCMS): 445.1 (M + 1). |
| 5-Bromo-1-cyclopropyl-4-phenyl-hexa- 2,4-dien-1-one 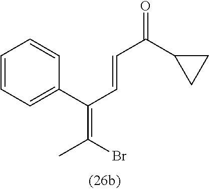 [SMILES: C/C(Br)=C(/C=C/C(=O)C1CC1)c1ccccc1] | ¹HNMR (CDCl₃, 400 MHz) δ: 8.00 (d, 1H, J = 15.6 Hz), 7.43-7.33 (m, 3H), 7.10-7.08 (m, 2H), 5.73 (d, 1H, J = 15.6 Hz), 2.27 (s, 3H), 2.15-2.09 (m, 1H), 1.08-1.04 (m, 2H), 0.91-0.85 (m, 2H); Mass (LCMS): 292.2 (M + 1). |
| 1-(p-tolyl)-2-(triphenyl-l5- phosphanylidene)ethan-1-one 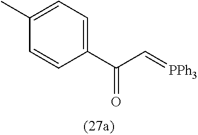 | ¹HNMR (CDCl₃, 400 MHz) δ: 7.75(d, 2H, J = 8.4 Hz), 7.70-7.62 (m, 9H), 7.59-7.54 (m, 6H), 7.15 (d, 2H, J = 8 Hz), 4.43 (d, 1H, J = 24.8 Hz), 2.31 (s, 3H); (LCMS): 395.2 (M + 1). |
| 5-Bromo-4-phenyl-1-p-tolyl-hexa-2,4- dien-1-one 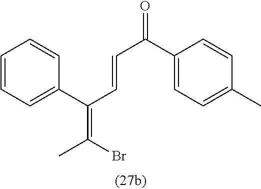 [SMILES: C/C(Br)=C(/C=C/C(=O)c1ccc(C)cc1)c1ccccc1] | ¹HNMR (CDCl₃, 400 MHz) δ: 8.14 (d, 0.65H, J = 15.2 Hz), 7.96 (d, 0.35H, J = 15.2 Hz), 7.67-7.64 (m, 1H), 7.47-7.34 (m, 4H), 7.20-7.12 (m, 4H), 6.44 (d, 0.35H, J = 15.2 Hz), 6.37 (d, 0.65H, J = 15.2 Hz), 2.49 (s, 1H), 2.37 (s, 2H), 2.30 (s, 2H), 2.15 (s, 1H); Mass (LCMS): 341.1. |
| 1-(3-methoxyphenyl)-2-(triphenyl-l5- phosphaneylidene)ethan-1-one 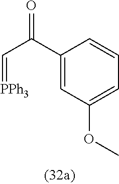 | ¹HNMR (CDCl₃, 400 MHz) δ: 7.83 (dd. 1H, J = 2, 7.6 Hz), 7.77-7.72 (m, 6H), 7.64-7.52 (m, 3H), 7.48- 7.43 (m, 6H), 7.35-7.29 (m, 1H), 6.94-6.89 (m, 2H), 5.29 (s, 1H), 3.88 (s, 3H); Mass (LCMS): 411.2 (M + 1). |
| (2E,4Z)-5-bromo-1-(2-methoxyphenyl)- 4-phenylhexa-2,4-dien-1-one 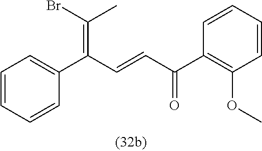 [SMILES: COc1ccccc1C(=O)/C=C/C(=C(\C)Br)c1ccccc1] | ¹HNMR (CDCl₃, 400 MHz) δ: 8.40 (d, 1H, J = 15.6 Hz, 7.59-7.55 (m, 1H), 7.42-7.34 (m, 4H), 7.15-7.12 (m 2H), 6.99-6.95 (m, 1H), 6.88-6.85 (m, 1H), 6.33 (d, 1H, J = 15.6 Hz), 3.68 (s, 3H), 2.28 (s, 3H). |
| 1-(3-methoxyphenyl)-2-(triphenyl-l5- phosphaneylidene)ethan-1-one 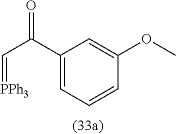 | Mass (LCMS): 411.2 (M + 1). |
| 5-bromo-1-(3-methoxyphenyl)-4- phenylhexa-2,4-dien-1-one 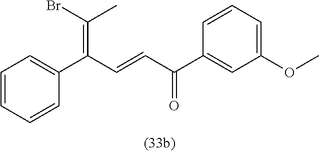 [SMILES: COc1cccc(C(=O)/C=C/C(=C(\C)Br)c2ccccc2)c1] | ¹HNMR (CDCl₃, 400 MHz) δ: 8.15 (d, 1H, J = 15.2 Hz), 7.29-7.27 (m, 3H), 7.16-7.12 (m, 6H), 6.49 (d, 1H, J = 15.2 Hz), 2.99 (s, 3H), 2.16 (s, 3H); Mass (LCMS): 357, 359 (M⁺, M + 2). |
| 1-(4-fluorophenyl)-2-(triphenyl-l5- phosphaneylidene)ethan-1-one 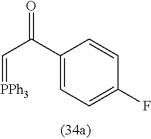 | ¹HNMR (CDCl₃, 400 MHz) δ: 7.97-7.93 (m, 2H), 7.73-7.68 (m, 6H), 7.58-7.54 (m, 3H), 7.49-7.45 (m, 6H), 7.03-6.98 (m, 2H), 4.42 (d, 1H, J = 23.6 Hz). |
| 5-bromo-1-(4-fluorophenyl)-4- phenylhexa-2,4-dien-1-one 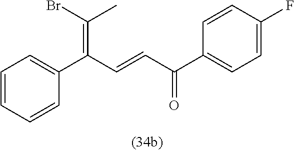 [SMILES: C/C(Br)=C(\C=C\C(=O)c1ccc(F)cc1)c1ccccc1] | ¹HNMR (CDCl₃, 400 MHz) δ: 8.15 (d, 0.7H. J = 15.2 Hz), 7.99 (d, 0.3H, J = 15.2 Hz), 7.80-7.75 (m, 2H), 7.47-7.38 (m, 3H), 7.16-7.14 (m, 2H), 7.10-7.04 (m, 2H), 6.40 (d, 0.3H. J = 15.2 Hz), 6.34 (d, 0.7H. J = 15.2 Hz), 2.30 (s, 3H); Mass (LCMS): 345, 347 (M + 1). |
| 1-(2,5-difluorophenyl)-2-(triphenyl-l5- phosphaneylidene)ethan-1-one 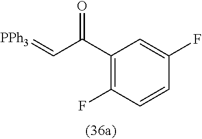 | ¹HNMR (CDCl₃, 400 MHz) δ: 7.74-7.65 (m, 7H), 7.58-7.52 (m, 2H), 7.50-7.44 (m, 6H), 7.36 (s, 1H), 6.99-6.93 (m, 2H), 4.58 (d, 1H, J = 25.6 Hz). |
| 5-bromo-1-(2,5-difluorophenyl)-4- methyl-5-phenylpenta-2,4-dien-1-one 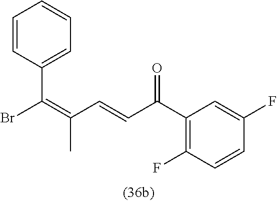 [SMILES: CC(/C=C/C(=O)c1cc(F)ccc1F)=C(\Br)c1ccccc1] | ¹HNMR (CDCl₃, 400 MHz) δ: 8.10 (d, 1H, J = 15.2 Hz), 7.44-7.35 (m, 5H), 7.16-7.11 (m, 2H), 7.07-6.99 (m, 1H), 6.23 (dd, 1H, J = 2.8, 15.2 Hz), 2.30 (s, 3H); LCMS: (m/Z): 363.1. |
| 1-(4-methoxyphenyl)-2-(triphenyl-l5- phosphaneylidene)ethan-1-one 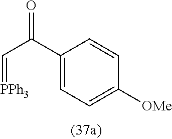 | ¹HNMR (CDCl₃, 400 MHz) δ: 7.92 (d. 2H, J = 8.8 Hz), 7.74-7.68 (m, 6H), 7.55-7.53 (m, 3H), 7.48-7.44 (m, 6H), 6.86 (d, 2H, J = 9.2 Hz), 4.34 (d, 1H, J = 23.6 Hz), 3.82 (s, 3H); Mass (LCMS): 411.1 (M + 1). |
| 5-bromo-1-(4-methoxyphenyl)-4- phenylhexa-2,4-dien-1-one 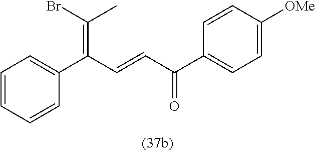 [SMILES: COc1ccc(C(=O)/C=C/C(=C(\C)Br)c2ccccc2)cc1] | ¹HNMR (CDCl₃, 400 MHz) δ: 8.14 (d, 1H, J = 15.2 Hz), 7.76 (d, 2H, J = 8.8 Hz), 7.48-7.37 (m, 3H), 7.17-7.14 (m, 2H), 6.88 (d, 2H, J = 8.0 Hz), 6.38 (d, 1H, J = 15.2 Hz), 3.83 (s, 3H), 2.29 (s, 3H); Mass (LCMS): 356.0, 358.0 (M⁺, M + 2). |
| 1-(4-chlorophenyl)-2-(triphenyl-l5- phosphaneylidene)ethan-1-one 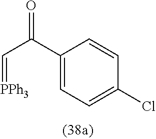 | ¹HNMR (CDCl₃, 400 MHz) δ: 7.89 (d. 2H, J = 8.4 Hz), 7.73-7.67 (m, 6H), 7.58-7.55 (m, 3H), 7.50-7.45 (m, 6H), 7.30 (d, 2H, J = 8.0 Hz), 4.38 (d, 1H, J = 22.4 Hz); Mass (LCMS): 415.1 (M +1). |
| 5-bromo-1-(4-chlorophenyl)-4- phenylhexa-2,4-dien-1-one 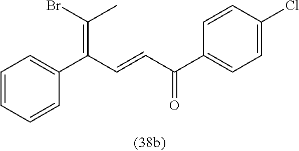 [SMILES: C/C(Br)=C(\C=C\C(=O)c1ccc(Cl)cc1)c1ccccc1] | 1HNMR (CDCl₃, 400 MHz) δ: 8.16 (d, 1H, J = 15.2 Hz), 7.69 (d, 2H, J = 8.8 Hz), 7.47-7.40 (m, 3H), 7.37 (d, 2H, J = 8.5 Hz), 7.16-7.14 (m, 2H), 7.32 (d, 1H, J = 15.2 Hz), 2.30 (s, 3H); Mass (LCMS): 361.1, 362.9 (M⁺ and M + 2). |
| 1-(3-chloro-4-fluorophenyl)-2-(triphenyl- l5-phosphaneylidene)ethan-1-one 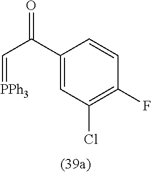 | 1HNMR (CDCl₃, 400 MHz) δ: 8.01 (dd, 1H, J = 2.0 and 7.2 Hz), 7.84-7.80 (m, 1H), 7.72-7.66 (m, 6H), 7.60-7.54 (m, 3H), 7.51-7.46 (m, 6H), 7.09 (t, 1H, J = 8.8 Hz), 4.35 (d, 1H, J = 26.3 Hz); Mass (LCMS): 333.1 (M + 1). |
| (2E,4Z)-5-bromo-1-(3-chloro-4- fluorophenyl)-4-phenylhexa-2,4-dien-1- one 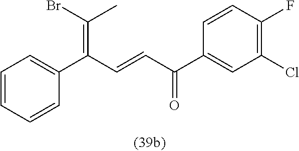 [SMILES: C/C(Br)=C(\C=C\C(=O)c1ccc(F)c(Cl)c1)c1ccccc1] | 1HNMR (CDCl₃, 400 MHz) δ: 8.17 (d, 1H, J = 15.2 Hz), 7.85 (dd, 1H, J = 2.0 and 7.2 Hz), 7.65-7.61 (m, 1H), 7.40-7.37 (m, 3H), 7.08-7.06 (m, 3H), 6.29 (d, 1H, J = 15.2 Hz), 2.31 (s, 3H); Mass (LCMS): 379.0 and 381.0 (M⁺, M + 2). |
| Ethyl-8-bromo-4-oxo-7-phenylnona-5,7- dienoate 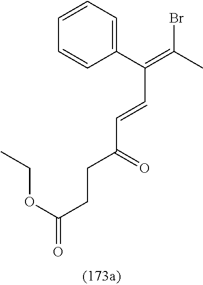 [SMILES: CCOC(=O)CCC(=O)/C=C/C(=C(\C)Br)c1ccccc1] | ¹HNMR (CDCl₃, 400 MHz) δ: 7.96 (d, 1H, J = 15.6 Hz), 7.40-7.35 (m, 3H), 7.058-7.06 (m, 2H), 5.63 (dd, 1H, J = 0.4, 15.6 Hz), 4.11 (q, 2H, J = 7.2 Hz), 2.88 (t, 2H, J = 6.8 Hz), 2.60 (t, 2H, J = 6.8 Hz), 2.27 (s, 3H), 1.24 (t, 3H, J = 7.2 Hz). |
| 1-(naphthalen-1-yl)-2-(triphenyl-l5- phosphaneylidene)ethan-1-one 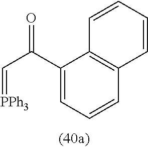 | ¹HNMR (CDCl₃, 400 MHz) δ: 8.67-8.64 (m, 1H), 7.83-7.77 (m, 8H), 7.70-7.64 (m, 1H), 7.61-7.57 (m, 3H), 7.53-7.48 (m, 7H), 7.44-7.39 (m, 3H), 4.24 (d, 1H, J = 22 Hz); Mass (LCMS): 431.1 (M + 1). |
| 5-bromo-1-(naphthalen-1-yl)-4- phenylhexa-2,4-dien-1-one 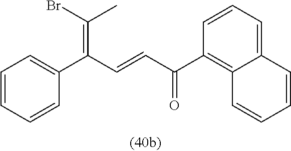 [SMILES: C/C(Br)=C(\C=C\C(=O)c1cccc2ccccc12)c1ccccc1] | ¹HNMR (CDCl₃, 400 MHz) δ: 8.50-8.45 (m, 1H), 7.90 (d, 1H, J = 8.0 Hz), 7.85-7.51 (m, 4H), 7.48- 7.46 (m, 2H), 7.16-7.08 (m, 3H), 7.07-7.05 (m, 2H), 6.19 (d, 1H, J = 16.8 Hz), 2.28 (s, 3H); Mass (LCMS): 377.1, 379.0. |
| 1-([1,1′-biphenyl]-4-yl)-2-(triphenyl-l5- phosphaneylidene)ethan-1-one 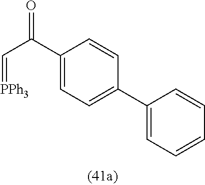 | ¹HNMR (CDCl₃, 400 MHz) δ: 8.05-8.02 (m, 3H), 7.76-7.38 (m, 14H), 7.36-7.28 (m, 7H), 4.92 (br. s, 1H); Mass (LCMS): 457.2 (M + 1). |
| 1-([1,1′-biphenyl]-4-yl)-5-bromo-4- phenylhexa-2,4-dien-1-one 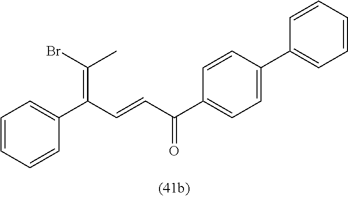 [SMILES: C/C(Br)=C(\C=C\C(=O)c1ccc(-c2ccccc2)cc1)c1ccccc1] | Mass (LCMS): 403 (M + 1). |
| 1-(thiophen-3-yl)-2-(triphenyl-l5- phosphaneylidene)ethan-1-one 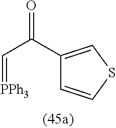 | ¹HNMR (CDCl₃, 400 MHz) δ: 7.82 (m, 1H), 7.74- 7.67 (m, 8H), 7.58-7.54 (m, 4H), 7.35 (s, 1H), 7.32- 7.29 (m, 3H), 7.22-7.20 (m, 1H), 4.25 (d, 1H, J = 22 Hz); Mass (LCMS): 387.1 (M + 1). |
| 5-bromo-4-phenyl-1-(thiophen-3- yl)hexa-2,4-dien-1-one 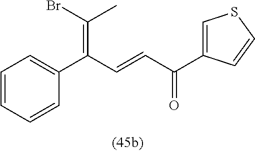 [SMILES: C/C(Br)=C(\C=C\C(=O)c1ccsc1)c1ccccc1] | ¹HNMR (CDCl₃, 400 MHz) δ: 8.16 (d, 1H, J = 15.2 Hz), 7.82-7.80 (m, 1H), 7.48-7.39 (m, 4H), 7.28- 7.27 (m, 1H), 7.16-7.14 (m, 2H), 6.24 (d, 1H, J = 15.2 Hz), 2.29 (s, 3H); Mass (LCMS): 332.1 (M + 1) |
| 1-(4-(dimethylamino)phenyl)-2- (triphenyl-l5-phosphaneylidene)ethan-1- one 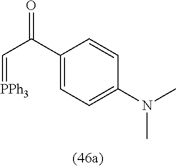 | ¹HNMR (CDCl₃, 400 MHz) δ: 7.88 (d, 2H, J = 8.4 Hz), 7.74-7.69 (m, 6H), 7.53-7.51 (m, 3H), 7.46-7.43 (m, 6H), 6.67 (d, 2H, J = 8.8 Hz), 4.32 (br. s, 1H), 2.97 (s, 6H); Mass (LCMS): 424.0 (M + 1). |
| 5-bromo-1-(4-(dimethylamino)phenyl)- 4-phenylhexa-2,4-dien-1-one 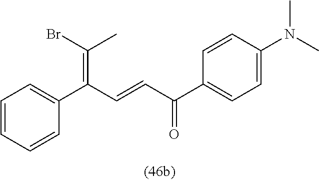 [SMILES: C/C(Br)=C(\C=C\C(=O)c1ccc(N(C)C)cc1)c1ccccc1] | ¹HNMR (CDCl₃, 400 MHz) δ: 8.11 (d, 1H, J = 15.2 Hz), 7.71 (d, 2H, J = 9.2 Hz), 7.45-7.38 (m, 3H), 7.17-7.15 (m, 2H), 6.59 (d, 2H, J = 9.1 Hz), 6.43 (d, 1H, J = 15.2 Hz), 3.02 (s, 6H), 2.28 (s, 3H); Mass (LCMS): 370.0 and 371.9 (M⁺, M + 2). |
| 1-(5,6,7,8-tetrahydronaphthalen-2-yl)-2- (triphenyl-l5-phosphaneylidene)ethan-1- one 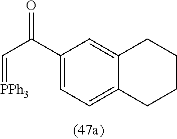 | ¹HNMR (CDCl₃, 400 MHz) δ: 7.73-7.64 (m, 8H), 7.56-7.52 (m, 3H), 7.47-7.40 (m, 6H), 7.03 (d, 1H, J = 3.0 Hz), 4.41 (br. s, 1H), 2.78 (br. s, 4H), 1.78 (br. s, 4H); Mass (LCMS): 435.0 (M + 1). |
| 5-bromo-4-phenyl-1-(5,6,7,8- tetrahydronaphthalen-2-yl)hexa-2,4-dien- 1-one 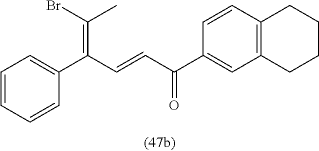 [SMILES: C/C(Br)=C(\C=C\C(=O)c1ccc2c(c1)CCCC2)c1ccccc1] | ¹HNMR (CDCl₃, 400 MHz) δ: 8.12 (d, 1H, J = 15.6 Hz), 7.50-7.49 (m, 1H), 7.45-7.38 (m, 4H), 7.16-7.13 (m, 2H), 7.06 (d, 1H, J = 7.6 Hz), 6.35 (d, 1H, J = 15.6 Hz), 2.79-2.74 (m, 4H), 2.29 (s, 3H), 1.81-1.76 (m, 4H); Mass (LCMS): 380.9 and 382.9 (M⁺ + and M + 2). |
| 1-(4-phenoxyphenyl)-2-(triphenyl-l5- phosphaneylidene)ethan-1-one 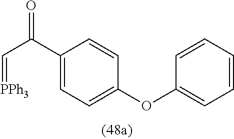 | ¹HNMR (CDCl₃, 400 MHz) δ: 7.95 (d, 2H, J = 8.4 Hz), 7.74-7.64 (m, 9H), 7.58-7.52 (m, 5H), 7.34-7.30 (m, 2H), 7.10-7.06 (m, 1H), 7.02-6.95 (m, 5H), 4.39 (s, 1H); Mass (LCMS): 473.1 (M + 1). |
| 5-bromo-1-(4-phenoxyphenyl)-4- phenylhexa-2,4-dien-1-one 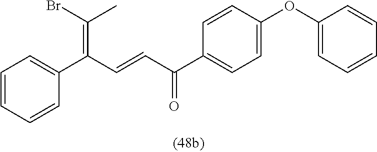 [SMILES: C/C(Br)=C(\C=C\C(=O)c1ccc(Oc2ccccc2)cc1)c1ccccc1] | ¹HNMR (CDCl₃, 400 MHz) δ: 8.46 (d, 1H, J = 14.8 Hz), 7.77-7.73 (m, 2H), 7.50-7.35 (m, 5H), 7.20-7.14 (m, 3H), 7.05-7.02 (m, 2H), 6.95-6.92 (m, 2H), 6.37 (d, 1H, J = 14.8 Hz), 2.29 (s, 3H); Mass (LCMS): 420.1 (M + 1). |
| 1-(4-bromophenyl)-2-(triphenyl-l5- phosphaneylidene)ethan-1-one 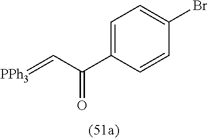 | ¹HNMR (DMSO-d₆, 400 MHz) δ: 7.82 (d, 2H, J = 8.8 Hz), 7.72-7.63 (m, 12H), 7.57-7.51 (m 5H), 4.38 (d, 1H, J = 20.8 Hz); Mass (LCMS): 459.1, 461.1 (M + 1). |
| 5-bromo-1-(4-bromophenyl)-4- phenylhexa-2,4-dien-1-one 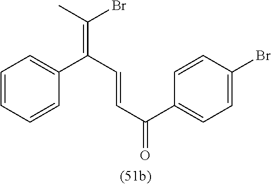 [SMILES: C/C(Br)=C(/C=C/C(=O)c1ccc(Br)cc1)c1ccccc1] | ¹HNMR (CDCl₃, 400 MHz) δ: 8.15 (d, 1H, J = 15.6 Hz), 7.60-7.59 (m, 3H), 7.55-7.52 (m, 3H), 7.16-7.11 (m, 3H), 6.36 (d, 1H, J = 15.6 Hz), 2.30 (s, 3H); Mass (LCMS): 406.1, 408.1 (M + 1). |
| 2-(2-Fluorophenyl)-1-(4-(2-(triphenyl-l5- phosphaneylidene)acetyl)piperidin-1- yl)ethan-1-one 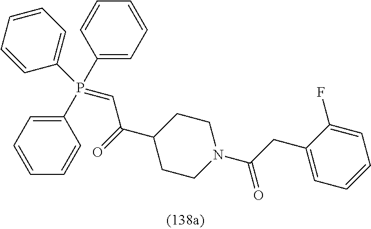 [SMILES: O=C(C=P(c1ccccc1)(c1ccccc1)c1ccccc1)C1CCN(C(=O)Cc2ccccc2F)CC1] | Mass (LCMS): 524.1 (M + 1) |
| 5-Bromo-1-(1-(2-(2- fluorophenyl)acetyl)piperidin-4-yl)-4- phenylhexa-2,4-dien-1-one 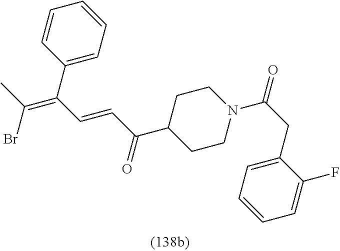 [SMILES: C/C(Br)=C(/C=C/C(=O)C1CCN(C(=O)Cc2ccccc2F)CC1)c1ccccc1] | Mass (LCMS): 471.9 (M + 1). |
| ethyl 4-(2-(triphenyl-λ⁵- phosphanylidene)acetyl)benzoate 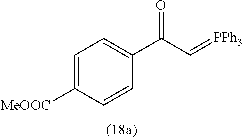 | ¹HNMR (CDCl₃, 400 MHz) δ: 8.03-7.98 (m, 4H), 7.74-7.64 (m, 6H), 7.59-7.53 (m, 3H), 7.50-7.44 (m, 6H), 4.47 (br.s, 1H), 3.91 (s, 3H); Mass (LCMS): 339.2 (M + 1). |
| 4-(5-Bromo-4-phenyl-hexa-2,4-dienoyl)- benzoic acid methyl ester 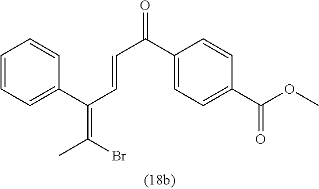 [SMILES: COC(=O)c1ccc(C(=O)/C=C/C(=C(/C)Br)c2ccccc2)cc1] | ¹HNMR (CDCl₃, 400 MHz) δ: 8.16 (d, 1H, J = 15.2 Hz), 8.07-8.04 (m, 2H), 7.80-7.77 (m, 2H), 7.48-7.36 (m, 3H), 7.17-7.12 (m, 2H), 6.34 (d, 1H, J = 15.2 Hz), 3.92 (s, 3H), 2.31 (s, 3H); Mass (LCMS): 386.2 (M + 1) |

### Example 19: Preparation of 1-(3-Chloro-phenyl)-2-(7-methyl-6-phenyl-5H-pyrrolo[1,2-c]imidazol-5-yl)-ethanol

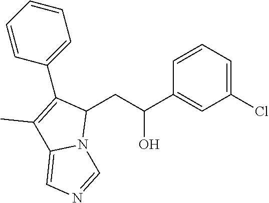 [SMILES: CC1=C(c2ccccc2)C(CC(O)c2cccc(Cl)c2)n2cncc21]

To a stirred solution of compound 1-(3-Chloro-phenyl)-2-(7-methyl-6-phenyl-5H-pyrrolo[1,2-c]imidazol-5-yl)-ethanone (Example 17, 10 mg, 0.0287 mmol) in methanol (1 mL) was added sodium borohydride (1.1 mg, 0.0287 mmol) at about 0° C. under nitrogen atmosphere and the reaction mixture was stirred at room temperature for about 0.5-1 hours. After completion of reaction, solvent was evaporated under reduced pressure to give the crude product. Water (5 mL) was added to the crude product and was extracted with ethyl acetate (3×10 mL). Combined organic layer was evaporated under reduced pressure and crude product was purified by trituration with n-hexanes to give pure compound 1-(3-Chloro-phenyl)-2-(7-methyl-6-phenyl-5H-pyrrolo[1,2-c]imidazol-5-yl)-ethanol (7 mg, 69.65%) as off white solid.

¹HNMR (CDCl₃, 400 MHz) δ: 7.45-7.39 (m, 2H), 7.37-7.29 (m, 2.5H), 7.24-7.16 (m, 5.5H) 7.10-7.01 (m, 1.4H), 6.86 (br. s, 0.6H), 5.46-5.43 (m, 0.4H), 5.22-5.17 (m, 0.6H), 5.01-4.98 (m, 0.4H), 4.74-4.71 (m, 0.6H), 2.15 (s, 3H), 2.11-2.07 (m, 2H); Mass (LCMS): 281.1 (M+1). Purity=93.97%.

Following examples have been synthesized by the above procedure described in Examples 17 & 19 with their corresponding intermediates in similar reaction conditions:

| Example | IUPAC | |
|---|---|---|
| No. | Name/Structure | Analytical Data |
| 20. | 1-(3-Fluoro-phenyl)-2- (7-methyl-6-phenyl-5H- pyrrolo[1,2-c]imidazol- 5-yl)-ethanone 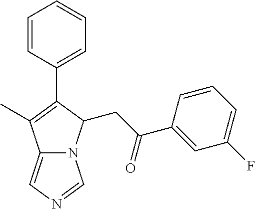 [SMILES: CC1=C(c2ccccc2)C(CC(=O)c2cccc(F)c2)n2cncc21] | ¹HNMR (CDCl₃, 400 MHz) δ: 7.70 (s, 1H), 7.65 (d, 1H, J = 2.4 Hz), 7.58-7.55 (m, 1H), 7.46-7.37 (m, 3H), 7.33-7.29 (m, 3H), 7.29-7.21 (m, 1H), 6.90 (s, 1H), 5.74 (d, 1H, J = 10.4 Hz), 3.32 (dd, 1H, J = 1.6 Hz, 18.8 Hz), 3.06 (dd, 1H, J = 10.4, 18.8 Hz), 2.22 (s, 3H); (LCMS): 333.0 (M + 1), Purity = 93.47%. |
| 28. | 1-Benzo[1,3]dioxol-5- yl-2-(7-methyl-6- phenyl-5H-pyrrolo[1,2- c]imidazol-5-yl)- ethanone 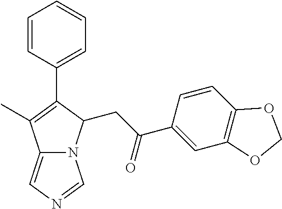 [SMILES: CC1=C(c2ccccc2)C(CC(=O)c2ccc3c(c2)OCO3)n2cncc21] | ¹HNMR (CDCl₃, 400 MHz) δ: 7.66(s, 1H), 7.45- 7.38 (m, 4H), 7.34-7.31 (m, 3H), 6.88 (s, 1H), 6.77 (d, 1H, J = 8.0 Hz), 6.02 (s, 2H), 5.75-5.72 (m, 1H), 3.25 (dd, 1H, J = 1.6 Hz & 8.4 Hz), 3.00 (dd, 1H, J = 10.8 Hz & 18.0 Hz), 2.20 (s, 3H); Mass (LCMS): 359.2 (M + 1), Purity = 94.78%. |
| 29. | 2-(7-Methyl-6-phenyl- 5H-pyrrolo[1,2- c]imidazol-5-yl)-1-(3- trifluoromethyl-phenyl)- ethanone 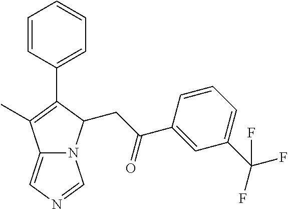 | ¹HNMR (CDCl₃, 400 MHz) δ: 8.10 (s, 1H), 8.04 (d, 1H, J = 7.6 Hz), 7.81 (d, 1H, J = 8.0 Hz), 7.66 (s, 1H), 7.57 (t, 1H, J = 8.0 Hz), 7.46- 7.42 (m, 2H), 7.35-7.32 (m, 3H), 6.89 (s, 1H), 5.78-5.75 (m, 1H), 3.36 (dd, 1H, J = 2.0 Hz & 18.4 Hz), 3.10 (dd, 1H, J = 10.4 Hz & 18.4 Hz), 2.23 (d, 3H, J = 1.6 Hz); Mass (LCMS): 363.2 (M + 1), Purity = 93.55% |
| 88. | 1-Cyclohexyl-2-(7- methyl-6-phenyl-5H- pyrrolo[1,2-c]imidazol- 5-yl)-ethanone 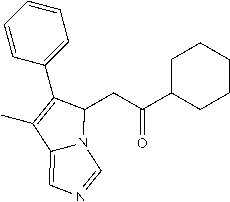 [SMILES: CC1=C(c2ccccc2)C(CC(=O)C2CCCCC2)n2cncc21] | ¹HNMR (CDCl₃, 400 MHz) δ: 7.57 (s, 1H), 7.44-7.41 (m, 2H), 7.34-7.31 (m, 1H), 7.28-7.27 (m, 2H), 6.88 (s, 1H), 5.57-5.54 (m, 1H), 2.84 (dd, 1H, J = 2.0, 18.4 Hz), 2.54 (dd, 1H, J = 10.7, 18.4 Hz), 2.18 (d, 3H, J = 1.8 Hz), 1.74- 1.72 (m, 5H), 1.64-1.60 (m, 1H), 1.21-1.16 (m, 5H); Mass (LCMS): 321.2 (M + 1). Purity = 94.42%. |
| 89. | 1-Cyclohexyl-2-(7- methyl-6-phenyl-5H- pyrrolo[1,2-c]imidazol- 5-yl)-ethane-1-ol 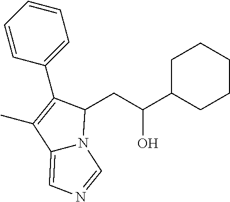 [SMILES: CC1=C(c2ccccc2)C(CC(O)C2CCCCC2)n2cncc21] | ¹HNMR (CDCl₃, 400 MHz) δ: 7.82 (s, 1H), 7.44-7.41 (m, 2H), 7.36-7.28 (m, 3H), 6.88 (s, 1H), 5.23 (s, 1H), 3.33-3.29 (m, 1H), 2.19 (d, 0.5H, J = 1.6 Hz), 2.16 (d, 2.5H, J = 2 Hz), 2.01- 1.94 (m, 1H), 1.80-1.73 (m, 2H), 1.29-1.27 (m, 2H), 1.21-1.0 (m, 5H), 0.97-0.81 (m, 4H); (LCMS): 323.3 (M + 1); Purity = 90.69%. |
| 90. | 1-Cyclohexyl-2-(7- methyl-6-phenyl-5H- pyrrolo[1,2-c]imidazol- 5-yl)ethan-1-ol, Isomer- I 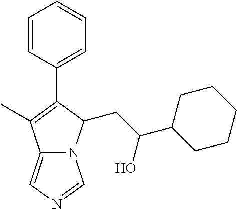 [SMILES: CC1=C(c2ccccc2)C(CC(O)C2CCCCC2)n2cncc21] | ¹HNMR (CDCl₃, 400 MHz) δ: 7.97 (s, 1H), 7.44 (t, 2H, J = 7.2 Hz), 7.35-7.30 (m, 3H), 6.92 (s, 1H), 5.45 (d, 1H, J = 10.8 Hz), 3.71-3.66 (m, 1H), 2.19 (d, 3H, J = 1.6 Hz), 1.89-1.60 (m, 6H), 1.51-1.39 (m, 2H), 1.26-1.21 (m, 1H), 1.18-1.08 (m, 3H), 0.97-0.84 (m, 2H); Mass (LCMS): 323.1 (M + 1); Purity: 94.45%. |
| 91. | 1-Cyclohexyl-2-(7- methyl-6-phenyl-5H- pyrrolo[1,2-c]imidazol- 5-yl)ethan-1-ol, Isomer- II 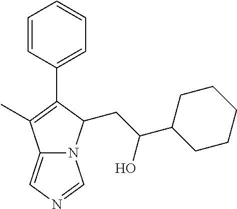 [SMILES: CC1=C(c2ccccc2)C(CC(O)C2CCCCC2)n2cncc21] | ¹HNMR (CDCl₃, 400 MHz) δ: 8.02 (s, 1H), 7.43 (t, 2H, J = 7.2 Hz), 7.33 (t, 1H, J = 7.6 Hz), 7.29 (d, 2H, J = 7.2 Hz), 6.90 (s, 1H), 5.27-5.24 (m, 1H), 3.39-3.35 (m, 1H), 2.16 (d, 3H, J = 1.6 Hz), 2.09-2.07 (m, 1H), 2.01-1.97 (m, 1H), 1.97-1.94 (m, 1H), 1.77-1.63 (m, 4H), 1.50 (d, 1H, J = 12.4 Hz), 1.24-1.02 (m, 4H), 0.94-0.84 (m, 2H); Mass (LCMS): 323.1 (M + 1); Purity: 95.73%. |
| 92. | 1-Cyclohexyl-2-(7- methyl-6-phenyl-5H- pyrrolo[1,2-c]imidazol- 5-yl)ethan-1-ol, Isomer- III 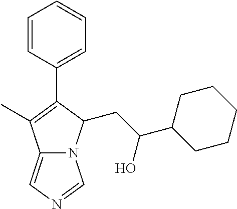 [SMILES: CC1=C(c2ccccc2)C(CC(O)C2CCCCC2)n2cncc21] | ¹HNMR (CDCl₃, 400 MHz) δ: 7.92 (s, 1H), 7.43 (t, 2H, J = 7.6 Hz), 7.35-7.03 (m, 3H), 6.91 (s, 1H), 5.44 (d, 1H, J = 10.4 Hz), 3.71-3.66 (m, 1H), 2.19 (d, 3H, J = 1.6 Hz), 1.89-1.60 (m, 6H), 1.51-1.39 (m, 2H), 1.28-1.21 (m, 1H), 1.18-1.05 (m, 3H), 0.97-0.84 (m, 2H). Mass (LCMS): 323.1 (M + 1); Purity: 91.85%. |
| 93. | 1-Cyclohexyl-2-(7- methyl-6-phenyl-5H- pyrrolo[1,2-c]imidazol- 5-yl)ethan-1-ol, Isomer- IV 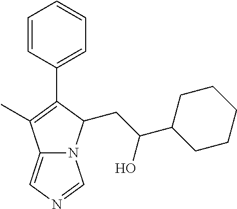 [SMILES: CC1=C(c2ccccc2)C(CC(O)C2CCCCC2)n2cncc21] | ¹HNMR (CDCl₃, 400 MHz): 8.10 (s, 1H), 7.44 (t, 2H, J = 7.6 Hz), 7.33 (t, 1H, J = 7.6 Hz), 7.29 (d, 2H, J = 7.2 Hz), 6.91 (s, 1H), 5.29-5.26 (m, 1H), 3.54-3.37 (m, 1H), 2.16 (d, 3H, J = 1.6 Hz), 1.99-1.94 (m, 2H), 1.76-1.48 (m, 6H), 1.22-1.03 (m, 4H), 0.94-0.85 (m, 2H). Mass (LCMS): 323.1 (M + 1); Purity: 96.91%. |
| 15. | 2-[6-(3-Fluoro- phenyl)-7-methyl-5H- pyrrolo[1,2-c] imidazol-5-yl]-1- phenyl-ethanone 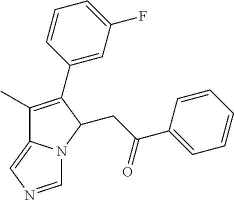 [SMILES: CC1=C(c2cccc(F)c2)C(CC(=O)c2ccccc2)n2cncc21] | ¹HNMR (CDCl₃, 400 MHz) δ: 7.88-7.86 (m, 2H), 7.68 (s, 1H), 7.59-7.56 (m, 1H), 7.45-7.39 (m, 3H), 7.10-7.01 (m, 3H), 6.91 (s, 1H), 5.74-5.72 (m, 1H), 3.34 (dd, 1H, J = 2.0, 18.3 Hz), 3.09 (dd, 1H, J = 10.7, 18.5 Hz), 2.24 (d, 3H, J = 1.8 Hz); Mass (LCMS): 333.2 (M + 1), Purity: 99.61% |
| 16. | 2-[6-(3-Fluoro- phenyl)-7-methyl-5H- pyrrolo[1,2-c] imidazol-5-yl]-1- phenyl-ethanol 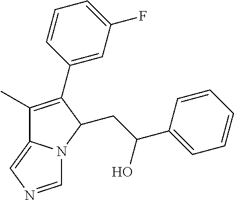 [SMILES: CC1=C(c2cccc(F)c2)C(CC(O)c2ccccc2)n2cncc21] | ¹HNMR (CDCl₃, 400 MHz) δ: 7.65-7.62 (m, 1H), 7.41-7.26 (m, 6H), 7.03-6.84 (m, 4H), 5.42-5.39 (m, 0.4H), 5.14-5.11 (m, 0.6H), 5.04-5.01 (m, 0.4H), 4.82-4.79 (m, 0.6H), 2.16 (s, 3H), 2.14- 2.01 (m, 2H); Mass (LCMS): 335.2 (M + 1); Purity: 90.95% |
| 23. | 3-[2-(7-Methyl-6- phenyl-5H-pyrrolo[1,2- c]imidazol-5-yl)- acetyl]-benzoic acid methyl ester 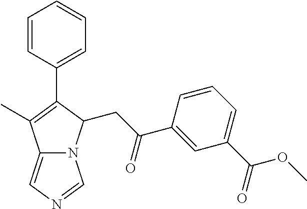 | ¹HNMR (CDCl₃, 400 MHz) δ: 8.43 (t, 1H, J = 2 Hz), 8.23 (dt, 1H, J = 2, 8 Hz), 8.11 (dt, 1H, J = 2, 8 Hz), 8.02 (s, 1H), 7.54 (t, 1H, J = 8 Hz), 7.46 (t, 2H, J = 7 Hz), 7.30-7.32(m, 3H), 7.07 (br. s, 1H), 5.84-5.81 (m, 1H), 3.92 (s, 3H), 3.43 (dd, 1H, J = 2.0, 18.8 Hz), 3.14 (dd, 1H, J = 10.8, 18.4 Hz), 2.22 (d, 3H, J = 1.7 Hz); Mass (LCMS): 373.1 (M + 1), Purity: 99.36%. |
| 24. | 4-[2-(7-Methyl-6- phenyl-5H-pyrrolo[1,2- c] imidazol-5-yl)- acetyl]-benzoic acid 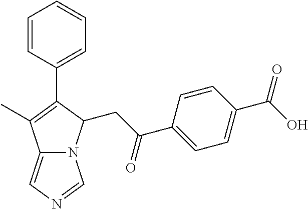 [SMILES: CC1=C(c2ccccc2)C(CC(=O)c2ccc(C(=O)O)cc2)n2cncc21] | ¹HNMR (MeOD, 400 MHz) δ: 8.39 (s, 1H), 8.33 (d, 2H, J = 8.8 Hz), 7.94 (d, 2H, J = 8.4 Hz), 7.51- 7.38 (m, 5H), 7.20 (s, 1H), 6.03 (d, 1H, J = 8.8 Hz), 3.60-3.59 (m, 1H), 3.57-3.55 (m, 1H), 2.24 (d, 3H, J = 2.0 Hz); LCMS (m/Z): 359.2, Purity: 90.10%. |
| 25. | 2-(7-Methyl-6-phenyl- 5H-pyrrolo[1,2- c]imidazol-5-yl)-1- naphthalen-2-yl- ethanone 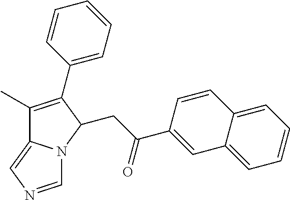 [SMILES: CC1=C(c2ccccc2)C(CC(=O)c2ccc3ccccc3c2)n2cncc21] | ¹HNMR (CDCl₃, 400 MHz) δ: 8.31 (br. s, 1H), 7.98-7.95 (m, 1H), 7.88-7.83 (m, 4H), 7.59 (t, 1H, J = 7.2 Hz), 7.52 (t, 1H, J = 7.6 Hz), 7.46 (t, 2H, J = 8.6 Hz), 7.73-7.33 (m, 3H), 6.97 (s, 1H), 5.85- 5.82(m, 1H), 3.49 (dd, 1H, J = 1.6, 18.3 Hz), 3.24 (dd, 1H, J = 10.8, 18.4 Hz), 2.23 (s, 3H); Mass (LCMS): 365.1 (M + 1); Purity: 95.17% |
| 26. | 1-Cyclopropyl-2-(7- methyl-6-phenyl-5H- pyrrolo[1,2-c]imidazol- 5-yl)-ethanone 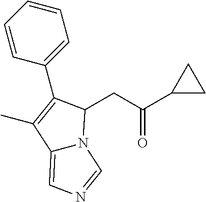 [SMILES: CC1=C(c2ccccc2)C(CC(=O)C2CC2)n2cncc21] | ¹HNMR (CDCl₃, 400 MHz) δ: 7.58 (s, 1H), 7.45- 7.42 (m, 2H), 7.36-7.32 (m, 1H), 7.29-7.27 (m, 2H), 6.88 (s, 1H), 5.56-5.53 (m, 1H), 3.01 (dd, 1H, J = 2, 18.4 Hz), 2.64 (dd, 1H, J = 10.8, 18.4 Hz), 2.18 (d, 3H, J = 1.8 Hz), 1.84-1.81 (m, 1H), 1.12-1.08 (m, 2H), 0.92-0.88 (m, 2H); Mass (LCMS): 278.2 (M + 1); Purity: 96.90% |
| 27. | 2-(7-Methyl-6-phenyl- 5H-pyrrolo[1,2- c]imidazol-5-yl)-1-p- tolyl-ethanone 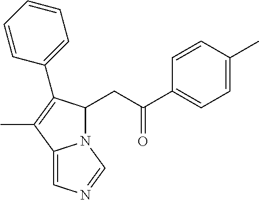 | ¹HNMR (CDCl₃, 400 MHz) δ: 8.62 (s, 1H), 7.75 (d, 2H, J = 8 Hz), 7.53-7.49 (m, 2H), 7.46-7.43 (m, 1H), 7.34 (d, 2H, J = 8 Hz), 7.28-7.22 (m, 3H), 5.95-5.92 (m, 1H), 3.53-3.49 (m, 1H), 3.09 (dd, 1H, J = 11.2, 18.4 Hz), 2.39 (s, 3H), 2.26 (s, 3H); Mass (LCMS): 329 (M + 1); Purity: 93.37%. |
| 32. | 1-(2-methoxyphenyl)- 2-(7-methyl-6-phenyl- 5H-pyrrolo[1,2- c]imidazol-5-yl)ethan- 1-one 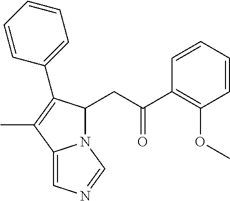 | ¹HNMR (CDCl₃) δ: 8.61 (s, 1H), 7.89-7.88 (m, 1H), 7.53-7.48 (m, 2H), 7.45-7.41 (m, 1H), 7.34- 7.32 (m, 2H), 7.23 (s, 1H), 7.06-7.02 (m, 1H), 7.04 (t, 1H, J = 7.6 Hz), 6.90 (d, 1H, J = 8.8 Hz), 5.89 (d, 1H, J = 10.8 Hz), 3.80 (s, 3H), 3.57 (dd, 1H, J = 1.6, 19.2 Hz), 3.12 (dd, 1H, J = 10.8, 19.2 Hz), 2.23 (s, 3H, J = 1.6 Hz); Mass (LCMS): 345.1 (M + 1); Purity: 91.5%. |
| 33. | 1-(3-Methoxy-phenyl)- 2-(7-methyl-6-phenyl- 5H-pyrrolo[1,2- c]imidazol-5-yl)- ethanone 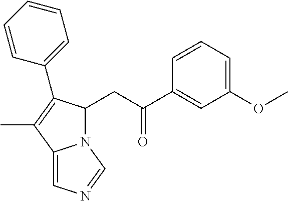 | ¹HNMR (CDCl₃) δ: 8.63 (s, 1H), 7.53-7.50 (m, 2H), 7.47-7.43 (m, 2H), 7.37-7.28 (m, 4H), 7.24 (br. s, 1H), 7.15-7.13 (m, 1H), 5.92 (d, 1H, J = 10.8 Hz), 3.85 (s, 3H), 3.53 (dd, 1H, J = 1.2, 18.8 Hz), 3.10 (dd, 1H, J = 10.4, 18.8 Hz), 2.26 (d, 3H, J = 1.2 Hz); Mass (LCMS): 345.2 (M + 1); Purity: 90.89%. |
| 34. | 1-(4-fluorophenyl)-2- (7-methyl-6-phenyl- 5H-pyrrolo[1,2- c]imidazol-5-yl)ethan- 1-one 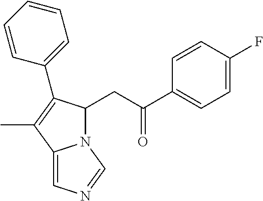 [SMILES: CC1=C(c2ccccc2)C(CC(=O)c2ccc(F)cc2)n2cncc21] | ¹HNMR (CDCl₃, 400 MHz) δ: 7.91-7.87 (m, 2H), 7.66 (s, 1H), 7.46-7.42 (m, 2H), 7.35-7.31 (m, 3H), 7.09 (t, 2H, J = 8.4 Hz), 6.88 (s, 1H), 5.74 (d, 1H, J = 10.4 Hz), 3.31 (dd, 1H, J = 1.6, 18.4 Hz), 3.05 (dd, 1H, J = 10.8, 18.4 Hz), 2.22 (d, 3H, J = 1.6 Hz); Mass (LCMS): 333.1 (M + 1); Purity: 92.8%. |
| 35. | 1-(4-fluorophenyl)-2- (7-methyl-6-phenyl- 5H-pyrrolo[1,2- c]imidazol-5-yl)ethan- 1-ol 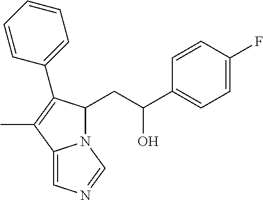 [SMILES: CC1=C(c2ccccc2)C(CC(O)c2ccc(F)cc2)n2cncc21] | ¹HNMR (CDCl₃, 400 MHz) δ: 7.87 (s, 0.4H), 7.68 (s, 0.6H), 7.44-7.29 (m, 4H), 7.23-7.16 (m, 3H), 7.02-6.97 (m, 2H), 6.91 (s, 0.4H), 6.85 (s, 0.6H), 5.43-5.41 (m, 0.4H), 5.17-5.14 (m, 0.6H), 5.0 (dd, 0.4H, J = 3.6, 10 Hz), 4.73-4.70 (m, 0.6H), 2.17- 2.15 (m, 5H). Mass (LCMS): 335.1 (M + 1); Purity: 93%. |
| 36. | 1-(2,5-difluorophenyl)- 2-(7-methyl-6-phenyl- 5H-pyrrolo[1,2- c]imidazol-5-yl)ethan- 1-one 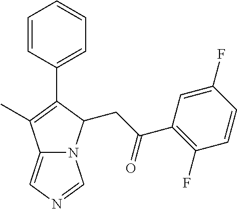 [SMILES: CC1=C(c2ccccc2)C(CC(=O)c2cc(F)ccc2F)n2cncc21] | ¹HNMR (CDCl₃, 400 MHz) δ: 8.64 (s, 1H), 7.71- 7.67 (m, 1H), 7.53-7.43 (m, 3H), 7.34-7.32 (m, 2H), 7.26-7.22 (m, 2H), 7.10-7.05 (m, 1H), 5.91- 5.89 (m, 1H), 3.55-3.49 (m, 1H), 3.16-3.08 (m, 1H), 2.24 (d, 3H, J = 2.0 Hz); LCMS: 351.1 (M + 1); Purity: 97.34%. |
| 37. | 1-(4-methoxyphenyl)- 2-(7-methyl-6-phenyl- 5H-pyrrolo[1,2- c]imidazol-5-yl)ethan- 1-one 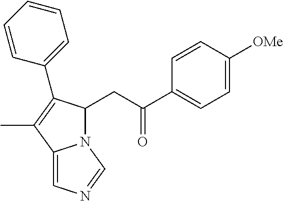 [SMILES: COc1ccc(C(=O)CC2C(c3ccccc3)=C(C)c3cncn32)cc1] | ¹HNMR (CDCl₃, 400 MHz) δ: 7.84 (d, 2H, J = 8.8 Hz), 7.67 (br. s, 1H), 7.45-7.41 (m, 2H), 7.34- 7.32 (m, 3H), 6.89-6.87 (m, 3H), 5.77-5.74 (m, 1H), 3.84 (s, 3H), 3.28 (dd, 1H, J = 2.4 and 18.0 Hz), 3.03 (dd, 1H, J = 10.8 and 18.0 Hz), 2.22 (s, 3H); Mass (LCMS): 345.1 (M + 1); Purity: 97.68%. |
| 38. | 1-(4-chlorophenyl)-2- (7-methyl-6-phenyl- 5H-pyrrolo[1,2- c]imidazol-5-yl)ethan- 1-one 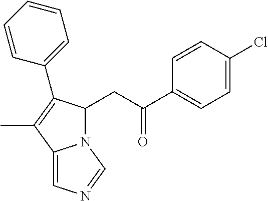 [SMILES: CC1=C(c2ccccc2)C(CC(=O)c2ccc(Cl)cc2)n2cncc21] | ¹HNMR (CDCl₃, 400 MHz) δ: 7.80 (d, 2H, J = 8.4 Hz), 7.67 (br. s, 1H), 7.46-7.38 (m, 4H), 7.35- 7.31 (m, 3H), 6.89 (br. s, 1H), 5.75-5.73 (m, 1H), 3.31 (dd, 1H, J = 2.0 and 18.4 Hz), 3.04 (dd, 1H, J = 10.8 and 18.4 Hz), 2.22 (d, 3H, 1.2 Hz); Mass (LCMS): 349.1 (M + 1); Purity: 94.18%. |
| 39. | 1-(3-chloro-4- fluorophenyl)-2-(7- methyl-6-phenyl-5H- pyrrolo[1,2-c]imidazol- 5-yl)ethan-1-one 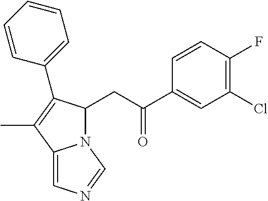 [SMILES: CC1=C(c2ccccc2)C(CC(=O)c2ccc(F)c(Cl)c2)n2cncc21] | ¹HNMR (CDCl₃, 400 MHz) δ: 7.95-7.93 (m, 1H), 7.79-7.75 (m, 1H), 7.65 (br. s, 1H), 7.45 (t, 2H, J = 7.5 Hz), 7.36-7.31 (m, 3H), 7.18 (t, 1H, J = 8.4 Hz), 6.89 (br. s, 1H), 5.74-5.72 (m, 1H), 3.29 (dd, 1H, J = 2.0 and 18.4 Hz), 3.03 (dd, 1H, J = 10.8 and 18.4 Hz), 2.22 (d, 3H, J = 1.6 Hz); Mass (LCMS): 367.0 (M + 1); Purity: 96.74%. |
| 40. | 2-(7-methyl-6-phenyl- 5H-pyrrolo[1,2- c]imidazol-5-yl)-1- (naphthalen-1-yl)ethan- 1-one 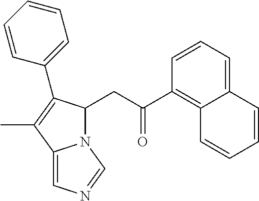 [SMILES: CC1=C(c2ccccc2)C(CC(=O)c2cccc3ccccc23)n2cncc21] | ¹HNMR (CDCl₃, 400 MHz) δ: 8.78 (d, 1H, J = 8.8 Hz), 7.98 (d, 1H, J = 8.0 Hz), 7.86 (d, 1H, J = 8.0 Hz), 7.81 (s, 1H), 7.73-7.63 (m, 2H), 7.55 (t, 1H, J = 7.2 Hz), 7.44-7.39 (m, 3H), 7.37- 7.31(m, 3H), 6.91 (s, 1H), 5.84 (d, 1H, J = 10.4 Hz), 3.42 (d, 1H, J = 18.0 Hz), 3.20 (dd, 1H, J = 10.4, 18 Hz Hz), 2.22 (s, 3H); Mass (LCMS): 365.1 (M + 1); Purity: 93.57%. |
| 41. | 1-([1,1′-biphenyl]-4- yl)-2-(7-methyl-6- phenyl-5H-pyrrolo[1,2- c]imidazol-5-yl)ethan- 1-one 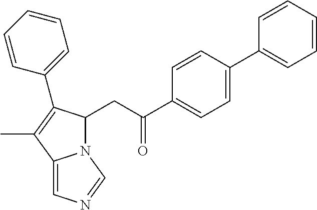 [SMILES: CC1=C(c2ccccc2)C(CC(=O)c2ccc(-c3ccccc3)cc2)n2cncc21] | ¹HNMR (CDCl₃, 400 MHz) δ: 7.93 (d, 2H, J = 8.0 Hz), 7.73 (d, 1H, J = 8.0 Hz), 7.64 (d, 2H, J = 8.0 Hz), 7.58 (d, 2H, J = 8.0 Hz), 7.47-7.43 (m, 5H), 7.35-7.33 (m, 3H), 6.90 (s, 1H), 5.79 (d, 1H, J = 10.4 Hz), 3.40-3.05 (m, 1H), 3.11 (dd, 1H, J = 10.8, 18.4 Hz), 2.23 (s, 3H); Mass (LCMS): 391 (M + 1); Purity: 91.76%. |
| 42. | 1-([1,1′-biphenyl]-4- yl)-2-(7-methyl-6- phenyl-5H-pyrrolo[1,2- c]imidazol-5-yl)ethan- 1-one, Isomer-I 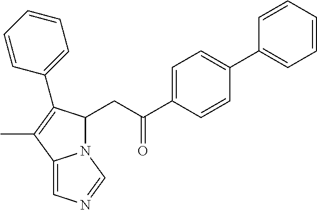 [SMILES: CC1=C(c2ccccc2)C(CC(=O)c2ccc(-c3ccccc3)cc2)n2cncc21] | ¹HNMR (CDCl₃, 400 MHz) δ: 8.25 (s, 1H), 7.93 (d, 2H, J = 8.0 Hz), 7.65 (d, 2H, J 8.0 Hz), 7.58 (d, 2H, J = 6.8 Hz), 7.51-7.41 (m, 6H), 7.40-7.36 (m, 2H), 7.10 (s, 1H), 5.89 (d, 1H, J = 10.4 Hz), 3.54 (d, 1H, J = 18.0 Hz), 3.15 (dd, 1H, J = 10.8 and 18.4 Hz), 2.25 (s, 3H); Mass (LCMS): 391.0 (M + 1); Purity: 97.37%. |
| 43. | 1-(1,1′-biphenyl]-4- yl)-2-(7-methyl-6- phenyl-5H-pyrrolo[1,2- c]imidazol-5-yl)ethan- 1-one, Isomer-II 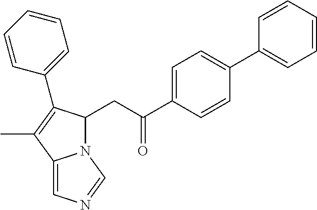 [SMILES: CC1=C(c2ccccc2)C(CC(=O)c2ccc(-c3ccccc3)cc2)n2cncc21] | ¹HNMR (CDCl₃, 400 MHz) δ: 8.26 (s, 1H), 7.93 (d, 2H, J = 8.0 Hz), 7.65 (d, 2H, J 8.0 Hz), 7.58 (d, 2H, J = 6.8 Hz), 7.51-7.40 (m, 8H), 7.10 (s, 1H), 5.89 (d, 1H, J = 10.4 Hz), 3.54 (d, 1H, J = 18.0 Hz), 3.15 (dd, 1H, J = 10.8, 18.4 Hz), 2.25 (s, 3H), Mass (LCMS): 391.0 (M + 1); Purity: 97%. |
| 173. | Ethyl 5-(7-methyl-6- phenyl-5H- pyrrolo[1,2- c]imidazol-5-yl)-4- oxopentanoate 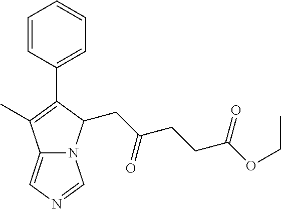 [SMILES: CCOC(=O)CCC(=O)CC1C(c2ccccc2)=C(C)c2cncn21] | ¹HNMR (CDCl₃, 400 MHz) δ: 7.59 (s, 1H), 7.44- 7.41 (m, 2H), 7.35-7.31 (m, 1H), 7.27-7.25 (m, 2H), 6.87 (s, 1H), 5.55 (d, 1H, J = 10.4 Hz), 4.13 (m, 2H), 2.91 (dd, 1H, J = 2, 18.2 Hz), 2.69-2.51 (m, 5H), 2.18 (d, 3H, J = 2 Hz), 1.25 (t, 3H, J = 4.4 Hz); Mass (LCMS): 339.1 (M + 1); Purity: 96.69%. |
| 44. | 1-([1,1′-biphenyl]-4- yl)-2-(7-methyl-6- phenyl-5H-pyrrolo[1,2- c]imidazol-5-yl)ethan- 1-ol 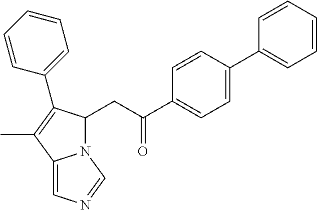 [SMILES: CC1=C(c2ccccc2)C(CC(=O)c2ccc(-c3ccccc3)cc2)n2cncc21] | ¹HNMR (CDCl₃, 400 MHz) δ: 7.74 (br. s, 0.4H), 7.56-7.50 (m, 3.6H), 7.44-7.39 (m, 3H), 7.37- 7.28 (m, 5H), 7.27 (s, 2H), 7.20 (d, 1H, J = 7.2 Hz), 6.94 (br. s, 0.4H), 6.88 (s, 0.6H), 5.48-5.46 (m, 0.4H), 5.19-5.17 (m, 0.6H), 5.09-5.06 (m, 0.4H), 4.84-4.81 (m, 0.6H), 2.2-2.16 (m, 4H), 1.72-1.64 (m, 1H); Mass (LCMS): 393.1 (M + 1); Purity: 93.8%. |
| 45. | 2-(7-methyl-6-phenyl- 5H-pyrrolo[1,2- c]imidazol-5-yl)-1- (thiophen-3-yl)ethan-1- one 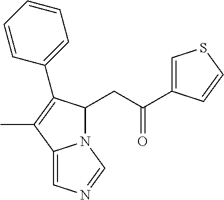 [SMILES: CC1=C(c2ccccc2)C(CC(=O)c2ccsc2)n2cncc21] | ¹HNMR (CDCl₃, 400 MHz) δ: 7.94 (d, 1H, J = 8.0 Hz), 7.67 (s, 1H), 7.50 (d, 1H, J = 8.0 Hz), 7.45-7.41 (m, 2H), 7.34-7.29 (m, 4H), 6.88 (s, 1H), 5.75 (d, 1H, J = 10.8 Hz), 3.27 (d, 1H, J = 2 Hz), 2.99 (dd, 1H, J = 10.8 Hz and 18.8 Hz), 2.22 (d, 3H, J = 1.6 Hz); Mass (LCMS): 320.9 (M + 1); Purity: 97.65%. |
| 46. | 1-(4- (dimethylamino)phenyl)- 2-(7-methyl-6- phenyl-5H-pyrrolo[1,2- c]imidazol-5-yl)ethan- 1-one 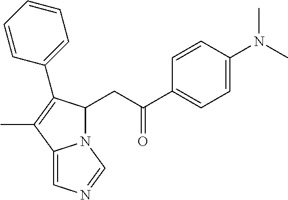 | ¹HNMR (CDCl₃, 400 MHz) δ: 7.75 (d, 2H, J = 9.2 Hz), 7.68 (br. s, 1H), 7.45-7.41 (m, 2H), 7.34-7.32 (m, 3H), 6.87 (br. s, 1H), 6.58 (d, 2H, J = 9.2 Hz), 5.80-5.77 (m, 1H), 3.22 (dd, 1H, J = 2.0, 18.0 Hz), 3.03 (s, 6H), 2.99 (dd, 1H, J = 10.8, 18.0 Hz), 2.22 (d, 3H, J = 2.0 Hz); Mass (LCMS): 358.0 (M + 1); Purity: 97.1%. |
| 47. | 2-(7-methyl-6-phenyl- 5H-pyrrolo[1,2- c]imidazol-5-yl)-1- (5,6,7,8- tetrahydronaphthalen- 2-yl)ethan-1-one 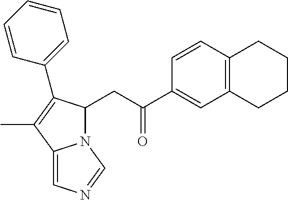 [SMILES: CC1=C(c2ccccc2)C(CC(=O)c2ccc3c(c2)CCCC3)n2cncc21] | ¹HNMR (CD₃OD, 400 MHz) δ: 8.78 (br. s, 1H), 7.50-7.49 (m, 2H), 7.42-7.35 (m, 6H), 7.02 (d, 1H, J = 8.0 Hz), 6.08-6.06 (m, 1H), 3.46 (dd, 1H, J = 2.4, 18.4 Hz), 3.27 (dd, 1H, J = 10.0, 18.4 Hz), 2.68-2.65 (m, 4H), 2.17 (d, 3H, J = 2.0 Hz), 1.70-1.67 (m, 4H); Mass (LCMS): 369.1 (M + 1); Purity: 94.95%. |
| 48. | 2-(7-methyl-6-phenyl- 5H-pyrrolo[1,2- c]imidazol-5-yl)-1-(4- phenoxyphenyl)ethan- 1-one 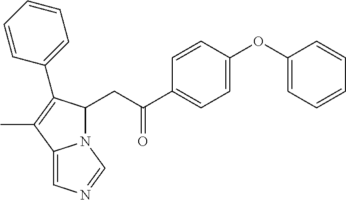 [SMILES: CC1=C(c2ccccc2)C(CC(=O)c2ccc(Oc3ccccc3)cc2)n2cncc21] | ¹HNMR (CDCl₃, 400 MHz) δ: 8.62 (s, 1H), 7.83 (d, 2H, J = 8.8 Hz), 7.53-7.44 (m, 3H), 7.41-7.32 (m, 4H), 7.23-7.19 (m, 2H), 7.36 (dd, 2H, J = 8.6 Hz), 6.95 (d, 2H, J = 8.8 Hz), 5.94 (d, 1H, J = 11.2 Hz), 3.48 (dd, 1H, J = 2, 18.4 Hz), 3.63 (dd, 1H, J = 11.2, 18.4 Hz), 2.25 (d, 3H, J = 1.2 Hz); Mass (LCMS): 408.1 (M + 1); Purity: 98%. |
| 49. | 2-(7-methyl-6-phenyl- 5H-pyrrolo[1,2- c]imidazol-5-yl)-1-(4- morpholinophenyl)ethan- 1-one 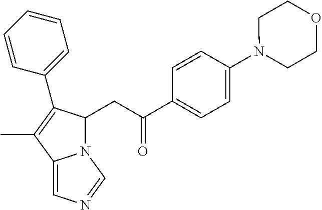 [SMILES: CC1=C(c2ccccc2)C(CC(=O)c2ccc(N3CCOCC3)cc2)n2cncc21] | ¹HNMR (CDCl₃, 400 MHz) δ: 7.78 (d, 2H, J = 9.2 Hz), 7.67 (s, 1H), 7.45-7.41 (m, 2H), 7.34- 7.29 (m, 3H), 6.87 (s, 1H), 6.79 (d, 2H, J = 9.2 Hz), 5.77 (d, 1H, J = 9.6 Hz), 3.83 (t, 4H, 4.8 Hz), 3.29 (t, 4H, J = 4.8 Hz), 3.22-3.21 (m, 1H), 3.00 (dd, 1H, J = 10.8, 18.6 Hz), 2.22 (d, 3H, J = 1.6 Hz), Mass (LCMS): 400.1 and 401.1 (M + 1); Purity: 89%. |
| 50. | 2-(7-methyl-6-phenyl- 5H-pyrrolo[1,2- c]imidazol-5-yl)-1-(4- (piperidin-1- yl)phenyl)ethan-1-one 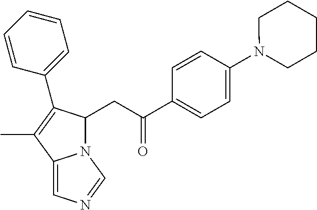 [SMILES: CC1=C(c2ccccc2)C(CC(=O)c2ccc(N3CCCCC3)cc2)n2cncc21] | ¹HNMR (CDCl₃, 400 MHz) δ: 7.74 (d, 2H, J = 8.8 Hz), 7.69 (br. s, 1H), 7.43 (t, 2H, J = 8.0 Hz), 7.34-7.30 (m, 3H), 6.87 (br. s, 1H), 6.77 (d, 2H, J = 9.2 Hz), 5.79-5.76 (m, 1H), 3.34 (br. s, 4H), 3.22 (dd, 1H, J = 2.0, 18.0 Hz), 2.99 (dd, 1H, J = 10.8, 18.0 Hz), 2.22 (s, 3H), 1.70 (br. s, 2H), 1.64 (br. s, 4H); Mass (LCMS): 398.1 (M + 1); Purity: 99%. |
| 51. | 1-(4-bromophenyl)-2- (7-methyl-6-phenyl- 5H-pyrrolo[1,2- c]imidazol-5-yl)ethan- 1-one 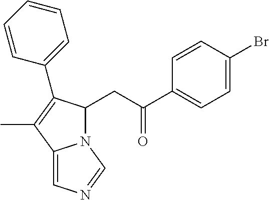 | ¹HNMR (CDCl₃, 400 MHz) δ: 7.72 (d, 2H, J = 8.8 Hz), 7.66 (s, 1H), 7.56 (d, 2H, J = 8.4 Hz), 7.44 (t, 2H, J = 7.6 Hz), 7.35-7.31 (m, 3H), 6.88 (s, 1H), 5.73 (d, 1H, 10.4 Hz), 3.30 (d, 1H, J = 18.8 Hz), 3.03 (dd, 1H, J = 11.2, 18.8 Hz), 2.22 (d, 3H, J = 1.6 Hz ); Mass (LCMS): 363.1. 364 (M + 1); Purity: 100%. |
| 52. | 1-(4-bromophenyl)-2- (7-methyl-6-phenyl- 5H-pyrrolo[1,2- c]imidazol-5-yl)ethan- 1-ol 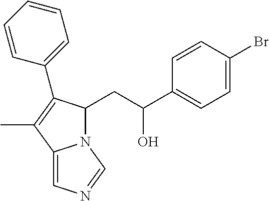 [SMILES: CC1=C(c2ccccc2)C(CC(O)c2ccc(Br)cc2)n2cncc21] | ¹HNMR (CDCl₃, 400 MHz) δ: 7.86 (s, 0.35H), 7.70 (s, 0.65H), 7.44-7.41 (m, 2H), 7.39-7.29 (m, 3H), 7.24 (s, 1H), 7.18 (d, 1H, J = 7.6 Hz), 7.10 (d, 2H, J = 7.6 Hz), 6.87 (s, 0.35H), 6.83 (s, 0.65H), 5.42 (d, 0.35H, J = 9.2 Hz), 5.16-5.14 (m, 0.65H), 4.97 (dd, 0.35H, J = 2.4, 9.2 Hz), 4.71- 4.67 (m, 0.65H), 2.15 (s, 3H). 2.10-2.06 (m, 2H); Mass (LCMS): 396.1 (M + 1); Purity: 99.99%. |
| 138. | 2-(2-Fluorophenyl)-1- (4-(2-(7-methyl-6- phenyl-5H-pyrrolo[1,2- c]imidazol-5- yl)acetyl)piperidin-1- yl)ethan-1-one 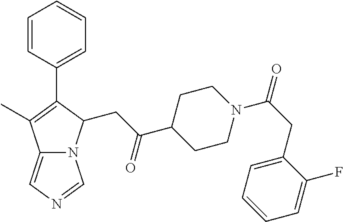 [SMILES: CC1=C(c2ccccc2)C(CC(=O)C2CCN(C(=O)Cc3ccccc3F)CC2)n2cncc21] | ¹HNMR (CDCl₃, 400 MHz) δ: 7.59 (s, 1H), 7.43 (t, 2H, J = 7.6 Hz), 7.34 (t, 1H, J = 7.2 Hz), 7.25- 7.21(m, 3H), 7.11-7.02 (m, 3H), 6.90 (s, 1H), 5.53 (d, 1H, J = 7.6 Hz), 4.53 (d, 1H, J = 13.6 Hz), 3.88 (d, 1H, J = 13.2 Hz), 3.69 (s, 2H), 3.03-2.96 (m 1H), 2.85 (t, 1H, J = 16.8 Hz), 2.66-2.41 (m, 3H), 2.18 (s, 3H), 1.81-1.68 (m, 2H), 1.47-1.37 (m, 2H); Mass (LCMS): 458.3 (M + 1); Purity: 90.81%. |
| 139. | 2-(2-Fluorophenyl)-1- (4-(1-hydroxy-2-(7- methyl-6-phenyl-5H- pyrrolo[1,2-c]imidazol- 5-yl)ethyl)piperidin-1- yl)ethan-1-one 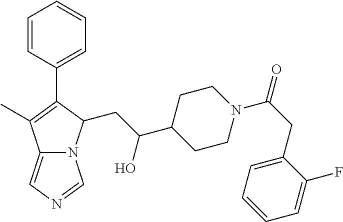 [SMILES: CC1=C(c2ccccc2)C(CC(O)C2CCN(C(=O)Cc3ccccc3F)CC2)n2cncc21] | ¹HNMR (CDCl₃, 400 MHz) δ: 7.91 (br. s, 1H), 7.44 (t, 2H, J = 7.6 Hz), 7.34 (t, 1H, J = 7.2 Hz), 7.29 (d, 2H, J = 8 Hz), 7.24-7.19 (m, 2H), 7.09- 6.99 (m, 2H), 6.89 (bs, 1H), 5.25 (s, 1H), 4.66- 4.57 (m, 1H), 3.91-3.80 (m, 1H), 3.71-3.61 (m, 3H), 3.32 (br. s, 1H), 2.88 (t, 1H, J = 13.2 Hz), 2.43 (t, 2H, J = 12.4 Hz), 2.18 (d, 3H, J = 12 Hz), 1.47-1.38 (m, 3H), 1.33-1.29(m, 1H), 1.13-0.95 (m, 2H); Mass (LCMS): 460.1 (M + 1); Purity: 91.07%. |
| 18. | 4-[2-(7-Methyl-6- phenyl-5H-pyrrolo[1,2- c] imidazol-5-yl)- acetyl]-benzoic acid methyl ester 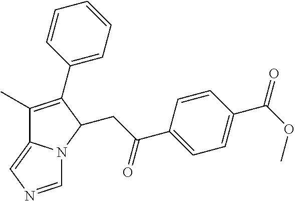 [SMILES: COC(=O)c1ccc(C(=O)CC2C(c3ccccc3)=C(C)c3cncn32)cc1] | ¹HNMR (CDCl₃, 400 MHz) δ: 8.08 (d, 2H, J = 8.0 Hz), 7.91 (d, 2H, J = 8 Hz), 7.68 (s, 1H), 7.46- 7.42 (m, 2H), 7.35-7.32 (m, 3H), 6.89 (s, 1H), 5.76-5.74 (m, 1H), 3.93 (s, 3H), 3.37 (dd, 1H, J = 2.0, 18.4 Hz), 3.10 (dd, 1H, J = 10.7, 18.6 Hz), 2.22 (d, 3H, J = 1.6 Hz); Mass (LCMS): 373.2 (M + 1), Purity: 95.3% |
| 71. | 4-[2-(7-Methyl-6- phenyl-5H-pyrrolo[1,2- c]imidazol-5-yl)- acetyl]-benzoic acid methyl ester, Isomer-I 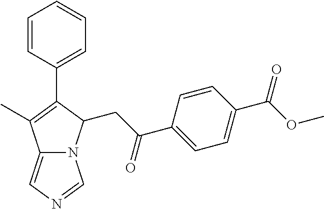 | ¹HNMR (CDCl₃, 400 MHz) δ: 8.24 (s, 1H), 8.00 (d, 2H, J = 8.4 Hz), 7.91 (d, 2H, J = 8.4 HZ), 7.49 (t, 2H, J = 8 Hz), 7.42-7.40 (m, 1H), 7.34 (d, 2H, J = 7.2 Hz), 7.09 (s, 1H), 5.86 (d, 1H, J = 10.8 Hz), 3.93 (s, 3H), 3.48 (dd, 1H, J = 2, 18.8 Hz), 3.14 (dd, 1H, J = 11.2, 18.8 Hz), 2.24 (d, 3H, J = 1.6 Hz); Mass (LCMS): 373.2 (M + 1), Purity = 98.55%. |
| 72. | 4-12-(7-Methyl-6- phenyl-5H-pyrrolo[1,2- c]imidazol-5-yl)- acetyl]-benzoic acid methyl ester, Isomer-II 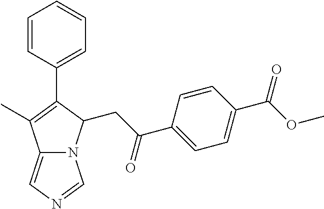 | ¹HNMR (CDCl₃, 400 MHz) δ: 8.50 (s, 1H), 8.09 (d, 2H, J = 8.4 Hz), 7.91 (d, 2H, J = 8.4 HZ), 7.51 (t, 2H, J = 8 Hz), 7.45-7.42 (m, 1H), 7.34 (d, 2H, J = 7.2 Hz), 7.18 (s, 1H), 5.90 (d, 1H, J = 10.8 Hz), 3.93 (s, 3H), 3.53 (dd, 1H, J = 2, 18.8 Hz), 3.15 (dd, 1H, J = 11.2, 18.8 Hz), 2.25 (d, 3H, J = 1.6 Hz); Mass (LCMS): 373.2 (M + 1), Purity = 99.09%. |
| 68. | 1-(3-Bromo-phenyl)-2- (7-methyl-6-phenyl- 5H-pyrrolo[1,2- c]imidazol-5-yl)- ethanone 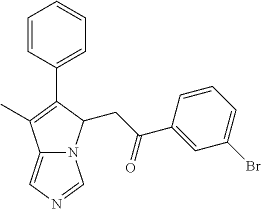 [SMILES: CC1=C(c2ccccc2)C(CC(=O)c2cccc(Br)c2)n2cncc21] | ¹HNMR (CDCl₃, 400 MHz) δ: 7.98-7.97 (m, 1H), 7.78 (d, 1H, J = 8.0 Hz), 7.69-7.67 (m, 2H), 7.46- 7.44 (m, 3H), 7.35-7.31 (m, 3H), 6.89 (s, 1H), 5.73 (d, 1H, J = 10.8 Hz), 3.30 (dd, 1H, J = 2.0, 16 Hz), 3.09-3.02 (m, 1H), 2.26 (d, 3H, J = 1.6 Hz); Mass (LCMS): 393.1; Purity: 90.43%. |
| 75. | 1-(4- (benzyloxy)phenyl)-2- (7-methyl-6-phenyl- 5H-pyrrolo[1,2- c]imidazol-5-yl)ethan- 1-one 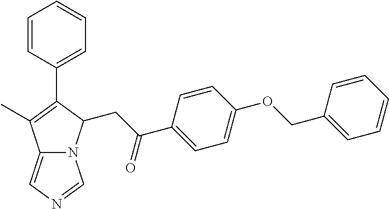 [SMILES: CC1=C(c2ccccc2)C(CC(=O)c2ccc(OCc3ccccc3)cc2)n2cncc21] | ¹HNMR (CDCl₃, 400 MHz) δ: 7.84 (dd, 1H, J = 1.6, 6.8 Hz), 7.67 (s, 1H), 7.45-7.35 (m, 5H), 7.34-7.23 (m, 5H), 7.17-7.13 (m, 1H), 6.97-6.94 (m, 2H), 6.88 (s, 1H), 5.75 (d, 1H, J = 10.4 Hz), 5.10 (s, 2H), 3.29 (dd, 1H, J = 2, 18 Hz), 3.04 (dd, 1H, J = 10.8, 18 Hz), 2.22 (d, 3H, J = 1.6 Hz); Mass (LCMS): 421.1 (M + 1); Purity: 88.36%. |
| 76. | 1-(4-(2-fluorophenoxy) phenyl)-2-(7-methyl-6- phenyl-5H-pyrrolo[1,2- c]imidazol-5-yl)ethan- 1-one 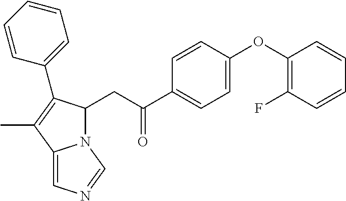 [SMILES: CC1=C(c2ccccc2)C(CC(=O)c2ccc(Oc3ccccc3F)cc2)n2cncc21] | ¹HNMR (CDCl₃, 400 MHz) δ: 7.87-7.79 (m, 2H), 7.75 (s, 1H), 7.45-7.39 (m, 2H), 7.38-7.33 (m, 3H), 7.23-7.18 (m, 2H), 7.17-7.10 (m, 2H), 6.93- 6.87 (m, 3H), 5.75 (d, 1H, J = 10.4 Hz), 3.30 (dd, 1H, J = 1.6, 18 Hz), 3.05 (dd, 1H, J = 10.8, 18 Hz), 2.21 (d, 3H, J = 1.6 Hz). Mass (LCMS): 425.1 (M + 1); Purity: 92.99%. |

### Example 53: Preparation of 2-(7-methyl-6-phenyl-5H-pyrrolo[1,2-c]imidazol-5-yl)-1-(4-(pyridin-4-yl)phenyl)ethan-1-one

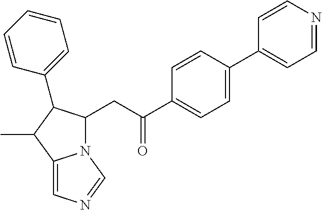 [SMILES: CC1c2cncn2C(CC(=O)c2ccc(-c3ccncc3)cc2)C1c1ccccc1]

To a mixture of bromo compound 1-(4-bromophenyl)-2-(7-methyl-6-phenyl-5H-pyrrolo[1,2-c]imidazol-5-yl)ethan-1-one (Example 51, 50 mg, 0.137 mmol) and pyridine-4-boronic acid (22 mg, 0.179 mmol) in 1,4-dioxane:water (4:1; 10 mL) was added K₂CO₃ (38 mg, 0.27 mmol) at room temperature. The reaction mixture was degassed for 10 min using nitrogen gas. To this suspension Pd(dppf)Cl₂-DCM complex (12 mg, 0.00137 mmol) was added and reaction mixture was degassed for another five minutes. Reaction mixture was heated at 100° C. for 3-5 h. After completion of reaction, solvent was evaporated under reduced pressure and residue was diluted with water (25 mL) and extracted with ethyl acetate (3×25 mL). Combined organic layer was dried over anhydrous sodium sulfate and concentrated to give crude product which was purified by column chromatography to yield title compound 2-(7-methyl-6-phenyl-5H-pyrrolo[1,2-c]imidazol-5-yl)-1-(4-(pyridin-4-yl)phenyl)ethan-1-one (24 mg, 45%) as pale yellow fluffy solid.

¹HNMR (CDCl₃, 400 MHz) δ: 8.70-8.68 (m, 2H), 7.98 (d, 2H, J=8.0 Hz), 7.70 (d, 2H, J=7.6 Hz), 7.67 (s, 1H), 7.50-7.43 (m, 4H), 7.36-7.34 (m, 3H), 6.90 (s, 1H), 5.78 (d, 1H, J=10.4 Hz), 3.38 (dd, 1H, J=2, 18.4 Hz), 3.12 (dd, 1H, J=10.8, 18.4 Hz), 2.23 (d, 3H, J=1.6 Hz); Mass (LCMS): 392, 393 (M+1); Purity: 100%.

Following examples have been synthesized by the above procedure described in Example 53 with their corresponding intermediates in similar reaction conditions:

| | | |
|---|---|---|
| 54. | 2-(7-methyl-6-phenyl- 5H-pyrrolo[1,2- c]imidazol-5-yl)-1-(4- (pyrimidin-5- yl)phenyl)ethan-1-one 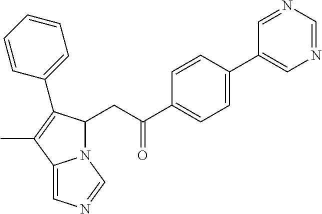 [SMILES: CC1=C(c2ccccc2)C(CC(=O)c2ccc(-c3cncnc3)cc2)n2cncc21] | ¹HNMR (CDCl₃, 400 MHz) δ: 9.24 (s, 1H), 8.95 (s, 2H), 8.01 (d, 2H, J = 8.4 Hz), 7.72 (s, 1H), 7.65 (d, 2H, 8.4 Hz), 7.45 (t, 2H, J = 7.6 Hz), 7.36-7.33 (m, 3H), 6.91 (s, 1H), 5.78 (d, 1H, J = 10.4 Hz), 3.39 (dd, 1H, J = 2, 18.4 Hz), 3.13 (dd, 1H, J = 10.4, 18.4 Hz), 2.23 (d, 3H, J = 1.6 Hz); Mass (LCMS): 393.1 (M + 1); Purity: 95.16%. |
| 55 | 2-(7-methyl-6-phenyl- 5H-pyrrolo[1,2- c]imidazol-5-yl)-1-(4- (pyridin-3- yl)phenyl)ethan-1-one 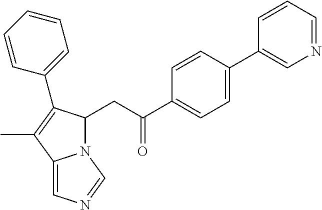 [SMILES: CC1=C(c2ccccc2)C(CC(=O)c2ccc(-c3cccnc3)cc2)n2cncc21] | ¹HNMR (CDCl₃, 400 MHz) δ: 8.84 (d, 1H, J = 8.8 Hz), 8.63 (dd, 1H, J = 1.6, 8.8 Hz), 7.97 (d, 2H, J = 8.4 Hz), 7.89-7.86 (m, 1H), 7.72 (s, 1H), 7.64 (d, 2H, J = 8.4 Hz), 7.45 (t, 2H, J = 7.6 Hz), 7.41-7.38 (m, 1H), 7.35-7.33 (m, 3H), 6.91 (s, 1H), 5.79 (d, 1H, J = 10.4 Hz), 3.38 (dd, 1H, J = 2, 18.4 Hz), 3.12 (dd, 1H, J = 10.8, 18.4 Hz), 2.23 (d, 3H, J = 1.64 Hz). Mass (LCMS): 392.1 (M + 1); Purity: 88.51%. |
| 56. | 2-(7-methyl-6-phenyl- 5H-pyrrolo[1,2- c]imidazol-5-yl)-1-(4- (thiophen-3- yl)phenyl)ethan-1-one 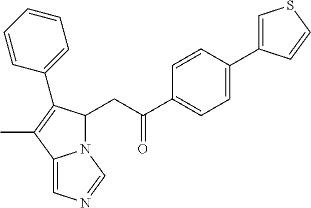 [SMILES: CC1=C(c2ccccc2)C(CC(=O)c2ccc(-c3ccsc3)cc2)n2cncc21] | ¹HNMR (CDCl₃, 400 MHz) δ: 7.89 (d, 2H, J = 8.4 Hz), 7.73 (s, 1H), 7.63 (d, 2H, J = 8.4 Hz), 7.56-7.55 (m, 1H), 7.46-7.40 (m, 4H), 7.35-7.33 (m, 3H), 6.91 (s, 1H), 5.78 (d, 1H, J = 10.8 Hz), 3.36 (dd, 1H, J = 2, 18.4 Hz), 3.09 (dd, 1H, J = 10.8, 18.4 Hz), 2.23 (d, 3H, J = 1.6 Hz). Mass (LCMS): 397.1 (M + 1); Purity: 98.33%. |
| 57. | 1-(4′-hydroxy-[1,1′- biphenyl]-4-yl)-2-(7- methyl-6-phenyl-5H- pyrrolo[1,2-c]imidazol- 5-yl)ethan-1-one 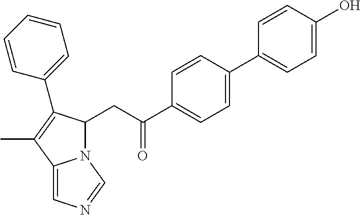 [SMILES: CC1=C(c2ccccc2)C(CC(=O)c2ccc(-c3ccc(O)cc3)cc2)n2cncc21] | ¹HNMR (DMSO-d₆, 400 MHz) δ: 9.43 (s, 1H), 7.96 (s, 1H), 7.85 (d, 2H, J = 8.0 Hz), 7.57-7.55 (m, 3H), 7.42-7.33 (m, 5H), 7.27 (t, 1H, J = 7.2 Hz), 6.81 (d, 2H, J = 8.4 Hz), 6.75 (s, 1H), 5.75 (d, 1H, J = 10.0 Hz), 3.33 (dd, 1H, J = 1.6, 16.4 Hz), 3.13 (dd, 1H, J = 10.4, 18.0 Hz), 2.16 (s, 3H); Mass (LCMS): 407.1 (M + 1); Purity: 96.11%. |
| 58. | 1-(2′-fluoro-[1,1′- biphenyl]-4-yl)-2-(7- methyl-6-phenyl-5H- pyrrolo[1,2-c]imidazol- 5-yl)ethan-1-one 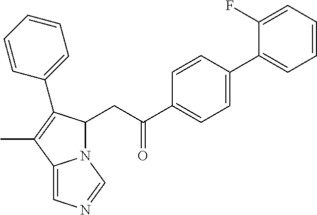 [SMILES: CC1=C(c2ccccc2)C(CC(=O)c2ccc(-c3ccccc3F)cc2)n2cncc21] | ¹HNMR (CDCl₃, 400 MHz) δ: 7.93 (d, 2H, J = 8.4 Hz), 7.70 (s, 1H), 7.61 (m, 2H), 7.46-7.41 (m, 3H), 7.35-7.32 (m, 4H), 7.22-7.13 (m, 2H), 6.89 (s, 1H), 5.78 (d, 1H, J = 10.4 Hz), 3.38 (dd, 1H, J = 2, 18.4 Hz), 3.12 (dd, 1H, J = 10.8, 18.4 Hz), 2.23 (d, 3H, J = 1.6 Hz); Mass (LCMS): 409.1 (M + 1); Purity: 94.83%. |
| 59. | 2-(7-methyl-6-phenyl- 5H-pyrrolo[1,2- c]imidazol-5-yl)-1-(4- (pyridin-4- yl)phenyl)ethan-1-ol 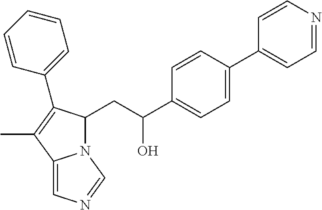 [SMILES: CC1=C(c2ccccc2)C(CC(O)c2ccc(-c3ccncc3)cc2)n2cncc21] | ¹HNMR (CDCl₃, 400 MHz) δ: 8.61-8.58 (m, 2H), 7.91 (s, 0.3H), 7.73 (s, 0.7H), 7.61-7.55 (m, 2H), 7.46-7.40 (m, 3H), 7.39-7.35 (m, 3H), 7.29-7.27 (m, 1.3H), 7.20 (d, 0.7, J = 7.6 Hz), 6.85 (s, 0.3H), 6.79 (s, 0.7H), 5.47 (d, 0.3H, J = 10 Hz), 5.19 (br. s, 0.7H), 5.10-5.07 (m, 0.3H), 4.83 (dd, 0.7H, J = 3.9, 8 Hz), 2.21-2.16 (m, 4H), 2.12-20.4 (m, 1H); Mass (LCMS): 394.1 (M + 1); Purity: 98.51%. |
| 60. | 2-(7-methyl-6-phenyl- 5H-pyrrolo[1,2- c]imidazol-5-yl)-1-(4- (thiophen-3- yl)phenyl)ethan-1-ol 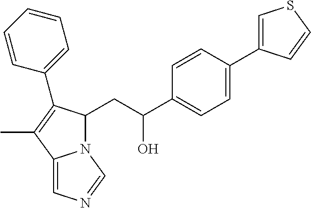 [SMILES: CC1=C(c2ccccc2)C(CC(O)c2ccc(-c3ccsc3)cc2)n2cncc21] | ¹HNMR (CDCl₃, 400 MHz) δ: 7.86 (s, 0.4H), 7.71 (s, 0.6H), 7.55 (d, 1H, J = 8 Hz), 7.50 (d, 1H, J = 8.4 Hz), 7.43-7.41 (m, 2H), 7.39-7.34 (m, 3H), 7.33-7.23 (m 4H), 7.19 (d, 1H J = 7.6 Hz), 6.87 (s, 0.4 H), 6.81 (s, 0.6H), 5.46-5.44 (m, 0.4H), 5.13 (br. s, 0.6H), 5.04-5.01 (m, 0.4H), 4.82-4.78 (m, 0.6H), 2.22-2.13 (m, 4H), 2.11-2.02 (m, 1H); Mass (LCMS): 399.1 (M + 1); Purity: 93.93%. |
| 61. | 2-(7-methyl-6-phenyl- 5H-pyrrolo[1,2- c]imidazol-5-yl)-1-(4′- (trifluoromethyl)-[1,1′- biphenyl]-4-yl)ethan-1- one 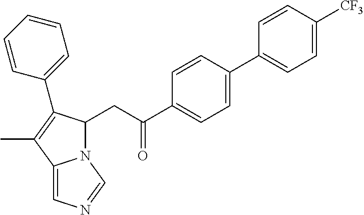 [SMILES: CC1=C(c2ccccc2)C(CC(=O)c2ccc(-c3ccc(C(F)(F)F)cc3)cc2)n2cncc21] | ¹HNMR (CDCl₃, 400 MHz) δ: 7.96 (d, 2H, J = 8.8 Hz), 7.72-7.69 (m, 4H), 7.64 (d, 2H, J = 8.4 Hz), 7.47-7.43 (m, 3H), 7.36-7.32 (m, 3H), 6.90 (s, 1H), 5.79 (d, 1H, J = 10.4 Hz), 3.38 (dd, 1H, J = 2, 18.4 Hz), 3.13 (dd, 1H, J = 10.8, 18.4 Hz), 2.23 (d, 3H, J = 1.6 Hz); Mass (LCMS): 459.4 (M + 1); Purity: 90.78%. |
| 62. | 2-(7-methyl-6-phenyl- 5H-pyrrolo[1,2- c]imidazol-5-yl)-1-(4′- methyl-[1,1′-biphenyl] 4-yl)ethan-1-one 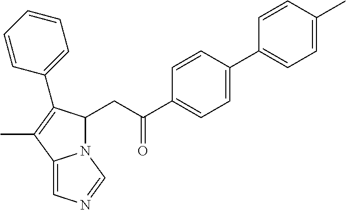 | ¹HNMR (CDCl₃, 400 MHz) δ: 7.91 (d, 2H, J = 8.8 Hz), 7.70 (s, 1H), 7.62 (d, 2H, J = 7.6 Hz), 7.49 (d, 2H, J = 8.0 Hz), 7.46-7.42 (m, 2H), 7.35- 7.27 (m, 5H), 6.89 (s, 1H), 5.78 (d, 1H, J = 10.8 Hz), 3.36 (dd, 1H, J = 2, 18 Hz), 3.11 (dd, 1H, J = 10.8, 18 Hz), 2.39 (s, 3H), 2.23 (d, H, J = 1.6 Hz); Mass (LCMS): 405.1 (M + 1); Purity: 92.95%. |
| 63. | 1-(4′-fluoro-[1,1′- biphenyl]-4-yl)-2-(7- methyl-6-phenyl-5H- pyrrolo[1,2-c]imidazol- 5-yl)ethan-1-one 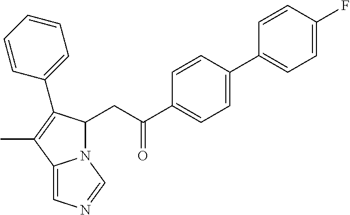 [SMILES: CC1=C(c2ccccc2)C(CC(=O)c2ccc(-c3ccc(F)cc3)cc2)n2cncc21] | ¹HNMR (CDCl₃, 400 MHz) δ: 7.92 (d, 2H, J = 7.6 Hz), 7.70 (s, 1H), 7.59-7.55 (m, 4H), 7.44- 7.43 (m, 2H), 7.35-7.34 (m, 3H), 7.16-7.12 (m, 2H), 6.89 (s, 1H), 5.78 (d, 1H, J = 9.6 Hz), 3.36 (d, 1H, J = 18 Hz), 3.12 (dd, 1H, J = 10.4, 15.6 Hz), 2.23 (s, 3H); Mass (LCMS): 408.1 (M + 1); Purity: 96.38%. |
| 64. | 2-(7-methyl-6-phenyl- 5H-pyrrolo[1,2- c]imidazol-5-yl)-1-(4-(5- methylthiophen-2- yl)phenyl)ethan-1-one 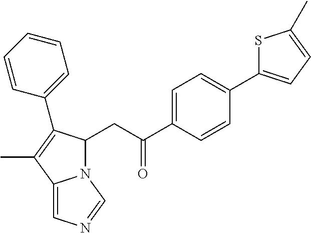 [SMILES: CC1=C(c2ccccc2)C(CC(=O)c2ccc(-c3ccc(C)s3)cc2)n2cncc21] | ¹HNMR (CDCl₃, 400 MHz) δ: 7.83 (d, 2H, J = 8.8 Hz), 7.69 (s, 1H), 7.56 (d, 2H, J = 8.4 Hz), 7.46-7.42 (m, 2H), 7.34-7.31 (m, 3H), 7.22 (d, 1H, J = 3.6 Hz), 6.88 (s, 1H), 6.75 (d, 1H, J = 3.6 Hz), 5.77 (d, 1H, J = 10.8 Hz), 3.32 (dd, 1H, J = 2, 18.4 Hz), 3.07 (dd, 1H, J = 10.4, 18.4 Hz), 2.51 (s, 3H), 2.23 (d, 3H, J = 1.6 Hz). Mass (LCMS): 411.1 (M + 1); Purity: 91.92%. |
| 65. | 1-([1,1′:4′,1″-terphenyl]- 4-yl)-2-(7-methyl-6- phenyl-5H-pyrrolo[1,2- c]imidazol-5-yl)ethan-1- one 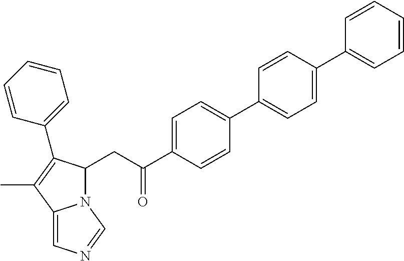 [SMILES: CC1=C(c2ccccc2)C(CC(=O)c2ccc(-c3ccc(-c4ccccc4)cc3)cc2)n2cncc21] | ¹HNMR (CDCl₃, 400 MHz) δ: 7.95 (d, 2H, J = 8.4 Hz), 7.72-7.70 (m, 2H), 7.68-7.66 (m, 4H), 7.64-7.62 (m, 2H), 7.48-7.43 (m, 5H), 7.39-7.32 (m, 4H), 6.90 (s, 1H), 5.80 (d, 1H, J = 10.8 Hz), 3.39 (dd, 1H, J = 2.0, 18.4 Hz), 3.13 (dd, 1H, J = 10.8, 18.4 Hz), 2.24 (d, 3H, 1.6 Hz); Mass (LCMS): 467.1 (M + 1); Purity: 92.32%. |
| 66. | 2-(7-methyl-6-phenyl- 5H-pyrrolo[1,2- c]imidazol-5-yl)-1-(4′- (methylsulfonyl)[1,1′- biphenyl]-4-yl)ethan-1- one 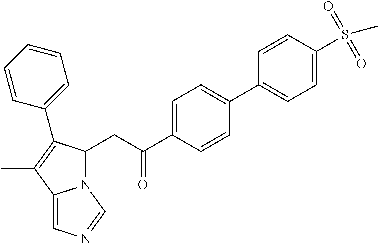 | ¹HNMR (CDCl₃, 400 MHz) δ: 8.21 (dd, 1H, J = 1.2, 8.0 Hz), 7.95 (d, 2H, J = 8.4 Hz), 7.71 (s, 1H), 7.66-7.59 (m, 3H), 7.53 (d, 2H, J = 8.4 Hz), 7.45- 7.43 (m, 2H), 7.36-7.32 (m, 3H), 6.90 (s, 1H), 5.78 (d, 1H, J = 10.0 Hz), 3.40 (dd, 1H, J = 1.6, 18.4 Hz), 3.14 (dd, 1H, J = 10.4, 18.4 Hz), 2.64 (s, 3H), 2.24 (d, 3H, J = 1.6 Hz); Mass (LCMS): 469.1 (M + 1); Purity: 89.16%. |
| 67. | 1-(4-(1H-imidazol-5- yl)phenyl)-2-(7-methyl- 6-phenyl-5H- pyrrolo[1,2-c]imidazol- 5-yl)ethan-1-one 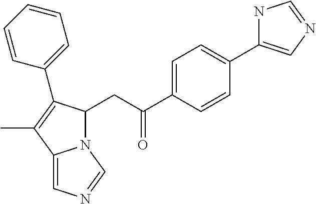 [SMILES: CC1=C(c2ccccc2)C(CC(=O)c2ccc(-c3cnc[nH]3)cc2)n2cncc21] | ¹HNMR (CDCl₃, 400 MHz) δ: 7.95 (d, 2H, J = 8.4 Hz), 7.72-7.70 (m, 2H), 7.68-7.66 (m, 4H), 7.64-7.62 (m, 2H), 7.39-7.32 (m, 2H), 6.90 (s, 1H), 5.80 (d, 1H, J = 10.8 Hz), 3.38-3.33 (m, 1H), 3.13 (dd, 1H, J = 10.8, 18.4 Hz), 2.24 (d, 3H, J = 1.6 Hz); Mass (LCMS): 381.1 (M + 1); Purity: 96.91%. |
| 69. | 1-Biphenyl-3-yl-2-(7- methyl-6-phenyl-5H- pyrrolo[1,2-c]imidazol- 5-yl)-ethanone 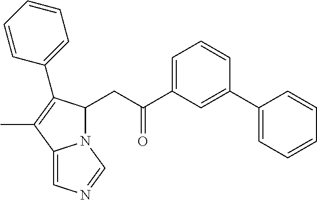 [SMILES: CC1=C(c2ccccc2)C(CC(=O)c2cccc(-c3ccccc3)c2)n2cncc21] | ¹HNMR (CDCl₃, 400 MHz) δ: 8.64 (s, 1H), 8.07 (s, 1H), 7.80 (d, 2H, J = 7.6 Hz), 7.56-7.47 (m, 5H), 7.44 (t, 3H, J = 7.6 Hz), 7.39 (d, 1H, J = 7.2 Hz), 7.35 (d, 2H, J = 7.6 Hz), 7.25 (s, 1H), 5.95 (d, 1H, J = 10.8 Hz), 3.57 (d, 1H, J = 18.4 Hz), 3.19 (dd, 1H, J = 11.2, 18.4 Hz), 2.26 (s, 3H); Mass (LCMS): 391.1 (M + 1); Purity: 94.66%. |
| 70. | 1-(2′-Fluoro-biphenyl-3- yl)-2-(7-methyl-6- phenyl-5H-pyrrolo[1,2- c]imidazol-5-yl)- ethanone 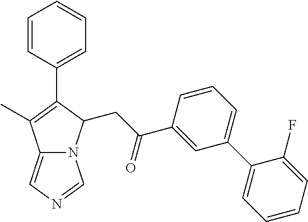 [SMILES: CC1=C(c2ccccc2)C(CC(=O)c2cccc(-c3ccccc3F)c2)n2cncc21] | ¹HNMR (CDCl₃, 400 MHz) δ: 8.64 (br. s, 1H), 8.01 (s, 1H), 7.85 (d, 1H, J = 7.6 Hz), 7.77 (d, 1H, J = 7.6 Hz), 7.54-7.49 (m, 3H), 7.45-7.38 (m, 3H), 7.35-7.33 (m, 3H), 7.23 (t, 1H, J = 7.6 Hz,), 7.15 (t, 1H, J = 8.4 Hz), 5.96 (d, 1H, J = 9.6 Hz), 3.56 (d, 1H, J = 18.4 Hz), 3.18 (dd, 1H, J = 10.8, 18.4 Hz,), 2.26 (s, 3H); Mass (LCMS): 409.1 (M + 1); Purity: 91.26%. |

### Example 77: Preparation of N-methyl-4-(2-(7-methyl-6-phenyl-5H-pyrrolo[1,2-c]imidazol-5-yl)acetyl)benzamide

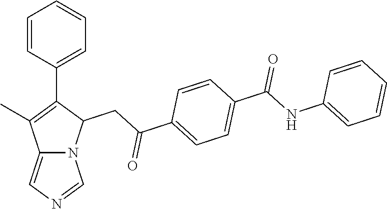 [SMILES: CC1=C(c2ccccc2)C(CC(=O)c2ccc(C(=O)Nc3ccccc3)cc2)n2cncc21]

In a DCM solution of compound of (4-[2-(7-Methyl-6-phenyl-5H-pyrrolo[1,2-c]imidazol-5-yl)-acetyl]-benzoic acid) (Example 24, 45 mg, 0.125 mmole) under nitrogen atmosphere added aniline (13 mg, 0.1381 mmole, 1.1 eq) at about 0° C., followed by DIPEA (49 mg, 0.376 mmole, 3 eq) and Propylphosphonic anhydride (50% solution in Ethyl acetate) (0.12 mL, 0.188 mmole, 1.5 eq) drop wise. Stirred the reaction solution at room temperature for about 1 hour. Diluted the reaction mixture with DCM (15 mL) and washed the organics with water (20 mL×3), brine and organic layer was dried over anhydrous sodium sulphate, filtered and concentrated to get crude compound of N-methyl-4-(2-(7-methyl-6-phenyl-5H-pyrrolo[1,2-c]imidazol-5-yl)acetyl)benzamide. This was then purified by silica gel flash column chromatography to give pure compound N-methyl-4-(2-(7-methyl-6-phenyl-5H-pyrrolo[1,2-c]imidazol-5-yl)acetyl)benzamide (10 mg, 18%) as a pale yellow solid.

¹HNMR (CDCl₃, 400 MHz) δ: 7.93-7.84 (m, 4H), 7.68-7.66 (m, 2H), 7.46-7.31 (m, 8H), 7.15 (t, 1H, J=7.2 Hz), 6.98 (s, 1H), 5.77-5.74 (m, 1H), 3.37 (dd, 1H, J=1.2, 18.0 Hz), 3.11-3.03 (m, 1H), 2.21 (s, 3H); LCMS=434.1 (M+1); Purity=94.31%.

Following examples have been synthesized by the above procedure described in Example 77 with their corresponding intermediates in similar reaction conditions:

| Example | | |
|---|---|---|
| No. | IUPAC Name/Structure | Analytical Data |
| 78. | N-methyl-4-(2-(7- methyl-6-phenyl-5H- pyrrolo[1,2-c]imidazol-5- yl)acetyl)benzamide 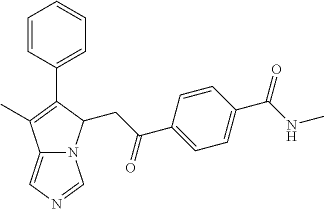 | ¹HNMR (CDCl₃, 400 MHz) δ: 7.90 (d, 2H, J = 8.4 Hz), 7.80 (d, 2H, J = 8.4 Hz), 7.67 (s, 1H), 7.44 (t, 2H, J = 8.0 Hz), 7.35-7.32 (m, 3H), 6.88 (s, 1H), 6.28-6.27 (m, 1H), 5.76-5.74 (m, 1H), 3.36 (dd, 1H, J = 2.0, 18.4 Hz), 3.12 (dd, 1H, J = 10.8, 18.4 Hz), 3.01 (d, 3H, J = 4.8 Hz), 2.22 (d, 3H, J = 1.6 Hz); LCMS: 372.1 (M + 1); Purity: 97.66%. |
| 79. | 4-(2-(7-methyl-6-phenyl- 5H-pyrrolo[1,2- c]imidazol-5-yl)acetyl)- N-(pyridin-3- yl)benzamide 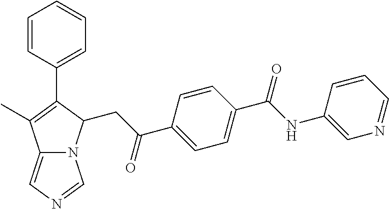 [SMILES: CC1=C(c2ccccc2)C(CC(=O)c2ccc(C(=O)Nc3cccnc3)cc2)n2cncc21] | ¹HNMR (CDCl₃, 400 MHz) δ: 9.01 (s, 1H), 8.76 (s, 1H), 8.38-8.37 (m, 1H), 8.30-8.28 (m, 1H), 7.95 (m, 3H), 7.71 (s, 1H), 7.47-7.39 (m, 2H), 7.37-7.26 (m, 4H), 6.90 (s, 1H), 5.75 (d, 1H, J = 9.6 Hz), 3.42-3.37 (m, 1H), 3.10 (dd, 1H, J = 10.4, 18.0 Hz), 2.22 (s, 3H); LCMS: 435.1 (M + 1); Purity: 90.43%. |
| 80. | N-(4-chlorophenyl)-4-(2- (7-methyl-6-phenyl-5H- pyrrolo[1,2-c]imidazol-5- yl)acetyl)benzamide 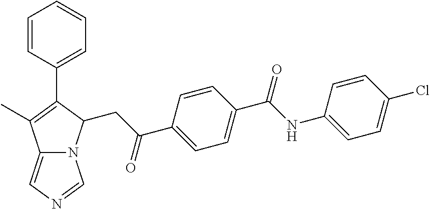 [SMILES: CC1=C(c2ccccc2)C(CC(=O)c2ccc(C(=O)Nc3ccc(Cl)cc3)cc2)n2cncc21] | ¹HNMR (CDCl₃, 400 MHz) δ: 8.27 (s, 1H), 7.95-7.90 (m, 4H), 7.67 (s, 1H), 7.61 (d, 2H, J = 8.4 Hz), 7.45 (t, 2H, J = 7.6 Hz), 7.36-7.30 (m, 5H), 6.89 (s, 1H), 5.75 (d, 1H, J = 10.4 Hz), 3.38 (dd, 1H, J = 1.6, 18.4 Hz), 3.09 (dd, 1H, J = 10.8, 18.4 Hz), 2.22 (d, 3H, J = 1.2 Hz); LCMS: 468.1; Purity: 90.34%. |
| 81. | N-(2,4-difluorophenyl)- 4-(2-(7-methyl-6-phenyl- 5H-pyrrolo[1,2- c]imidazol-5- yl)acetyl)benzamide 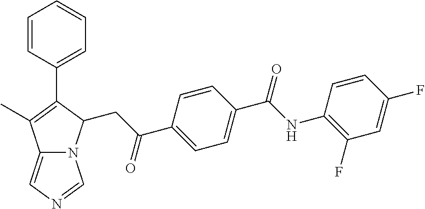 [SMILES: CC1=C(c2ccccc2)C(CC(=O)c2ccc(C(=O)Nc3ccc(F)cc3F)cc2)n2cncc21] | 1HNMR (CDCl₃, 400 MHz) δ: 8.34-8.32 (m, 1H), 8.03 (s, 1H), 7.98 (d, 2H, J = 8.4 Hz), 7.92 (d, 2H, J = 8.4 Hz), 7.69 (s, 1H), 7.47-7.43 (m, 2H), 7.36-7.32 (m, 2H), 6.95-6.89 (m, 3H), 5.76 (d, 1H, J = 10.4 Hz), 3.39 (dd, 1H, J = 2, 18.4 Hz), 3.12 (dd, 1H, J = 10.8, 18.4 Hz), 2.22 (d, 3H, J = 2 Hz); LCMS: 470.1 (M + 1); Purity: 93.37%. |
| 82. | N-(4-chlorophenyl)-4-(1- hydroxy-2-(7-methyl-6- phenyl-5H-pyrrolo[1,2- c]imidazol-5- yl)ethyl)benzamide 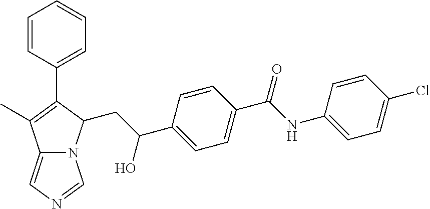 [SMILES: CC1=C(c2ccccc2)C(CC(O)c2ccc(C(=O)Nc3ccc(Cl)cc3)cc2)n2cncc21] | ¹HNMR (CDCl₃, 400 MHz) δ: 8.60 (s, 0.8H), 8.40 (s, 0.2H), 7.72 (d, 2H, J = 7.2 Hz), 7.60 (d, 2H, J = 8 Hz), 7.40 (t, 2H, J = 7.6 Hz), 7.38- 7.32 (m, 2H), 7.29-7.28 (m, 3H), 7.16 (d, 1H, J = 7.6 Hz), 7.07 (dd, 1H, J = 2.4, 8.4 Hz), 6.83 (s, 0.2H), 6.73 (s, 0.8H), 5.86-5.76 (m, 1H), 5.17 (br. s, 1H), 5.02-4.88 (m, 2H), 4.70-4.67 (m, 1H), 2.14 (s, 3H); Mass (LCMS): 470.1 (M + 1), HPLC = 90.52%. |
| 83. | N-isobutyl-4-(2-(7- methyl-6-phenyl-5H- pyrrolo[1,2-c]imidazol-5- yl)acetyl)benzamide 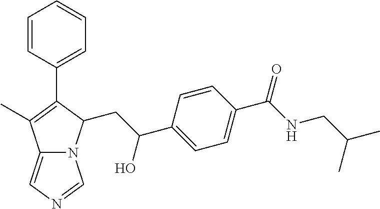 [SMILES: CC1=C(c2ccccc2)C(CC(O)c2ccc(C(=O)NCC(C)C)cc2)n2cncc21] | ¹HNMR (CDCl₃, 400 MHz) δ: 7.90 (d, 2H, J = 8.4 Hz), 7.80 (d, 2H, J = 8.4 Hz), 7.21 (s, 1H), 7.45 (t, 2H, J = 7.2 Hz), 7.36-7.32 (m, 3H), 6.89 (s, 1H), 6.30 (br. s, 1H), 5.76 (d, 1H, J = 13.2 Hz), 3.36 (dd, 1H, J = 2, 18.4 Hz), 3.28 (t, 2H, J = 6.4 Hz), 3.09 (dd, 1H, J = 10.8, 18.4 Hz), 2.22 (d, 3H, J = 1.6 Hz), 1.95-1.86 (m, 1H), 0.98 (d, 6H, J = 6.8 Hz); LCMS: 414.1 (M + 1); Purity: 95.55%. |
| 84. | N-(2,4-dimethylphenyl)- 4-(2-(7-methyl-6-phenyl- 5H-pyrrolo[1,2- c]imidazol-5- yl)acetyl)benzamide 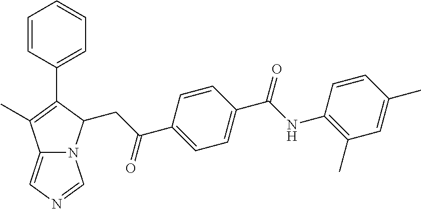 | ¹HNMR (CDCl₃, 400 MHz) δ: 7.98-7.91 (m, 4H), 7.75-7.66 (m, 3H), 7.47-7.44 (m, 2H), 7.37-7.33 (m, 3H), 7.06-7.04 (m, 2H), 6.92 (s, 1H), 5.77 (d, 1H, J = 10.4 Hz), 3.39 (dd, 1H, J = 2.0, 18.4 Hz), 3.13 (dd, 1H, J = 10.8, 18.4 Hz), 2.31 (s, 3H), 2.27 (s, 3H), 2.23 (d, 3H, J = 1.6 Hz); Mass (LCMS): 462.1 (M + 1); Purity: 94.91%. |
| 85. | 4-(2-(7-methyl-6-phenyl- 5H-pyrrolo[1,2- c]imidazol-5-yl)acetyl)- N-phenylbenzamide 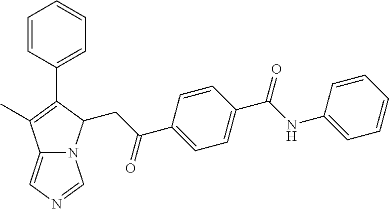 [SMILES: CC1=C(c2ccccc2)C(CC(=O)c2ccc(C(=O)Nc3ccccc3)cc2)n2cncc21] | ¹HNMR (CDCl₃, 400 MHz) δ: 8.32 (s, 1H), 8.08 (d, 1H, J = 8 Hz), 8.02 (d, 1H, J = 8 Hz), 7.73 (s, 1H), 7.64 (d, 2H, J = 7.6 Hz), 7.56 (t, 1H, J = 8.0 Hz), 7.47-7.42 (m, 2H), 7.39-7.32 (m, 5H), 7-18-7.14 (m, 2H), 6.90 (s, 1H), 5.78 (d, 1H, J = 10.8 Hz), 3.39 (dd, 1H, J = 2, 18.4 Hz), 3.20 (dd, 1H, J = 11.2, 18.4 Hz), 2.22 (d, 3H, J = 1.6 Hz); Mass (LCMS): 434.1 (M + 1), Purity = 90.42%. |

### Example 86: Preparation of 1-Cyclohexyl-2-(7-methyl-6-(p-tolyl)-5H-pyrrolo[1,2-c]imidazol-5-yl)ethan-1-one

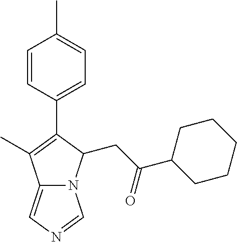 [SMILES: CC1=C(c2ccc(C)cc2)C(CC(=O)C2CCCCC2)n2cncc21]

### Step 1: Preparation of Dimethyl (2-cyclohexyl-2-oxoethyl)phosphonate

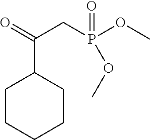

At about −78° C., to a solution of dimethyl methylphophonate (34.92 g, 281.69 mmol) in dry THF (150 ml) was added n-BuLi solution (116 mL, 281.69 mmol) 2.5 M in THF dropwise. The reaction mass was allowed to stir at about −78° C. for about 30 minutes and then a solution of compound methyl cyclohexanecarboxylate in THF (50 mL) was added to reaction mixture at same temperature. The resulting mixture was kept stirring at about −78° C. for about 30 minutes and then slowly warmed up to about 0° C. in about 1 hour. After completion, reaction mass was quenched by the addition of water (150 mL) and the mixture was extracted with ethyl acetate (2×200 mL). Combined organic layer was evaporated under reduced pressure to give compound Dimethyl (2-cyclohexyl-2-oxoethyl)phosphonate (32 g, 97%) as clear thick liquid.

¹HNMR (CDCl₃, 400 MHz) δ: 3.74 (s, 3H), 3.71 (s. 3H), 3.10 (s, 1H), 3.04 (s, 1H), 2.52-2.49 (m, 1H), 1.85-1.82 (m, 2H), 1.74-1.71 (m, 2H), 1.63-1.60 (m, 2H), 1.29-1.20 (m, 4H); Mass (LCMS): 235 (M+1).

### Step 2: Preparation of 5-Bromo-1-cyclohexyl-4-(p-tolyl)hexa-2,4-dien-1-one

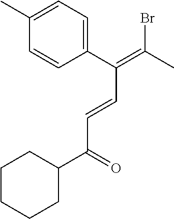 [SMILES: C/C(Br)=C(\C=C\C(=O)C1CCCCC1)c1ccc(C)cc1]

At about 0° C., to a suspension of sodium hydride (60%, 0.176 g, 4.18 mmol) in dry THF (5 mL) was added a solution of phosphonate compound Dimethyl (2-cyclohexyl-2-oxoethyl)phosphonate (Step 1, 2.01 g, 4.80 mmol) in THF (5 mL) slowly. The reaction mass was allowed to stir at about 0° C. for about 30 minutes and then a solution of compound 3-Bromo-2-(p-tolyl)but-2-enal (1 g, 4.18 mmol) in THF (5 mL) was added to reaction mixture at same temperature. The resulting mixture was stirred at room temperature for about 2 hours. After completion, reaction mass was quenched by the addition of water (50 mL) and the mixture was extracted with ethyl acetate (3×50 mL). Combined organic layer was evaporated under reduced pressure and the residue was purified by flash chromatography eluting with 2% ethyl acetate and n-hexanes to give pure compound 5-Bromo-1-cyclohexyl-4-(p-tolyl)hexa-2,4-dien-1-one (1.05 g, 72.41%) as pale brown thick liquid.

¹HNMR (CDCl₃, 400 MHz) δ: 7.96 (d, 0.6H, J=15.6 Hz), 7.75 (d, 0.4H, J=15.6 Hz), 7.23-7.19 (m, 2H), 6.98-6.95 (m, 2H), 5.77-5.65 (m, 1H), 2.72 (s, 1H), 2.54-2.48 (m, 1H), 2.40 (s, 1H), 2.39 (s, 2H), 2.26 (s, 2H), 1.76-1.71 (m, 4H), 1.66-1.59 (m, 2H), 1.32-1.27 (m, 2H), 1.24-1.17 (m, 2H); Mass (LCMS): 349 (M+1).

### Step 3 & 4: Preparation of 1-Cyclohexyl-2-(7-methyl-6-(p-tolyl)-5H-pyrrolo[1,2-c]imidazol-5-yl)ethan-1-one

The Step 2 product was subjected to similar experimental procedure as described in Steps 3 & 4 for the synthesis of Example 17 to generate final said compound 1-Cyclohexyl-2-(7-methyl-6-(p-tolyl)-5H-pyrrolo[1,2-c]imidazol-5-yl)ethan-1-one.

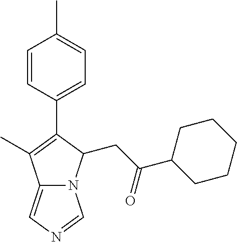 [SMILES: CC1=C(c2ccc(C)cc2)C(CC(=O)C2CCCCC2)n2cncc21]

¹HNMR (CDCl₃, 400 MHz) δ: 7.55 (br. s, 1H), 7.23 (d, 2H, J=8 Hz), 7.16 (d, 2H, J=8 Hz), 6.85 (br. s, 1H), 5.53 (d, 1H, J=10.4 Hz), 2.81 (dd, 1H, J=2.4, 18.8 Hz), 2.52 (dd, 1H, J=10.4, 18.8 Hz), 2.38 (s, 3H), 2.27-2.21 (m, 1H), 2.17 (d, 3H, J=1.6 Hz), 1.73-1.69 (m, 4H), 1.34-1.16 (m, 1H), 1.20-1.16 (m, 5H); Mass (LCMS): 335.1 (M+1)

Following intermediates have been synthesized as described in the above process of Example 86-Step 1-3:

| Intermediate No. | Analytical Data |
|---|---|
| Methyl 4-(5-bromo-4-phenylhexa-2,4- dienoyl)cyclohexane-1-carboxylate 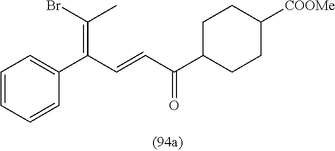 [SMILES: COC(=O)C1CCC(C(=O)/C=C/C(=C(\C)Br)c2ccccc2)CC1] | 1HNMR (CDCl₃, 400 MHz) δ: 7.96 (d, 1H, J = 15.6 Hz), 7.41-7.36 (m, 3H), 7.09-7.06 (m, 2H), 5.67 (d, 1H, J = 15.6 Hz), 3.65 (s, 3H), 2.56-2.59 (m, 2H), 2.26 (s, 3H), 2.56-2.59 (m, 2H), 1.67-1.55 (m, 6H); Mass (LCMS): 390.9 and 392.9 (M⁺, M + 2). |
| 1-(3-Chlorophenyl)propan-2-one 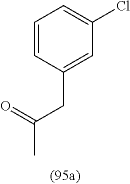 | ¹HNMR (CDCl₃, 400 MHz) δ: 7.27-7.25 (m, 2H), 7.19 (s, 1H), 7.09-7.07 (m, 1H), 3.68 (s, 2H), 2.17 (s, 3H). |
| 3-Bromo-2-(3-chlorophenyl)but-2-enal 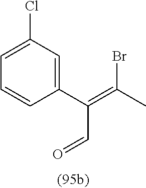 | ¹HNMR (CDCl₃, 400 MHz) δ: 10.22 (s, 0.6H), 10.08 (s, 0.4H), 7.34 (m, 2H), 7.13 (s, 0.4H), 7.08 (s, 0.6H), 7.03-7.00 (m, 0.4H), 6.97-6.95 (m, 0.6H), 2.99 (s, 1H), 2.50 (s, 2H). |
| 5-Bromo-4-(3-chlorophenyl)-1- cyclohexylhexa-2,4-dien-1-one 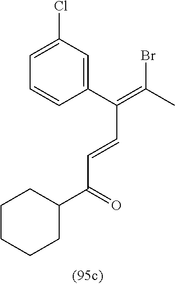 [SMILES: C/C(Br)=C(\C=C\C(=O)C1CCCCC1)c1cccc(Cl)c1] | ¹HNMR (CDCl₃, 400 MHz) δ: 7.92 (d, 0.5H, J = 15.6 Hz), 7.71 (d, 0.5H, J = 15.0 Hz), 7.37-7.33 (m, 2H), 7.10-7.09 (m, 1H), 6.99-6.96 (m, 1H), 5.73-5.61 (m, 1H), 2.72 (s, 1.5H), 2.54-2.49 (m, 0.5H), 2.38- 2.32 (m, 0.5H), 2.27 (s, 1.5H), 1.76-1.63 (m, 4H), 1.30-1.14 (m, 6H); Mass (LCMS): 368.9 (M + 1). |
| (Z)-3-bromo-2-(2-fluorophenyl)but-2-enal 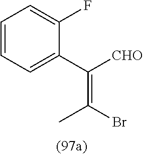 | 1HNMR (CDCl₃, 400 MHz) δ: 10.21 (s, 1H), 7.40- 7.34 (m, 1H), 7.20-7.16 (m, 1H), 7.13-7.04 (m, 2H), 2.48 (d, 3H, J = 0.8 Hz). |
| (2E,4E)-5-bromo-4-phenyl-1- (tetrahydro-2H-pyran-4-yl)hexa-2,4- dien-1-one 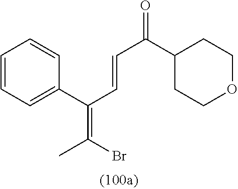 [SMILES: C/C(Br)=C(/C=C/C(=O)C1CCOCC1)c1ccccc1] | ¹HNMR (CDCl₃, 400 MHz) δ: 8.01 (d, 1H, J = 15.6 Hz), 7.43-7.35 (m, 3H), 7.09-7.07 (m, 2H), 5.67 (d, 1H, J = 15.6 Hz), 3.99-3.94 (m, 2H), 3.44-3.38 (m, 2H), 2.74-2.67 (m, 1H), 2.27 (s, 3H), 1.73-1.62 (m, 4H); Mass (LCMS): 335.0 and 336.9 (M⁺, M + 2). |
| (2E,4E)-5-bromo-1-cyclohexyl-4-(2- fluorophenyl)hexa-2,4-dien-1-one 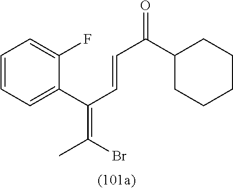 [SMILES: C/C(Br)=C(/C=C/C(=O)C1CCCCC1)c1ccccc1F] | ¹HNMR (CDCl₃, 400 MHz) δ: 7.72 (d, 1H, J = 15.2 Hz), 7.41-7.36 (m, 1H), 7.23-7.18 (m, 1H), 7.16-7.11 (m, 1H), 7.09-7.05 (m,1H), 5.72 (d, 1H, J = 15.2 Hz), 2.75 (s, 3H), 2.39-2.32 (m, 1H), 1.79-1.72 (m, 4H), 1.29-1.20 (m, 6H); Mass (LCMS): 350.9 and 353.0 (M⁺, M + 2). |
| (Z)-3-bromo-2,3-diphenylacrylaldehyde 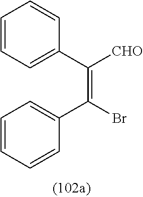 [SMILES: O=C/C(=C(\Br)c1ccccc1)c1ccccc1] | ¹HNMR (CDCl₃, 400 MHz) δ: 9.60 (s, 1H), 7.57.754 (m, 2H), 7.49-7.40 (m, 6H), 7.29-7.25 (m, 2H); Mass (LCMS): 286.9 and 289.0 (M⁺, M + 2). |
| (2E,4E)-5-bromo-1-cyclohexyl-4,5- diphenylpenta-2,4-dien-1-one 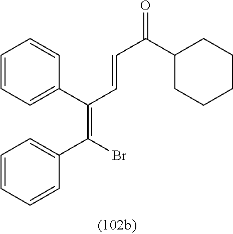 [SMILES: O=C(/C=C/C(=C(\Br)c1ccccc1)c1ccccc1)C1CCCCC1] | ¹HNMR (CDCl₃, 400 MHz) δ: 7.49-7.45 (m, 5H), 7.43-7.39 (m, 4H), 7.25-7.23 (m, 2H), 5.74 (d, 1H, J = 15.6 Hz), 2.32-2.25 (m, 1H), 1.72-1.64 (m, 4H), 1.28-1.12 (m, 6H); Mass (LCMS): 395.0 and 397.0 (M⁺, M + 2). |
| 3-bromo-3-(4-methoxyphenyl)-2- methylacrylaldehyde 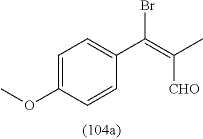 | ¹HNMR (CDCl₃, 400 MHz) δ: 10.25 (s, 0.6H), 10.09 (s, 0.4H), 7.09-6.98 (m, 2H), 6.94-6.91 (m, 2H), 3.82 (s, 3H), 2.97 (s, 1.2H), 2.51 (s, 1.8H). |
| 5-bromo-1-cyclohexyl-5-(4- methoxyphenyl)-4-methylpenta-2,4-dien- 1-one 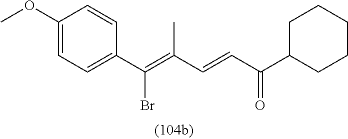 [SMILES: COc1ccc(/C(Br)=C(C)/C=C/C(=O)C2CCCCC2)cc1] | ¹HNMR (CDCl₃, 400 MHz) δ: 7.96 (d, 0.7H, J = 15.6 Hz), 7.75 (d, 0.3H, J = 15.6 Hz), 7.02-6.97 (m, 2H), 6.95-6.92 (m, 2H), 5.76 (d, 0.3H, J = 15.6 Hz), 5.68 (d, 0.7H, J = 15.6 Hz), 3.85 (s, 1H), 3.81 (s, 2H), 2.71 (s, 1H), 2.52-2.47 (m, 0.7H), 2.35-2.30 (m, 0.3H), 2.27 (s, 2H), 1.76-1.59 (m, 5H), 1.36-1.16 (m, 5H). |
| 1-(o-tolyl)propan-2-one 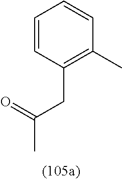 | ¹HNMR (CDCl₃, 400 MHz) δ: 7.20-7.13 (m, 4H), 3.72 (s, 2H), 2.26 (s, 3H), 2.15 (s, 3H) |
| 3-Bromo-2-(o-tolyl)but-2-enal 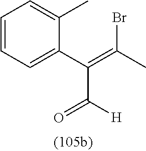 | Mass (LCMS): 240.7(M + 1) |
| 5-Bromo-1-cyclohexyl-4-(o-tolyl)hexa- 2,4-dien-1-one 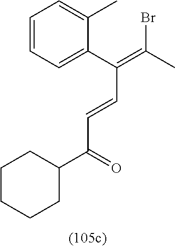 [SMILES: C/C(Br)=C(\C=C\C(=O)C1CCCCC1)c1ccccc1C] | ¹HNMR (CDCl₃, 400 MHz) δ: 7.94 (d, 0.4H, J = 15.6 Hz), 7.73 (d, 0.6H, J = 14.8 Hz), 7.31-7.27 (m, 1H), 7.24-7.20 (m, 2H), 6.94 (t, 1H, J = 7.2 Hz), 5.62 (d, 0.6H, J = 14.8 Hz), 5.53 (dd, 0.4H, J = 0.4, 15.6 Hz), 2.74 (s, 2H), 2.35-2.28 (m, 1H), 2.18 (s, 1H), 2.10 (s, 2H), 2.08 (s, 1H), 1.74-1.72 (m, 4H), 1.64- 1.62 (m, 1H), 1.31-1.30 (m, 1H), 1.24-1.17 (m, 4H); Mass (LCMS): 347.0. |
| (Z)-3-bromo-4-methyl-2-phenylpent-2- enal 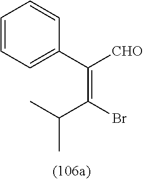 | ¹HNMR (CDCl₃, 400 MHz) δ: 10.29 (s, 1H), 7.40- 7.38 (m, 3H), 7.06-7.04 (m, 2H), 2.94-2.87 (m, 1H), 1.10 (d, 6H, J = 6.8 Hz). |
| (2E,4E)-5-bromo-1-cyclohexyl-6- methyl-4-phenylhepta-2,4-dien-1-one 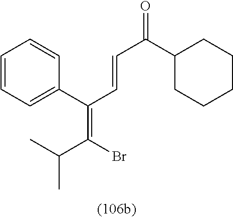 [SMILES: CC(C)/C(Br)=C(/C=C/C(=O)C1CCCCC1)c1ccccc1] | ¹HNMR (CDCl₃, 400 MHz) δ: 8.01 (d, 1H, J = 16.0 Hz), 7.43-7.35 (m, 3H), 7.08-7.06 (m, 2H), 5.60 (d, 1H, J = 15.6 Hz), 2.64-2.57 (m, 1H), 2.54-2.49 (m, 1H), 1.75-1.73 (m, 4H), 1.29-1.20 (m, 6H), 1.01 (d, 6H, J = 6.4 Hz); Mass (LCMS): 361.0 and 363.0 (M ⁺, M + 2). |
| Dimethyl (2-((3r,5r,7r)-adamantan-1-yl)- 2-oxoethyl)phosphonate 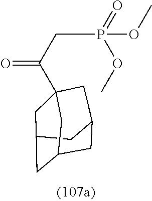 [SMILES: COP(=O)(CC(=O)C12CC3CC(CC(C3)C1)C2)OC] | ¹HNMR (CDCl₃, 400 MHz) δ: 3.75 (s, 3H), 3.73 (s, 3H), 3.13 (s, 1H), 3.07 (s, 1H), 1.83 (d, 1H, J = 2.8 Hz), 1.77-1.75 (m, 6H), 1.72-1.61 (m, 8H); Mass (LCMS): 287(M + 1) |
| 1-((3r,5r,7r)-Adamantan-1-yl)-5-bromo- 4-phenylhexa-2,4-dien-1-one 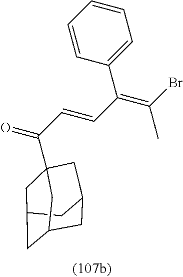 [SMILES: C/C(Br)=C(\C=C\C(=O)C12CC3CC(CC(C3)C1)C2)c1ccccc1] | ¹HNMR (CDCl₃, 400 MHz) δ: 8.00 (d, 0.6H, J = 15.2 Hz), 7.79 (d, 0.4H, J = 15.2 Hz), 7.44-7.35 (m, 3H), 7.11-7.05 (m, 2H), 6.01 (d, 0.4H, J = 15.2 Hz), 5.99 (d, 0.6H, J = 15.2 Hz), 2.72 (s, 1H), 2.25 (s, 2H), 2.01-1.88 (m, 4H), 1.70-1.63 (m, 11H); Mass (LCMS): 385.0 |
| (2E,4E)-5-bromo-1-(4-((tert- butyldimethylsilyl)oxy)cyclohexyl)-4- phenylhexa-2,4-dien-1-one 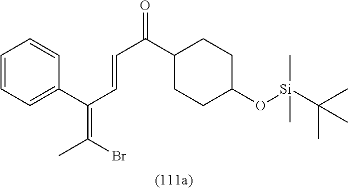 [SMILES: C/C(Br)=C(/C=C/C(=O)C1CCC(O[Si](C)(C)C(C)(C)C)CC1)c1ccccc1] | ¹HNMR (CDCl₃, 400 MHz) δ: 7.96 (d, 1H, J = 16.0 Hz), 7.43-7.36 (m, 3H), 7.08-7.06 (m, 2H), 5.63 (d, 1H, J = 16.0 Hz), 3.56-3.49 (m, 1H), 2.51-2.44 (m, 1H), 2.26 (s, 3H), 1.90-1.77 (m, 4H), 1.39-1.25 (m, 4H), 0.86 (s, 9H), 0.03 (s, 6H); Mass (LCMS): 463.0 and 465.1 (M⁺, M + 2). |
| 1-(4-((tert- butyldimethylsilyl)oxy)cyclohexyl)-2-(7- methyl-6-phenyl-5H-pyrrolo[1,2- c]imidazol-5-yl)ethan-1-one 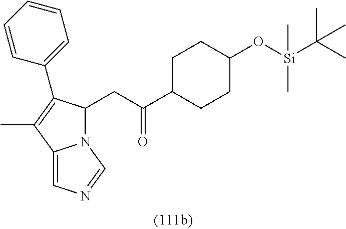 [SMILES: CC1=C(c2ccccc2)C(CC(=O)C2CCC(O[Si](C)(C)C(C)(C)C)CC2)n2cncc21] | ¹HNMR (CDCl₃, 400 MHz) δ: 7.53 (br. s, 1H), 7.45- 7.41 (m, 2H), 7.35-7.31 (m, 1H), 7.28-7.27 (m, 2H), 6.87 (br. s, 1H), 5.55-5.53 (m, 1H), 3.54-3.47 (m, 1H), 2.88 (dd, 1H, J = 2.0, 18.8 Hz), 2.54 (dd, 1H, J = 10.4, 18.8 Hz), 2.20-2.18 (m, 4H), 1.88-1.74 (m, 4H), 1.42-1.24 (m, 4H), 0.85 (s, 9H), 0.03 (s, 6H); Mass (LCMS): 451.2 (M + 1). |
| 1-(4-hydroxycyclohexyl)-2-(7-methyl-6- phenyl-5H-pyrrolo[1,2-c]imidazol-5- yl)ethan-1-one 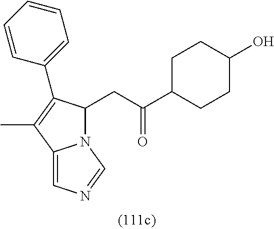 [SMILES: CC1=C(c2ccccc2)C(CC(=O)C2CCC(O)CC2)n2cncc21] | ¹HNMR (CDCl₃, 400 MHz) δ: 7.55 (br. s, 1H), 7.45- 7.41 (m, 2H), 7.35-7.31 (m, 1H), 7.27-7.25 (m, 2H), 6.87 (br. s, 1H), 5.56-5.53 (m, 1H), 3.60-3.52 (m, 1H), 2.84 (dd, 1H, J = 2.0, 18.4 Hz), 2.55 (dd, 1H, J = 10.8, 18.4 Hz), 2.18 (d, 3H, J = 2.0 Hz), 2.02-1.98 (m, 1H), 1.86-1.79 (m, 4H), 1.23-1.16 (m, 4H); Mass (LCMS): 337.1 (M + 1). |
| Methyl tosyl-L-prolinate 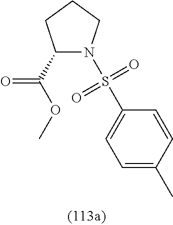 [SMILES: COC(=O)[C@@H]1CCCN1S(=O)(=O)c1ccc(C)cc1] | ¹HNMR (CDCl₃, 400 MHz) δ: 7.71 (d, 2H, J = 8 Hz), 7.43 (d, 2H, J = 8 Hz), 4.17 (dd, 1H, J = 2, 4 Hz), 3.85 (s, 1H), 3.64 (s, 3H), 3.17-3.07 (m, 1H), 2.39 (s, 3H), 1.94-1.76 (m, 3H), 1.61-1.52 (m, 1H); Mass (LCMS): 284.0 (M + 1) |
| Dimethyl (S)-(2-oxo-2-(1- tosylpyrrolidin-2-yl)ethyl)phosphonate 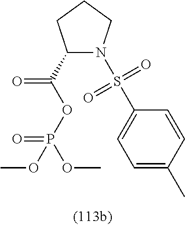 | ¹HNMR (CDCl₃, 400 MHz) δ: 7.71 (d, 2H, J = 8 Hz), 7.34 (d, 2H, J = 8 Hz), 3.84 (s, 1.5H), 3.81 (s, 3H), 3.78 (s, 1.5H), 3.76-3.70 (m, 1H), 3.67-3.62 (m, 1H), 3.56-3.51 (m, 1H), 3.39 (d, 0.5H, J = 14.8 Hz), 3.33 (d, 0.5H, J = 14.8 Hz), 3.28-3.22 (m, 1H), 2.43(s, 3H), 2.16-2.09(m, 1H), 1.89-1.73(m, 2H), 1.55- 1.48(m, 1H); Mass (LCMS): 376.0 (M + 1) |
| 5-Bromo-4-phenyl-1-((S)-1- tosylpyrrolidin-2-yl)hexa-2,4-dien-1-one 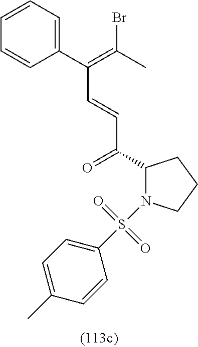 | ¹HNMR (CDCl₃, 400 MHz) δ: 8.07 (d, 0.4H, J = 15.6 Hz), 7.91-7.86 (m, 0.6H), 7.72-7.64 (m, 2H), 7.44-7.23 (m, 5H), 7.13-7.03 (m, 2H), 6.00-5.88 (m, 1H), 4.47-4.07 (m, 1H), 3.31-3.20 (m, 2H), 2.75 (s, 1.4H), 2.42 (s, 3H), 2.29 (s, 1.6H), 1.93-1.55 (m, 4H); Mass (LCMS): 475.9 (M + 1) |
| Methyl benzoyl-L-prolinate 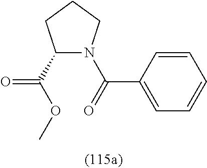 [SMILES: COC(=O)[C@@H]1CCCN1C(=O)c1ccccc1] | ¹HNMR (CDCl₃, 400 MHz) δ: 7.56 (dd, 2H, J = 1.2, 8 Hz), 7.43-7.35 (m, 3H), 3.77 (s, 3H), 3.66-3.62 (m, 1H), 3.56-3.50 (m, 2H), 2.33-2.28 (m, 1H), 2.02-1.98 (m, 2H), 1.90-1.86 (m, 1H); Mass (LCMS): 234.1 (M + 1). |
| Dimethyl (S)-(2-(1-benzoylpyrrolidin-2- yl)-2-oxoethyl)phosphonate 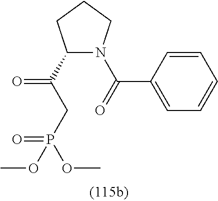 [SMILES: COP(=O)(CC(=O)[C@@H]1CCCN1C(=O)c1ccccc1)OC] | Mass (LCMS): 326.1 (M + 1). |
| 1-((S)-1-benzoylpyrrolidin-2-yl)-5- bromo-4-phenylhexa-2,4-dien-1-one 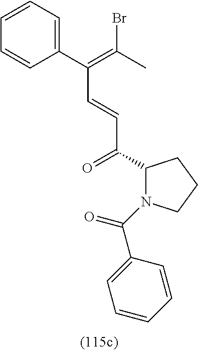 | ¹HNMR (CDCl₃, 400 MHz) δ: 8.11 (d, 0.5H, J = 16 Hz), 7.91 (d, 0.5H, J = 15.2 Hz), 7.46-7.34 (m, 8H), 7.11-7.08 (m, 2H), 5.84 (d, 0.5H, J = 15.2 Hz), 5.78 (d, 0.5H, J = 16 Hz), 4.91 (dd, 0.5H, J = 5.2, 8.8 Hz), 4.74 (dd, 0.5H, J = 6, 8.8 Hz), 3.54-3.45 (m, 2H), 2.74 (s, 1.5H), 2.27 (s, 1.5H), 1.93-1.78 (m, 4H); Mass (LCMS): 426 (M + 2). |
| 1-(4-Hydroxycyclohexyl)-2-(7-methyl-6- phenyl-5H-pyrrolo[1,2-c]imidazol-5- yl)ethan-1-one 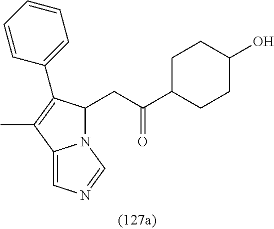 [SMILES: CC1=C(c2ccccc2)C(CC(=O)C2CCC(O)CC2)n2cncc21] | ¹HNMR (CDCl₃, 400 MHz) δ: 7.58 (br. s, 1H), 7.45- 7.41 (m, 2H), 7.35-7.31 (m, 1H), 7.28-7.27 (m, 2H), 6.87 (br. s, 1H), 5.58-5.55 (m, 1H), 3.96-3.91 (m, 1H), 2.83 (dd, 1H, J = 2.4, 18.4 Hz), 2.56 (dd, 1H, J = 10.8, 18.4 Hz), 2.32-2.25 (m, 1H), 2.18 (d, 3H, J = 1.6 Hz), 1.86-1.75 (m, 4H), 1.59-1.52 (m, 4H); Mass (LCMS): 337.1 (M + 1). |
| Dimethyl (2-(4-cyclohexylphenyl)-2- oxoethyl)phosphonate 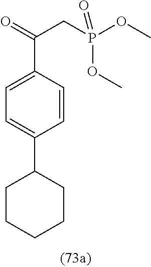 [SMILES: COP(=O)(CC(=O)c1ccc(C2CCCCC2)cc1)OC] | ¹HNMR (CDCl₃, 400 MHz) δ: 7.97-7.92 (m, 2H), 7.32-7.26 (m, 2H), 3.80 (s, 3H), 3.77 (s, 3H), 3.62 (d, 2H, J = 22.4 Hz), 2.58-2.54 (m, 1H), 1.87-1.83 (m, 4H), 1.79-1.73 (m, 1H), 1.45-1.35 (m, 5H); Mass (LCMS): 311 (M + 1). |
| 5-Bromo-1-(4-cyclohexylphenyl)-4- phenylhexa-2,4-dien-1-one 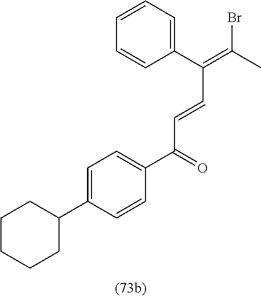 [SMILES: C/C(Br)=C(\C=C\C(=O)c1ccc(C2CCCCC2)cc1)c1ccccc1] | ¹HNMR (CDCl₃, 400 MHz) δ: 8.15 (d, 0.4H, J = 15.2 Hz), 7.99-7.94 (m, 1.6H), 7.70-7.67 (m, 1.5H), 7.46-7.39 (m, 2.5H), 7.28-7.21 (m, 2.5H), 7.16-7.13 (m, 1.5H), 6.44 (d, 0.5H, J = 14.8 Hz), 6.38 (d, 0.5H, J = 15.2 Hz), 3.89 (s, 2H), 2.78 (s, 1H), 2.29 (s, 1H), 1.44-1.34 (m, 7H), 1.28-1.22 (m, 3H). |
| (2E,4Z)-5-bromo-1-(4,4- difluorocyclohexyl)-4-phenylhexa-2,4- dien-1-one 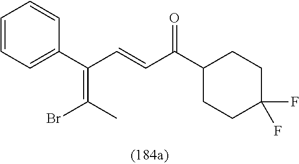 [SMILES: C/C(Br)=C(\C=C\C(=O)C1CCC(F)(F)CC1)c1ccccc1] | LCMS: 369.1. |
| 2-(7-methyl-6-phenyl-5H-pyrrolo[1,2- c]imidazol-5-yl)-1-(4-nitrophenyl)ethan- 1-one 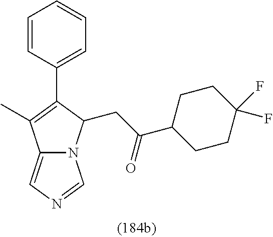 [SMILES: CC1=C(c2ccccc2)C(CC(=O)C2CCC(F)(F)CC2)n2cncc21] | LCMS: 357.1 (M + 1). |
| dimethyl (2-oxo-2-(4- phenylcyclohexyl)ethyl)phosphonate 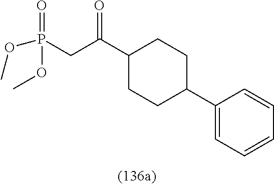 [SMILES: COP(=O)(CC(=O)C1CCC(c2ccccc2)CC1)OC] | ¹HNMR (CDCl₃, 400 MHz) δ: 7.31-7.25 (m, 2H), 7.22-7.15 (m, 3H), 3.18 (d, 2H, J = 22.5 Hz), 2.69- 2.46 (m, 2H), 2.27-1.96 (m, 4H), 1.79-1.62 (m, 4H), 1.49 (s, 3H), 1.45 (s, 3H); Mass (LCMS): 311.1 (M + 1). |
| 5-bromo-4-phenyl-1-(4- phenylcyclohexyl)hexa-2,4-dien-1-one 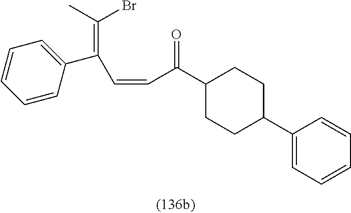 [SMILES: C/C(Br)=C(/C=C\C(=O)C1CCC(c2ccccc2)CC1)c1ccccc1] | ¹HNMR (CDCl₃, 400 MHz) δ: 8.00 (d, 1H, J = 15.6 Hz), 7.43-7.36 (m, 4H), 7.21-7.13 (m, 5H), 7.10-7.09 (m, 1H), 5.75 (d, 1H, J = 15.6 Hz), 4.19-4.14 (m, 1H), 2.76-2.75 (m, 1H), 2.51-2.49 (m, 2H), 2.26 (s, 3H), 2.07-2.03 (m, 2H), 1.74-1.68 (m, 4H); (LCMS): 410.1 (M + 1). |

### Example 87: 1-Cyclohexyl-2-(7-methyl-6-(p-tolyl)-5H-pyrrolo[1,2-c]imidazol-5-yl)ethan-1-ol

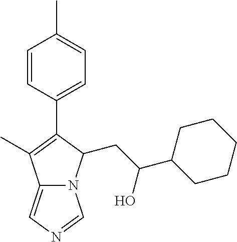 [SMILES: CC1=C(c2ccc(C)cc2)C(CC(O)C2CCCCC2)n2cncc21]

Final product of Example 86 (1-Cyclohexyl-2-(7-methyl-6-(p-tolyl)-5H-pyrrolo[1,2-c]imidazol-5-yl)ethan-1-one) was subjected to similar experimental procedure as described in synthesis of Example 19 to generate final compound 1-Cyclohexyl-2-(7-methyl-6-(p-tolyl)-5H-pyrrolo[1,2-c]imidazol-5-yl)ethan-1-ol.

¹HNMR (CDCl₃, 400 MHz) δ: 7.18 (br. s, 1H), 7.24-7.17 (m, 4H), 6.86 (br. s, 1H), 5.42-5.35 (m, 0.2H), 5.23-5.17 (m, 0.8H), 3.71-3.64 (m, 1H), 3.32-3.28 (m, 1H), 2.38 (s, 3H), 2.17 (d, 1H, J=1.6 Hz), 2.15 (d, 2H, J=1.2 Hz), 2.00-1.95 (m, 1H), 1.78-1.66 (m, 3H), 1.57-1.37 (m, 4H), 1.20-1.03 (m, 5H); Mass (LCMS): 337.1 (M+1)

Following examples have been synthesized by the above procedure described in Example 86 and 87 with their corresponding intermediates in similar reaction conditions:

| Example | | |
|---|---|---|
| No. | IUPAC Name/Structure | Analytical Data |
| 94. | 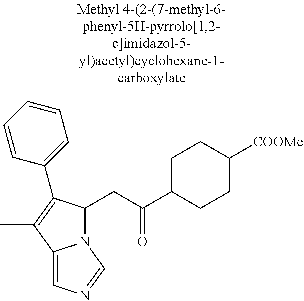 | 1HNMR (CDCl₃, 400 MHz) δ: 7.58 (br. s, 1H), 7.45-7.41 (m, 2H), 7.73 (d, 1H, J = 8.0 Hz), 7.35-7.30 (m, 1H), 7.27-7.25 (m, 1H), 6.88 (br. s, 1H), 5.57-5.54 (m, 1H), 3.67 (s, 3H), 2.81 (dd, 1H, J = 2.4, 18.8 Hz), 2.87-2.79 (m, 1H), 2.52 (dd, 1H, J = 10.4, 18.4 Hz), 2.34-2.27 (m, 1H), 2.18 (d, 3H, J = 2 Hz), 2.04-1.94 (m, 2H), 1.67- 1.52 (m, 6H). Mass (LCMS): 391.1 (M + 1); Purity: 96.52%. |
| 95. | 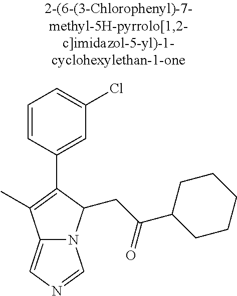 [SMILES: CC1=C(c2cccc(Cl)c2)C(CC(=O)C2CCCCC2)n2cncc21] | ¹HNMR (CDCl₃, 400 MHz) δ: 7.56 (s, 1H), 7.38- 7.29 (m, 3H), 7.14-7.12 (m, 1H), 6.89 (s, 1H), 5.51 (d, 1H, J = 10.4 Hz), 2.82 (d, 0.4H, J = 2.4 Hz), 2.78 (d, 0.6H, J = 2.4 Hz), 2.54 (dd, 1H, J = 10.4, 18.8 Hz), 2.29-2.23 (m, 1H), 2.18 (d, 3H, J = 1.92 Hz), 1.76-1.63 (m, 6H), 1.35-1.30 (m, 2H), 1.22-1.17 (m, 2H). Mass (LCMS): 355 (M + 1); Purity: 94.68%. |
| 96. | 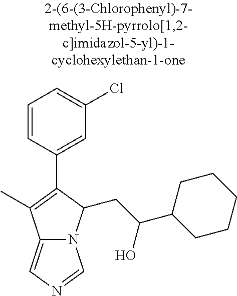 [SMILES: CC1=C(c2cccc(Cl)c2)C(CC(O)C2CCCCC2)n2cncc21] | ¹HNMR (CDCl₃, 400 MHz) δ: 7.83 (br. s, 1H), 7.36 (t, 1H, J = 7.6 Hz), 7.31-7.28 (m, 2H), 7.17 (d, 1H, J = 7.6 Hz), 6.91 (br. s, 1H), 5.39-5.36 (br. s, 0.4H), 5.20 (br. s, 0.6H), 3.72-3.64 (m, 0.4H), 3.33-3.32 (m, 0.6H), 2.33-2.29 (m, 1H), 2.19 (d, 1H, J = 1.2 Hz), 2.16 (d, 2H, J = 1.6 Hz), 1.98-1.97 (m, 0.5H), 1.95-1.93 (m, 0.5H), 1.81-1.66 (m, 5H), 1.54-1.50 (m, 2H), 1.29-1.25 (m, 1H), 1.23-1.04 (m, 4H). Mass (LCMS): 357.1 (M + 1); Purity: 96.43%. |
| 97. | 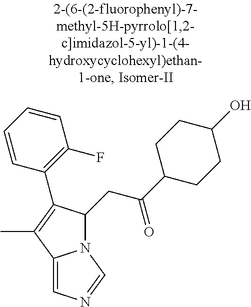 [SMILES: CC1=C(c2ccccc2F)C(CC(=O)C2CCC(O)CC2)n2cncc21] | ¹HNMR (CD₃OD, 400 MHz) δ: 7.81 (br. s, 1H), 7.38-7.30 (m, 2H), 7.22-7.12 (m, 2H), 6.91 (br. s, 1H), 5.58-5.56 (m, 1H), 4.50 (br. s, 1H), 3.75- 3.70 (m, 1H), 2.84 (dd, 1H, J = 2.8, 18.4 Hz), 2.65 (dd, 1H, J = 10.0, 18.4 Hz), 2.32-2.26 (m, 1H), 1.99 (s, 3H), 1.67-1.52 (m, 4H), 1.46-1.39 (m, 4H); Mass (LCMS): 355.1 (M + 1); Purity: 90.88%. |
| 98. | 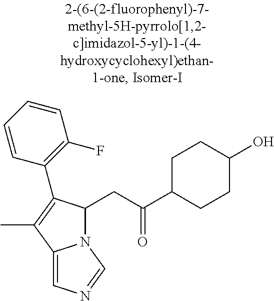 [SMILES: CC1=C(c2ccccc2F)C(CC(=O)C2CCC(O)CC2)n2cncc21] | ¹HNMR (CD₃OD, 400 MHz) δ: 7.72 (br. s, 0.44H), 7.66 (br. s, 0.49H), 7.38-7.34 (m, 1H), 7.24-7.13 (m, 3H), 6.95 (br. s, 0.45H), 6.93 (br. s, 0.52H), 5.61-5.55 (m, 1H), 3.91 (br. s, 1H), 3.60-3.53 (m, 1H), 2.79-2.73 (m, 1H), 2.63-2.54 (m, 1H), 2.33-2.19 (m, 1H), 2.08 (br. s, 3H), 2.06-1.99 (m, 4H), 1.88-1.77 (m, 2H), 1.44.1.27 (m, 2H); Mass (LCMS): 355.1 (M + 1); Purity: 94.51%. |
| 99. | 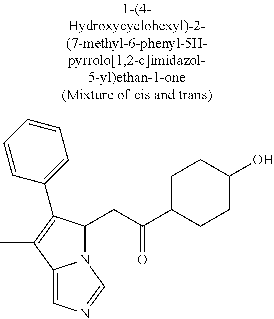 [SMILES: CC1=C(c2ccccc2)C(CC(=O)C2CCC(O)CC2)n2cncc21] | ¹HNMR (CDCl₃, 400 MHz) δ: 7.55 (br. s, 1H), 7.43 (t, 2H, J = 7.2 Hz), 7.35-7.31 (m, 1H), 7.28- 7.24 (m, 2H), 6.87 (br. s, 1H), 5.56-5.53 (m, 1H), 3.60-3.52 (m, 1H), 2.83 (dd, 1H, J = 2.4, 18.0 Hz), 2.55 (dd, 1H, J = 10.4, 18.4 Hz), 2.26- 2.21 (m, 1H), 2.18 (d, 3H, J = 1.6 Hz), 2.02-1.98 (m, 2H), 1.86-1.77 (m, 2H), 1.44-1.19 (m, 4H); Mass (LCMS): 337.1 (M + 1); Purity: 98.53%. |
| 100. | 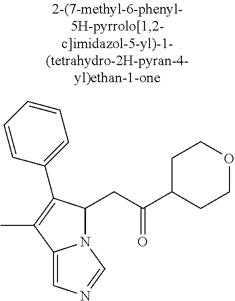 [SMILES: CC1=C(c2ccccc2)C(CC(=O)C2CCOCC2)n2cncc21] | ¹HNMR (CDCl₃, 400 MHz) δ: 7.59 (br. s, 1H), 7.45-7.42 (m, 2H), 7.35-7.32 (m, 1H), 7.28 (br. s, 2H), 6.89 (br. s, 1H), 5.58-5.55 (m, 1H), 3.97- 3.94 (m, 2H), 3.36-3.30 (m, 2H), 2.88-2.83 (m, 1H), 2.54 (dd, 1H, J = 10.4, 18.4 Hz), 2.49-2.43 (m, 1H), 2.18 (s, 3H), 1.66-1.63 (m, 4H); Mass (LCMS): 323.1 (M + 1); Purity: 91.79%. |
| 101. | 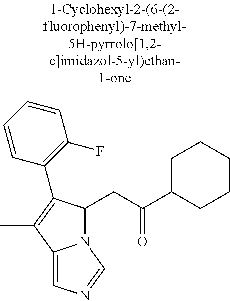 [SMILES: CC1=C(c2ccccc2F)C(CC(=O)C2CCCCC2)n2cncc21] | ¹HNMR (CDCl₃, 400 MHz) δ: 7.58 (br. s, 1H), 7.37-7.32 (m, 1H), 7.22-7.12 (m, 3H), 6.89 (br. s, 1H), 5.56-5.54 (m, 1H), 2.74-2.70 (m, 1H), 2.56 (dd, 1H, J = 10.4, 18.4 Hz), 2.28-2.23 (m, 1H), 2.07 (s, 3H), 1.81-1.73 (m, 6H), 1.26-1.16 (m, 4H); Mass (LCMS): 339.1 (M + 1); Purity: 91.19%. |
| 102. | 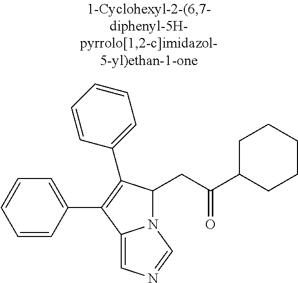 [SMILES: O=C(CC1C(c2ccccc2)=C(c2ccccc2)c2cncn21)C1CCCCC1] | ¹HNMR (CDCl₃, 400 MHz) δ: 7.62 (br. s, 1H), 7.41-7.39 (m, 2H), 7.32-7.29 (m, 6H), 7.19-7.17 (m, 2H), 6.94 (br. s, 1H), 5.70-5.68 (m, 1H), 2.84 (dd, 1H, J = 2.4, 18.4 Hz), 2.66 (dd, 1H, J = 10.4, 18.4 Hz), 2.31-2.24 (m, 1H), 1.80-1.73 (m, 4H), 1.33-1.25 (m, 6H); Mass (LCMS): 383.1 (M + 1); Purity: 93.16%. |
| 103. | 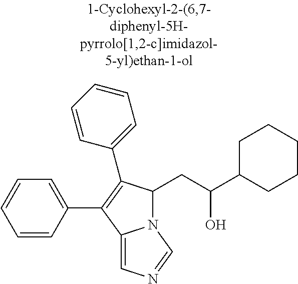 [SMILES: OC(CC1C(c2ccccc2)=C(c2ccccc2)c2cncn21)C1CCCCC1] | ¹HNMR (CDCl₃, 400 MHz) δ: 7.42-7.38 (m, 2H), 7.32-7.29 (m, 6H), 7.22-7.20 (m, 2H), 6.96-6.94 (m, 2H), 5.55-5.53 (m, 1H), 3.78-3.74 (m, 1H), 3.39-3.37 (m, 1H), 1.90-1.79 (m, 4H), 1.55-1.48 (m, 2H), 1.29-1.22 (m, 2H), 1.17-1.10 (m, 4H); Mass (LCMS): 385.1 (M + 1); Purity: 99.39%. |
| 104. | 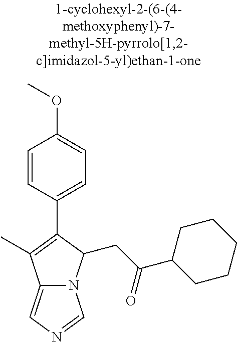 [SMILES: COc1ccc(C2=C(C)c3cncn3C2CC(=O)C2CCCCC2)cc1] | ¹HNMR (CDCl₃, 400 MHz) δ: 7.54 (s, 1H), 7.19 (d, 2H, J = 8.4 Hz), 6.95 (d, 2H, J = 8.4 Hz), 6.83 (s, 1H), 5.50 (m, 1H), 3.84 (s, 3H), 2.80 (dd, 1H, J = 2, 18.4 Hz), 2.52 (dd, 1H, J = 10.8, 18.4 Hz), 2.24-2.22 (m, 1H), 2.15 (d, 3H, J = 1.6 Hz), 1.74-1.72 (m, 5H), 1.36-1.14 (m, 4H); LCMS: 351.1 (M + 1); Purity: 99.27%. |
| 105. | 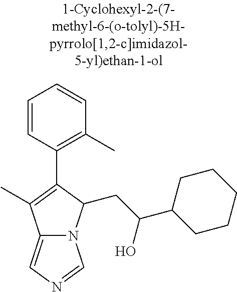 [SMILES: CC1=C(c2ccccc2C)C(CC(O)C2CCCCC2)n2cncc21] | ¹HNMR (CDCl₃, 400 MHz) δ: 7.84-7.74 (m, 1H), 7.41-7.27 (m, 2H), 7.23-7.17 (m, 1H), 7.13-7.03 (m, 1H), 6.94-6.79 (m, 1H), 5.22-5.07 (m, 1H), 3.96-3.92 (m, 0.2H), 3.75-3.59 (m, 1H), 3.14-3.09 (m, 0.8H), 2.33 (s, 0.7H), 2.29- 2.26 (m, 2.3H), 1.88 (s, 3H), 1.82-1.74 (m, 2H), 1.54-1.45 (m, 2H), 1.21-1.00 (m, 5H), 0.97-0.76 (m, 4H). Mass (LCMS): 337.1 (M + 1); Purity: 90.62%. |
| 106. | 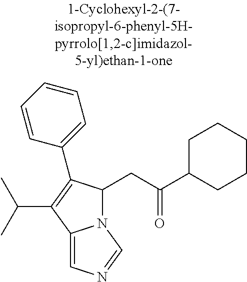 [SMILES: CC(C)C1=C(c2ccccc2)C(CC(=O)C2CCCCC2)n2cncc21] | ¹HNMR (CDCl₃, 400 MHz) δ: 7.58 (br. s, 1H), 7.44-7.40 (m, 2H), 7.36-7.32 (m, 1H), 7.24-7.22 (m, 2H), 6.88 (br. s, 1H), 5.50-5.47 (m, 1H), 3.09-3.03 (m, 1H), 2.76-2.72 (m, 1H), 2.52 (dd, 1H, J = 10.4, 18.4 Hz), 2.26-2.21 (m, 1H), 1.71- 1.64 (m, 4H), 1.39-1.37 (m, 2H), 1.32-1.28 (m, 4H), 1.18 (d, 6H, J = 6.8 Hz); Mass (LCMS): 349.1 (M + 1); Purity: 90.21%. |
| 107. | 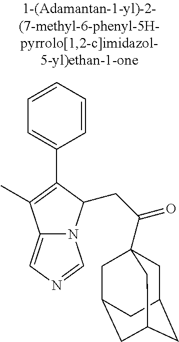 [SMILES: CC1=C(c2ccccc2)C(CC(=O)C23CC4CC(CC(C4)C2)C3)n2cncc21] | ¹HNMR (CDCl₃, 400 MHz) δ: 7.50 (s, 1H), 7.43 (t, 2H, J = 7.2 Hz), 7.34-7.31 (m, 1H), 7.28 (d, 1H, J = 1.2 Hz), 7.27-7.26 (m, 1H), 6.87 (s, 1H), 5.58 (d, 1H, J = 10.4 Hz), 2.74 (dd, 1H, J = 2.4, 18.4 Hz), 2.61 (dd, 1H, J = 10.4, 18.4 Hz), 2.19 (d, 3H, J = 1.6 Hz), 2.01-1.96 (m, 3H), 1.77- 1.42 (m, 12H). Mass (LCMS): 373.2 (M + 1); Purity: 99.42%. |
| 108. | 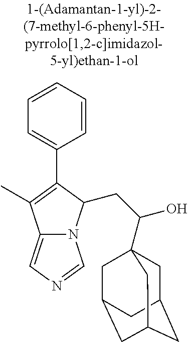 [SMILES: CC1=C(c2ccccc2)C(CC(O)C23CC4CC(CC(C4)C2)C3)n2cncc21] | ¹HNMR (CDCl₃, 400 MHz) δ: 7.83 (br. s, 1H), 7.45-7.40 (m, 2H), 7.34-7.27 (m, 3H), 6.90-6.86 (m, 1H), 5.26-5.23 (m, 1H), 2.92 (d, 1H, J = 10 Hz), 2.20 (d, 0.3H, J = 1.6 Hz), 2.17 (d, 2.7H, J = 2 Hz), 2.04-2.01 (m, 1H), 1.94-1.87 (m, 3H), 1.75-1.66 (m, 3H), 1.57-1.49 (m, 4H), 1.39-1.28 (m, 6H). Mass (LCMS): 375.2 (M + 1); Purity: 98.44%. |
| 111. | 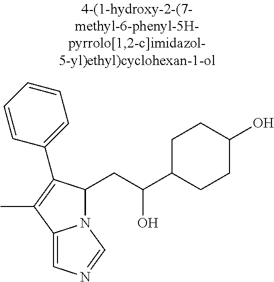 [SMILES: CC1=C(c2ccccc2)C(CC(O)C2CCC(O)CC2)n2cncc21] | 1HNMR (CDCl₃, 400 MHz) δ: 7.85 (br. s, 1H), 7.44-7.41 (m, 2H), 7.34-7.27 (m, 3H), 6.87 (br. s, 1H), 5.26-5.21 (m, 1H), 3.50-3.43 (m, 1H), 3.37-3.32 (m, 1H), 2.16 (d, 3H, J = 1.6 Hz), 1.99-1.93 (m, 3H), 1.81-1.86 (m, 4H), 1.22-1.10 (m, 4H); Mass (LCMS): 339.1 (M + 1); Purity: 94.72%. |
| 121. | 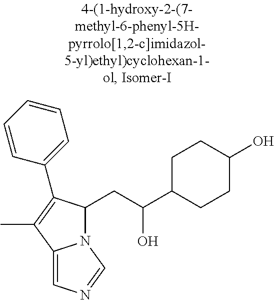 [SMILES: CC1=C(c2ccccc2)C(CC(O)C2CCC(O)CC2)n2cncc21] | 1HNMR (CDCl₃, 400 MHz) δ: 7.88 (br. s, 1H), 7.45-7.41 (m, 2H), 7.35-7.27 (m, 3H), 6.87 (br. s, 1H), 5.27-5.22 (m, 1H), 3.50-3.42 (m, 1H), 3.37-3.33 (m, 1H), 2.16 (d, 3H, J = 1.6 Hz), 2.00-1.93 (m, 4H), 1.81-1.73 (m, 1H), 1.70-1.66 (m, 1H), 1.57-1.53 (m, 1H), 1.18-1.12 (m, 2H), 1.06-0.95 (m, 2H); Mass (LCMS): 339.1 (M + 1); Purity: 94.92%. |
| 122. | 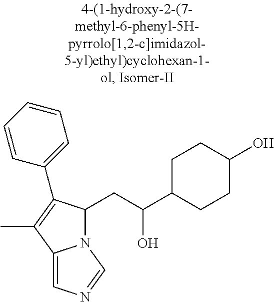 [SMILES: CC1=C(c2ccccc2)C(CC(O)C2CCC(O)CC2)n2cncc21] | ¹HNMR (CDCl₃, 400 MHz) δ: 7.85 (br. s, 1H), 7.44-7.41 (m, 2H), 7.34-7.27 (m, 3H), 6.87 (br. s, 1H), 5.26-5.22 (m, 1H), 3.50-3.43 (m, 1H), 3.36-3.32 (m, 1H), 2.16 (d, 3H, J = 1.6 Hz), 1.99-1.94 (m, 4H), 1.82-1.74 (m, 1H), 1.70-1.65 (m, 1H), 1.57-1.53 (m, 1H), 1.20-1.12 (m, 2H), 1.06-0.95 (m, 2H); Mass (LCMS): 339.1 (M + 1); Purity: 93.03%. |
| 134. | 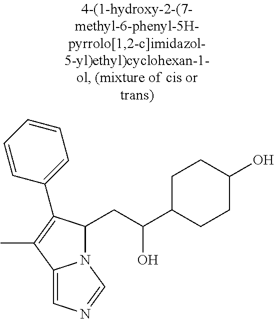 [SMILES: CC1=C(c2ccccc2)C(CC(O)C2CCC(O)CC2)n2cncc21] | ¹HNMR (CDCl₃, 400 MHz) δ: 7.74 (br. s, 1H), 7.45-7.41 (m, 2H), 7.34-7.27 (m, 3H), 6.89 (br. s, 1H), 5.42-5.40 (m, 1H), 3.37-3.67 (m, 1H), 3.52-3.45 (m, 1H), 2.19 (d, 3H, J = 1.6 Hz), 2.00-1.89 (m, 4H), 1.83-1.81 (m, 1H), 1.58-1.52 (m, 1H), 1.48-1.40 (m, 1H), 1.21-1.11 (m, 2H), 1.07-0.95 (m, 2H); Mass (LCMS): 339.1 (M + 1); Purity: 94.41%. |
| 113. | 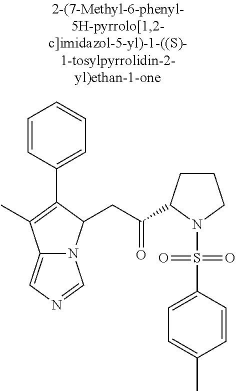 | ¹HNMR (CDCl₃, 400 MHz) δ: 7.67-7.61 (m, 2H), 7.54-7.52 (m, 1H), 7.47-7.42 (m, 2H), 7.36-7.28 (m, 5H), 6.89 (s, 1H), 5.61 (d, 0.5H, J = 10.4 Hz), 5.56 (d, 0.5H, J = 9.6 Hz), 4.12-4.09 (m, 0.5H), 3.96-3.93 (m, 0.5H), 3.43-3.32 (m, 1.5 H), 3.22-3.16 (m, 1H), 3.07-3.02 (m, 0.5H), 2.92 (dd, 0.5H, J = 9.6, 18.8 Hz), 2.66 (dd, 0.5H, J = 10.8, 18.8 Hz), 2.43 (d, 3H, J = 2.4 Hz), 2.21-2.17 (m, 3H), 1.75-1.67 (m, 2H), 1.59-1.53 (m, 1H), 1.52-1.45 (m, 1H); Mass (LCMS); 462.1; Purity: 96.03%. |
| 114. | 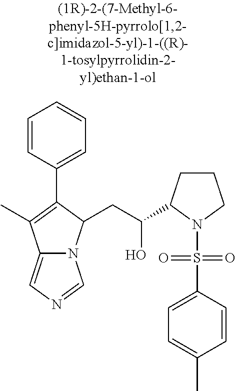 [SMILES: CC1=C(c2ccccc2)C(C[C@@H](O)C2CCCN2S(=O)(=O)c2ccc(C)cc2)n2cncc21] | ¹HNMR (CDCl₃, 400 MHz) δ: 7.95-7.85 (m, 1H), 7.73-7.67 (m, 1H), 7.58 (d, 1H, J = 7.6 Hz), 7.47-7.40 (m, 2H), 7.34-7.29 (m, 5H), 6.95-6.87 (m, 1H), 5.53-5.49 (m, 0.3H), 5.38-5.34 (m, 0.4H), 5.30-5.25 (m, 0.3H), 4.11-4.04 (m, 0.4H), 3.77-3.71 (m, 0.6H), 3.68-3.60 (m, 1H), 3.41-3.36 (m, 1H), 3.34-3.14 (m, 2H), 2.45-2.38 (m, 3H), 2.21-2.15 (m, 3H), 1.94-1.84 (m, 2H), 1.45-1.35 (m, 2H), 1.22-1.11 (m, 2H). Mass (LCMS): 464.1 (M + 1); Purity: 92.26%. |
| 115. | 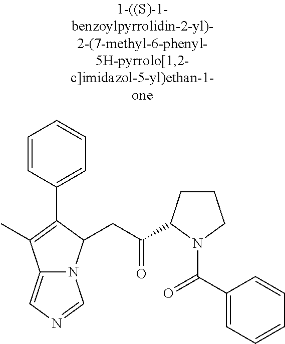 | ¹HNMR (CDCl₃, 400 MHz) δ: 7.72 (s, 0.5H), 7.59 (s, 0.5H), 7.55-7.53 (m, 1H), 7.46-7.36 (m, 6H), 7.34-7.27 (m, 3H), 6.88 (s, 1H), 5.62 (t, 1H, J = 13.2 Hz), 4.67-4.62 (m, 1H), 3.69-3.63 (m, 0.5H), 3.57-3.43 (m, 1.5H), 3.25 (dd, 0.5H, J = 2, 18.4 Hz), 2.97 (dd, 0.5H, J = 2.8, 18.4 Hz), 2.76 (dd, 0.5H, J = 10, 18.4 Hz), 2.57 (dd, 0.5H, J = 11.2, 18.8 Hz), 2.19 (d, 1.5H, J = 1.6 Hz), 2.18 (d, 1.5H, J = 1.6 Hz), 2.14-2.07 (m, 1H), 2.04-1.96 (m, 1H), 1.93-1.81 (m, 2H), Mass (LCMS): 412.1 (M + 1); Purity: 94.1%. |
| 116. | 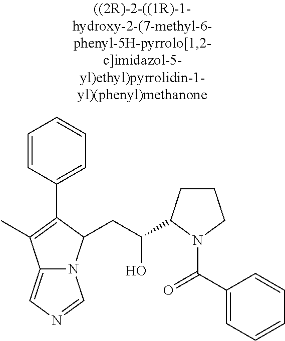 [SMILES: CC1=C(c2ccccc2)C(C[C@@H](O)C2CCCN2C(=O)c2ccccc2)n2cncc21] | ¹HNMR (CDCl₃, 400 MHz) δ: 7.96-7.88 (m, 1H), 7.52-7.27 (m, 10H), 6.92-6.88 (m, 1H), 5.33-5.29 (m, 1H), 4.30-4.22 (m, 1H), 3.71-3.63 (m, 1H), 3.49-3.38 (m, 1H), 3.35-3.24 (m, 1H), 2.23-2.15 (m, 3H), 1.97-1.90 (m, 2H), 1.86-1.74 (m, 1H), 1.73-1.53 (m, 2H), 1.50-1.29 (m, 2H). Mass (LCMS): 414.1 (M + 1); Purity: 93.24%. |
| 127. | 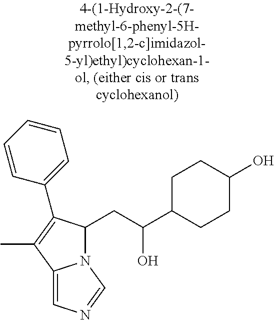 [SMILES: CC1=C(c2ccccc2)C(CC(O)C2CCC(O)CC2)n2cncc21] | ¹HNMR (CDCl₃, 400 MHz) δ: 7.84 (br. s, 1H), 7.44-7.40 (m, 2H), 7.34-7.27 (m, 3H), 6.87 (br. s, 1H), 5.28-5.23 (m, 1H), 3.66-3.63 (m, 1H), 3.40-3.35 (m, 1H), 2.16 (d, 3H, J = 1.6 Hz), 2.03-1.95 (m, 2H), 1.69-1.64 (m, 4H), 1.46-1.39 (m, 5H); Mass (LCMS): 339.1 (M + 1); Purity: 92.94%. |
| 130. | 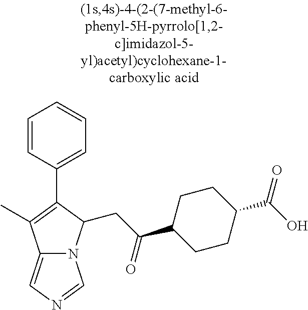 | ¹HNMR (MeOD, 400 MHz) δ: 8.78 (s, 1H), 7.54-7.46 (m, 2H), 7.45-7.42 (m, 4H), 5.98 (d, 1H, J = 10.8 Hz), 3.13 (dd, 1H, J = 2.4, 19.2 Hz), 2.87 (dd, 1H, J = 10, 18.8 Hz), 2.35-2.34 (m, 1H), 2.23 (d, 3H, J = 1.6 Hz), 2.21-2.18 (m, 1H), 2.01-1.98 (m, 2H), 1.86-1.83 (m, 2H), 1.42-1.34 (m, 4H); Mass (LCMS): 365.1 (M + 1), Purity: 96.31% |
| 131. | 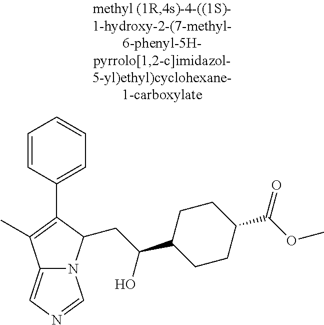 | ¹HNMR (CDCl₃, 400 MHz) δ: ¹HNMR (CDCl₃, 400 MHz) δ: 7.41 (s, 1H), 7.42 (t, 2H, J = 7.6 Hz), 7.34-7.27 (m, 3H), 6.88 (s, 1H), 5.27-5.21 (m, 1H), 3.67-3.64 (m, 1H), 3.63 (s, 3H), 3.34- 3.31 (m, 1H), 2.19-2.17 (m, 1H), 2.16 (d, 3H, J = 1.6 Hz), 2.14-2.10 (m, 1H), 2.02-1.94 (m, 4H), 1.85-1.58 (m, 2H), 1.42-1.16 (m, 4H), Mass (LCMS): 381.1 (M + 1), Purity: 96.64%. |
| 133. | 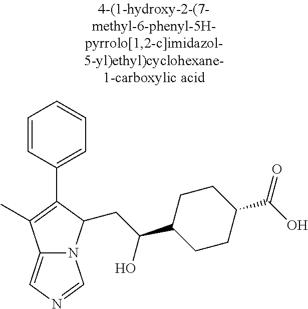 | ¹HNMR (CDCl₃, 400 MHz) δ: 8.52 (s, 0.4H), 8.34 (s, 0.6H), 7.42 (t, 2H, J = 7.2 Hz), 7.35- 7.26 (m, 3H), 6.92 (s, 0.4H), 6.88 (s, 0.6H), 5.54 (d, 1H, J = 10.8 Hz), 5.29-5.27 (m, 1H), 3.68- 3.64 (m, 1H), 3.42-3.37 (m, 1H), 2.17-2.12 (m, 4H), 2.05 (s, 3H), 1.97-1.85 (m, 4H), 1.75-1.66 (m, 1H), 1.58-1.55 (m, 1H); Mass (LCMS): 367.1 (M + 1), Purity: 95.96% |
| 136. | 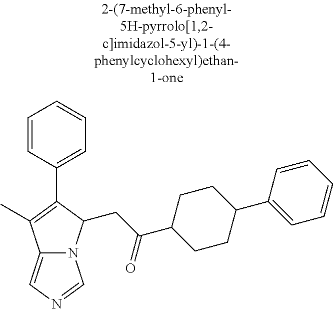 [SMILES: CC1=C(c2ccccc2)C(CC(=O)C2CCC(c3ccccc3)CC2)n2cncc21] | ¹HNMR (CDCl₃, 400 MHz) δ: 7.65 (s, 1H), 7.46-7.42 (m, 2H), 7.36-7.32 (m, 1H), 7.29-7.27 (m, 3H), 7.19-7.14 (m, 4H), 6.91 (s, 1H), 5.59 (d, 1H, J = 10.0 Hz), 2.87 (dd, 1H, J = 1.6, 18.4 Hz), 2.60 (dd, 1H, J= 10.8, 18.4 Hz), 2.49- 2.44 (m, 1H), 2.36-2.31 (m, 1H), 2.20 (d, 3H, J = 1.2 Hz), 2.80-2.01 (m, 1H), 1.99-1.94 (m, 1H), 1.90-1.89 (m, 1H), 1.64-1.58 (m, 1H), 1.53-1.41 (m, 4H), Mass (LCMS): 397.1 (M + 1); Purity: 91.62%. |
| 186. | 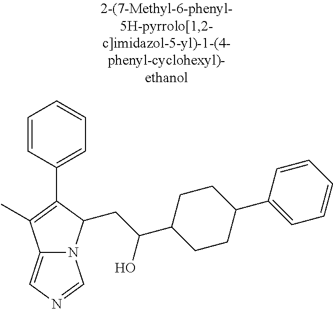 [SMILES: CC1=C(c2ccccc2)C(CC(O)C2CCC(c3ccccc3)CC2)n2cncc21] | ¹HNMR (CDCl₃, 400 MHz) δ: 7.89 (s, 1H), 7.43 (t, 2H, J = 7.6 Hz), 7.35-7.27 (m, 4H), 7.27-7.24 (m, 1H), 7.18-7.13 (m, 3H), 6.93 (s, 0.3H), 6.90 (s, 0.7H), 5.30-5.22 (m, 1H), 3.78-3.75 (m, 0.3H), 3.43-3.36 (m, 0.7H), 2.42-2.29 (m, 2H), 2.20 (d, 0.9H, J = 1.6 Hz), 2.17 (d, 2.1H, J = 1.6H), 2.06-1.99 (m, 2H), 1.96-1.77 (m, 5H), 1.67-1.64 (m, 3H); LCMS (M + 1): 397.1; Purity: 92.42%. |
| 73. | 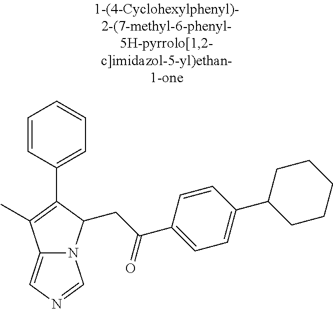 [SMILES: CC1=C(c2ccccc2)C(CC(=O)c2ccc(C3CCCCC3)cc2)n2cncc21] | ¹HNMR (CDCl₃, 400 MHz) δ: 7.79 (d, 2H, J = 8.4 Hz), 7.69 (s, 1H), 7.45-7.41 (m, 2H), 7.34- 7.30 (m, 3H), 7.25 (s, 1H), 7.23 (s, 1H), 6.89 (s, 1H), 5.76 (d, 1H, J = 10.8 Hz), 3.31 (dd, 1H, J = 2, 18.4 Hz), 3.10-3.03 (m, 1H), 2.55-2.50 (m, 1H), 2.22 (d, 3H, J = 1.6 Hz), 1.86-1.82 (m, 5H), 1.76-1.73 (m, 1H), 1.41-1.35 (m, 4H); LCMS: 397.1 (M + 1); Purity: 98.07%. |
| 74. | 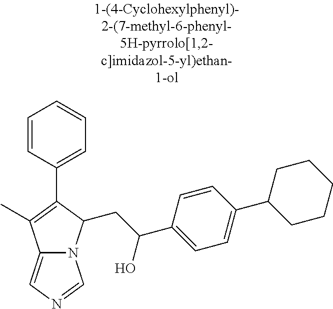 [SMILES: CC1=C(c2ccccc2)C(CC(O)c2ccc(C3CCCCC3)cc2)n2cncc21] | ¹HNMR (CDCl₃, 400 MHz) δ: 8.20 (br. s, 0.4H), 7.83 (br. s, 0.6H), 7.43-7.38 (m, 1H), 7.36-7.33 (m, 1H), 7.31-7.27 (m, 1H), 7.24-7.22 (m, 1H), 7.19-7.14 (m, 4H), 7.10 (d, 1H, J = 8.4 Hz), 7.00 (br. s, 0.4H), 6.88 (br. s, 0.6H), 5.47 (m, 0.4H), 5.17-5.16 (m, 0.6H), 5.00 (dd, 0.4H, J = 3.6, 10 Hz), 4.82-4.79 (m, 0.6H), 2.49-2.42 (m, 1H), 2.16 (d, 1.5H, J = 1.6 Hz), 2.15 (d, 1.5H, J = 1.6 Hz), 2.13-2.11 (m, 1H), 1.84-1.80 (m, 4H), 1.75-1.71 (m, 1H), 1.68-1.61 (m, 2H), 1.43-1.32 (m, 5H); Mass (LCMS): 399.1 (M + 1); Purity: 99.39%. |
| 184. | 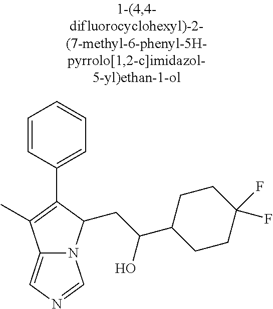 [SMILES: CC1=C(c2ccccc2)C(CC(O)C2CCC(F)(F)CC2)n2cncc21] | ¹HNMR (CDCl₃, 400 MHz) δ: 8.11 (s, 1H), 7.43 (t, 2H, J = 8 Hz), 7.36-7.26 (m, 3H), 7.04 (s, 0.3H), 6.94 (s, 0.7H), 5.53-5.49 (m, 0.3H), 5.30- 5.26 (m, 1H), 5.12-5.03 (m, 0.7H), 3.76-3.73 (m, 0.3H), 3.51-3.48 (m, 0.7H), 2.18 (d, 0.9H, J = 1.2 Hz), 2.16 (d, 2.1H, J = 1.6 Hz), 2.12-1.94 (m, 3H), 1.80-1.48 (m, 8H); LCMS: 359.1 (M + 1); Purity: 95.82%. |
| 185. | 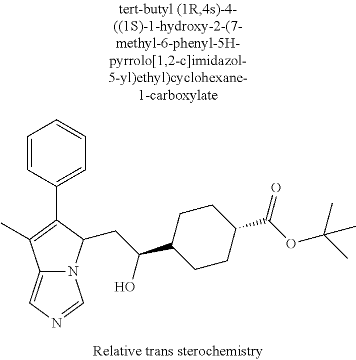 | ¹HNMR (CDCl₃, 400 MHz) δ: 7.82 (s, 0.8H), 7.78 (s, 0.2H), 7.44-7.39 (m, 2H), 7.34-7.27 (m, 3H), 6.90 (s, 0.2H), 6.88 (s, 0.8H), 5.42-5.39 (m, 0.2H), 5.27-5.21 (m, 0.8H), 3.33-3.29 (m, 1H), 2.19 (d, 0.6H, J = 1.6 Hz), 2.16 (d, 2.4H, J = 1.6 Hz), 2.08-1.55 (m, 12H), 1.41 (s, 1.8H), 1.40 (s, 7.2H); LCMS: 423.1 (M + 1); Purity: 91.67%. |
| 189. | 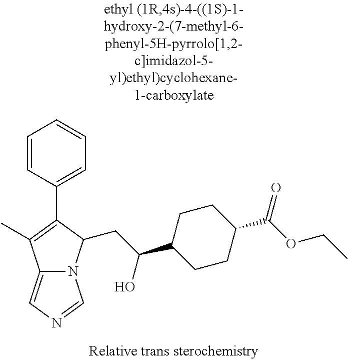 | ¹HNMR (CDCl₃, 400 MHz) δ: 7.85 (br. s, 1H), 7.44-7.40 (m, 2H), 7.34-7.27 (m, 3H), 6.89 (br. s, 1H), 5.45-5.41 (m, 0.3H), 5.28-5.22 (m, 0.7H), 4.10 (q, 2H, J = 7.2, 14 Hz), 3.72-3.63 (m, 1H), 3.37-3.30 (m, 1H), 2.19 (d, 0.9H, J = 1.6 Hz), 2.16 (d, 2.1H, J = 1.6 Hz), 2.11-1.87 (m, 7H), 1.63-1.58 (m, 3H), 1.21 (t, 3H, J = 7.2Hz), 0.99-0.91 (m, 2H); LCMS: 395.1 (M + 1); Purity: 96.88%. |

### Example 109: Preparation of 4-(2-(7-methyl-6-phenyl-5H-pyrrolo[1,2-c]imidazol-5-yl)acetyl)-N-phenylcyclohexane-1-carboxamide

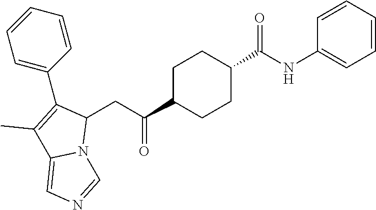

To a solution of 4-(2-(7-methyl-6-phenyl-5H-pyrrolo[1,2-c]imidazol-5-yl)acetyl)cyclohexane-1-carboxylic acid (Example 130, 100 mg, 0.274 mmol) in DMF (3 ml) was added aniline (31 mg, 0.329 mmol) and DIPEA (0.15 ml, 0.824 mmol) at room temperature. HATU (157 mg, 0.412 mmol) was added to the reactin mixture and stirred at room temperature for 2 hours. Water (10 ml) and ethyl acetate (15 ml) were added to the reaction mixture and layers were separated. Aqueous layer was extracted with ethyl acetate (2×15 ml). Combined organic extracts were dried over sodium sulfate and concentrated to give crude residue, which was purified silica gel column chromatography to give the title compound 4-(2-(7-methyl-6-phenyl-5H-pyrrolo[1,2-c]imidazol-5-yl)acetyl)-N-phenylcyclohexane-1-carboxamide (25 mg, 21%) as yellow solid.

¹HNMR (CDCl₃, 400 MHz) δ: 7.57 (br. s, 1H), 7.52-7.48 (m, 3H), 7.46-7.42 (m, 2H), 7.36-7.28 (m, 4H), 7.08 (t, 1H, J=7.2 Hz), 6.88 (br. s, 1H), 5.57-5.54 (m, 1H), 2.87 (dd, 1H, J=2.4, 18.4 Hz), 2.59 (dd, 1H, J=10.8, 18.4 Hz), 2.26-2.28 (m, 1H), 2.25-2.15 (m, 4H), 2.04-1.98 (m, 2H), 1.94-1.89 (m, 2H), 1.58-1.42 (m, 4H); Mass (LCMS): 440.1 (M+1); Purity: 99.6%.

Following intermediates have been synthesized as described in the above process of Example 109:

| Intermediate No. | Analytical Data |
|---|---|
| 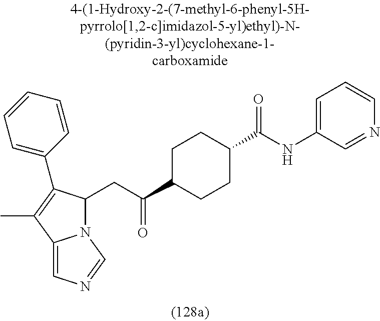 | ¹HNMR (CDCl₃, 400 MHz) δ: 8.70 (br. s, 1H), 8.61 (d, 1H, J = 2.4 Hz), 8.32-8.30 (m, 1H,), 8.20-8.17 (m, 1H), 7.62 (br. s, 1H), 7.47-7.43 (m, 2H), 7.37-7.33 (m, 1H), 7.29-7.27 (m, 1H), 7.25-7.23 (m, 1H), 6.89 (br. s, 1H), 5.56-5.54 (m, 1H), 2.90 (dd, 1H, J = 2.0, 18.4 Hz), 2.60 (dd, 1H, J = 10.8, 18.4 Hz), 2.38-2.28 (m, 2H), 2.19 (d, 3H, J = 1.6 Hz), 2.08-2.01 (m, 2H), 1.96-1.90 (m, 2H), 1.60-1.30 (m, 4H); Mass (LCMS): 440.1 (M + 1). |
| 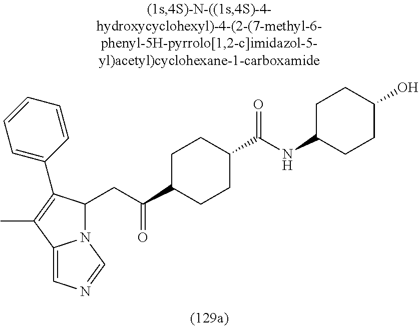 | ¹HNMR (CDCl₃, 400 MHz) δ: 7.56 (br. s, 1H), 7.45- 7.41 (m, 2H), 7.36-7.31 (m, 1H), 7.28-725 (m, 2H), 6.87 (br. s, 1H), 5.56-5.53 (m, 1H), 5.27-5.25 (m, 1H), 3.76-3.66 (m, 1H), 3.63-3.55 (m, 1H), 2.84 (dd, 1H, J = 2.4, 18.8 Hz), 2.56 (dd, 1H, J = 10.8, 18.8 Hz), 2.31-2.24 (m, 1H), 2.18 (d, 3H, J = 1.6 Hz), 1.90-1.93 (m, 6H), 1.91-1.80 (m, 6H), 1.46-1.34 (m, 5H); Mass (LCMS): 462.1 (M + 1). |

Following examples have been synthesized by coupling of acid compound from example 130 and appropriate amine/aniline counterpart using similar conditions as described in Example 109 and their corresponding following hydroxy compounds also synthesized as described in above procedure.

| Example | | |
|---|---|---|
| No. | IUPAC Name/Structure | Analytical Data |
| 110. | 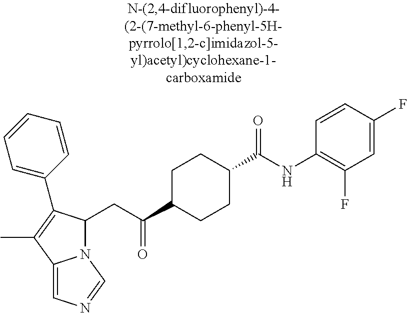 | ¹HNMR (CDCl₃, 400 MHz) δ: 8.24-8.18 (m, 1H), 7.56 (br. s, 1H), 7.46-7.42 (m, 2H), 7.36- 7.28 (m, 3H), 6.87-6.83 (m, 3H), 5.57-5.54 (m, 1H), 2.87 (dd, 1H, J = 2.4, 18.4 Hz), 2.58 (dd, 1H, J = 10.8, 18.4 Hz), 2.36-2.21 (m, 2H), 2.19 (d, 3H, J = 1.6 Hz), 2.07-1.99 (m, 2H), 1.96- 1.87 (m, 2H), 1.58-1.44 (m, 4H); Mass (LCMS): 476.1 (M + 1); Purity: 99.59%. |
| 112. | 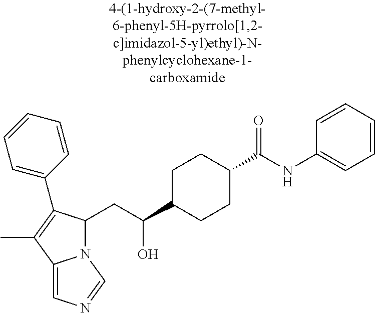 | ¹HNMR (CDCl₃, 400 MHz) δ: 7.56 (br. s, 1H), 7.52-7.50 (m, 2H), 7.45-7.41 (m, 2H), 7.35- 7.28 (m, 5H), 7.03 (t, 1H, J = 7.4 Hz), 6.88 (br. s, 1H), 5.26-5.21 (m, 1H), 3.66-3.64 (m, 1H), 3.40-3.38 (m, 1H), 2.17 (d, 3H, J = 1.6 Hz), 2.01-1.93 (m, 4H), 1.66-1.44 (m, 5H), 1.12-1.01 (m, 2H); Mass (LCMS): 442.1 (M + 1); Purity: 97.38%. |
| 117. | 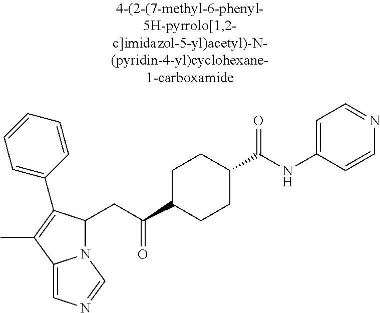 | ¹HNMR (CDCl₃, 400 MHz) δ: 9.24 (br. s, 1H), 8.45 (d, 2H, J = 6.0 Hz), 7.65 (br. s, 1H), 7.56 (d, 2H, J = 6.0 Hz), 7.47-7.43 (m, 2H), 7.38- 7.34 (m, 1H), 7.29-7.27 (m, 2H), 6.90 (br. s, 1H), 5.57-5.54 (m, 1H), 2.90 (dd, 1H, J = 2.4, 18.4 Hz), 2.61 (dd, 1H, J = 10.8, 18.4 Hz), 2.36- 2.30 (m, 2H), 2.20 (d, 3H, J = 1.6 Hz), 2.04- 2.00 (m, 2H), 1.92-1.89 (m, 2H), 1.57-1.47 (m, 4H); Mass (LCMS): 441.1 (M + 1); Purity: 93.35%. |
| 118. | 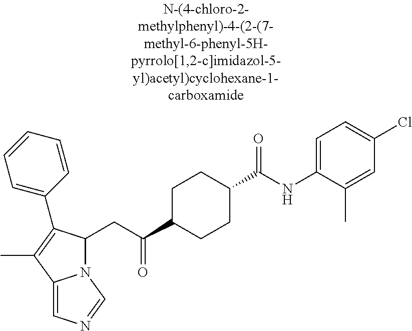 | ¹HNMR (CDCl₃, 400 MHz) δ: 7.70 (d, 1H, J = 8.8 Hz), 7.56 (br. s, 1H), 7.43 (t, 2H, J = 7.6 Hz), 7.35-7.32 (m, 1H), 7.29-7.27 (m, 2H), 7.16-7.14 (m, 2H), 6.87 (br. s, 1H), 5.56-5.54 (m, 1H), 2.86 (dd, 1H, J = 2.4, 18.4 Hz), 2.58 (dd, 1H, J = 10.8, 18.4 Hz), 2.35-2.29 (m, 2H), 2.21 (s, 3H), 2.18 (d, 3H, J = 1.6 Hz), 2.07-2.00 (m, 2H), 1.94-1.88 (m, 2H), 1.61-1.46 (m, 4H); Mass (LCMS): 488.1 (M + 1); Purity: 95.16%. |
| 119. | 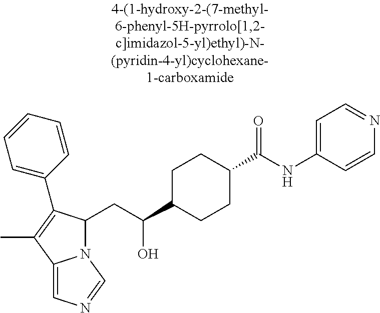 | ¹HNMR (CDCl₃, 400 MHz) δ: 8.42 (br. s, 2H), 8.01 (br. s, 1H), 7.58-7.53 (m, 2H), 7.45-7.42 (m, 2H), 7.36-7.32 (m, 1H), 7.30-7.26 (m, 2H), 6.87 (br. s, 1H), 5.23 (br. s, 1H), 3.65-3.53 (m, 1H), 3.57-3.50 (m, 1H), 2.26-2.23 (m, 2H), 2.17 (br. s, 3H), 1.99-1.90 (m, 4H), 1.76-1.65 (m, 2H), 1.54-1.45 (m, 4H); Mass (LCMS): 443.1 (M + 1); Purity: 90.17%. |
| 120. | 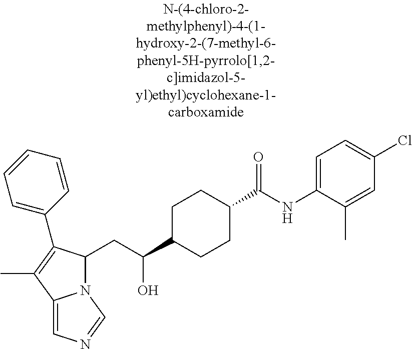 | ¹HNMR (CDCl₃, 400 MHz) δ: 7.97 (br. s, 1H), 7.45 (t, 2H, J = 7.2 Hz), 7.40-7.32 (m, 3H), 7.23-7.21 (m, 2H), 7.15-7.12 (m, 1H), 6.83 (br. s, 1H), 5.39-5.35 (m, 1H), 3.38-3.34 (m, 1H), 2.30-2.24 (m, 1H), 2.17 (s, 3H), 2.15 (d, 3H, J = 1.6 Hz), 2.03-1.98 (m, 1H), 1.90-1.88 (m, 3H), 1.79-1.75 (m, 1H), 1.70-1.61 (m, 2H), 1.52-1.42 (m, 4H); Mass (LCMS): 490.1 (M + 1); Purity: 96.99%. |
| 123. | 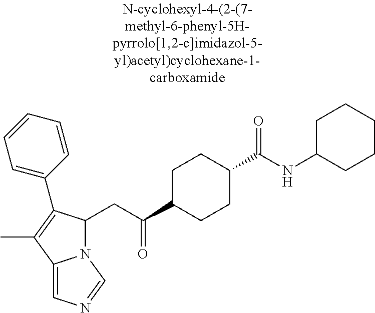 | ¹HNMR (CDCl₃, 400 MHz) δ: 7.55 (br. s, 1H), 7.45-7.41 (m, 2H), 7.35-7.31 (m, 1H), 7.28-727 (m, 2H), 6.87 (br. s, 1H), 5.56-5.53 (m, 1H), 5.28-5.25 (m, 1H), 3.75-3.68 (m, 1H), 2.84 (dd, 1H, J = 2.4, 18.4 Hz), 2.56 (dd, 1H, J = 10.8, 18.4 Hz), 2.31-2.24 (m, 1H), 2.18 (br. s, 3H), 1.97-1.82 (m, 7H), 1.50-1.25 (m, 7H), 1.22- 1.03 (m, 4H); Mass (LCMS): 446.1 (M + 1); Purity: 97.18%. |
| 124. | 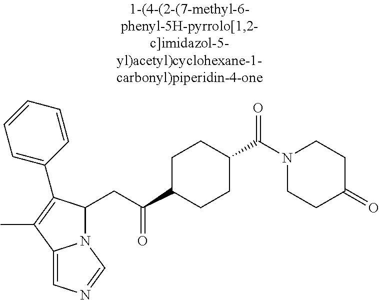 | ¹HNMR (CDCl₃, 400 MHz) δ: 7.54 (br. s, 1H), 7.45-7.41 (m, 2H), 7.35-7.31 (m, 1H), 7.28-727 (m, 2H), 6.87 (br. s, 1H), 5.57-5.54 (m, 1H), 3.87-3.84 (m, 2H), 3.78-3.74 (m, 2H), 2.85 (dd, 1H, J = 2.4, 18.4 Hz), 2.58 (dd, 1H, J = 10.8, 18.4 Hz), 2.53-2.44 (m, 4H), 2.38-2.30 (m, 1H), 2.19 (d, 3H, J = 1.6 Hz), 1.91-1.82 (m, 4H), 1.58-1.43 (m, 5H); Mass (LCMS): 446.1 (M + 1); Purity: 97.52%. |
| 125. | 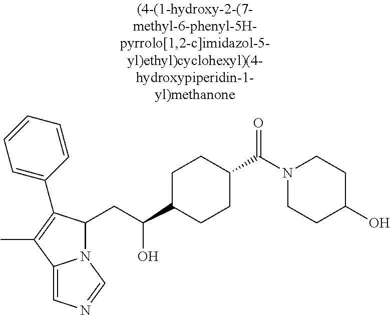 | ¹HNMR (CDCl₃, 400 MHz) δ: 7.84 (br. s, 1H), 7.44-7.41 (m, 2H), 7.34-7.28 (m, 3H), 6.87 (br. s, 1H), 5.26-5.21 (m, 1H), 4.19-4.05 (m, 1H), 3.93-3.89 (m, 1H), 375-3.64 (m, 2H), 3.37-3.32 (m, 1H), 3.21-3.10 (m, 2H), 2.39-2.33 (m, 1H), 2.16 (br. s, 3H), 2.03-1.82 (m, 8H), 1.78-1.67 (m, 2H), 1.52-1.42 (m, 5H); Mass (LCMS): 450.1 (M + 1); Purity: 93.49%. |
| 126. | 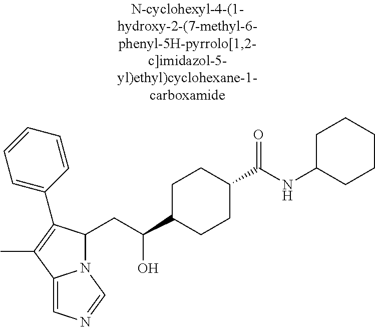 | ¹HNMR (CDCl₃, 400 MHz) δ: 7.82 (br. s, 1H), 7.44-7.40 (m, 2H), 7.34-7.27 (m, 3H), 6.87 (br. s, 1H), 5.28-5.23 (m, 1H), 3.75-3.67 (m, 1H), 3.36-3.32 (m, 1H), 2.16 (d, 3H, J = 1.6 Hz), 1.99-1.74 (m, 8H), 1.70-1.59 (m, 6H), 1.41- 1.23 (m, 4H), 1.15-0.94 (m, 4H); Mass (LCMS): 448.2 (M + 1); Purity: 100%. |
| 128. | 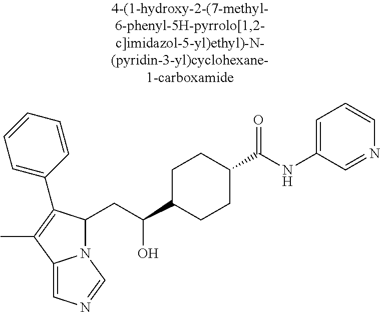 | ¹HNMR (CDCl₃, 400 MHz) δ: 8.87 (br. s, 1H), 8.65-8.63 (m, 1H,), 8.30-8.27 (m, 1H), 8.21- 8.17 (m, 1H), 7.96 (br. s, 1H), 7.45-7.42 (m, 2H), 7.36-7.31 (m, 1H), 7.29-7.27 (m, 1H), 7.25-7.21 (m, 1H), 6.86 (br. s, 1H), 5.25-5.22 (m, 1H), 3.67-3.64 (m, 1H), 3.53-3.49 (m, 1H), 2.19-2.17 (m, 2H), 2.16 (d, 3H, J = 1.6 Hz), 2.01-1.91 (m, 4H), 1.80-1.66 (m, 3H), 1.56- 1.45 (m, 3H); Mass (LCMS): 443.1 (M + 1); Purity: 99.46%. |
| 129. | 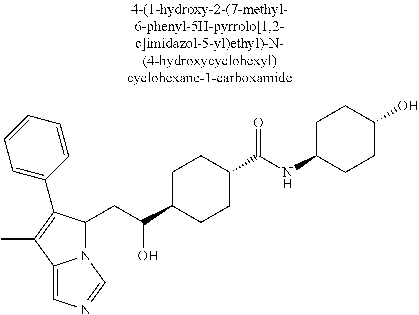 [SMILES: CC1=C(c2ccccc2)C(CC(O)[C@H]2CC[C@H](C(=O)N[C@H]3CC[C@H](O)CC3)CC2)n2cncc21] | ¹HNMR (CDCl₃, 400 MHz) δ: 7.84 (br. s, 1H), 7.44-7.40 (m, 2H), 7.34-7.27 (m, 3H), 6.87 (br. s, 1H), 5.25-5.23 (m, 2H), 3.74-3.63 (m, 1H), 3.67-3.63 (m, 1H), 3.61-3.55 (m, 1H), 3.35- 3.31 (m, 1H), 2.16 (d, 3H, J = 1.6 Hz), 2.03- 1.88 (m, 10H), 1.45-1.24 (m, 6H), 1.22-1.10 (m, 4H); Mass (LCMS): 464.1 (M + 1); Purity: 98.21%. |
| 132. | 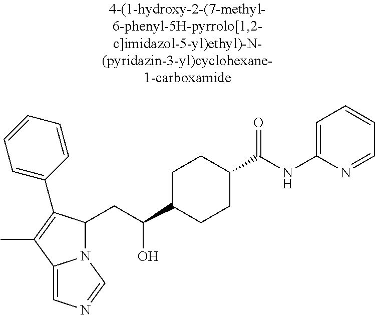 | ¹HNMR (CDCl₃, 400 MHz) δ: 8.22-8.18 (m, 2H), 7.86 (br. s, 1H), 7.70-7.65 (m, 1H), 7.45- 7.41 (m, 2H), 7.35-7.28 (m, 3H), 7.03-6.99 (m, 1H), 6.89 (br. s, 1H), 5.27-5.22 (m, 1H), 3.67 (br. s, 1H), 3.39-3.34 (m, 1H), 2.16 (d, 3H, J = 1.6 Hz), 2.02-1.96 (m, 3H), 1.84-1.77 (m, 2H), 1.67-1.64 (m, 2H), 1.52-1.45 (m, 3H), 1.11- 0.98 (m, 2H); Mass (LCMS): 443.2 (M + 1); Purity: 98.54%. |
| 135. | 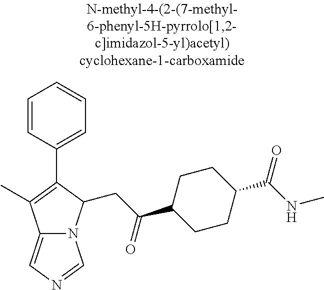 | ¹HNMR (CDCl₃, 400 MHz) δ: 7.54 (s, 1H), 7.44-7.34 (m, 2H), 7.35-7.31 (t, 2H, J = 7.6 Hz), 7.29-7.27 (m, 1H), 6.87 (s, 1H), 5.54 (d, 1H, J = 10 Hz), 5.45 (br s, 1H), 2.84 (dd, 1H, J = 2, 18.8 Hz), 2.77 (d, 3H, J = 4.8 Hz), 2.55 (dd, 1H, J = 10.8, 18.8 Hz), 2.28-2.24 (m, 1H), 2.17 (d, 3H, J = 1.6 Hz), 2.04-1.83 (m, 7H), 1.50-1.42 (m, 2H); Mass (LCMS): 378.1 (M + 1), Purity: 92.11%. |

### Example 137: Preparation of 4-[1-Hydroxy-2-(7-methyl-6-phenyl-5H-pyrrolo[1,2-c]imidazol-5-yl)-ethyl]-piperidine-1-carboxylic acid benzyl ester

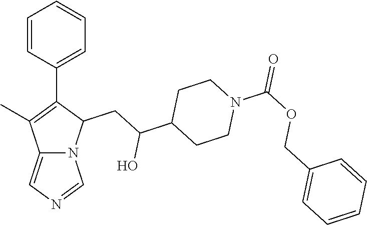 [SMILES: CC1=C(c2ccccc2)C(CC(O)C2CCN(C(=O)OCc3ccccc3)CC2)n2cncc21]

### Step-1: Preparation of Piperidine-1,4-dicarboxylic acid 1-benzyl ester 4-methyl ester

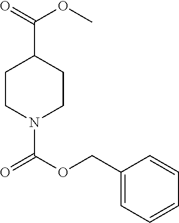 [SMILES: COC(=O)C1CCN(C(=O)OCc2ccccc2)CC1]

To a stirred solution of NaHCO₃ (25.24 g, 300 mmol) in 1,4-dioxane:water (1:1.8, 700 ml) was added compound methyl piperidine-4-carboxylate hydrochloride (20 g, 11.13 mmol) portion wise at about 0-5° C. Reaction mixture was stirred for about 10-15 minutes, then benzyl chloroformate (41.74 g, 244 mmol) was added slowly at about 0-5° C. Reaction stirred at room temperature for about 24 hours. After completion, reaction was partitioned between ethyl acetate & water, layers were separated and aqueous layer was extracted with ethyl acetate (2×300 mL). Combined organic layers was dried over anhydrous Na₂SO₄ and distilled off to get crude product, which was column purified using ethyl acetate: hexanes (2:8) as eluent to get pure compound Piperidine-1,4-dicarboxylic acid 1-benzyl ester 4-methyl ester (22.62 g, 85%).

¹H NMR (CDCl₃, 400 MHz) δ: 7.36-7.33 (m, 5H), 5.11 (s, 2H), 4.12-4.03 (m, 2H), 3.68 (s, 3H), 2.92 (t, 2H, J=10.2 Hz), 2.50-2.43 (m, 1H), 1.94-1.84 (m, 2H), 1.69-1.60 (m, 2H)

### Step-2: Preparation of 4-[2-(Dimethoxy-phosphoryl)-acetyl]-piperidine-1-carboxylic acid benzyl ester

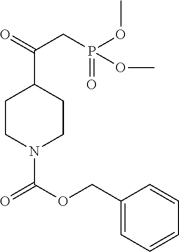 [SMILES: COP(=O)(CC(=O)C1CCN(C(=O)OCc2ccccc2)CC1)OC]

To a stirred solution of dimethylmethyl phosphonate (13.42 g, 108 mmol) in THF (150 mL) was added n-BuLi (7.22 g, 112 mmol) at about −78° C. and stirred for about 30 minutes. THF solution of piperidine-1,4-dicarboxylic acid 1-benzyl ester 4-methyl ester (Step 1, 15 g, 54.0 mmol) was then added drop-wise to above reaction mixture at about −78° C. Temperature of reaction was raised to room temperature and stirred for about 1-1.5 hours. Reaction was monitored by TLC. After completion of reaction was quenched with DM water, and extracted with ethyl acetate (3×300 mL). Combined organic layers were dried on anhydrous Na₂SO₄ and evaporated under vacuum to get compound 4-[2-(Dimethoxy-phosphoryl)-acetyl]-piperidine-1-carboxylic acid benzyl ester (16 g, 80%) as yellowish liquid.

¹H NMR (CDCl₃, 400 MHz) δ: 7.39-7.30 (m, 5H), 5.12 (s, 2H), 4.19-4.17 (m, 2H), 3.79 (s, 3H), 3.77 (s, 3H), 3.13 (d, 2H, J=22.8 Hz), 2.88 (t, 2H, J=10.8 Hz), 2.80-2.72 (m, 1H), 1.60-1.53 (m, 2H), 1.51-1.43 (m, 2H). Mass (LCMS): 370.1 (M+1)

### Step-3: Preparation of benzyl 4-(5-bromo-4-phenylhexa-2,4-dienoyl)piperidine-1-carboxylate

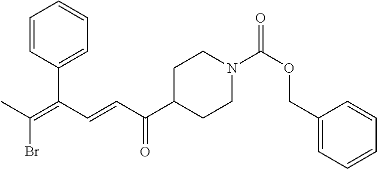 [SMILES: C/C(Br)=C(/C=C/C(=O)C1CCN(C(=O)OCc2ccccc2)CC1)c1ccccc1]

In the suspension of NaH (0.98 g, 24.64 mmol) in THF at about 0° C. was added solution of compound 4-[2-(Dimethoxy-phosphoryl)-acetyl]-piperidine-1-carboxylic acid benzyl ester (Step 2, 12.31 g, 33.30 mmol) in THF drop wise and reaction mixture stirred for about 30 minutes. Solution of benzeneacetaldehyde, α-(1-bromoethylidene) (E&Z) (5.0 g, 22.40 mmol) in THF was added slowly in above reaction mixture at about 0° C. Temperature of reaction mixture was raised to room temperature and stirred for about 1-1.5 hours. Reaction was monitored by TLC. After completion of reaction was quenched with DM water and extracted with ethyl acetate (3×100 mL). Combined organic layers were dried on anhydrous Na₂SO₄, then evaporated to get crude product which was purified by column chromatography to obtain semi-solid compound benzyl 4-(5-bromo-4-phenylhexa-2,4-dienoyl)piperidine-1-carboxylate (6.3 g, 61%). This compound was taken for the next step without further purification.

Mass (LCMS): 468 (M), 470 (M+2).

### Step-4: Preparation of benzyl 4-(4-phenyl-5-(1-trityl-1H-imidazol-4-yl)hexa-2,4-dienoyl)piperidine-1-carboxylate

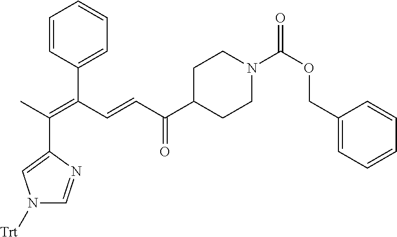

To a mixture of compound benzyl 4-(5-bromo-4-phenylhexa-2,4-dienoyl)piperidine-1-carboxylate (Step 3, 2.5 g, 5.33 mmol) and N-trityl imidazole-4-boronic acid (3.40 g, 9.59 mmol) in 1,4-dioxane:water (4:1; 80 ml) was added K₂CO₃ (1.84 g, 13.3 mmol) at room temperature. The reaction mixture was degassed for about 20 minutes using nitrogen gas. To this suspension Pd(dppf)Cl₂-DCM complex (0.435 g, 0.5 mmol) was added and reaction mixture was degassed for another about 15 minutes. Reaction mixture was heated at about 95° C. for about 1.5-2 hours. After completion of reaction, solvent was evaporated under reduced pressure and residue was diluted with water (50 ml) and extracted with ethyl acetate (3×100 mL). Combined organic layer was dried over anhydrous Na₂SO₄ and concentrated to give crude product which was purified by column chromatography to yield compound benzyl 4-(4-phenyl-5-(1-trityl-1H-imidazol-4-yl)hexa-2,4-dienoyl)piperidine-1-carboxylate (0.82 g, 22.1%) as pale yellow solid. Product formation was confirmed by LCMS.

### Step-5: Preparation of 4-[2-(7-Methyl-6-phenyl-5H-pyrrolo[1,2-c]imidazol-5-yl)-acetyl]-piperidine-1-carboxylic acid benzyl ester

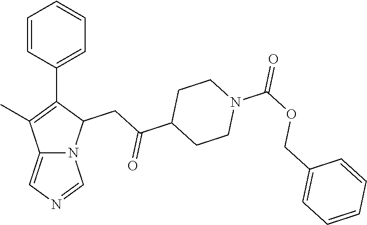 [SMILES: CC1=C(c2ccccc2)C(CC(=O)C2CCN(C(=O)OCc3ccccc3)CC2)n2cncc21]

To a mixture of methanol and acetic acid (3:1; 80 mL) was added compound benzyl 4-(4-phenyl-5-(1-trityl-1H-imidazol-4-yl)hexa-2,4-dienoyl)piperidine-1-carboxylate (Step 4, 0.8 g, 1.14 mmol) and the reaction mixture was heated at about 90° C. for about 12 hours. After completion of the reaction, solvents were evaporated; reaction mixture was quenched with aqueous sodium bicarbonate (50 mL) and extracted with ethyl acetate (3×100 mL). Combined organic layer was dried over anhydrous Na₂SO₄ and concentrated to give crude product which was purified by column chromatography to yield compound 4-[2-(7-Methyl-6-phenyl-5H-pyrrolo[1,2-c]imidazol-5-yl)-acetyl]-piperidine-1-carboxylic acid benzyl ester (0.31 mg, 59.3%) as pale yellow solid.

¹H NMR (CDCl₃, 400 MHz) δ: 7.55 (s, 1H), 7.43 (t, 2H, J=7.6 Hz), 7.37-7.28 (m, 6H), 7.27-7.25 (m, 2H), 6.87 (s, 1H), 5.54 (d, 1H, J=10.4 Hz), 5.10 (s, 2H), 4.15 (br. s, 2H), 2.85 (dd, 1H, J=2.4, 18.4 Hz), 2.76 (br. s, 2H), 2.54 (dd, 1H, J=10.4, 18.4 Hz), 2.43-2.35 (m, 1H), 2.18 (d, 3H, J=1.6 Hz), 1.97-1.93 (m, 1H), 1.80-1.73 (m, 1H), 1.57-1.46 (m, 2H); Mass (LCMS): 456.1 (M+1)

### Step-6: Preparation of 4-[1-Hydroxy-2-(7-methyl-6-phenyl-5H-pyrrolo[1,2-c]imidazol-5-yl)-ethyl]-piperidine-1-carboxylic acid benzyl ester

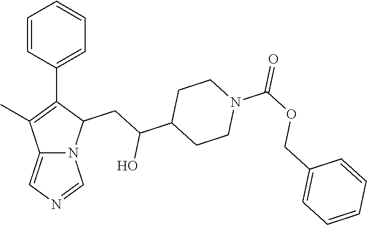 [SMILES: CC1=C(c2ccccc2)C(CC(O)C2CCN(C(=O)OCc3ccccc3)CC2)n2cncc21]

To a solution of compound 4-[2-(7-Methyl-6-phenyl-5H-pyrrolo[1,2-c]imidazol-5-yl)-acetyl]-piperidine-1-carboxylic acid benzyl ester (Step 5, 50 mg, 0.109 mmol) in MeOH (10 ml) was added NaBH₄ (6.2 mg, 0.165 mmol) at about 5-10° C. Reaction was stirred at the same temperature for about 15-20 minutes. Reaction was monitored by TLC. After completion of reaction solvents were removed under vacuum. Residue was taken in water and extracted with ethyl acetate (3×50 mL). Combined ethyl acetate was dried over anhydrous Na₂SO₄ and concentrated to give compound 4-[1-Hydroxy-2-(7-methyl-6-phenyl-5H-pyrrolo[1,2-c]imidazol-5-yl)-ethyl]-piperidine-1-carboxylic acid benzyl ester (24 mg, 47.8%) as pale yellow solid.

¹H NMR (CDCl₃, 400 MHz) δ: 7.81 (s, 0.8H), 7.74 (s, 0.2H), 7.42 (t, 2H, J=7.6 Hz), 7.36-7.27 (m, 8H), 6.86 (s, 1H), 5.44 (d, 0.2H, J=10.4 Hz), 5.23 (s, 0.8H), 5.08 (s, 2H), 4.16 (br. s, 2H), 3.66-3.64 (m, 0.2H), 3.36-3.32 (m, 0.8H), 2.64 (br. s, 2H), 2.19 (d, 0.7H, J=1.2 Hz,), 2.16 (d, 2.3H, J=1.2 Hz), 2.08-2.03 (m, 1H), 2.01-1.94 (m, 1H), 1.81-1.73 (m, 1H), 1.61 (d, 1H, J=12.4 Hz), 1.44 (d, 1H, J=12.4 Hz), 1.38-1.32 (m, 1H), 1.29-1.24 (m, 1H), 1.16-1.07 (m, 1H); Mass (LCMS): 458.1 (M+1)

Following intermediates have been synthesized as described in the above process of Example 137-Step 1-5:

| Intermediate No. | Analytical Data |
|---|---|
| 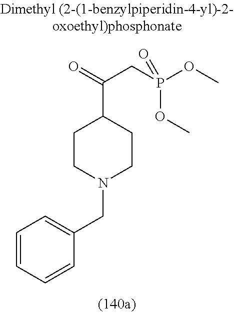 [SMILES: COP(=O)(CC(=O)C1CCN(Cc2ccccc2)CC1)OC] | ¹HNMR (CDCl₃, 400 MHz) δ: 7.33-7.29 (m, 4H), 7.27-7.24 (m, 1H), 3.78 (s, 3H), 3.76 (s, 3H), 3.49 (s, 2H), 3.15 (s, 1H), 3.09 (s, 1H), 2.92-2.87 (m, 2H), 2.57-2.51 (m, 1H), 2.01 (d, 1H, J = 2 Hz), 1.86-1.82 (m, 2H), 1.71-1.61 (m, 3H). Mass (LCMS): 326 (M + 1) |
| 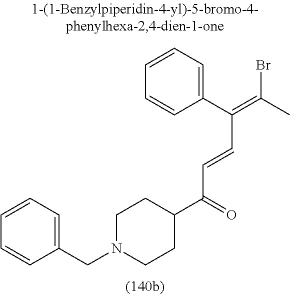 [SMILES: C/C(Br)=C(\C=C\C(=O)C1CCN(Cc2ccccc2)CC1)c1ccccc1] | ¹HNMR (CDCl₃, 400 MHz) δ: 7.98 (d, 0.5H, J = 15.6 Hz), 7.77 (d, 0.5H, J = 15.2 Hz), 7.43-7.34 (m, 3H), 7.31-7.28 (m, 4H), 7.25-7.21 (m, 1H), 7.08-7.06 (m, 2H), 5.76-5.65 (m, 1H), 3.74 (d, 2H, J = 6 Hz), 2.90-2.83 (m, 2H), 2.73 (s, 1H), 2.47-2.44 (m, 0.5H), 2.31-2.28 (m, 0.5H), 2.26 (s, 2H), 2.17 (s, 1H), 2.04- 1.92 (m, 2H), 1.72-1.65 (m, 3H). Mass (LCMS): 426 (M + 1) |
| 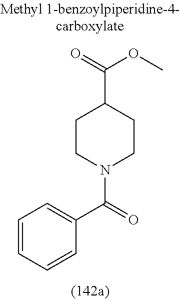 [SMILES: COC(=O)C1CCN(C(=O)c2ccccc2)CC1] | ¹HNMR (CDCl₃, 400 MHz) δ: 7.39-7.35 (m, 5H), 4.50 (br. s, 1H), 3.68 (s, 3H), 3.03 (br. s, 2H), 2.61- 2.54 (m, 1H), 2.02 (s, 1H), 1.84 (s, 2H), 1.70 (br. s, 2H); Mass (LCMS): 248 (M + 1). |
| 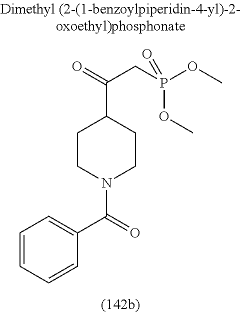 [SMILES: COP(=O)(CC(=O)C1CCN(C(=O)c2ccccc2)CC1)OC] | ¹HNMR (CDCl₃, 400 MHz) δ: 7.40-7.34 (m, 5H), 3.78 (s, 3H), 3.75 (s, 3H), 3.13-3.11 (m, 2H,), 2.98- 2.93 (m, 2H), 1.99-1.75 (m, 3H), 1.39-1.20 (m, 2H), 0.94-0.86 (m, 2H); Mass (LCMS): 340 (M + 1). |
| 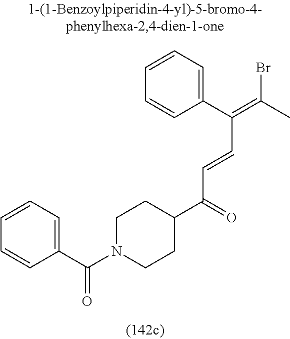 [SMILES: C/C(Br)=C(\C=C\C(=O)C1CCN(C(=O)c2ccccc2)CC1)c1ccccc1] | ¹HNMR (CDCl₃, 400 MHz) δ: 8.03 (d, 0.5H, J = 15.6 Hz), 7.82 (d, 0.5H, J = 15.2 Hz), 7.45-7.34 (m, 8H), 7.08 (d, 2H, J = 7.2 Hz), 5.74 (d, 0.5H, J = 15.2 Hz), 5.67 (d, 0.5H, J = 15.6 Hz), 4.59-4.58 (m, 1H), 3.79-3.70 (m, 1H), 3.11-2.91 (m, 2H), 2.75 (s, 1.5H), 2.61-2.55 (m, 1H), 2.27 (s, 1.5H), 1.89-1.59 (m, 4H). Mass (LCMS): 438, 440 (M + 2) |
| 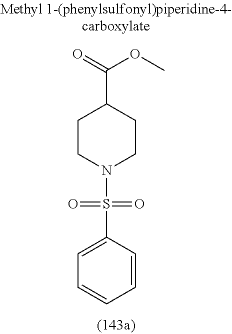 [SMILES: COC(=O)C1CCN(S(=O)(=O)c2ccccc2)CC1] | ¹HNMR (CDCl₃, 400 MHz) δ: 7.76-7.75 (m, 2H), 7.62-7.58 (m, 1H), 7.53 (t, 2H, J = 8 Hz), 3.65-3.62 (m, 5H), 2.49 (dt, 2H, J = 2.8, 11.6 Hz), 2.30-2.23 (m, 1H), 1.99-1.95 (m, 2H), 1.86-1.79 (m, 2H). Mass (LCMS): 284 (M + 1) |
| 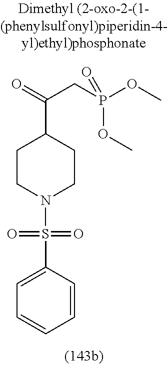 [SMILES: COP(=O)(CC(=O)C1CCN(S(=O)(=O)c2ccccc2)CC1)OC] | ¹HNMR (CDCl₃, 400 MHz) δ: 7.75-7.73 (m, 2H), 7.62-7.58 (m, 1H), 7.54-7.51 (m, 2H), 3.74 (s, 3H), 3.72 (s, 3H), 3.10 (s, 1H), 3.04 (s, 1H), 2.57-2.50 (m, 1H), 2.40 (dt, 2H, J = 2.4, 11.6 Hz), 1.94 (dd, 2H, J = 2.8, 14 Hz), 1.75-1.65 (m, 3H), 1.48-1.42 (m, 1H). Mass (LCMS): 376 (M + 1) |
| 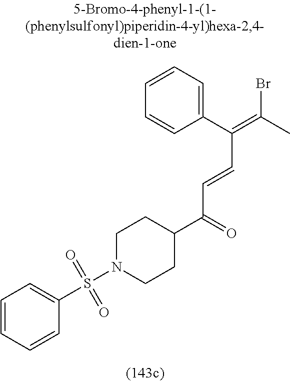 [SMILES: C/C(Br)=C(\C=C\C(=O)C1CCN(S(=O)(=O)c2ccccc2)CC1)c1ccccc1] | ¹HNMR (CDCl₃, 400 MHz) δ: 7.95 (d, 1H, J = 15.6 Hz), 7.78-7.72 (m, 2H), 7.61-7.56 (m, 1H), 7.55- 7.49 (m, 2H), 7.41-7.35 (m, 3H), 7.05-7.03 (m, 2H), 5.65 (d, 0.5H, J = 15.2 Hz), 5.60 (d, 0.5H, J = 15.6 Hz), 3.70-3.65 (m, 2H), 2.72 (s, 1H), 2.47-2.35 (m, 3H), 2.25 (s, 2H), 1.81-1.63 (m, 4H). Mass (LCMS): 374, 376 (M + 2). |
| 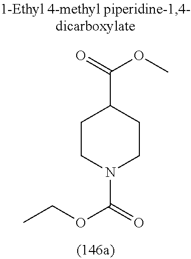 | ¹HNMR (CDCl₃, 400 MHz) δ: 4.10 (q, 2H, J = 6.8 Hz), 4.06-3.98 (m, 2H), 3.66 (s, 3H), 2.86 (t, 2H, J = 11.6 Hz), 2.47-2.42 (m, 1H), 1.87-1.84 (s, 2H), 1.66- 1.56 (m, 2H), 1.22 (t, 3H, J = 6.8 Hz). Mass (LCMS): 216.1 (M + 1) |
| 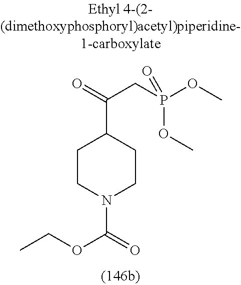 [SMILES: CCOC(=O)N1CCC(C(=O)CP(=O)(OC)OC)CC1] | ¹HNMR (CDCl₃, 400 MHz) δ: 4.08 (q, 2H, J = 7.2 Hz), 3.76 (s, 3H), 3.73 (s, 3H), 3.10 (d, 2H, J = 22.4 Hz), 2.19 (t, 2H, J = 12 Hz), 2.75-2.69 (m, 1H), 2.00- 1.97 (m, 1H), 1.83 (d, 2H, J = 12.4 Hz), 1.55-1.39 (m, 3H), 1.21 (t, 3H, J = 7.2 Hz). Mass (LCMS): 308.1 (M + 1) |
| 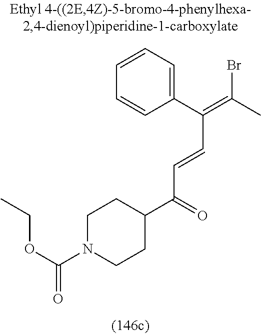 [SMILES: CCOC(=O)N1CCC(C(=O)/C=C/C(=C(\C)Br)c2ccccc2)CC1] | ¹HNMR (CDCl₃, 400 MHz) δ: 8.00 (d, 0.5H, J = 15.6 Hz), 7.80 (d, 0.5H, J = 15.2 Hz), 7.44-7.36 (m, 3H), 7.08 (d, 2H, J = 7.6 Hz), 5.73 (d, 0.5H, J = 15.2 Hz), 5.67 (d, 0.5H, J = 15.6 Hz), 4.14-4.08 (m, 4H), 2.85- 2.78 (m, 2H), 2.74 (s, 1H), 2.68-2.60 (m, 0.5H), 2.49- 2.43 (m, 0.5H), 2.26 (s, 2H), 1.69 (t, 2H, J = 11.6 Hz), 1.57-1.47 (m, 2H), 1.25-1.22 (m, 3H). Mass (LCMS): 408 (M + 2) |
| 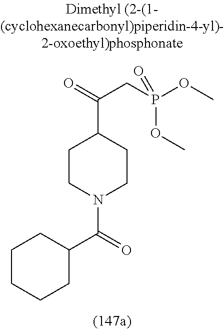 [SMILES: COP(=O)(CC(=O)C1CCN(C(=O)C2CCCCC2)CC1)OC] | ¹HNMR (CDCl₃, 400 MHz) δ: 4.54 (d, 1H, J = 12.8 Hz), 3.99-3.89 (m, 1H), 3.77-3.68 (m, 6H), 3.21-3.00 (m, 3H), 3.84-2.77 (m, 1H), 2.66-2.64 (m, 1H), 2.46- 2.38 (m, 1H), 1.92-1.88 (m, 2H), 1.77-1.75 (m, 2H), 1.68-1.64 (m, 3H), 1.57-1.41 (m, 4H), 1.25-1.15 (m, 3H). Mass (LCMS): 346.1 (M + 1) |
| 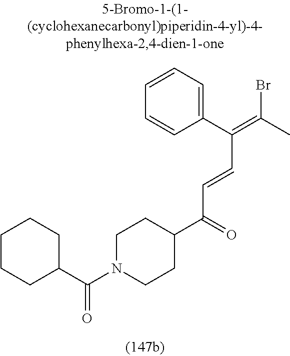 [SMILES: C/C(Br)=C(\C=C\C(=O)C1CCN(C(=O)C2CCCCC2)CC1)c1ccccc1] | ¹HNMR (CDCl₃, 400 MHz) δ: 8.00 (d, 0.6H, J = 15.6 Hz), 7.80 (d, 0.4H, J = 14.8 Hz), 7.44-7.35 (m, 3H), 7.07 (d, 2H, J = 6.8 Hz), 5.72 (d, 0.4H, J = 15.2 Hz), 5.66 (d, 0.6H, J = 15.6 Hz), 4.53-4.44 (m, 1H), 3.94- 3.84 (m, 1H), 3.08-3.01 (m, 1H), 2.74 (s, 1H), 2.72- 2.39 (m, 3H), 2.27 (s, 2H), 1.79-1.71 (m, 4H), 1.70- 1.57 (m, 3H), 1.53-1.43 (m, 3H), 1.29-1.17 (m, 4H). Mass (LCMS): 446.1 (M + 2) |

Following examples have been synthesized by the above procedure described in Example 137 with their corresponding intermediates in similar reaction conditions:

| Example | | |
|---|---|---|
| No. | IUPAC Name/Structure | Analytical Data |
| 140. | 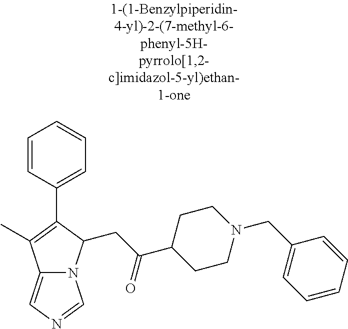 [SMILES: CC1=C(c2ccccc2)C(CC(=O)C2CCN(Cc3ccccc3)CC2)n2cncc21] | ¹HNMR (CDCl₃, 400 MHz) δ: 7.57 (s, 1H), 7.42 (t, 2H, J = 7.2 Hz), 7.34-7.27 (m, 5H), 7.25-7.21 (m, 3H), 6.87 (s, 1H), 5.55 (d, 1H, J = 10.4 Hz), 3.45 (s, 2H), 2.85-2.80 (m, 2H), 2.57-2.50 (m, 1H), 2.18 (d, 3H, J = 2 Hz), 1.95-1.88 (m, 4H), 1.70-1.60 (m, 4H). Mass (LCMS): 412.1 (M + 1); Purity: 93.27%. |
| 141. | 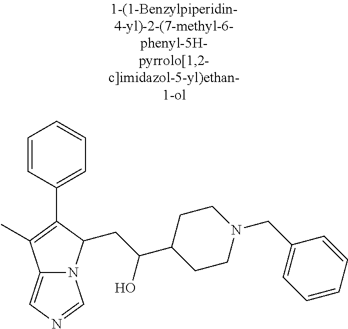 [SMILES: CC1=C(c2ccccc2)C(CC(O)C2CCN(Cc3ccccc3)CC2)n2cncc21] | ¹HNMR (CDCl₃, 400 MHz) δ: 7.89 (s, 1H), 7.41 (t, 2H, J = 7.2 Hz), 7.33-7.26 (m, 5H), 7.24-7.20 (m, 3H), 6.97 (s, 0.2H), 6.91 (s, 0.8H), 5.49-5.46 (m, 0.3H), 5.25 (br. s, 0.7H), 3.49-3.41 (m, 2H), 2.88-2.81 (m, 2H), 2.17 (d, 1H, J = 1.6 Hz), 2.15 (d, 2H, J = 1.6 Hz), 2.08-2.04 (m, 2H), 2.01-1.94 (m, 3H), 1.87-1.80 (m, 2H), 1.75-1.68 (m, 1H), 1.63-1.58 (m, 1H), 1.44-1.39 (m, 1H), 1.23-1.19 (m, 1H). Mass (LCMS): 414.1 (M + 1); Purity: 93.13%. |
| 142. | 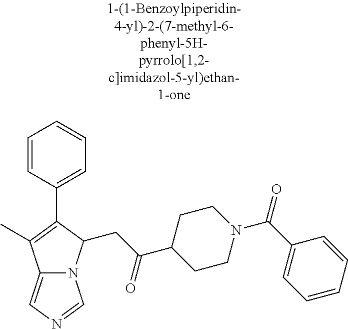 [SMILES: CC1=C(c2ccccc2)C(CC(=O)C2CCN(C(=O)c3ccccc3)CC2)n2cncc21] | ¹HNMR (CDCl₃, 400 MHz) δ: 7.56 (s, 1H), 7.45- 7.32 (m, 8H), 7.28-7.25 (m, 2H), 7.26 (s, 1H), 5.55 (d, 1H, J = 10 Hz), 4.66-4.57 (m, 1H), 3.83- 3.72 (m, 1H), 2.88 (d, 3H, J = 18 Hz), 2.60-2.47 (m, 2H), 2.18 (d, 3H, J = 1.6 Hz), 1.90-1.72 (m, 2H), 1.62-1.55 (m, 2H). Mass (LCMS): 426.1 (M + 1); Purity: 99.46%. |
| 143. | 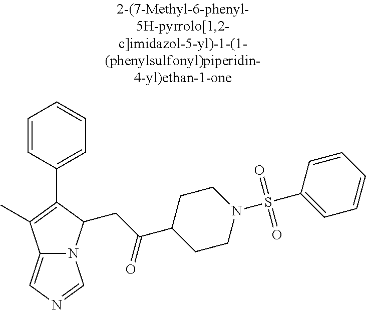 [SMILES: CC1=C(c2ccccc2)C(CC(=O)C2CCN(S(=O)(=O)c3ccccc3)CC2)n2cncc21] | ¹HNMR (CDCl₃, 400 MHz) δ: 7.73 (d, 2H, J = 7.2 Hz), 7.59 (t, 1H, J = 7.2 Hz), 7.54-7.49 (m, 3H), 7.41 (t, 2H, J = 7.6 Hz), 7.32 (t, 1H, J = 7.2 Hz), 7.23 (d, 2H, J = 7.2 Hz), 6.86 (s, 1H), 5.50 (d, 1H, J = 10 Hz), 3.71 (d, 2H, J = 12 Hz), 2.79 (dd, 1H, J = 2.4, 18.4 Hz), 2.47 (dd, 1H, J = 10.4, 18.4 Hz), 2.37-2.31 (m, 2H), 2.16 (d, 3H, J = 1.6 Hz), 1.83-1.76 (m, 2H), 1.69-1.65 (m, 1H), 1.29-1.25 (m, 2H). Mass (LCMS): 462.1 (M + 1); Purity: 99.4%. |
| 144. | 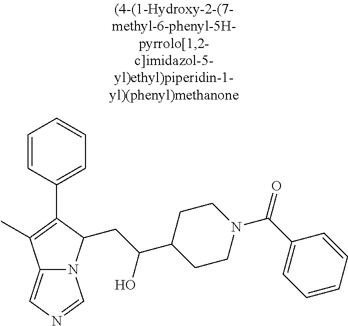 [SMILES: CC1=C(c2ccccc2)C(CC(O)C2CCN(C(=O)c3ccccc3)CC2)n2cncc21] | ¹HNMR (CDCl₃, 400 MHz) δ: 7.89-7.78 (m, 1H), 7.45-7.27 (m, 10H), 6.99-6.90 (m, 1H), 5.45-5.43 (m, 0.3H), 5.25 (br. s, 0.7H), 4.76-4.64 (m, 1H), 3.80-3.64 (m, 2H), 3.38-3.37 (m, 1H), 2.92-2.56 (m, 3H), 2.19 (d, 0.7H, J = 0.8 Hz), 2.17 (dd, 2.3H, J = 1.2 Hz), 1.91-1.81 (m, 2H), 1.55-1.39 (m, 2H), 1.19-1.08 (m, 2H). Mass (LCMS): 428.1 (M + 1); Purity: 98.48%. |
| 145. | 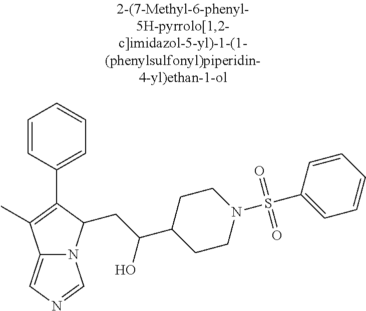 [SMILES: CC1=C(c2ccccc2)C(CC(O)C2CCN(S(=O)(=O)c3ccccc3)CC2)n2cncc21] | ¹HNMR (CDCl₃, 400 MHz) δ: 7.85-7.80 (m, 1H), 7.72 (d, 2H, J = 8.4 Hz), 7.59-7.40 (m, 5H), 7.35- 7.30 (m, 1H), 7.27-7.26 (m, 2H), 6.95-6.88 (m, 1H), 5.42-5.32 (m, 0.3H), 5.22-5.23 (m, 0.7H), 3.80-3.74 (m, 2H), 3.65-3.64 (m, 0.5H), 3.33-3.26 (m, 0.5H), 2.18-2.09 (m, 4H), 1.93-1.88 (m, 1H), 1.84-1.81 (m, 1H), 1.65-1.58 (m, 2H), 1.49-1.47 (m, 1H), 1.33-1.29 (m, 1H), 1.21-1.18 (m, 1H), 1.15-1.06 (m, 1H). Mass (LCMS): 464.1 (M + 1); Purity: 98.32%. |
| 146. | 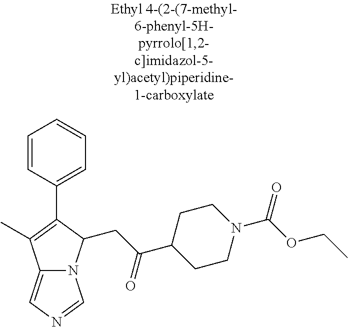 [SMILES: CCOC(=O)N1CCC(C(=O)CC2C(c3ccccc3)=C(C)c3cncn32)CC1] | ¹HNMR (CDCl₃, 400 MHz) δ: 7.55 (s, 1H), 7.43 (t, 2H, J = 7.2 Hz), 7.35-7.31 (m, 1H), 7.27-7.24 (m, 2H), 6.87 (s, 1H), 5.54 (d, 1H, J = 10.4 Hz), 4.14-4.08 (m, 4H), 2.87 (d, 0.4H, J = 2.4 Hz), 2.83 (d, 0.6H, J = 2.4 Hz), 2.73 (t, 2H, J = 12.4 Hz), 2.54 (dd, 1H, J = 10.8, 18.4 Hz), 2.42-2.34 (m, 1H), 2.18 (d, 3H, J = 2 Hz), 1.75-1.69 (m, 2H), 1.56-1.45 (m, 2H), 1.23 (t, 3H, J = 6.8 Hz). Mass (LCMS): 394.1 (M + 1); Purity: 93.66%. |
| 147. | 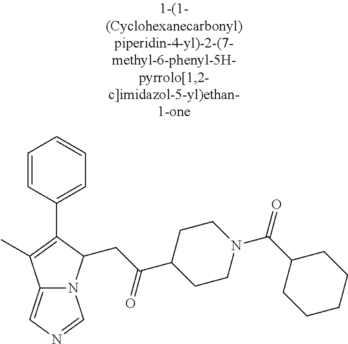 [SMILES: CC1=C(c2ccccc2)C(CC(=O)C2CCN(C(=O)C3CCCCC3)CC2)n2cncc21] | ¹HNMR (CDCl₃, 400 MHz) δ: 7.55 (s, 1H), 7.43 (t, 2H, J = 8 Hz), 7.36-7.32 (m, 1H), 7.27-7.25 (m, 2H), 6.88 (s, 1H), 5.55 (d, 1H, J = 10 Hz), 4.55- 4.51 (m, 1H), 3.92-3.89 (m, 1H), 3.01-2.94 (m, 1H), 2.60-2.52 (m, 2H), 2.50-2.39 (m, 2H), 2.18 (d, 3H, J = 1.6 Hz), 1.78-1.72 (m, 5H), 1.71-1.64 (m, 6H), 1.57-1.44 (m, 3H). Mass (LCMS): 432.2 (M + 1); Purity: 97.13%. |
| 148. | 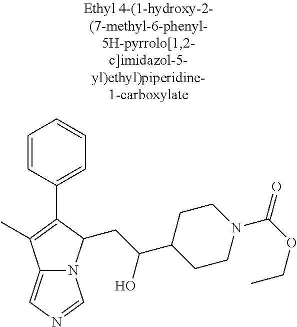 [SMILES: CCOC(=O)N1CCC(C(O)CC2C(c3ccccc3)=C(C)c3cncn32)CC1] | ¹HNMR (CDCl₃, 400 MHz) δ: 7.94 (br. s, 1H), 7.43 (t, 2H, J = 7.6 Hz), 7.35-7.26 (m, 3H), 6.96- 6.86 (m, 2H), 5.48-5.45 (m, 0.2H), 5.25 (br. s, 0.8H), 4.11-4.06 (m, 2H), 3.73-3.64 (m, 1H), 3.14-3.37 (m, 1H), 2.64-2.58 (m, 2H), 2.19 (s, 1H), 2.16 (s, 2H), 2.11-2.06 (m, 1H), 2.02-1.88 (m, 3H), 1.45-1.30 (m, 2H), 1.22 (t, 3H, J = 7.2 Hz), 1.17-1.00 (m, 2H). Mass (LCMS): 396.1 (M + 1); Purity: 96.51%. |
| 149. | 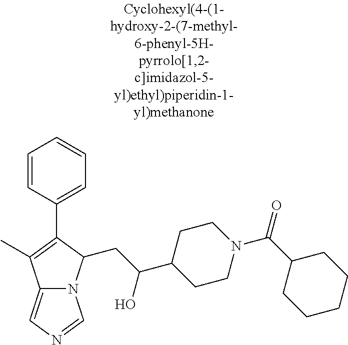 [SMILES: CC1=C(c2ccccc2)C(CC(O)C2CCN(C(=O)C3CCCCC3)CC2)n2cncc21] | ¹HNMR (CDCl₃, 400 MHz) δ: 7.97 (br. s, 1H), 7.43 (t, 2H, J = 7.6 Hz), 7.35-7.26 (m, 3H), 6.92- 6.89 (m, 1H), 5.49-5.47 (m, 0.2H), 5.25 (br. s, 0.8H), 4.65-4.55 (m, 1H), 3.88 (t, 1H, J = 12.8 Hz), 3.41 (br. s, 1H), 2.87 (t, 1H, J = 12.4 Hz), 2.64-2.29 (m, 6H), 2.19 (s, 0.8H), 2.16 (s, 2.2H), 1.99-1.91 (m, 1H), 1.76 (br. s, 2H), 1.55-1.38 (m, 4H), 1.26-1.18 (m, 5H), 1.17-1.02 (m, 2H). Mass (LCMS): 434.2 (M + 1); Purity: 97.36%. |
| 150. | 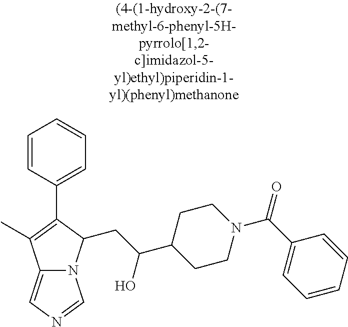 [SMILES: CC1=C(c2ccccc2)C(CC(O)C2CCN(C(=O)c3ccccc3)CC2)n2cncc21] | ¹HNMR (CDCl₃, 400 MHz) δ: 8.53 (s, 1H), 7.46 (t, 2H, J = 7.2 Hz), 7.39-7.28 (m, 8H), 6.94 (br. s, 1H), 5.59 (d, 0.1H, J = 10.4 Hz), 5.36 (br. s, 0.9H), 4.72-4.62 (m, 1H), 3.97-3.84 (m, 1H), 3.61- 3.56 (m, 1H), 2.89-2.80 (m, 1H), 2.63-2.59 (m, 1H), 2.20 (s, 0.3 H), 2.17 (s, 2.7 H), 1.98-1.90 (m, 1H), 1.74-1.62 (m, 2H), 1.53-1.37 (m, 2H), 1.32-1.09 (m, 2H). Mass (LCMS): 428.1 (M + 1); Purity: 95.5%. |
| 151. | 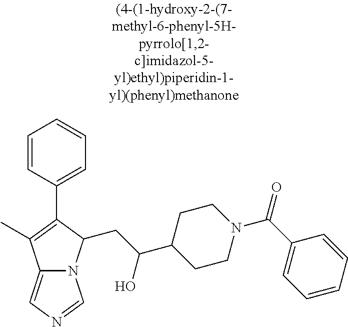 [SMILES: CC1=C(c2ccccc2)C(CC(O)C2CCN(C(=O)c3ccccc3)CC2)n2cncc21] | ¹HNMR (CDCl₃, 400 MHz) δ: 8.64 (br. s, 1H), 7.46 (t, 2H, J = 7.2 Hz), 7.40-7.26 (m, 8H), 7.00- 6.97 (m, 1H), 5.64-5.61 (m, 0.2H), 5.40-5.39 (m, 0.8H), 4.74-4.59 (m, 1H), 3.76-3.58 (m, 3H), 2.93-2.80 (m, 1H), 2.67-2.55 (m, 1H), 2.21 (s, 0.4H), 2.18 (s, 2.6H), 2.00-1.90 (m, 1H), 1.74- 1.62 (m, 2H), 1.54-1.39 (m, 2H), 1.33-1.11 (m, 2H). Mass (LCMS): 428.1 (M + 1); Purity: 94.62%. |
| 153. | 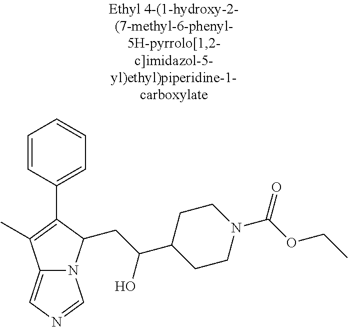 [SMILES: CCOC(=O)N1CCC(C(O)CC2C(c3ccccc3)=C(C)c3cncn32)CC1] | ¹HNMR (CDCl₃, 400 MHz) δ: 8.16 (br. s, 1H), 7.44 (t, 2H, J = 7.6 Hz), 7.36-7.32 (m, 1H), 7.29- 7.27 (m, 2H), 6.92 (br. s, 1H), 5.28 (br. s, 1H), 4.08 (q, 2H, J = 7.2 Hz), 3.76-3.61 (m, 1H), 3.47- 3.32 (m, 2H), 2.68-2.54 (m, 2H), 2.16 (d, 3H, J = 0.4 Hz), 1.98-1.92 (m, 1H), 1.80-1.68 (m, 1H), 1.62 (d, 1H, J = 12.8 Hz), 1.44-1.32 (m, 2H), 1.22 (t, 3H, J = 7.2 Hz), 1.44-1.04 (m, 2H). Mass (LCMS): 396.1 (M + 1); Purity: 99.7%. |
| 154. | 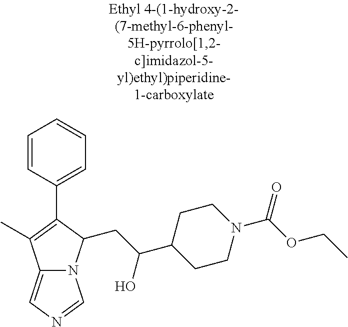 [SMILES: CCOC(=O)N1CCC(C(O)CC2C(c3ccccc3)=C(C)c3cncn32)CC1] | ¹HNMR (CDCl₃, 400 MHz) δ: 8.25 (s, 1H), 7.44 (t, 2H, J = 7.2 Hz), 7.35 (t, 1H, J = 7.2 Hz), 7.29 (d, 1H, J = 1.2 Hz), 7.27-7.26 (m, 1H), 6.93 (br. s, 1H), 5.30 (br. s, 1H), 4.08 (q, 2H, J = 7.2 Hz), 3.65 (d, 1H, J = 2.4 Hz), 3.49-3.43 (m, 2H), 2.63- 2.58 (m, 2H), 2.16 (d, 3H, J = 1.6 Hz), 2.00-1.92 (m, 1H), 1.75-1.61 (m, 2H), 1.44-1.32 (m, 2H), 1.22 (t, 3H, J = 7.2 Hz), 1.15-1.08 (m, 2H). Mass (LCMS): 396.1 (M + 1); Purity: 97.35%. |
| 155. | 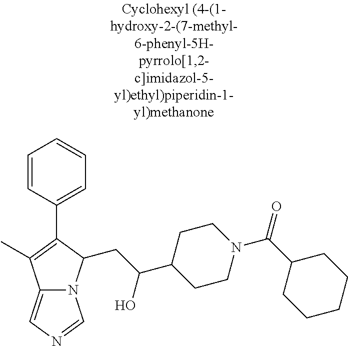 [SMILES: CC1=C(c2ccccc2)C(CC(O)C2CCN(C(=O)C3CCCCC3)CC2)n2cncc21] | ¹HNMR (CDCl₃, 400 MHz) δ: 8.00 (br. s, 1H), 7.44 (t, 2H, J = 7.2 Hz), 7.34 (t, 1H, J = 7.2 Hz), 7.29-7.27 (m, 2H), 6.91 (s, 1H), 5.27 (br. s, 1H), 4.66-4.54 (m, 1H), 3.94-3.83 (m, 1H), 3.74-3.64 (m, 1H), 3.38-3.35 (m, 1H), 2.92-2.28 (m, 1H), 2.43-2.19 (m, 4H), 2.16 (d, 3H, J = 1.2 Hz), 2.02- 1.93 (m, 1H). 1.79-1.74 (m, 3H), 1.69-1.61 (m, 3H), 1.52-1.38 (m, 4H), 1.23-1.18 (m, 2H), 1.17- 0.99 (m, 2H). Mass (LCMS): 434.2 (M + 1); Purity: 100%. |
| 156. | 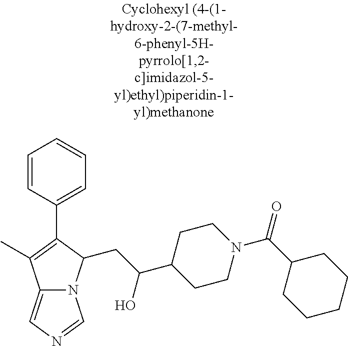 [SMILES: CC1=C(c2ccccc2)C(CC(O)C2CCN(C(=O)C3CCCCC3)CC2)n2cncc21] | ¹HNMR (CDCl₃, 400 MHz) δ: 8.09-7.98 (m, 1H), 7.44 (t, 2H, J = 7.2 Hz), 7.34 (t, 1H, J = 7.2 Hz), 7.29-7.27 (m, 2H), 6.92 (s, 1H), 5.27 (br. s, 1H), 4.67- 4.58 (m, 1H), 3.93-3.82 (m, 1H), 3.73-3.60 (m, 1H), 3.41-3.35 (m, 1H), 2.90-2.82 (m, 2H), 2.44-2.32 (m, 3H), 2.16 (d, 3H, J = 1.6 Hz), 2.09 (s, 2H), 2.00-1.92 (m, 1H), 1.67-1.58 (m, 3H), 1.57-1.44 (m, 4H), 1.24-1.19 (m, 2H), 1.16-0.99 (m, 2H). Mass (LCMS): 434.2 (M + 1); Purity: 99.12%. |

### Example 159: Preparation of 1-(1-Benzoyl-azetidin-3-yl)-2-(7-methyl-6-phenyl-5H-pyrrolo[1,2-c]imidazol-5-yl)-ethanone

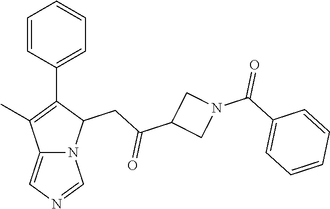 [SMILES: CC1=C(c2ccccc2)C(CC(=O)C2CN(C(=O)c3ccccc3)C2)n2cncc21]

This was synthesized as used in the above described experimental procedure Example 137 with methyl 1-benzylazetidine-3-carboxylate to give final compound.

¹H NMR (CDCl₃, 400 MHz) δ: 7.59 (d, 2H, J=7.2 Hz), 7.48-7.36 (m, 8H), 7.25-7.21 (m, 1H), 6.92 (s, 1H), 5.59 (d, 1H, J=9.6 Hz), 4.27 (t, 2H, J=9.2 Hz), 4.18-4.09 (m, 1H), 3.50-3.44 (m, 1H), 2.97-2.85 (m, 1H), 2.61-2.43 (m, 1H), 2.35-2.36 (m, 1H), 2.18 (s, 3H). Mass (LCMS): 398.1 (M+1); Purity: 90.53%.

### Example 158: Preparation of 4-[1-Hydroxy-2-(7-methyl-6-phenyl-5H-pyrrolo[1,2-c]imidazol-5-yl)-ethyl]-piperidine-1-carboxylic acid phenyl amide

### Step-1: Preparation of 2-(7-methyl-6-phenyl-5H-pyrrolo[1,2-c]imidazol-5-yl)-1-(piperidin-4-yl)ethan-1-one

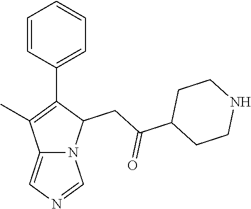 [SMILES: CC1=C(c2ccccc2)C(CC(=O)C2CCNCC2)n2cncc21]

To a solution of 4-[2-(7-Methyl-6-phenyl-5H-pyrrolo[1,2-c]imidazol-5-yl)-acetyl]-piperidine-1-carboxylic acid benzyl ester (Step 5; Example 137, 50 mg, 0.109 mmol) in MeOH (10 mL), was added 10% Pd/C (50% wet, 50 mg) and reaction mixture was stirred under H₂ pressure for 2-3 h. Reaction was monitored by TLC/LCMS. After completion of reaction was filtered through celite bed. Filtrate was evaporated to get compound 2-(7-methyl-6-phenyl-5H-pyrrolo[1,2-c]imidazol-5-yl)-1-(piperidin-4-yl)ethan-1-one (25 mg, 78%) as semi-solid. Product formation was confirmed by LCMS and used in next step without purification.

### Step-2: Preparation of 4-[2-(7-Methyl-6-phenyl-5H-pyrrolo[1,2-c]imidazol-5-yl)-acetyl]-piperidine-1-carboxylic acid phenylamide

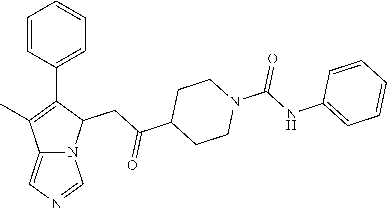 [SMILES: CC1=C(c2ccccc2)C(CC(=O)C2CCN(C(=O)Nc3ccccc3)CC2)n2cncc21]

To a solution of compound 2-(7-methyl-6-phenyl-5H-pyrrolo[1,2-c]imidazol-5-yl)-1-(piperidin-4-yl)ethan-1-one (Step 1, 25 mg, 0.077 mmol) in MDC (5 mL) was added phenyl isocyanate (11 mg, 0.85 mmol) and NEt₃ (19.6 mg, 0.19 mmol) at rt. Reaction mixture was stirred at rt for 12 h, progress monitored by TLC. After completion of reaction volatiles are removed under vacuum. Crude product was purified using column chromatography to get 4-[2-(7-Methyl-6-phenyl-5H-pyrrolo[1,2-c]imidazol-5-yl)-acetyl]-piperidine-1-carboxylic acid phenylamide (18 mg, 52.5%) as pale yellow solid.

¹H NMR (CDCl₃, 400 MHz) δ: 7.58 (s, 1H), 7.44 (t, 2H, J=7.6 Hz), 7.36-7.30 (m, 3H), 7.28-7.23 (m, 4H), 7.02 (t, 1H, J=7.2 Hz), 6.89 (s, 1H), 6.44 (s, 1H), 5.56 (d, 1H, J=10.4 Hz), 4.08-4.0 (m, 2H), 2.93-2.83 (m, 3H), 2.56 (dd, 1H, J=10.4, 18.4 Hz), 2.48-2.42 (m, 1H), 2.19 (d, 3H, J=1.6 Hz), 1.84-1.77 (m, 2H), 1.64-1.56 (m, 2H). Mass (LCMS): 441.1 (M+1)

### Step-3: Preparation of 4-[1-Hydroxy-2-(7-methyl-6-phenyl-5H-pyrrolo[1,2-c]imidazol-5-yl)-ethyl]-piperidine-1-carboxylic acid phenyl amide

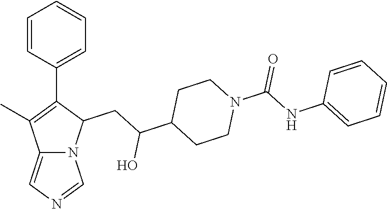 [SMILES: CC1=C(c2ccccc2)C(CC(O)C2CCN(C(=O)Nc3ccccc3)CC2)n2cncc21]

To a solution of compound 4-[2-(7-Methyl-6-phenyl-5H-pyrrolo[1,2-c]imidazol-5-yl)-acetyl]-piperidine-1-carboxylic acid phenylamide (Step 2, 15 mg, 0.034 mmol) in MeOH (5 mL) was added NaBH₄ (1.94 mg, 0.051 mmol) at 5-10° C. Reaction was stirred at the same temperature for 15-20 minutes. Reaction was monitored by TLC. After completion of reaction solvents were removed under vacuum. Residue was taken in water and extracted with ethyl acetate (3×30 mL). Combined ethyl acetate was dried over anhydrous Na₂SO₄ and concentrated to give compound 4-[1-Hydroxy-2-(7-methyl-6-phenyl-5H-pyrrolo[1,2-c]imidazol-5-yl)-ethyl]-piperidine-1-carboxylic acid phenyl amide (10 mg, 66.3%) as pale yellow solid.

¹H NMR (CDCl₃, 400 MHz) δ: 7.83 (s, 0.8H), 7.75 (s, 0.2H), 7.43 (t, 2H, J=7.6 Hz), 7.35-7.30 (m, 4H), 7.27-7.23 (m, 3H), 7.00 (t, 1H, J=7.6 Hz), 6.87 (s, 1H), 6.51 (s, 0.3H), 6.48 (s, 0.7H), 5.43 (s, 0.2H), 5.24 (s, 0.8H), 4.04 (d, 2H, J=10.4 Hz), 3.66-3.62 (m, 0.2H), 3.37-3.34 (m, 0.8H), 2.77-2.71 (m, 2H), 2.19 (s, 0.8H), 2.17 (s, 2.2H), 2.00-1.78 (m, 4H), 1.66 (d, 1H, J=12.4 Hz,), 1.52-1.36 (m, 3H). Mass (LCMS): 443.1 (M+1).

Following intermediates have been synthesized as described in the above process of Example 158-Step 2:

| Intermediate No. | Analytical Data |
|---|---|
| 4-[2-(7-Methyl-6-phenyl-5H- | ¹HNMR (CDCl₃, 400 MHz) δ: 8.18 (d, 1H, J = 4.4 Hz), |
| pyrrolo[1,2-c]imidazol-5-yl)-cetyl]- | 7.97 (d, 1H, J = 8.8 Hz), 7.63 (t, 1H, J = 7.2 Hz), |
| piperidine-1-carboxylic acid pyridin-2- | 7.56 (s, 1H), 7.43 (t, 2H, J = 7.6 Hz), 7.34 (t, 1H, J = |
| ylamide | 7.2 Hz), 7.28-7.26 (m, 2H), 7.18 (s, 1H), 6.93 (t, 1H, |
| 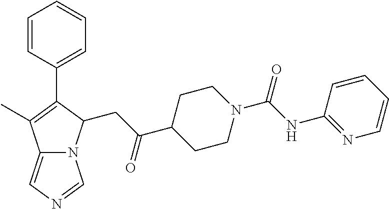 [SMILES: CC1=C(c2ccccc2)C(CC(=O)C2CCN(C(=O)Nc3ccccn3)CC2)n2cncc21] | J = 6.8 Hz), 6.88 (s, 1H), 5.55 (dd, 1H, J = 10.4 Hz), 4.07 (dd, 2H, J = 2.8, 13.2 Hz), 2.95-2.85 (m, 3H), 2.56 (dd, 1H, J = 10.4, 18.4 Hz), 2.49-2.42 (m, 1H), 2.18 (d, 3H, J = 1.6 Hz), 1.86-1.78 (m, 2H), 1.65- 1.55 (m, 2H): Mass (LCMS): 442.1 (M + 1). |
| (167a) | |
| N-cyclohexyl-4-(2-(7-methyl-6-phenyl- | ¹H NMR (CDCl₃, 400 MHz) δ: 7.54 (s, 1H), 7.43 (t, |
| 5H-pyrrolo[1,2-c]imidazol-5- | 2H, J = 7.6 Hz), 7.33 (t, 1H, J = 7.6 Hz), 7.27-7.25 |
| yl)acetyl)piperidine-1-carboxamide | (m, 2H), 6.87 (s, 1H), 5.55 (d, 1H, J = 10.4 Hz), 4.24 |
| 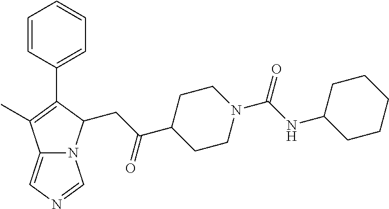 [SMILES: CC1=C(c2ccccc2)C(CC(=O)C2CCN(C(=O)NC3CCCCC3)CC2)n2cncc21] | (d, 1H, J = 7.6 Hz), 3.93-3.83 (m, 2H), 3.65-3.57 (m, 1H), 2.86 (dd, 1H, J = 2.4, 18.4 Hz), 2.77-2.68 (m, 2H), 2.54 (dd, 1H, J = 10.4, 18.4 Hz), 2.41-2.34 (m, 1H), 2.18 (d, 3H, J = 1.6 Hz), 1.94-1.90 (m, 2H), 1.76-1.65 (m 4H), 1.62-1.45 (m, 2H), 1.43-1.30 (m, 2H), 1.15-1.02 (m, 3H). Mass (LCMS): 447.1 (M + 1) |
| (179a) | |
| N-(3-chlorophenyl)-4-(2-(7-methyl-6- | ¹H NMR (CDCl₃, 400 MHz) δ: 7.50 (br. s, 1H), 7.45- |
| phenyl-5H-pyrrolo[1,2-c]imidazol-5- | 7.42 (m, 3H), 7.34 (t, 1H, J = 7.6 Hz), 7.29-7.26 (m, |
| yl)acetyl)piperidine-1-carboxamide | 2H), 7.18-7.15 (m, 2H), 7.00-6.97 (m, 1H), 6.88 (s, |
| 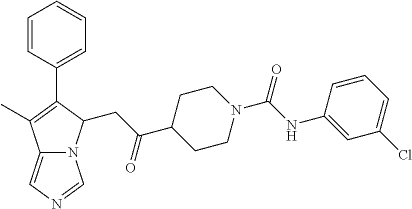 [SMILES: CC1=C(c2ccccc2)C(CC(=O)C2CCN(C(=O)Nc3cccc(Cl)c3)CC2)n2cncc21] | 1H), 6.58 (s, 1H), 5.57 (d, 1H, J = 10.4 Hz), 4.07- 3.98 (m, 2H), 2.93-2.84 (m, 3H), 2.56 (dd, 1H, J = 10.4, 18.4 Hz), 2.49-2.41 (m, 1H), 2.19 (d, 3H, J = 1.6 Hz), 1.82-1.78 (m, 2H), 1.62-1.57 (m, 2H) Mass (LCMS): 476.1 (M + 1) |
| (181a) | |
| N-(3-chloro-4-fluorophenyl)-4-(2-(7- | ¹H NMR (CDCl₃, 400 MHz) δ: 7.55 (s, 1H), 7.48- |
| methyl-6-phenyl-5H-pyrrolo[1,2- | 7.42 (m, 3H), 7.36-7.32 (m, 1H), 7.28-7.26 (m, 2H), |
| c]imidazol-5-yl)acetyl)piperidine-1- | 7.17-7.13 (m, 1H), 7.02 (t, 1H, J = 8.8 Hz), 6.87 (s, |
| carboxamide | 1H), 6.63 (s, 1H), 5.56 (d, 1H, J = 10.4 Hz), 4.07- |
| 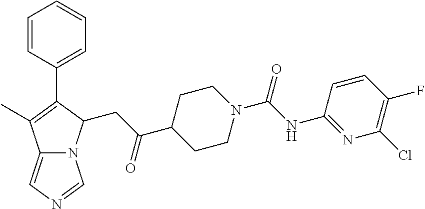 [SMILES: CC1=C(c2ccccc2)C(CC(=O)C2CCN(C(=O)Nc3ccc(F)c(Cl)n3)CC2)n2cncc21] | 3.98 (m, 2H), 2.93-2.83 (m, 3H), 2.56 (dd, 1H, J = 10.4, 18.4 Hz), 2.49-2.43 (m, 1H), 2.19 (d, 3H), J = 2.0 Hz), 1.83-1.79 (m, 2H), 1.60-1.54 (m, 2H); Mass (LCMS): 493.1 (M + 1) |
| (182a) | |
| 4-(2-(7-methyl-6-phenyl-5H-pyrrolo[1,2- | ¹H NMR (CDCl₃, 400 MHz) δ: 7.76 (d, 1H, J = |
| c]imidazol-5-yl)acetyl)-N-(6- | 8.4 Hz), 7.56 (s, 1H), 7.52 (t, 1H, J = 8.0 Hz), 7.46 (t, |
| methylpyridin-2-yl)piperidine-1- | 2H, J = 8.0 Hz), 7.33 (t, 1H, J = 7.6 Hz), 7.27-7.25 |
| carboxamide | (m, 2H), 7.18 (br. s, 1H), 6.88 (s, 1H), 6.78 (d, 1H, |
| 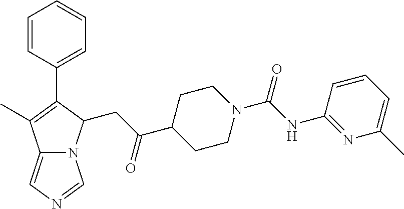 | J = 7.2 Hz), 5.55 (d, 1H, J = 10.4 Hz), 4.10-4.09 (m, 2H), 2.93-2.85 (m, 3H), 2.56 (dd, 1H, J = 10.8, 18.8 Hz), 2.48-2.42 (m, 1H), 2.40 (s, 3H), 2.18 (d, 3H, J = 1.6 Hz), 1.84-1.77 (m, 2H), 1.62-1.55 (m, 2H); Mass (LCMS): 456.1 (M + 1) |
| (183a) | |

Following examples have been synthesized by the above procedure described in Example 158 with their corresponding intermediates in similar reaction conditions:

| Example | IUPAC | |
|---|---|---|
| No. | Name/Structure | Analytical Data |
| 167. | 4-[1-Hydroxy-2-(7- | ¹H NMR (CDCl₃, 400 MHz) δ: 8.15 (d, 1H, J = |
| | methyl-6-phenyl-5H- | 3.6 Hz), 7.97 (d, 1H, J = 8.4 Hz), 7.86 (s, 1H), |
| | pyrrolo[1,2-c]imidazol- | 7.61 (t, 1H, J = 8.4 Hz), 7.42 (t, 2H, J = 7.6 Hz), |
| | 5-yl)-ethyl]-piperidine- | 7.34-7.27 (m, 4H), 6.92-6.88 (m, 2H), 5.46 (d, |
| | 1-carboxylic acid | 0.2H, J = 10 Hz), 5.25 (s, 0.8H), 4.09-4.07 (m, |
| | pyridin-2-ylamide | 2H), 3.39-3.37 (m, 1H), 2.78-2.76 (m, 2H), 2.19 |
| | 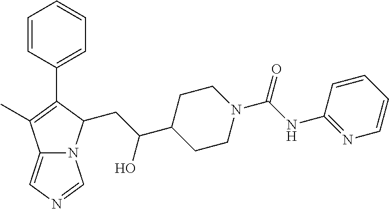 [SMILES: CC1=C(c2ccccc2)C(CC(O)C2CCN(C(=O)Nc3ccccn3)CC2)n2cncc21] | (d, 0.6 H, J = 1.6 Hz), 2.16 (d, 2.4H, J = 1.6 Hz), 2.01-1.95 (m, 1H), 1.86-1.75 (m, 3H), 1.71-1.67 (m, 1H), 1.53-1.49 (m, 1H), 1.45-1.40 (m, 2H) Mass (LCMS): 444.1 (M + 1); Purity: 93%. |
| 179. | N-cyclohexyl-4-(1- | |
| | hydroxy-2-(7-methyl- | |
| | 6-phenyl-5H- | ¹H NMR (CDCl₃, 400 MHz) δ: 7.85 (br. s, 1H), |
| | pyrrolo[1,2-c]imidazol | 7.42 (t, 2H, J = 7.6), 7.34-7.27 (m, 3H), 6.89 (s, |
| | 5-yl)ethyl)piperidine-1- | 1H), 5.44 (d, 0.2H, J = 10.4 Hz), 5.25 (s, 0.8H), |
| | carboxamide | 4.25 (br. s, 1H), 3.90-3.84 (m, 2H), 3.62-3.54 (m, |
| | 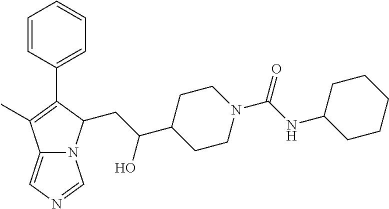 [SMILES: CC1=C(c2ccccc2)C(CC(O)C2CCN(C(=O)NC3CCCCC3)CC2)n2cncc21] | 1H), 3.38-3.32 (m, 1H), 2.64-2.55 (m, 2H), 2.19 (d, 0.6H, J = 1.6 Hz), 2.16 (d, 2.4H, J = 1.6 Hz), 2.00-1.95 (m, 1H), 1.94-1.88 (m, 2H), 1.80-1.72 (m, 1H), 1.70-1.65 (m, 3H), 1.48-1.42 (m, 1H), 1.39-1.30 (m, 3H), 1.17-1.04 (m, 6H). Mass (LCMS): 449.1 (M + 1); Purity: 93.84% |
| 181. | N-(3-chlorophenyl)-4- | ¹H NMR (CDCl₃, 400 MHz) δ: 7.87 (s, 0.8H), |
| | (1-hydroxy-2-(7- | 7.80 (s, 0.2H), 7.44-7.41 (m, 3H), 7.35-7.27 (m, |
| | methyl-6-phenyl-5H- | 3H), 7.19-7.12 (m, 2H), 6.96-6.94 (m, 1H), 6.86 |
| | pyrrolo[1,2-c]imidazol- | (s, 1H), 6.79 (s, 0.2H), 6.75 (s, 0.8H), 5.45 (d, |
| | 5-yl)ethyl)piperidine-1- | 0.2H, J = 10.0 Hz), 5.23 (s, 0.8H), 4.06-4.04 (m, |
| | carboxamide | 2H), 3.74-3.68 (m, 0.2H), 3.40-3.36 (m, 0.8H), |
| | 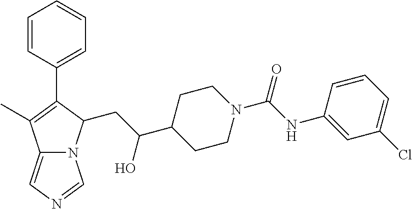 [SMILES: CC1=C(c2ccccc2)C(CC(O)C2CCN(C(=O)Nc3cccc(Cl)c3)CC2)n2cncc21] | 2.76-2.70 (m, 2H), 2.19 (d, 0.6H, J = 1.6 Hz), 2.16 (d, 2.4H, J = 1.6 Hz), 2.02-1.95 (m, 3H), 1.81-1.73(m, 1H), 1.68-1.64 (m, 1H), 1.1.50-1.49 (m, 1H), 1.40-1.35 (m, 1H). Mass (LCMS): 477.1 (M + 1); Purity; 94.18% |
| 182. | N-(3-chloro-4- | ¹H NMR (CDCl₃, 400 MHz) δ: 7.96 (br. S, 1H), |
| | fluorophenyl)-4-(1- | 7.48 (dd, 1H, J = 2.4, 6.8 Hz), 7.43 (t, 2H, J = |
| | hydroxy-2-(7-methyl- | 7.6 Hz), 7.35-7.33 (m, 3H), 7.16-7.13 (m, 1H), 6.99- |
| | 6-phenyl-5H- | 6.94 (m, 1H), 6.87 (s, 1H), 5.46 (d, 0.2H, J = |
| | pyrrolo[1,2-c]imidazol- | 10.4 Hz), 5.23 (s, 0.8H), 4.07-4.05 (m, 2H), 3.72- |
| | 5-yl)ethyl)piperidine-1- | 3.68 (m, 0.2H), 3.48-3.41 (m, 0.8H), 2.74-2.68 (m, |
| | carboxamide | 2H), 2.18 (d, 0.6H, J = 1.2 Hz), 2.16 (d, 2.4 H, |
| | 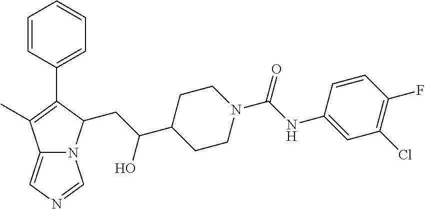 [SMILES: CC1=C(c2ccccc2)C(CC(O)C2CCN(C(=O)Nc3ccc(F)c(Cl)c3)CC2)n2cncc21] | J = 1.2 Hz), 1.99-1.82 (m, 2H), 1.78-1.70 (m, 1H), 1.68-1.60 (m, 1H), 1.51-1.36 (m, 3H). Mass (LCMS): 495.1 (M + 1); Purity: 97.77%. |
| 183. | 4-(1-hydroxy-2-(7- | ¹H NMR (CDCl₃, 400 MHz) δ: 7.91 (br. s, 1H), |
| | methyl-6-phenyl-5H- | 7.77 (d, 1H, J = 8.4 Hz), 7.52-7.47 (m, 1H), 7.42 |
| | pyrrolo[1,2-c]imidazol- | (t, 2H, J = 7.6 Hz), 7.34-7.27 (m, 3H), 7.18 (br. s, |
| | 5-yl)ethyl)-N-(6- | 1H), 6.88 (s, 1H), 6.76 (d, 1H, J = 7.6 Hz), 5.46 |
| | methylpyridin-2- | (d, 0.2H, J = 9.6 Hz), 5.24 (s, 0.8H), 4.08 (m, 2H), |
| | yl)piperidine-1- | 3.73-3.69 (m, 0.2H), 3.41-3.38 (m, 0.8H), 2.78- |
| | carboxamide | 2.70 (m, 2H), 2.39 (s, 2.1H), 2.38 (s, 0.9H), 2.18 |
| | 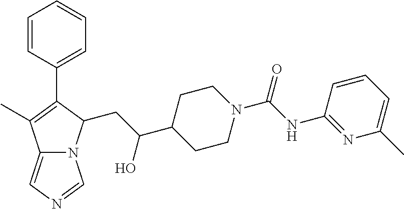 | (d, 0.9H, J = 1.6 Hz), 2.16 (d, 2.1H, J = 1.6 Hz), 2.00-1.90 (m, 1H), 1.88-1.73 (m, 2H), 1.70-1.67 (m, 1H), 1.51 -1.37 (m, 2H), 1.28-1.17 (m, 1H) Mass (LCMS): 458.1 (M + 1); Purity: 97.7% |

### Example 163: Preparation of 1-(1-Cyclohexanesulfonyl-piperidin-4-yl)-2-(7-methyl-6-phenyl-5H-pyrrolo[1,2-c]imidazol-5-yl)-ethanol

### Step-1: Preparation of 1-(1-Cyclohexanesulfonyl-piperidin-4-yl)-2-(7-methyl-6-phenyl-5H-pyrrolo[1,2-c]imidazol-5-yl)-ethanone

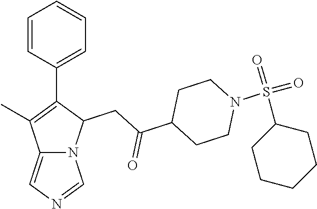 [SMILES: CC1=C(c2ccccc2)C(CC(=O)C2CCN(S(=O)(=O)C3CCCCC3)CC2)n2cncc21]

To a solution of compound 2-(7-methyl-6-phenyl-5H-pyrrolo[1,2-c]imidazol-5-yl)-1-(piperidin-4-yl)ethan-1-one (Example 158-Step 1, 50 mg, 0.155 mmol) in MDC (10 mL) was added NEt₃ (39.17 mg, 0.387 mmol) and Cyclohexyl sulfonyl chloride (31.23 mg, 0.171) at rt. Reaction mixture was stirred at about room temperature for about 12 hours. The reaction was monitored by TLC. After completion of reaction volatiles were removed under vacuum. Crude product was purified using column chromatography to get compound 1-(1-Cyclohexanesulfonyl-piperidin-4-yl)-2-(7-methyl-6-phenyl-5H-pyrrolo[1,2-c]imidazol-5-yl)-ethanone (19 mg, 26.1%) as pale yellow solid.

¹H NMR (CDCl₃, 400 MHz) δ: 7.58 (s, 1H), 7.43 (t, 2H, J=7.6 Hz,), 7.34 (t, 1H, J=7.6 Hz,), 7.27-7.25 (m, 2H), 6.89 (s, 1H), 5.55 (d, 1H, J=10.4 Hz), 3.75-3.73 (m, 2H), 2.88-2.81 (m, 4H), 2.55 (dd, 1H, J=10.4, 18.4 Hz), 2.40-2.32 (m, 1H), 2.18 (d, 3H, J=1.6 Hz), 2.08-2.05 (m, 3H), 1.88-1.76 (m, 5H), 1.70-1.57 (m, 3H), 1.49-1.39 (m, 3H).

### Step-2: Preparation of 1-(1-Cyclohexanesulfonyl-piperidin-4-yl)-2-(7-methyl-6-phenyl-5H-pyrrolo[1,2-c]imidazol-5-yl)-ethanol

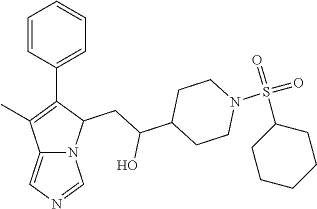 [SMILES: CC1=C(c2ccccc2)C(CC(O)C2CCN(S(=O)(=O)C3CCCCC3)CC2)n2cncc21]

To a solution of compound 1-(1-Cyclohexanesulfonyl-piperidin-4-yl)-2-(7-methyl-6-phenyl-5H-pyrrolo[1,2-c]imidazol-5-yl)-ethanone (Step 1, 10 mg, 0.021 mmol) in MeOH (5 ml) was added NaBH₄ (1.22 mg, 0.032 mmol) at about 5-10° C. the reaction was stirred at the same temperature for about 15-20 minutes. The reaction was monitored by TLC. After completion of reaction solvents were removed under vacuum. Residue was taken in water and extracted with ethyl acetate (3×30 mL). Combined ethyl acetate was dried over anhydrous Na₂SO₄ and concentrated to give compound 1-(1-Cyclohexanesulfonyl-piperidin-4-yl)-2-(7-methyl-6-phenyl-5H-pyrrolo[1,2-c]imidazol-5-yl)-ethanol (7 mg, 69.6%) as white solid.

¹H NMR (CDCl₃, 400 MHz) δ: 7.82 (s, 1H), 7.43 (t, 2H, J=7.6 Hz), 7.35-7.27 (m, 3H), 6.86 (s, 1H), 5.43 (dd, 0.2H, J=1.2, 9.6 Hz), 5.24 (s, 0.8H), 3.79-3.73 (m, 3H), 3.37-3.34 (m, 1H), 2.2.86-2.79 (m, 1H), 2.75-2.69 (m, 2H), 2.19 (s, 0.6H), 2.16 (s, 2.4H), 2.06-1.96 (m, 4H), 1.86-1.75 (m, 4H), 1.67 (d, 2H, J=10.4 Hz), 1.49-1.45 (m, 3H), 1.28-1.21 (m, 4H). Mass (LCMS): 470.1 (M+1); Purity: 90.8%.

Following intermediates have been synthesized as described in the above process of Example 163-Step 1:

| Intermediate No. | Analytical Data |
|---|---|
| 1-(1-Methanesulfonyl-piperidin-4-yl)-2- | ¹H NMR (CDCl₃, 400 MHz) δ: 7.91 (s, 1H), 7.46 (t, |
| (7-methyl-6-phenyl-5H-pyrrolo[1,2- | 2H, J = 7.6 Hz), 7.37 (t, 1H, J = 7.6 Hz), 7.29-7.27 |
| c]imidazol-5-yl)-ethanone | (m, 2H), 6.98 (s, 1H), 5.61 (d, 1H, J = 9.6 Hz), 3.73- |
| 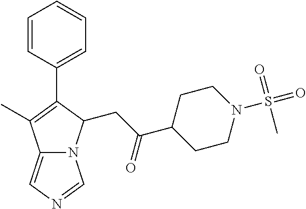 [SMILES: CC1=C(c2ccccc2)C(CC(=O)C2CCN(S(C)(=O)=O)CC2)n2cncc21] | 3.72 (m, 2H), 2.97-2.95 (m, 1H), 2.77 (s, 3H), 2.75- 2.64 (m, 3H), 2.42-2.36 (m, 1H), 2.19 (d, 3H, J = 1.2 Hz), 1.91-1.84 (m, 2H), 1.72-1.63 (m, 2H); Mass (LCMS): 400.1 (M + 1) |
| (165a) | |

Following example has been synthesized by the above procedure described in Example 163 with their corresponding intermediates in similar reaction conditions:

| Example | IUPAC | |
|---|---|---|
| No. | Name/Structure | Analytical Data |
| 165. | 1-(1-Methanesulfonyl- | ¹H NMR (CDCl₃, 400 MHz) δ: 7.88 (s, 1H), 7.43 |
| | piperidin-4-yl)-2-(7- | (t, 2H, J = 7.6 Hz), 7.35-7.27 (m, 3H), 687 (s, 1H), |
| | methyl-6-phenyl-5H- | 5.25 (s, 1H), 3.79-3.73 (m, 2H), 3.41-3.35 (m, |
| | pyrrolo[1,2- | 1H), 2.72 (s, 3H), 2.54-2.49 (m, 2H), 2.19 (s, |
| | c]imidazol-5-yl)- | 0.7H), 2.16 (s, 2.3H), 2.04-1.91 (m, 2H), 1.81- |
| | ethanol | 1.74 (m, 2H), 1.55 (d, 1H, J = 9.2 Hz), 1.34-1.27 |
| | 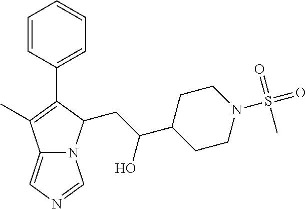 [SMILES: CC1=C(c2ccccc2)C(CC(O)C2CCN(S(C)(=O)=O)CC2)n2cncc21] | (m, 3H) Mass (LCMS): 402.1 (M + 1): Purity: 94.01%. |

### Example 164: Preparation of {4-[1-Hydroxy-2-(7-methyl-6-phenyl-5H-pyrrolo[1,2-c]imidazol-5-yl)-ethyl]-piperidin-1-yl}-(2-methyl-pyridin-4-yl)-methanone

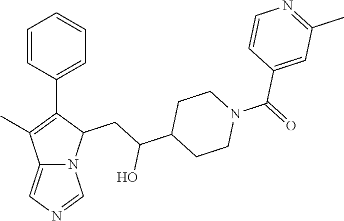

### Step-1: Preparation of 2-(7-Methyl-6-phenyl-5H-pyrrolo[1,2-c]imidazol-5-yl)-1-[1-(2-methyl-pyridine-4-carbonyl)-piperidin-4-yl]-ethanone

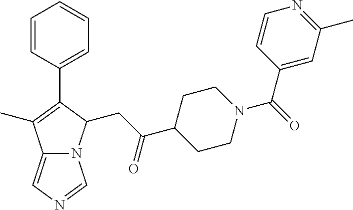

To a solution of 2-Methylisonicotinic acid (42.6 mg, 0.311 mmol) in DMF was added HATU (177.4 mg, 0.446 mmol) diisopropyl ethyl amine (0.135 mL, 0.777 mmol) at about 5-10° C. and reaction mixture was stirred for about 10-15 minutes. To the above reaction mixture was added DMF solution of compound 2-(7-methyl-6-phenyl-5H-pyrrolo[1,2-c]imidazol-5-yl)-1-(piperidin-4-yl)ethan-1-one (Step 1; Example 158, 100 mg, 0.311 mmol) at about 5-10° C. Reaction mixture was stirred at about room temperature for about 3-4 hours and monitored by TLC. After completion of reaction was diluted with water and product was extracted in ethyl acetate (3×50 mL). combined organic layer was washed with brine (30 ml) followed by water (30 mL). Organic layer was dried over Na₂SO₄ and concentrated to get crude product which was further purified using flash column chromatography (MDC/MeOH 2-3% as eluent). Desired compound 2-(7-Methyl-6-phenyl-5H-pyrrolo[1,2-c]imidazol-5-yl)-1-[1-(2-methyl-pyridine-4-carbonyl)-piperidin-4-yl]-ethanone was obtained as pale yellow solid (47 mg, 34.3%).

¹H NMR (CDCl₃, 400 MHz) δ: 8.55 (d, 1H, J=5.2 Hz), 7.59 (s, 1H), 7.45-7.42 (m, 2H), 7.36-7.31 (m, 1H), 7.29-7.23 (m, 2H), 7.11 (s, 1H), 7.03 (d, 1H, J=5.2 Hz), 6.89 (s, 1H), 5.56 (d, 1H, J=10.4 Hz), 4.61 (d, 1H, J=10.4 Hz), 3.63 (d, 1H, J=11.6 Hz), 2.98-2.82 (m, 4H), 2.58 (s, 3H), 2.55-2.49 (m, 1H), 2.18 (d, 3H, J=1.6 Hz), 1.96-1.82 (m, 4H). Mass (LCMS): 441.1 (M+1)

### Step-2: Preparation of {4-[1-Hydroxy-2-(7-methyl-6-phenyl-5H-pyrrolo[1,2-c]imidazol-5-yl)-ethyl]-piperidin-1-yl}-(2-methyl-pyridin-4-yl)-methanone

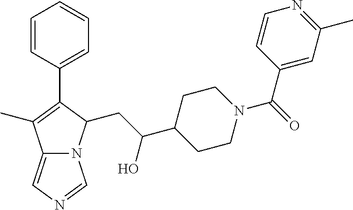

To a solution of compound 2-(7-Methyl-6-phenyl-5H-pyrrolo[1,2-c]imidazol-5-yl)-1-[1-(2-methyl-pyridine-4-carbonyl)-piperidin-4-yl]-ethanone (25 mg, 0.056 mmol) in MeOH (5 ml) was added NaBH₄ (3.2 mg, 0.085 mmol) at about 5-10° C. Reaction was stirred at the same temperature for about 15-20 minutes. The reaction was monitored by TLC. After completion of reaction solvents were removed under vacuum. Residue was taken in water and extracted with ethyl acetate (3×30 mL). Combined ethyl acetate was dried over anhydrous Na₂SO₄ and concentrated to give compound {4-[1-Hydroxy-2-(7-methyl-6-phenyl-5H-pyrrolo[1,2-c]imidazol-5-yl)-ethyl]-piperidin-1-yl}-(2-methyl-pyridin-4-yl)-methanone (12 mg, 47.8%) as white solid.

¹H NMR (CDCl₃, 400 MHz) δ: 8.52 (d, 1H, J=5.2 Hz), 7.43 (t, 2H, J=7.6 Hz), 7.36-7.27 (m, 4H), 7.08 (s, 1H), 7.00 (d, 1H, J=4.4 Hz), 6.89 (br. s, 1H), 5.43 (s, 0.2H), 5.25 (s, 0.8H), 4.74-4.64 (m, 1H), 3.65-3.55 (m, 2H), 3.37 (br. s, 1H), 2.93-2.84 (m, 1H), 2.64-2.58 (m, 1H), 2.56 (s, 3H), 2.20 (s, 0.6H), 2.17 (s, 2.4H), 2.06-1.97 (m, 2H), 1.82-1.76 (m, 5H). Mass (LCMS): 443.1 (M+1). Purity: 91.49%.

Following intermediates have been synthesized as described in the above process of Example 164-Step 1:

| Intermediate No. | Analytical Data |
|---|---|
| 2-(7-Methyl-6-phenyl-5H-pyrrolo[1,2- | ¹H NMR (CDCl₃, 400 MHz) δ: 8.56 (s, 1H), 7.79 (dt, |
| c]imidazol-5-yl)-1-[1-(pyridine-2- | 1H, J = 1.6, 7.6 Hz), 7.60 (d, 1H, J = 8 Hz), 7.55 (d, |
| carbonyl)-piperidin-4-yl]-ethanone | 1H, J = 8.8 Hz), 7.46-7.41 (m, 2H), 7.36-7.32 (m, |
| 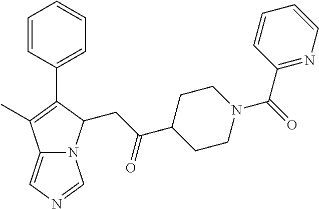 [SMILES: CC1=C(c2ccccc2)C(CC(=O)C2CCN(C(=O)c3ccccn3)CC2)n2cncc21] | 2H), 7.28-7.24 (m, 2H), 6.81 (d, 1H, J = 3.6 Hz), 5.59-5.53 (m, 1H), 4.66 (d, 1H, J = 14 Hz), 3.99 (d, 1H, J = 14 Hz), 3.05 (m, 1H), 2.91-2.81 (m, 2H), 2.62-2.50 (m, 2H), 2.18 (d, 3H, J = 1.6 Hz), 1.92- 1.84 (m, 1H), 1.73-1.68 (m, 3H). Mass (LCMS): 427.1 (M + 1) |
| (152a) | |
| 2-(7-Methyl-6-phenyl-5H-pyrrolo[1,2- | ¹H NMR (CDCl₃, 400 MHz) δ: 8.66 (dd, 1H, J = 1.6, |
| c]imidazol-5-yl)-1-[1-(pyridine-3- | 4.8 Hz), 8.62 (d, 1H, J = 1.6 Hz), 7.74-7.72 (m, 1H), |
| carbonyl)-piperidin-4-yl]-ethanone | 7.57 (s, 1H), 7.43 (t, 2H, J = 7.6 Hz), 7.37-7.32 (m, |
| 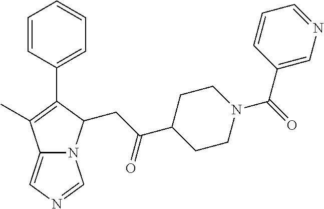 [SMILES: CC1=C(c2ccccc2)C(CC(=O)C2CCN(C(=O)c3cccnc3)CC2)n2cncc21] | 2H), 7.27-7.25 (m, 2H), 6.89 (s, 1H), 5.56 (d, 1H, J = 10 Hz), 4.65-4.55 (m, 1H), 3.76-3.70 (m, 1H), 3.10-2.98 (m, 1H), 2.90-2.88 (m, 2H), 2.57-2.49 (m, 2H), 2.18 (d, 3H, J = 1.6 Hz), 1.97-1.75 (m, 4H). Mass (LCMS): 427.1 (M + 1) |
| (157a) | |
| 2-(7-Methyl-6-phenyl-5H-pyrrolo[1,2- | ¹H NMR (CDCl₃, 400 MHz) δ: 8.68 (d, 2H, J = |
| c]imidazol-5-yl)-1-[1-(pyridine-4- | 6.0 Hz), 7.65 (s, 1H), 7.46-7.42 (m, 2H), 7.38-7.33 (m, |
| carbonyl)-piperidin-4-yl]-ethanone | 1H), 7.27-7.24 (m, 4H), 6.90 (s, 1H), 5.58 (d, 1H, |
| 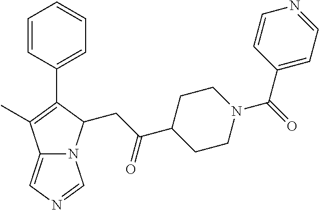 [SMILES: CC1=C(c2ccccc2)C(CC(=O)C2CCN(C(=O)c3ccncc3)CC2)n2cncc21] | J = 9.2 Hz), 4.61 (d, 1H, J = 12 Hz), 3.65-3.60 (m, 1H), 3.03-2.80 (m, 3H), 2.66-2.47 (m, 2H), 2.19 (d, 3H, J = 1.6 Hz), 1.94-1.86 (m, 1H), 1.77-1.69 (m, 1H), 1.62-1.52 (m, 2H). Mass (LCMS): 427.1 (M + 1) |
| (162a) | |
| 2-(7-methyl-6-phenyl-5H-pyrrolo[1,2- | ¹H NMR (CDCl₃, 400 MHz) δ: 7.53-7.50 (m, 1H), |
| c]imidazol-5-yl)-1-(1-(2- | 7.42 (t, 2H, J = 7.2 Hz), 7.35-7.28 (m, 4H), 7.24-7.19 |
| phenylacetyl)piperidin-4-yl)ethan-1-one | (m, 4H), 6.87 (s, 1H), 5.53-5.50 (m, 1H), 4.53 (d, 1H, |
| 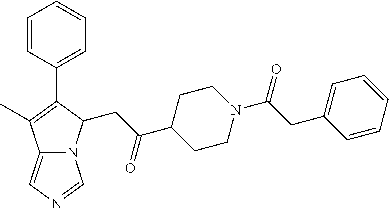 [SMILES: CC1=C(c2ccccc2)C(CC(=O)C2CCN(C(=O)Cc3ccccc3)CC2)n2cncc21] | J = 12.4 Hz), 3.86-3.81 (m, 1H), 3.70 (s, 2H), 2.97- 2.88 (m, 1H), 2.85-2.76 (m, 1H), 2.66-2.59 (m, 1H), 2.54-2.47 (m, 1H), 2.42-2.36 (m, 1H), 2.18 (d, 3H, J = 1.6 Hz), 1.80-1.68 (m, 4H). Mass (LCMS): 440.1 (M + 1) |
| (180a) | |
| 2-methyl-1-(4-(2-(7-methyl-6-phenyl- | ¹H NMR (CDCl₃) δ: 7.54 (s, 1H), 7.44-7.41 (m, 2H), |
| 5H-pyrrolo[1,2-c]imidazol-5- | 7.36-7.31 (m, 1H), 7.27-7.25 (m, 2H), 6.87 (s, 1H), |
| yl)acetyl)piperidin-1-yl)propan-1-one | 5.54 (d, 1H, J = 10.4 Hz), 4.55-4.52 (m, 1H), 3.93- |
| 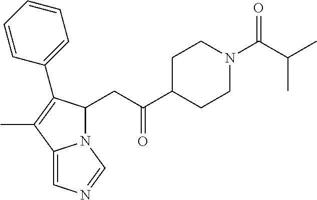 [SMILES: CC1=C(c2ccccc2)C(CC(=O)C2CCN(C(=O)C(C)C)CC2)n2cncc21] | 3.90 (m, 1H), 3.02-2.96 (m, 1H), 2.90-2.82 (m, 1H), 2.77-2.71 (m, 1H), 2.60-2.52 (m, 2H), 2.51-2.42 (m, 1H), 2.18 (d, 3H, J = 1.6 Hz), 1.83-1.80 (m, 1H), 1.58-1.40 (m, 3H), 1.09 (s, 3H), 1.08 (s, 3H); Mass (LCMS): 392.1 (M + 1). |
| (187a) | |

Following examples have been synthesized by the above procedure described in Example 164 with their corresponding intermediates in similar reaction conditions:

| Example | IUPAC | |
|---|---|---|
| No. | Name/Structure | Analytical Data |
| 152. | {4-[1-Hydroxy-2-(7- | ¹H NMR (CDCl₃, 400 MHz) δ: 8.55 (d, 1H, J = |
| | methyl-6-phenyl-5H- | 4.4 Hz), 7.84 (s, 0.8H), 7.77-7.75 (m, 1.2H), 7.54 |
| | pyrrolo[1,2-c]imidazol- | (d, 1H, J = 8.0 Hz), 7.42 (t, 2H, J = 7.6 Hz), 7.34- |
| | 5-yl)-ethyl]-piperidin- | 7.26 (m, 4H), 6.87 (s, 1H), 5.46 (br. s, 0.2H), 5.25 |
| | 1-yl}-pyridin-2-yl- | (br. s, 0.8H), 4.74-4.68 (m, 1H), 3.92-3.85 (m, |
| | methanone | 1H), 3.41-3.35 (m, 1H), 2.96-2.89 (m, 1H), 2.66- |
| | 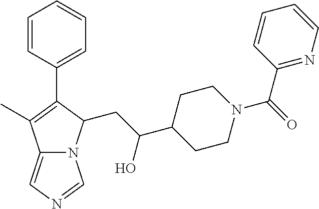 [SMILES: CC1=C(c2ccccc2)C(CC(O)C2CCN(C(=O)c3ccccn3)CC2)n2cncc21] | 2.64 (m, 1H), 2.19 (s, 0.6H), 2.16 (s, 2.4H), 2.08- 1.95 (m, 4H), 1.83-1.73 (m, 2H), 1.60-1.55 (m, 2H); Mass (LCMS): 429.1 (M + 1); Purity: 91.86%. |
| 160. | {4-[1-Hydroxy-2-(7- | ¹H NMR (CDCl₃, 400 MHz) δ: 8.55 (s, 1H), 7.81- |
| | methyl-6-phenyl-5H- | 7.75 (m, 2H), 7.56 (d, 1H, J = 7.2 Hz), 7.43 (t, 2H, |
| | pyrrolo[1,2-c]imidazol- | J = 7.2 Hz), 7.39-7.29 (m, 4H), 6.88 (s, 1H), 5.26 |
| | 5-yl)-ethyl]-piperidin- | (s, 1H), 4.76-4.69 (m, 1H), 3.92-3.86 (m, 1H), |
| | 1-yl}-pyridin-2-yl- | 3.67-3.64 (m, 1H), 3.36-3.31 (m, 1H), 2.96-2.88 |
| | methanone, Isomer-I | (m, 1H), 2.70-2.61 (m, 1H), 2.20 (s, 3H), 2.04- |
| | 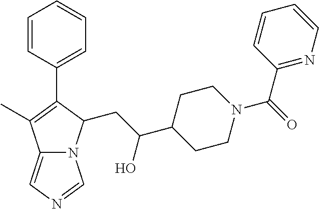 [SMILES: CC1=C(c2ccccc2)C(CC(O)C2CCN(C(=O)c3ccccn3)CC2)n2cncc21] | 1.95 (m, 1H), 1.88-1.77 (m, 2H), 1.62-1.55 (m, 2H), 1.50-1.40 (m, 2H). Mass (LCMS): 429.1 (M + 1); Purity: 99.21%. |
| 161. | {4-[1-Hydroxy-2-(7- | ¹H NMR (CDCl₃, 400 MHz) δ: 8.55 (s, 1H), 7.84 |
| | methyl-6-phenyl-5H- | (s, 1H), 7.76 (s, 1H), 7.55-7.27 (m, 7H), 6.86 (s, |
| | pyrrolo[1,2-c]imidazol- | 1H), 5.25 (s, 1H), 4.77-4.65 (m, 1H), 3.94-3.82 |
| | 5-yl)-ethyl]-piperidin- | (m, 1H), 3.43-3.35 (m, 1H), 2.99-2.87 (m, 1H), |
| | 1-yl}-pyridin-2-yl- | 2.71-2.59 (m, 1H), 2.16 (s, 3H), 2.10-1.96 (m, |
| | methanone, Isomer-II | 4H), 1.85-1.72 (m, 2H), 1.65-1.53 (m, 2H). Mass |
| | 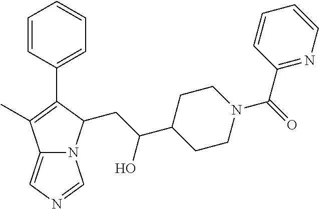 [SMILES: CC1=C(c2ccccc2)C(CC(O)C2CCN(C(=O)c3ccccn3)CC2)n2cncc21] | (LCMS): 429.1 (M + 1); Purity: 96.77%. |
| 157. | {4-[1-Hydroxy-2-(7- | ¹H NMR (CDCl₃, 400 MHz) δ: 8.63 (dd, 1H, J = |
| | methyl-6-phenyl-5H- | 1.6, 4.8 Hz), 8.59 (d, 1H, J = 1.2 Hz), 7.85 (br. s, |
| | pyrrolo[1,2-c]imidazol- | 1H), 7.71-7.69 (m, 1H), 7.43 (t, 2H, J = 8.0 Hz), |
| | 5-yl)-ethyl]-piperidin- | 7.35-7.31 (m, 2H), 7.28 (d, 2H, J = 7.2 Hz), 6.89 |
| | 1-yl}-pyridin-3-yl- | (s, 1H), 5.26 (br. s, 1H), 4.69 (br. s, 1H), 3.68- |
| | methanone | 3.61 (m, 1H), 3.39 (br. s, 1H), 2.94 (br. s, 1H), |
| | 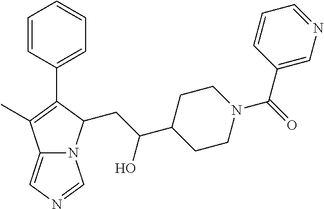 [SMILES: CC1=C(c2ccccc2)C(CC(O)C2CCN(C(=O)c3cccnc3)CC2)n2cncc21] | 2.65 (br. s, 1H), 2.19 (s, 0.6H), 2.17 (s, 2.4H), 2.06-1.97 (m, 4H), 1.87-1.75 (m, 2H), 1.68-1.53 (m, 2H). Mass (LCMS): 429.1 (M + 1); Purity: 98.8%. |
| 162. | {4-[1-Hydroxy-2-(7- | ¹H NMR (CDCl₃) δ: 8.65-8.63 (m, 2H), 7.43 (t, |
| | methyl-6-phenyl-5H- | 2H, J = 7.6 Hz), 7.34 (t, 1H, J = 7.6 Hz), 7.29- |
| | pyrrolo[1,2-c]imidazol- | 7.27 (m, 3H), 7.22-7.20 (m, 2H), 6.94 (s, 1H), |
| | 5-yl)-ethyl]-piperidin- | 5.28 (s, 1H), 4.69-4.64 (m, 1H), 3.65-3.43 (m, |
| | 1-yl}-pyridin-4-yl- | 3H), 2.94-2.86 (m, 1H), 2.66-2.58 (m, 1H), 2.19 |
| | methanone | (s, 0.6 H), 2.17 (s, 2.4H), 2.05-1.91 (m, 2H), 1.79- |
| | 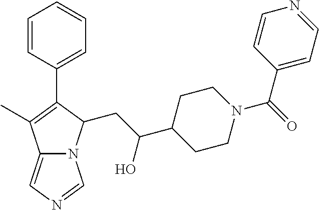 [SMILES: CC1=C(c2ccccc2)C(CC(O)C2CCN(C(=O)c3ccncc3)CC2)n2cncc21] | 1.63 (m, 5H); Mass (LCMS): 429.1 (M + 1); Purity: 92.9%. |
| 180. | 1-(4-(1-hydroxy-2-(7- | ¹H NMR (CDCl₃, 400 MHz) δ: 7.90 (br. s, 1H), |
| | methyl-6-phenyl-5H- | 7.42 (t, 2H, J = 7.6 Hz), 7.35-7.27 (m, 5H), 7.22- |
| | pyrrolo[1,2-c]imidazol- | 7.18 (m, 3H), 6.89 (s, 1H), 5.42 (d, 0.2H, J = |
| | 5-yl)ethyl)piperidin-1- | 10.4 Hz), 5.24 (s, 0.8H), 4.64-4.58 (m, 1H), 3.88- |
| | yl)-2-phenylethan-1- | 3.75 (m, 1H), 3.67 (s, 2H), 3.66-3.64 (m, 1H), 3.29 |
| | one | (br.s, 1H), 2.84-2.78 (m, 1H), 2.44-2.37 (m, 1H), |
| | 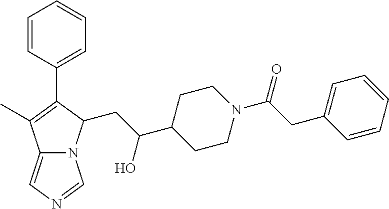 [SMILES: CC1=C(c2ccccc2)C(CC(O)C2CCN(C(=O)Cc3ccccc3)CC2)n2cncc21] | 2.18 (s, 0.6H), 2.16 (s, 2.4H), 1.92-1.86 (m, 3H), 1.63-1.51 (m, 2H), 1.40-1.32 (m, 2H). Mass (LCMS): 442.1 (M + 1); Purity: 91.37% |
| 187. | 1-(4-(1-hydroxy-2-(7- | ¹H NMR (CDCl₃) δ: 7.85 (s, 1H), 7.42 (t, 2H, J = |
| | methyl-6-phenyl-5H- | 7.2 Hz), 7.34-7.27 (m, 3H), 6.86 (s, 1H), 5.46 (s, |
| | pyrrolo[1,2-c]imidazol | 0.2H), 5.24 (s, 0.8H), 4.67-4.59 (m, 1H), 3.93- |
| | 5-yl)ethyl)piperidin-1- | 3.86 (m, 1H), 3.37 (br. s, 1H), 2.92-2.85 (m, 1H), |
| | yl)-2-methylpropan-1- | 2.75-2.73 (m, 1H), 2.41-2.32 (m, 3H), 2.19 (d, |
| | one | 0.6H, J = 1.2 Hz), 2.16 (d, 2.4H, J = 1.2 Hz), 1.99- |
| | 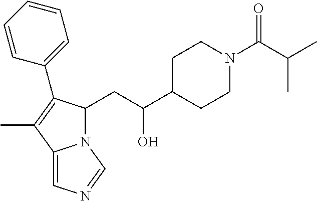 [SMILES: CC1=C(c2ccccc2)C(CC(O)C2CCN(C(=O)C(C)C)CC2)n2cncc21] | 1.95 (m, 1H), 1.82-1.74 (m, 2H), 1.52-1.41 (m, 2H), 1.28-1.26 (m, 1H), 1.07 (d, 6 H, J = 6.4 Hz); Mass (LCMS): 394.1 (M + 1), Purity: 96.57%. |

### Example 166: Preparation of 2-(7-Methyl-6-phenyl-5H-pyrrolo[1,2-c]imidazol-5-yl)-1-[1-(2-methyl-pyridine-4-carbonyl)-piperidin-4-yl]-ethanone

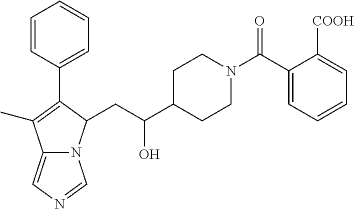 [SMILES: CC1=C(c2ccccc2)C(CC(O)C2CCN(C(=O)c3ccccc3C(=O)O)CC2)n2cncc21]

### Step-1: Preparation of 2-{4-[2-(7-Methyl-6-phenyl-5H-pyrrolo[1,2-c]imidazol-5-yl)-acetyl]-piperidine-1-carbonyl}-benzoic acid

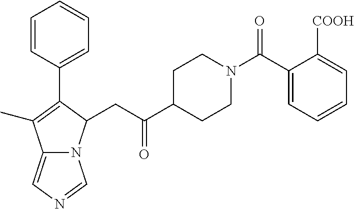 [SMILES: CC1=C(c2ccccc2)C(CC(=O)C2CCN(C(=O)c3ccccc3C(=O)O)CC2)n2cncc21]

To a solution of compound 2-(7-methyl-6-phenyl-5H-pyrrolo[1,2-c]imidazol-5-yl)-1-(piperidin-4-yl)ethan-1-one (Step 1; Example 158, 100 mg, 0.311) in THF (10 ml) was added phthalic anhydride (50.7 mg, 0.342 mmol) at about room temperature. Reaction mixture was stirred at rt for about 12 hours. Reaction monitored by TLC. After completion of reaction volatiles were removed under vacuum. Crude product was purified using preparative HPLC to get compound 2-{4-[2-(7-Methyl-6-phenyl-5H-pyrrolo[1,2-c]imidazol-5-yl)-acetyl]-piperidine-1-carbonyl}-benzoic acid (35 mg, 23.9%) as white solid. confirmed by LCMS.

Mass (LCMS): 470.1 (M+1).

### Step-2: Preparation of 2-{4-[1-Hydroxy-2-(7-methyl-6-phenyl-5H-pyrrolo[1,2-c]imidazol-5-yl)-ethyl]-piperidine-1-carbonyl}-benzoic acid

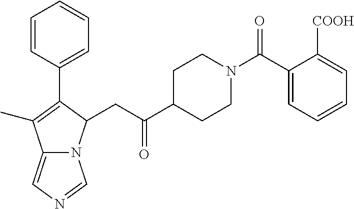 [SMILES: CC1=C(c2ccccc2)C(CC(=O)C2CCN(C(=O)c3ccccc3C(=O)O)CC2)n2cncc21]

To a solution of compound 2-{4-[2-(7-Methyl-6-phenyl-5H-pyrrolo[1,2-c]imidazol-5-yl)-acetyl]-piperidine-1-carbonyl}-benzoic acid (Step 1, 30 mg, 0.063 mmol) in MeOH (5 ml) was added NaBH₄ (3.2 mg, 0.095 mmol) at about 5-10° C. Reaction was stirred at the same temperature for about 15-20 minutes. Reaction was monitored by TLC. After completion of reaction solvents were removed under vacuum. Residue was purified using preparative HPLC to give compound 2-{4-[1-Hydroxy-2-(7-methyl-6-phenyl-5H-pyrrolo[1,2-c]imidazol-5-yl)-ethyl]-piperidine-1-carbonyl}-benzoic acid (6 mg, 19.9%) as white solid.

¹H NMR (CD₃OD, 400 MHz) δ: 8.55 (s, 0.4H), 7.93 (br. s, 0.6H), 7.89-7.85 (m, 1H), 7.46-7.42 (m, 2H), 7.39-7.31 (m, 5H), 7.10 (br. s, 1H), 6.84-6.83 (m, 0.6H), 6.78 (s, 0.4H), 5.48 (d, 0.4H, J=10 Hz), 5.35 (s, 0.6H), 4.62-4.54 (m, 1H), 2.93-2.79 (m, 1H), 2.70-2.61 (m, 1H), 2.16-2.13 (m, 3H), 2.02-1.97 (m, 1H), 1.88-1.82 (m, 1H), 1.69-1.57 (m, 2H), 1.50-1.41 (m, 3H), 1.22-1.10 (m, 1H), 1.05-0.95 (m, 1H); Mass (LCMS): 472 (M+1). Purity: 98.28%.

### Example 188: Preparation of 2-(7-methyl-6-phenyl-5H-pyrrolo[1,2-c]imidazol-5-yl)-1-(1-(pyridin-2-yl)piperidin-4-yl)ethan-1-ol

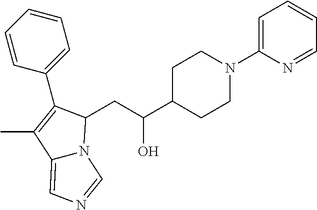 [SMILES: CC1=C(c2ccccc2)C(CC(O)C2CCN(c3ccccn3)CC2)n2cncc21]

### Step-1: Preparation of 2-(7-methyl-6-phenyl-5H-pyrrolo[1,2-c]imidazol-5-yl)-1-(1-(pyridin-2-yl)piperidin-4-yl)ethan-1-one

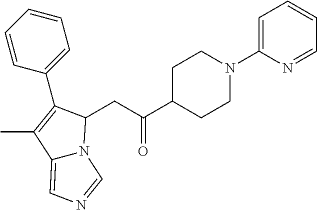 [SMILES: CC1=C(c2ccccc2)C(CC(=O)C2CCN(c3ccccn3)CC2)n2cncc21]

To a solution of 2-(7-methyl-6-phenyl-5H-pyrrolo[1,2-c]imidazol-5-yl)-1-(piperidin-4-yl)ethan-1-one (Step 1; Example 158, 150 mg, 0.467 mmol) and DIPEA (241 mg, 1.86 mmoL) in DMF (5 mL), 2-bromo-pyridine (81.2 mg, 0.513 mmoL) was added at about room temperature. This reaction mass was heated overnight at about 80° C. and monitored by TLC. After completion of the reaction was diluted with water and product was extracted in ethyl acetate (3×50 mL). Combined organic layer was washed with brine (30 mL) followed by water (30 mL). Organic layer was dried over anhydrous Na₂SO₄ and concentrated to get crude product which was further purified using flash column chromatography (MDC/MeOH 2-3% as eluent). Desired compound 2-(7-methyl-6-phenyl-5H-pyrrolo[1,2-c]imidazol-5-yl)-1-(1-(pyridin-2-yl)piperidin-4-yl)ethan-1-one was obtained as pale yellow solid (20 mg, 10.8%).

¹H NMR (CDCl₃, 400 MHz) δ: 7.97 (s, 1H), 7.54 (d, 1H, J=2.4 Hz), 7.45-7.4, (m, 3H), 7.35-7.31 (m, 2H), 7.27-7.25 (m, 3H), 6.87 (s, 1H), 5.54 (d, 1H, J=10.4 Hz), 4.32-4.21 (m, 1H), 3.63-3.60 (m, 1H), 3.07-2.99 (m, 1H), 2.91-2.83 (m, 1H), 2.69-2.45 (m, 3H), 2.18 (s, 3H), 1.85-1.75 (m, 2H), 1.57-1.47 (m, 2H), Mass (LCMS): 399.1 (M+1)

### Step-2: Preparation of 2-(7-methyl-6-phenyl-5H-pyrrolo[1,2-c]imidazol-5-yl)-1-(1-(pyridin-2-yl)piperidin-4-yl)ethan-1-ol

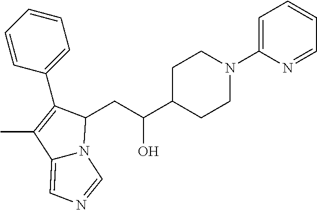 [SMILES: CC1=C(c2ccccc2)C(CC(O)C2CCN(c3ccccn3)CC2)n2cncc21]

To a solution of compound 2-(7-methyl-6-phenyl-5H-pyrrolo[1,2-c]imidazol-5-yl)-1-(1-(pyridin-2-yl)piperidin-4-yl)ethan-1-one (Step 1, 13 mg, 0.032 mmoL) in MeOH (5 mL) was added NaBH₄ (1.86 mg, 0.048 mmoL) at about 5-10° C. Reaction was stirred at the same temperature for about 15-20 minutes and monitored by TLC. After completion of the reaction, solvents were removed under vacuum. Residue was taken in water and extracted with ethyl acetate (3×30 mL). Combined ethyl acetate was dried over anhydrous Na₂SO₄ and concentrated to give compound 2-(7-methyl-6-phenyl-5H-pyrrolo[1,2-c]imidazol-5-yl)-1-(1-(pyridin-2-yl)piperidin-4-yl)ethan-1-ol (10 mg, 76.9%) as white solid.

¹H NMR (CDCl₃, 400 MHz) δ: 7.94 (d, 1H, J=3.2 Hz), 7.83 (br. s, 1H), 7.45-7.41 (m, 3H), 7.35-7.27 (m, 5H), 6.87 (s, 1H), 5.46-5.43 (m, 0.2H), 5.25 (s, 0.8H), 4.41-4.34 (m, 1H), 3.60-3.53 (m, 1H), 3.38 (br. s, 1H), 2.98-2.91 (m, 1H), 2.50-2.44 (m, 1H), 2.19 (s, 0.6H), 2.16 (s, 2.4H), 2.04-1.94 (m, 2H), 1.83-1.77 (m, 4H), 1.54-1.49 (m, 1H), 1.14-1.07 (m, 1H); Mass (LCMS): 401.1 (M+1), Purity: 95.52%.

### Example 168: Preparation of 5-(2-Cyclohexyl-2-fluoro-ethyl)-7-methyl-6-phenyl-5H-pyrrolo[1,2-c]imidazole

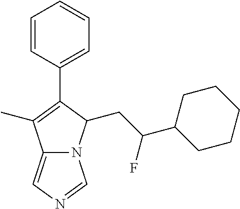 [SMILES: CC1=C(c2ccccc2)C(CC(F)C2CCCCC2)n2cncc21]

Diethylaminosulfur trifluoride (22.5 mg, 0.139 mmol) was added to a solution of compound 1-Cyclohexyl-2-(7-methyl-6-phenyl-5H-pyrrolo[1,2-c]imidazol-5-yl)-ethanol (Example 89, 15 mg, 0.046 mmol) in dichloromethane (5 ml) at about 0-5° C. The reaction mixture was stirred for about 4 hours at about 20-25° C. Reaction mixture was quenched by sodium bicarbonate solution (5 ml) and product was extracted by chloroform (2×15 ml). Organic layer was dried over anhydrous sodium sulphate, filtered and concentrated under vacuum to yield the crude residue which was purified by silica gel column chromatography to obtain desired compound (8 mg, 53%).

¹HNMR (CDCl₃, 400 MHz) δ: 7.75-7.64 (m, 1H), 7.45-7.40 (m, 2H), 7.36-7.27 (m, 3H), 6.91-6.87 (m, 1H), 5.32-5.10 (m, 1H), 2.20-2.15 (m, 3H), 2.11-2.01 (m, 1H), 1.97-1.83 (m, 2H), 1.76-1.56 (m, 3H), 1.54-1.44 (m, 3H), 1.40-1.31 (m 2H), 1.24-1.06 (m 3H); Mass (LCMS): 325.2 (M+1); Purity: 93.51%.

### Example 169: Preparation of 1-Phenyl-2-(6-phenyl-5H-pyrrolo[1,2-c]imidazol-5-yl)-ethanone

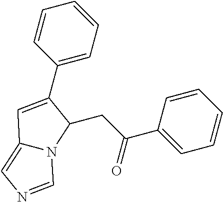 [SMILES: O=C(CC1C(c2ccccc2)=Cc2cncn21)c1ccccc1]

### Step 1: Preparation of 2-Phenyl-3-(3-trityl-3H-imidazol-4-yl)-acrylic acid methyl ester

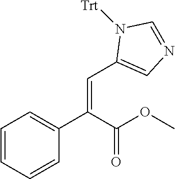

To a stirred solution of 1-triphenylmethyl-5-imidazolecarboxaldehyde (5.6 g, 16.66 mmol) and methyl benzeneacetate (2.5 g, 16.66 mmol) in (45 mL) dry THF, sodium hydride (60% suspension in oil) (1.0 g, 41.25 mmol) was added in portions at about 0° C. The mixture was stirred at room temperature for about 5 hours. After completion of reaction as monitored by TLC, saturated aqueous ammonium chloride solution was added and the product was extracted in ethyl acetate (50 mL×2). The combined organic layer was dried over anhydrous sodium sulphate and concentrated under reduced pressure to give the crude product which was purified by silica gel column chromatography to yield the desired compound (4.5 g, 51%) as off white solid.

¹HNMR (DMSO-d₆, 400 MHz) δ: 7.62 (s, 1H), 7.44-7.40 (m, 3H), 7.38-7.33 (m, 8H), 7.19-7.11 (m, 4H), 7.05-7.03 (m, 1H), 6.87-6.85 (m, 5H), 5.82 (s, 1H), 3.65 (s, 3H); (LCMS): 471 (M+1).

### Step 2: Preparation of 2-Phenyl-3-(3-trityl-3H-imidazol-4-yl)-prop-2-en-1-ol

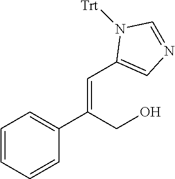

2-Phenyl-3-(3-trityl-3H-imidazol-4-yl)-acrylic acid methyl ester (Step 1, 2.5 gm, 5.31 mmol) was dissolved in dry THF (20.0 mL), cooled to about −78° C. under nitrogen and DIBAL-H (1M solution in Toluene) (5.0 mL, 35.21 mmol) was added. The mixture was allowed to stir for about 2 hours, warmed to room-temperature and EtOAc (20.0 mL) was added, followed by 3N HCl (3 mL) and water (25.0 mL). The mixture was extracted with EtOAc (3×20 mL). Organic phase was separated, washed with brine (3×20 mL), dried over anhydrous Na₂SO₄, and concentrated. The desired compound (2.0 gm, 71%) was isolated as oil.

¹HNMR (DMSO-d₆, 400 MHz) δ: 7.39-7.30 (m, 9H), 7.16-7.03 (m, 7H), 6.87-6.84 (m, 5H), 6.43 (s, 1H), 5.67 (s, 1H), 5.04-5.01 (t, 1H, J=5.9 Hz), 4.11-4.09 (m, 2H); (LCMS): 443 (M+1).

### Step 3: Preparation of 2-Phenyl-3-(3-trityl-3H-imidazol-4-yl)-propenal

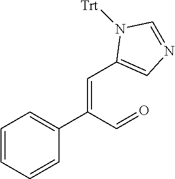

To a stirred solution of 2-phenyl-3-(3-trityl-3H-imidazol-4-yl)-prop-2-en-1-ol (Step 2, 2.0 g, 4.54 mmol) in DCM (20 mL) was added Dess-Martin periodinane (DMP), (2.3 g, 9.64 mmol) portion wise at about 10° C. The reaction mixture was allowed to stir at room temperature for about 2 hours. After completion of reaction, solvent was evaporated under reduced pressure and 10% ethyl acetate in n-hexanes was added to the reaction mass. The precipitate obtained was filtered and washed with 10% ethyl acetate in n-hexanes (3×50 mL). The filtrate was concentrated, water (50 mL) was added to the residue and the mixture was extracted with ethyl acetate (3×50 mL). The combined organic layer was dried over anhydrous sodium sulphate and concentrated under reduced pressure to obtain the crude product which was further purified by flash silica gel column chromatography to yield desired compound (0.8 g, 40.0%) as white solid.

¹HNMR (CDCl₃, 400 MHz) δ: 9.68 (s, 1H), 7.46 (s, 1H), 7.41 (d, 1H, J=1.2 Hz), 7.31-7.28 (m, 8H), 7.18-7.16 (m, 4H), 7.05-7.03 (m, 2H), 6.94-6.92 (m, 6H), 6.21 (s, 1H); (LCMS): 441 (M+1).

### Step 4: Preparation of 5-(3-Methyl-3H-imidazol-4-yl)-1,4-diphenyl-penta-2,4-dien-1-one

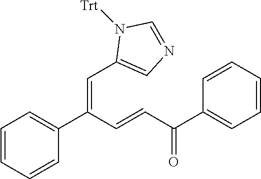

To a stirred solution of 2-phenyl-3-(3-trityl-3H-imidazol-4-yl)-propenal (Step 3, 0.700 g, 1.59 mmol) in toluene (30 mL) was added (Benzoylmethylene)triphenylphosphorane (0.665 g, 1.74 mmol) at room temperature and the reaction mixture was stirred at about 110° C. for about 17 hours. After completion of reaction, solvent was evaporated under reduced pressure and crude product was washed with 5% ethyl acetate/n-hexane to give desired compound (0.160 g, 13%) as yellow solid, which was used in the next step without further purification.

### Step 5: Preparation of 1-Phenyl-2-(6-phenyl-5H-pyrrolo[1,2-c]imidazol-5-yl)-ethanone

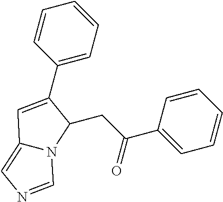 [SMILES: O=C(CC1C(c2ccccc2)=Cc2cncn21)c1ccccc1]

To a mixture of AcOH:MeOH (1:4) (20 mL) was added compound 5-(3-Methyl-3H-imidazol-4-yl)-1,4-diphenyl-penta-2,4-dien-1-one (Step 4, 160 mg, 0.295 mmol) and the reaction mixture was heated at about 90° C. for about 24 hours. After completion of the reaction, solvents were evaporated and reaction mixture was quenched with aqueous sodium bicarbonate (10 mL) and extracted with ethyl acetate (3×15 mL). Combined organic layer was dried over anhydrous sodium sulphate and concentrated to give crude product which was purified by column chromatography to yield compound desired compound (3.0 mg, 5.5%) as pale yellow solid.

¹HNMR (CDCl₃, 400 MHz) δ: 7.91 (d, 2H, J=8.0 Hz), 7.72 (br. s, 1H), 7.58 (t, 1H, J=7.2 Hz), 7.45 (d, 2H, J=8 Hz), 7.42-7.41 (m, 4H), 7.35-7.26 (m, 2H), 6.90 (s, 1H), 5.87 (d, 1H, J=10.4 Hz), 3.62 (dd, 1H, J=1.6, 18.4 Hz), 3.15 (dd, 1H, J=10.4, 18.4 Hz); (LCMS): 301.0 (M+1); Purity: 95.75%.

### Example 170: Preparation of 1-(7-Methyl-6-phenyl-5H-pyrrolo[1,2-c]imidazole-5-yl)-hexan-2-ol

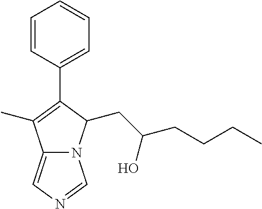 [SMILES: CCCCC(O)CC1C(c2ccccc2)=C(C)c2cncn21]

### Step 1: Preparation of 5-Bromo-4-phenyl-hexa-2,4-dienoic acid ethyl ester

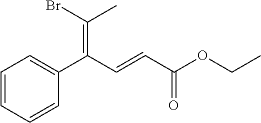 [SMILES: CCOC(=O)/C=C/C(=C(\C)Br)c1ccccc1]

To a solution of compound 3-Bromo-2-phenyl-but-2-enal (Step 2, Example 11; 2 g, 9 mmol) and compound (Carbethoxymethylene)triphenylphosphorane (3.1 g, 9.4 mmol) in acetonitrile (10 mL) was refluxed for about 2 hours and reaction mixture was concentrated in vaccuo. Hexane (20 mL) was added to the residue and triturated. Reaction mixture was filtered and filtrate was concentrated to give desired compound (2.5 g, 95%) as viscous oil.

¹HNMR (CDCl₃, 400 MHz) δ: 8.05 (d, 0.6H, J=15.6 Hz), 7.82 (d, 0.4H, J=15.6 Hz), 7.41-7.34 (m, 3H), 7.09-7.07 (m, 2H), 5.38-5.32 (m, 1H), 4.20-4.13 (m, 2H), 2.73 (s, 1H), 2.26 (s, 2H), 1.28-1.23 (m, 3H); (LCMS): 296 (M+1).

### Step 2: Preparation of 4-Phenyl-5-(4,4,5,5-tetramethyl-[1,3,2]dioxaborolan-2-yl)-hexa-2,4-dienoic acid ethyl ester

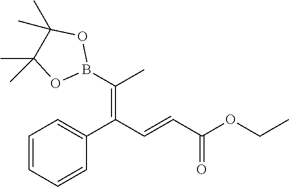 [SMILES: CCOC(=O)/C=C/C(=C(\C)B1OC(C)(C)C(C)(C)O1)c1ccccc1]

Nitrogen gas was purged through a solution of compound 5-Bromo-4-phenyl-hexa-2,4-dienoic acid ethyl ester (step 1, 2.5 g, 8.4 mmol) in 1,4-dioxane (30 mL) for about 10 min at room temperature. Bispinacolatodiboroane (3.22 g, 12.7 mmol) was added to the reaction mixture followed by addition of potassium acetate (2.5 g, 25.3 mmol). Pd(dppf)₂ (343 mg, 0.42 mmol) was added to the reaction mixture and temperature was raised to about 85-90° C. and maintained for about 2 hours. Reaction mixture was cooled to room temperature and filtered through hyflow bed. Filtrate was diluted with ethyl acetate (50 mL) organic layer was washed with DM water (1×10 mL). Layers were separated, organic layer was dried over anhydrous sodium sulphate, filtered and concentrated in vaccuo to give the crude residue which was purified by silica gel column chromatography to give desired compound (2.15 g, 74%)

¹HNMR (CDCl₃, 400 MHz) δ: 8.31 (d, 1H, 15.2 Hz), 7.36 (t, 2H, J=7.6 Hz), 7.28 (d, 1H, J=7.6 Hz), 7.03 (t, 2H, J=7.6 Hz), 5.30 (d, 1H, J=15.2 Hz), 4.14, q, 2H, J=7.2 Hz), 1.67 (s, 3H), 1.37 (s, 12H), 1.26 (t, 3H, J=7.2 Hz).

### Step 3: Preparation of 4-Phenyl-5-(3-trityl-3H-imidazol-4-yl)-hexa-2,4-dienoic acid ethyl ester

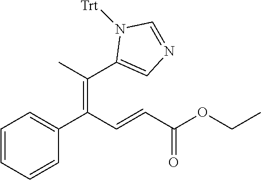

To a mixture of 1,4-dioxane (32 mL) and DM water (8 mL) was added compound 4-Phenyl-5-(4,4,5,5-tetramethyl-[1,3,2]dioxaborolan-2-yl)-hexa-2,4-dienoic acid ethyl ester (step 2, 2.1 g, 6 mmol), compound 7 (2.9 g, 6.7 mmol) and potassium carbonate (2.5 g, 18.4 mmol). Reaction mixture was deoxygenated by purging nitrogen gas for about 15 minutes at room temperature. Pd(dppf)Cl₂. DCM complex (500 mg, 0.6 mmol) was added to the reaction mixture and temperature was raised to about 95-100° C. Heating was continued for about 3 hours. Reaction mixture was cooled to room temperature and filtered through hyflo. Organic layer was separated from the filtrate, dried over anhydrous sodium sulphate, filtered and concentrated to give crude product. Crude product was purified by silica gel column chromatography to give desired compound as viscous oil (1.1 g, 34%).

¹HNMR (CDCl₃, 400 MHz) δ: 8.55 (d, 1H, J=15.6 Hz), 7.51 (d, 1H, J=1.6 Hz), 7.39-7.30 (m, 12H), 7.22-7.19 (m, 6H), 7.13-7.11 (m, 2H), 6.98 (d, 1H, J 1.6 Hz), 5.28 (d, 1H, J=15.6 Hz), 4.11 (q, 2H, J=7.2 Hz), 1.94 (s, 3H), 1.18 (t, 3H, J=7.2 Hz).

### Step 4: Preparation of (7-Methyl-6-phenyl-5H-pyrrolo[1,2-c]imidazole-5-yl)-acetic acid ethyl ester

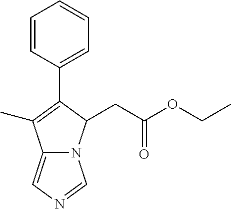 [SMILES: CCOC(=O)CC1C(c2ccccc2)=C(C)c2cncn21]

To a solution of acetic acid (15 mL) in methanol (30 mL) was added compound 4-Phenyl-5-(3-trityl-3H-imidazol-4-yl)-hexa-2,4-dienoic acid ethyl ester (step 3, 1.1 g, 2.1 mmol) at room temperature. Reaction mixture was heated at about 90° C. for about 12 h. Reaction mixture was cooled to room temperature and concentrated under vacuum. The residue was partitioned between ethyl acetate (30 m) and saturated sodium bicarbonate solution (10 mL). The layers were separated, organic layer was dried over anhydrous sodium sulphate, filtered and concentrated to give crude product which was purified using silica gel column chromatography to give desired compound as viscous oil (300 mg, 56%).

¹HNMR (CDCl₃, 400 MHz) δ: 7.73 (s, 1H), 7.42 (d, 2H, J=7.6 Hz), 7.34-7.26 (m, 3H), 6.89 (s, 2H), 5.47 (d, 1H, J=10.4 Hz), 4.21-4.14 (m, 2H), 2.79 (dd, 1H, J=2.8, 17.2 Hz), 2.31 (dd, 1H, J=10.4, 17.2 Hz), 2.17 (d, 3H, 2 Hz), 1.24 (t, 3h, J=3.6 Hz); (LCMS): 283.2 (M+1).

### Step 5: Preparation of (7-Methyl-6-phenyl-5H-pyrrolo[1,2-c]imidazol-5-yl)-acetaldehyde

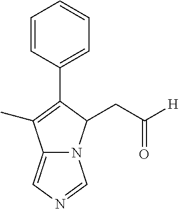 [SMILES: CC1=C(c2ccccc2)C(CC=O)n2cncc21]

A solution of compound (7-Methyl-6-phenyl-5H-pyrrolo[1,2-c]imidazol-5-yl)-acetic acid ethyl ester (step 4, 100 mg, 0.35 mmol) in toluene (20 mL) under nitrogen atmosphere was cooled to about −78° C. DIBAL-H (0.6 mL, 0.6 mmol, 1M solution in toluene) was added to the reaction mixture in a drop wise manner and reaction was continued at the same temperature for about 1 hour. Reaction mixture was quenched by addition of methanol (0.5 mL) followed by addition of 10% citric acid solution (5 mL) at about −78° C. Temperature of the reaction mixture was raised to room temperature and layers were separated. Saturated sodium bicarbonate solution (20 mL) was added to aqueous layer and it was extracted with ethyl acetate (2×10 mL). Combined organic layer was dried over anhydrous sodium sulphate, filtered and concentrated to give desired compound (70 mg, 83%) as pale yellow solid.

¹HNMR (CDCl₃, 400 MHz) δ: 9.81 (s, 1H), 7.68 (s, 1H), 7.45-7.40 (m, 2H), 7.36-7.26 (m, 2H), 7.18-7.13 (m, 1H), 6.91 (s, 1H), 5.54 (d, 1H, J=10 Hz), 2.91 (dd, 1H, J=2.4, 19.2 Hz), 2.61 (dd, J=10, 19.2 Hz), 2.18 (d, 3H, 1.6 Hz); (LCMS): 239.2 (M+1).

### Step 6: Preparation of 1-(7-Methyl-6-phenyl-5H-pyrrolo[1,2-c]imidazol-5-yl)-hexan-2-ol

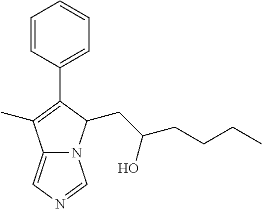 [SMILES: CCCCC(O)CC1C(c2ccccc2)=C(C)c2cncn21]

To a solution of compound (7-Methyl-6-phenyl-5H-pyrrolo[1,2-c]imidazol-5-yl)-acetaldehyde (step 5, 30 mg, 0.1 mmol) in THF (10 mL) at about 0-5° C. was added n-butylmagnesium bromide (1M solution in THF, 0.2 mL, 0.2 mmol) and reaction mixture was allowed to attain room temperature and stirred for about 2 hours. Reaction mixture was cooled to about 10° C. and quenched using saturated ammonium chloride (5 mL). Reaction mixture was extracted with ethyl acetate (2×10 mL). Combined organic extracts were dried over anhydrous sodium sulphate, filtered and concentrated to give crude residue, which was purified using silica gel flash chromatography (1-5% methanol in chloroform) to give desired compound as viscous oil (10 mg, 27%)

¹HNMR (CDCl₃, 400 MHz) δ: 7.79 (s, 1H), 7.43 (t, 2H, J=8 Hz), 7.34-7.30 (m, 3H), 6.9 (s, 1H), 5.41 (d, 1H, J=10.4 Hz), 3.96-3.94 (m, 1H), 2.18 (d, 3H, J=2 Hz), 1.86-1.78 (m, 2H), 1.45-1.42 (m, 2H), 1.29-1.25 (m, 2H), 0.89-0.83 (m, 5H); (LCMS): 297.2 (M+1); Purity: 91.63%.

Following example 171 has been synthesized by the above procedure described in Example 170 with its corresponding intermediate in similar reaction conditions:

| Example | IUPAC | |
|---|---|---|
| No. | Name/Structure | Analytical Data |
| 171. | 1-(7-Methyl-6-phenyl- | ¹H NMR (CDCl₃, 400 MHz) δ: 9.21 (s, 1H), 7.52- |
| | 5H-pyrrolo[1,2- | 7.47 (m, 2H), 7.45-7.41 (m, 1H), 7.34-7.29 (m, |
| | c]imidazol-5-yl)-decan- | 2H), 7.13 (s, 0.6H), 7.11 (s, 0.4 H), 5.72 (d, 0.6H, |
| | 2-ol | J = 12.4 Hz), 5.45 (d, 0.4H, 10.4 Hz), 3.90 (br. s, |
| | 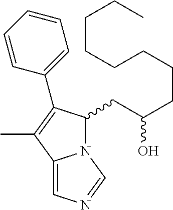 [SMILES: CCCCCCCCC(O)CC1C(c2ccccc2)=C(C)c2cncn21] | 1H), 2.23 (d, 2H, J = 0.8 Hz), 2.20 (d, 1H, J = 1.6 Hz), 1.97-1.94 (m, 1H), 1.53-1.39 (m, 3H), 1.28- 1.24 (m, 12H), 0.90-0.84 (m, 3H): (LCMS): 353.3 (M + 1): Purity = 94.72%. |

### Example 172: Preparation of 2-(7-Methyl-6-phenyl-5H-pyrrolo[1,2-c]imidazol-5-yl)-N-propylacetamide

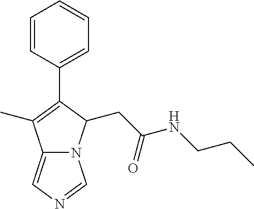 [SMILES: CCCNC(=O)CC1C(c2ccccc2)=C(C)c2cncn21]

### Step 1: Preparation of lithium 2-(7-methyl-6-phenyl-5H-pyrrolo[1,2-c]imidazol-5-yl)acetate

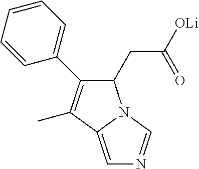

To a stirred solution of (7-Methyl-6-phenyl-5H-pyrrolo[1,2-c]imidazol-5-yl)-acetic acid ethyl ester (Example 22, 50 mg, 0.177 mmol) in THF (10 mL), was added LiOH.H₂O (15.2 mg, 0.353 mmol) solution in DM water (0.5 mL) at room temperature. Reaction mass was stirred at room temperature for about 2 hours. Solvents were evaporated under reduced pressure and the crude residue obtained was used as such for the next step.

### Step 2: Preparation of 2-(7-Methyl-6-phenyl-5H-pyrrolo[1,2-c]imidazol-5-yl)-N-propylacetamide

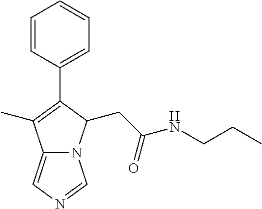 [SMILES: CCCNC(=O)CC1C(c2ccccc2)=C(C)c2cncn21]

To a stirred suspension of lithium 2-(7-methyl-6-phenyl-5H-pyrrolo[1,2-c]imidazol-5-yl)acetate (Crude product form Step 1 used as such considering 100% conversion) in MDC (10 ml) was added n-propyl amine (21 mg, 0.35 mmol), DIPEA (114 mg, 0.88 mmol) and Propylphosphonic anhydride 50% solution in ethyl acetate (0.3 ml, 0.53 mmol) at room temperature and reaction mixture was allowed to stir for about 2 hours. After completion of reaction volatiles were removed under reduced pressure and crude residue was purified using column chromatography to give 2-(7-Methyl-6-phenyl-5H-pyrrolo[1,2-c]imidazol-5-yl)-N-propylacetamide as a pure product (10 mg, 26% over two steps).

¹HNMR (CDCl₃, 400 MHz) δ: 7.72 (s, 1H), 7.42 (t, 2H, J=7.6 Hz), 7.34-7.30 (m, 3H), 6.88 (s, 1H), 5.62 (d, 1H, J=10.4 Hz), 5.53 (s, 1H), 3.26-3.20 (m, 2H), 2.74-2.67 (m, 2H), 2.18 (d, 3H, J=1.6 Hz), 1.52-1.43 (m, 2H), 0.-0.88 (t, 3H, J=6.8 Hz); Mass (LCMS): 296.1 (M+1); Purity: 94.72%.

Following examples have been synthesized by the above procedure described in Example 172 with its corresponding intermediate in similar reaction conditions:

| Example | IUPAC | |
|---|---|---|
| No. | Name/Structure | Analytical Data |
| 175. | 1-(4- | ¹H NMR (CDCl₃, 400 MHz) δ: 7.81 (s, 0.4H), |
| | Hydroxypiperidin-1- | 7.79 (s, 0.6H), 7.46-7.41 (m, 2H), 7.35-7.30 (m, |
| | yl)-2-(7-methyl-6- | 3H), 6.89 (s, 1H), 5.68 (d, 1H, J = 10.4 Hz), 4.19- |
| | phenyl-5H- | 4.09 (m, 1H), 4.05-3.99 (m, 0.5H), 3.95-3.83 (m, |
| | pyrrolo[1,2- | 1H), 3.51-3.34 (m, 2H), 3.24-3.19 (m, 0.5H), |
| | c]imidazol-5-yl)ethan- | 3.08-2.96 (m, 1H), 2.71-2.67 (m, 1H), 2.36-2.27 |
| | 1-one | (m, 1H), 2.20 (d, 3H, J = 1.6 Hz), 2.08-2.02 (m, |
| | 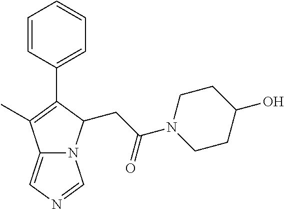 [SMILES: CC1=C(c2ccccc2)C(CC(=O)N2CCC(O)CC2)n2cncc21] | 2H), 1.75-1.70 (m, 2H). Mass (LCMS): 338.1 (M + 1); Purity: 97.3%. |

### Example 174: Preparation of 4-Hydroxy-1-(4-hydroxypiperidin-1-yl)-5-(7-methyl-6-phenyl-5H-pyrrolo[1,2-c]imidazol-5-yl)pentan-1-one

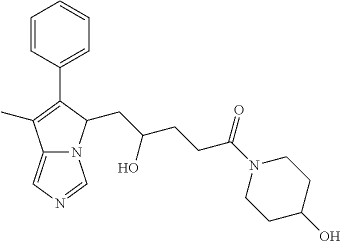 [SMILES: CC1=C(c2ccccc2)C(CC(O)CCC(=O)N2CCC(O)CC2)n2cncc21]

Example-174 was synthesized starting from Example-173. Experimental procedure for acid hydrolysis and amide formation are analogous to that described for Example 172. The ketone to alcohol reduction is performed in a similar manner as described for Example-19.

¹HNMR (CDCl₃, 400 MHz) δ: 7.95-7.83 (m, 1H), 7.43-7.39 (m, 2H), 7.32-7.26 (m, 3H), 6.82 (s, 1H), 5.46-5.20 (m, 1H), 4.12-4.02 (m, 1H), 3.96-3.87 (m, 1H), 3.79-3.64 (m, 2H), 3.28-3.09 (m, 4H), 2.56-2.32 (m, 2H), 2.17-2.14 (m, 3H), 1.92-1.60 (m, 4H), 1.49-1.39 (m, 2H), 1.32-1.24 (m, 2H). Mass (LCMS): 396.2 (M+1); Purity: 90.38%.

### Example 176: Preparation of 2-(2-fluorophenyl)-N-(4-(2-(7-methyl-6-phenyl-5H-pyrrolo[1,2-c]imidazol-5-yl)acetyl)phenyl)acetamide

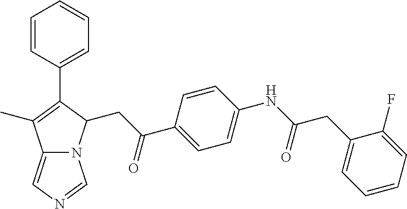 [SMILES: CC1=C(c2ccccc2)C(CC(=O)c2ccc(NC(=O)Cc3ccccc3F)cc2)n2cncc21]

### Step 1: Preparation of 1-(4-nitrophenyl)-2-(triphenyl-15-phosphaneylidene)ethan-1-one

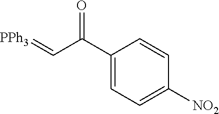

Step 1 compound has been synthesized by the procedure described in Example 17; Step 1 with its corresponding intermediate in similar reaction conditions.

1HNMR (CDCl₃, 400 MHz) δ: 8.19 (d, 2H, J=7.2 Hz), 8.07 (d, 2H, J=7.2 Hz), 7.73-7.68 (m, 6H), 7.61-7.57 (m, 3H), 7.52-7.48 (m, 6H), 4.49 (d, 1H, J=22.8 Hz).

### Step 2: Preparation of (2E,4E)-5-bromo-4-methyl-1-(4-nitrophenyl)-5-phenylpenta-2,4-dien-1-one

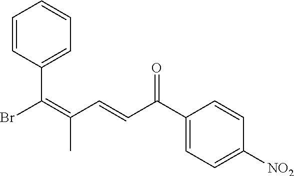 [SMILES: CC(/C=C/C(=O)c1ccc([N+](=O)[O-])cc1)=C(\Br)c1ccccc1]

Step 2 compound has been synthesized by the procedure described in Example 17; Step 2 with its corresponding intermediate in similar reaction conditions.

¹HNMR (CDCl₃, 400 MHz) δ: 8.24 (d, 2H, J=8.4 Hz), 8.20 (d, 1H, J=15.2 Hz), 7.88 (d, 2H, J=8.4 Hz), 7.48-7.39 (m, 4H), 7.21-7.19 (m, 1H), 6.31 (d, 1H, J=15.2 Hz), 2.32 (s, 3H).

### Step 3 & 4: Preparation of 2-(7-methyl-6-phenyl-5H-pyrrolo[1,2-c]imidazol-5-yl)-1-(4-nitrophenyl)ethan-1-one

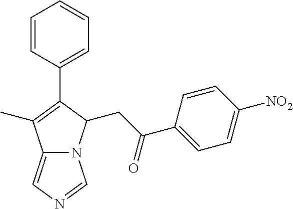 [SMILES: CC1=C(c2ccccc2)C(CC(=O)c2ccc([N+](=O)[O-])cc2)n2cncc21]

Step 4 compound has been synthesized by the procedure described in Example 17; Step 3 & 4 with its corresponding intermediate in similar reaction conditions.

¹HNMR (CDCl₃, 400 MHz) δ: 8.27 (d, 2H, J=8.8 Hz), 8.02 (d, 2H, J=8.8 Hz), 7.68 (s, 1H), 7.47-7.43 (m, 3H), 7.36-7.31 (m, 2H), 6.90 (s, 1H), 5.76-5.73 (m, 1H), 3.39 (dd, 1H, J=1.6 Hz and 18.8 Hz), 3.12 (dd, 1H, J=10.8 Hz and 18.8 Hz), 3.23 (s, 3H); LCMS: 360.1 (M+1).

### Step 5: Preparation of 1-(4-aminophenyl)-2-(7-methyl-6-phenyl-5H-pyrrolo[1,2-c]imidazol-5-yl)ethan-1-one

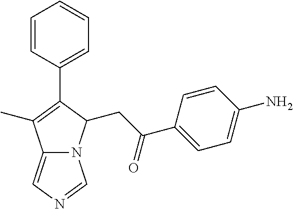 [SMILES: CC1=C(c2ccccc2)C(CC(=O)c2ccc(N)cc2)n2cncc21]

To a solution of compound 2-(7-methyl-6-phenyl-5H-pyrrolo[1,2-c]imidazol-5-yl)-1-(4-nitrophenyl)ethan-1-one (Step 4, 210 mg, 0.584 mmol) in MeOH (20 ml), was added 10% Pd/C (50% wet, 50 mg) and reaction mixture was stirred under H₂ pressure for about 2-3 hours. Reaction was monitored by TLC/LCMS. After completion, reaction was filtered through celite bed. Filtrate was evaporated to get compound 1-(4-aminophenyl)-2-(7-methyl-6-phenyl-5H-pyrrolo[1,2-c]imidazol-5-yl)ethan-1-one (160 mg crude) as semi-solid. Product formation was confirmed by LCMS and used in next step without purification.

LCMS: 330.1 (M+1).

### Step 6: Preparation of 2-(2-fluorophenyl)-N-(4-(2-(7-methyl-6-phenyl-5H-pyrrolo[1,2-c]imidazol-5-yl)acetyl)phenyl)acetamide

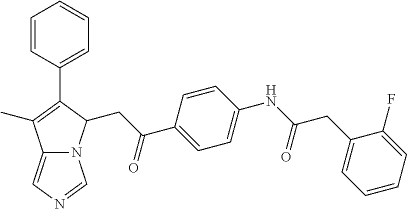 [SMILES: CC1=C(c2ccccc2)C(CC(=O)c2ccc(NC(=O)Cc3ccccc3F)cc2)n2cncc21]

In a DCM solution of compound 1-(4-aminophenyl)-2-(7-methyl-6-phenyl-5H-pyrrolo[1,2-c]imidazol-5-yl)ethan-1-one (Step 5, 40 mg, 0.1215 mmol) under nitrogen atmosphere added 2-Fluoro phenyl acetic acid (20 mg, 0.1337 mmol, 1.1 eq) at about 0° C., followed by DIPEA (47 mg, 0.3645 mmol, 3 eq) and propylphosphonic anhydride (50% solution in Ethyl acetate) (0.1 mL, 0.1822 mmol, 1.5 eq) drop wise. Stirred the reaction solution at room temperature for about 1 hour. Diluted the reaction mixture with DCM (15 mL) and washed the organic layer with water (20 mL×3 times), brine and organic layer was dried over anhydrous sodium sulphate, filtered and concentrated to get crude compound 2-(2-fluorophenyl)-N-(4-(2-(7-methyl-6-phenyl-5H-pyrrolo[1,2-c]imidazol-5-yl)acetyl)phenyl)acetamide. Crude compound was then purified by Preparative HPLC to affords pure 2-(2-fluorophenyl)-N-(4-(2-(7-methyl-6-phenyl-5H-pyrrolo[1,2-c]imidazol-5-yl)acetyl)phenyl)acetamide (4 mg, 7%) as a yellow solid.

¹HNMR (CDCl₃, 400 MHz) δ: 8.28 (br. s, 1H), 7.89 (br. s, 1H), 7.73-7.72 (m, 2H), 7.59-7.57 (m, 2H), 7.44 (t, 2H, J=7.6 Hz), 7.36-7.30 (m, 5H), 7.15-7.06 (m, 3H), 5.75-5.73 (m, 1H), 3.78 (s, 2H), 3.30 (dd, 1H, J=0.8, 18.0 Hz), 3.04-2.99 (m, 1H), 2.20 (s, 3H); LCMS: 466.1 (M+1); Purity: 93.74%.

### Example 177: Preparation of 4-[2-(7-Methyl-6-phenyl-5H-pyrrolo[1,2-c]imidazol-5-yl)-acetyl]-cyclohexanone oxime

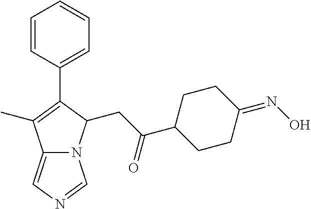 [SMILES: CC1=C(c2ccccc2)C(CC(=O)C2CCC(=NO)CC2)n2cncc21]

### Step 1: Preparation of Ethyl 4-(hydroxyimino)cyclohexane-1-carboxylate

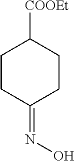 [SMILES: CCOC(=O)C1CCC(=NO)CC1]

To a solution of compound Ethyl 4-Oxocyclohexanecarboxylate (10 g, 58.82 mmol) in methanol (50 mL) was added hydroxylamine hydrochloride solution (12 mL) at room temperature and the reaction mixture was allowed to stir at about 80° C. for about 5 hours. After completion, volatiles were removed under reduced pressure to give the crude product. Water 100 mL was added to the crude product and was extracted with ethyl acetate (3×100 mL). Combined organic layer was evaporated under reduced pressure to give compound Ethyl 4-(hydroxyimino)cyclohexane-1-carboxylate (10.3 g, 95%) as pale yellow liquid. The compound was used in next step without further purification.

### Step 2: Preparation of Ethyl 4-(((tert-butyldimethylsilyl)oxy)imino)cyclohexane-1-carboxylate

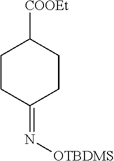 [SMILES: CCOC(=O)C1CCC(=NO[Si](C)(C)C(C)(C)C)CC1]

To a solution of compound Ethyl 4-(hydroxyimino)cyclohexane-1-carboxylate (Step 1, 10 g, 54.05 mmol) in dichloromethane (100 mL) was added triethylamine (15 mL 108.10 mmol) and TBDMSCl (12.16 g, 81.08 mmol) at room temperature successively. The resulting mixture was stirred at room temperature for about 16 hours. After completion, reaction mass was quenched by the addition of water (150 mL) and the mixture was extracted with ethyl acetate (2×150 mL). Combined organic layer was evaporated under reduced pressure and the residue was purified by flash chromatography eluting with 5% ethyl acetate and n-hexanes to give pure compound Ethyl 4-(((tert-butyldimethylsilyl)oxy)imino)cyclohexane-1-carboxylate (11.2 g, 69%) as clear thick liquid.

¹HNMR (CDCl₃, 400 MHz) δ: 4.14 (q, 2H, J=7.2 Hz), 3.25-3.18 (m, 1H), 2.56-2.44 (m, 2H), 2.17-1.94 (m, 4H), 1.77-1.60 (m, 2H), 1.25 (t, 3H, J=7.2 Hz), 0.92 (br. s, 9H), 0.15 (br. s, 3H), 0.14 (br. s, 3H); Mass (LCMS): 300.1 (M+1).

### Step 3: Preparation of Dimethyl (2-(4-(((tert-butyldimethylsilyl)oxy)imino)cyclohexyl)-2-oxoethyl)phosphonate

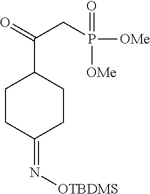 [SMILES: COP(=O)(CC(=O)C1CCC(=NO[Si](C)(C)C(C)(C)C)CC1)OC]

At about −78° C., to a solution of dimethyl methylphophonate (0.82 g, 6.68 mmol) in dry THF (10 ml) was added n-BuLi solution (2.7 mL, 6.68 mmol) 2.5 M in THF drop wise. The reaction mass was allowed to stir at about −78° C. for about 30 minutes and then a solution of compound Ethyl 4-(((tert-butyldimethylsilyl)oxy)imino)cyclohexane-1-carboxylate (Step-2, 1 g, 3.4 mmol) in THF (5 mL) was added to reaction mixture at same temperature. The resulting mixture was kept stirring at about −78° C. for about 30 minutes and then slowly warmed up to about 0° C. in about 1 hour. After completion, reaction mass was quenched by the addition of water (25 mL) and the mixture was extracted with ethyl acetate (2×50 mL). Combined organic layer was evaporated under reduced pressure to give compound dimethyl (2-(4-(((tert-butyldimethylsilyl)oxy)imino)cyclohexyl)-2-oxoethyl)phosphonate (1.0 g, 79%) as clear thick liquid. The compound dimethyl (2-(4-(((tert-butyldimethylsilyl)oxy)imino)cyclohexyl)-2-oxoethyl)phosphonate was used in next step without further purification. Product formation was confirmed by LCMS: 378.1 (M+1).

### Step 4: Preparation of 5-Bromo-1-(4-(((tert-butyldimethylsilyl)oxy)imino)cyclohexyl)-4-phenylhexa-2,4-dien-1-one

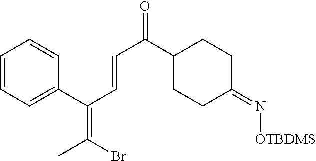 [SMILES: C/C(Br)=C(/C=C/C(=O)C1CCC(=NO[Si](C)(C)C(C)(C)C)CC1)c1ccccc1]

At about 0° C., to a suspension of sodium hydride (60%, 0.177 g, 4.44 mmol) in dry THF (5 mL) was added a solution of Dimethyl (2-(4-(((tert-butyldimethylsilyl)oxy)imino)cyclohexyl)-2-oxoethyl)phosphonate (Step 3, 2.01 g, 5.33 mmol) in THF (5 mL) slowly. The reaction mass was allowed to stir at about 0° C. for about 30 minutes and then a solution of compound Benzeneacetaldehyde, α-(1-bromoethylidene) (1 g, 4.44 mmol) in THF (5 mL) was added to reaction mixture at same temperature. The resulting mixture was stirred at room temperature for about 2 hours. After completion, reaction mass was quenched by the addition of water (50 mL) and the mixture was extracted with ethyl acetate (3×50 mL). Combined organic layer was evaporated under reduced pressure and the residue was purified by flash chromatography eluting with 2% ethyl acetate and n-hexanes to give pure compound 5-Bromo-1-(4-(((tert-butyldimethylsilyl)oxy)imino)cyclohexyl)-4-phenylhexa-2,4-dien-1-one (1.5 g, 71%) as pale yellow thick liquid.

¹HNMR (CDCl₃, 400 MHz) δ: 8.00 (d, 1H, J=15.6 Hz), 7.44-7.35 (m, 3H), 7.09-7.07 (m, 2H), 5.67 (d, 1H, J=15.6 Hz), 3.35-3.29 (m, 1H), 2.27 (s, 3H), 2.17-2.07 (m, 2H), 1.97-1.82 (m, 2H), 1.62-1.36 (m, 4H), 0.90 (br. s, 9H), 0.14 (br. s, 3H), 0.13 (br. s, 3H); Mass (LCMS): 476.0 and 478.0 (M⁺, M+2).

### Step 5: Preparation of compound 1-(4-(((tert-butyldimethylsilyl)oxy)imino)cyclohexyl)-4-phenyl-5-(1-trityl-1H-imidazol-5-yl)hexa-2,4-dien-1-one

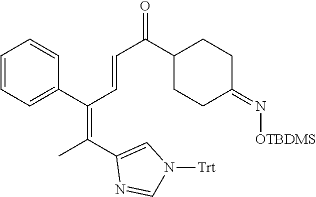

To a mixture of 5-Bromo-1-(4-(((tert-butyldimethylsilyl)oxy)imino)cyclohexyl)-4-phenylhexa-2,4-dien-1-one (Step 4, 1.5 g, 3.15 mmol) and N-trityl imidazole-4-boronic acid (1.67 g, 4.72 mmol) in 1,4-dioxane (24 mL) and water (6 mL) was added K₂CO₃ (1.08 mg, 7.87 mmol) at room temperature. The reaction mixture was degassed for about 10 minutes using nitrogen gas. To this suspension Pd(dppf)Cl₂-DCM complex (0.205 g, 0.252 mmol) was added and reaction mixture was again degassed for about 10 minutes. Reaction mixture was heated at about 95° C. for about 2 hours. After completion of reaction, solvent was evaporated under reduced pressure and residue was diluted with water (50 mL) and extracted with ethyl acetate (3×50 mL). Combined organic layer was dried over anhydrous sodium sulphate and concentrated to give crude product which was purified by column chromatography to give compound 1-(4-(((tert-butyldimethylsilyl)oxy)imino)cyclohexyl)-4-phenyl-5-(1-trityl-1H-imidazol-5-yl)hexa-2,4-dien-1-one (700 mg, 32%) as pale brown fluffy solid. Product formation was confirmed by LCMS; 706.3 (M+1).

### Step 6: Preparation of 4-[2-(7-Methyl-6-phenyl-5H-pyrrolo[1,2-c]imidazol-5-yl)-acetyl]-cyclohexanone oxime

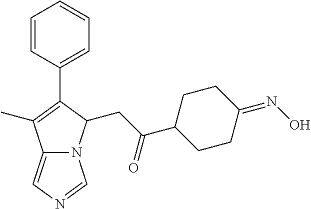 [SMILES: CC1=C(c2ccccc2)C(CC(=O)C2CCC(=NO)CC2)n2cncc21]

To a mixture of methanol (30 mL) and acetic acid (10 mL) was added 1-(4-(((tert-butyldimethylsilyl)oxy)imino)cyclohexyl)-4-phenyl-5-(1-trityl-1H-imidazol-5-yl)hexa-2,4-dien-1-one (Step 5, 700 mg, 0.992 mmol) and the reaction mixture was heated at about 90° C. for about 6 hours. After completion of the reaction, solvents were evaporated and reaction mixture was quenched with aqueous sodium bicarbonate (30 mL) and extracted with ethyl acetate (3×40 mL). Combined organic layer was dried over anhydrous sodium sulphate and concentrated to give crude product which was purified by column chromatography to give pure compound 4-[2-(7-Methyl-6-phenyl-5H-pyrrolo[1,2-c]imidazol-5-yl)-acetyl]-cyclohexanone oxime (277 mg, 80%) as pale yellow solid (over two steps).

¹HNMR (CDCl₃, 400 MHz) δ: 7.56 (br. s, 1H), 7.45-7.41 (m, 2H) 7.35-7.32 (m, 1H), 7.29-7.27 (m, 2H), 6.88 (br. s, 1H), 5.57-5.54 (m, 1H), 3.27-3.22 (m, 1H) 2.87 (dd, 1H, J=2.0, 18.4 Hz), 2.57 (dd, 1H, J=10.4, 18.4 Hz), 2.51-2.39 (m, 2H), 2.18 (d, 3H, J=2.0 Hz), 2.012.02 (m, 1H), 1.92-1.78 (m, 3H), 1.63-1.51 (m, 2H); Mass (LCMS): 350.1 (M+1); Purity: 96.79%.

### Example 178: Preparation of 4-[1-Hydroxy-2-(7-methyl-6-phenyl-5H-pyrrolo[1,2-c]imidazol-5-yl)-ethyl]-cyclohexanone oxime

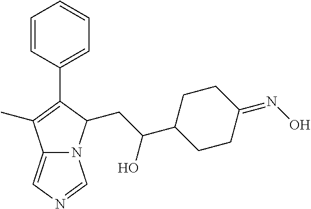 [SMILES: CC1=C(c2ccccc2)C(CC(O)C2CCC(=NO)CC2)n2cncc21]

To a stirred solution of compound 4-[2-(7-Methyl-6-phenyl-5H-pyrrolo[1,2-c]imidazol-5-yl)-acetyl]-cyclohexanone oxime (Example 177; 10 mg, 0.03 mmol) in methanol (2 mL) was added sodium borohydride (1.3 mg, 0.03 mmol) and the reaction mixture was stirred at room temperature for about 0.5 hour After completion, excess of water (20 mL) was added to reaction mass and extracted with ethyl acetate (3×15 mL). Combined organic layer was evaporated under reduced pressure and crude product was purified by trituration by n-hexanes give compound 4-[1-Hydroxy-2-(7-methyl-6-phenyl-5H-pyrrolo[1,2-c]imidazol-5-yl)-ethyl]-cyclohexanone oxime (8 mg, 80%) as off white solid.

¹HNMR (CDCl₃, 400 MHz) δ: 7.86 (br. s, 1H), 7.44-7.41 (m, 2H), 7.34-7.27 (m, 3H), 6.88 (br. s, 1H), 5.27-5.22 (m, 1H), 3.65-3.64 (m, 1H), 3.42-3.36 (m, 1H), 3.30-3.22 (m, 1H), 2.39-2.30 (m, 1H), 2.16 (br. s, 3H), 2.07-1.96 (m, 3H), 1.84-1.76 (m, 2H), 1.70-1.62 (m, 2H), 1.21-1.11 (m, 1H); Mass (LCMS): 352.1 (M+1); Purity: 96.59%.

### Method for Preparative Separation of Isomers:

Above exemplified isomer compounds were separated by using both reverse phase and normal phase chiral preparative HPLC methods. The column specification are as follows: Reverse phase preparative column: YMC C-18 (300×25 mm), 10 m, Normal Phase chiral preparative column: YMC-SA (250×20 mm), 10 m.

The following compounds can also be synthesized as described in the above experimental procedures:

N-(2-(4-(1-hydroxy-2-(7-methyl-6-phenyl-
5H-pyrrolo[1,2-c]imidazol-5-
yl)ethyl)piperidin-1-
yl)ethyl)methanesulfonamide

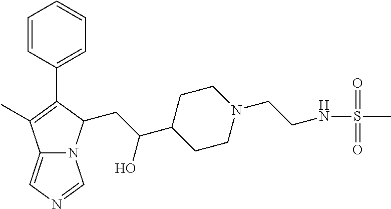

methyl 4-(4-(1-hydroxy-2-(7-methyl-6-
phenyl-5H-pyrrolo[1,2-c]imidazol-5-
yl)ethyl)piperidin-1-yl)benzoate

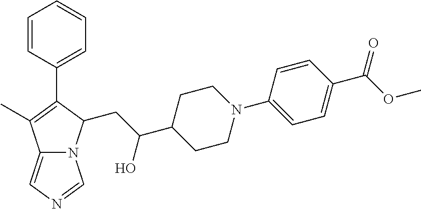

2-(7-methyl-6-phenyl-5H-pyrrolo[1,2-
c]imidazol-5-yl)-1-(1-phenylpiperidin-4-
yl)ethan-1-ol

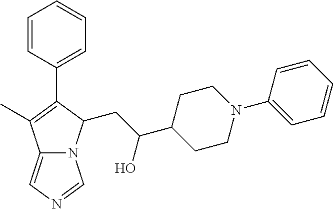 [SMILES: CC1=C(c2ccccc2)C(CC(O)C2CCN(c3ccccc3)CC2)n2cncc21]

methyl 3-(4-(1-hydroxy-2-(7-methyl-6-
phenyl-5H-pyrrolo[1,2-c]imidazol-5-
yl)ethyl)piperidin-1-yl)benzoate

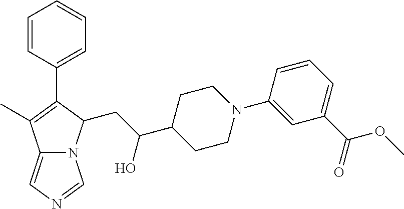

1-(1-isobutylpiperidin-4-yl)-2-(7-methyl-6-
phenyl-5H-pyrrolo[1,2-c]imidazol-5-
yl)ethan-1-ol

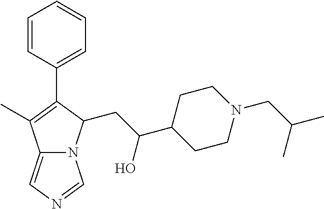 [SMILES: CC1=C(c2ccccc2)C(CC(O)C2CCN(CC(C)C)CC2)n2cncc21]

4-(1-amino-2-(7-methyl-6-phenyl-5H-
pyrrolo[1,2-c]imidazol-5-
yl)ethyl)cyclohexan-1-ol

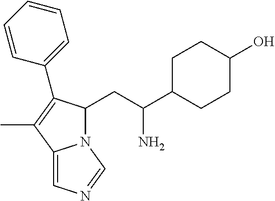 [SMILES: CC1=C(c2ccccc2)C(CC(N)C2CCC(O)CC2)n2cncc21]

(4-(1-hydroxy-2-(7-methyl-6-phenyl-5H-
pyrrolo[1,2-c]imidazol-5-
yl)ethyl)piperidin-1-yl)(4-
hydroxyphenyl)methanone

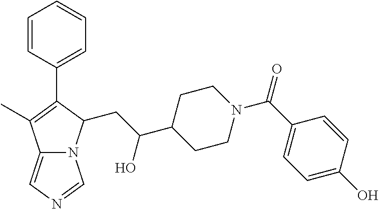 [SMILES: CC1=C(c2ccccc2)C(CC(O)C2CCN(C(=O)c3ccc(O)cc3)CC2)n2cncc21]

4-(1-hydroxy-2-(7-methyl-6-phenyl-5H-
pyrrolo[1,2-c]imidazol-5-
yl)ethyl)cyclohexane-1-sulfonamide

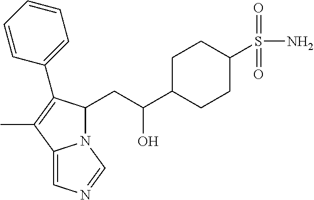 [SMILES: CC1=C(c2ccccc2)C(CC(O)C2CCC(S(N)(=O)=O)CC2)n2cncc21]

(2-fluorophenyl)(4-(1-hydroxy-2-(7-
methyl-6-phenyl-5H-pyrrolo[1,2-
c]imidazol-5-yl)ethyl)piperidin-1-
yl)methanone

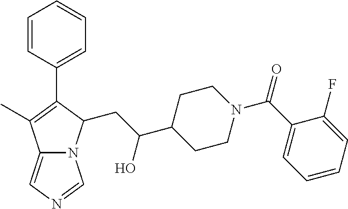 [SMILES: CC1=C(c2ccccc2)C(CC(O)C2CCN(C(=O)c3ccccc3F)CC2)n2cncc21]

4-(1-hydroxy-2-(7-methyl-6-phenyl-5H-
pyrrolo[1,2-c]imidazol-5-yl)ethyl)-1-
(hydroxymethyl)cyclohexan-1-ol

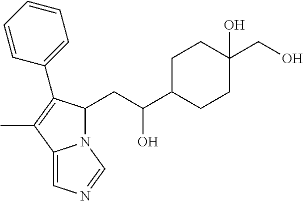 [SMILES: CC1=C(c2ccccc2)C(CC(O)C2CCC(O)(CO)CC2)n2cncc21]

(4-(1-hydroxy-2-(7-methyl-6-phenyl-5H-
pyrrolo[1,2-c]imidazol-5-
yl)ethyl)piperidin-1-yl)(piperidin-4-
yl)methanone

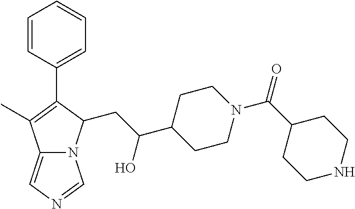 [SMILES: CC1=C(c2ccccc2)C(CC(O)C2CCN(C(=O)C3CCNCC3)CC2)n2cncc21]

4-(1-hydroxy-2-(7-methyl-6-phenyl-5H-
pyrrolo[1,2-c]imidazol-5-
yl)ethyl)cyclohexyl acetate

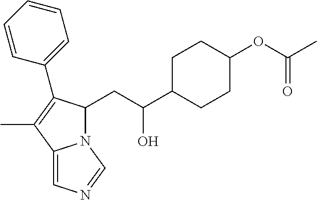 [SMILES: CC(=O)OC1CCC(C(O)CC2C(c3ccccc3)=C(C)c3cncn32)CC1]

azetidin-3-yl(4-(1-hydroxy-2-(7-methyl-6-
phenyl-5H-pyrrolo[1,2-c]imidazol-5-
yl)ethyl)piperidin-1-yl)methanone

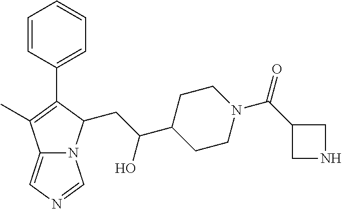 [SMILES: CC1=C(c2ccccc2)C(CC(O)C2CCN(C(=O)C3CNC3)CC2)n2cncc21]

4-(1-hydroxy-2-(7-methyl-6-phenyl-5H-
pyrrolo[1,2-c]imidazol-5-
yl)ethyl)cyclohexyl benzoate

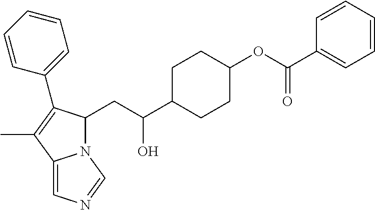 [SMILES: CC1=C(c2ccccc2)C(CC(O)C2CCC(OC(=O)c3ccccc3)CC2)n2cncc21]

(4-(1-hydroxy-2-(7-methyl-6-phenyl-5H-
pyrrolo[1,2-c]imidazol-5-
yl)ethyl)piperidin-1-yl)(2-
hydroxyphenyl)methanone

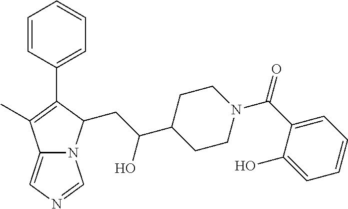 [SMILES: CC1=C(c2ccccc2)C(CC(O)C2CCN(C(=O)c3ccccc3O)CC2)n2cncc21]

4-(1-hydroxy-2-(7-methyl-6-phenyl-5H-
pyrrolo[1,2-c]imidazol-5-
yl)ethyl)cyclohexyl dihydrogen phosphate

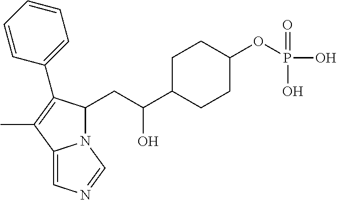 [SMILES: CC1=C(c2ccccc2)C(CC(O)C2CCC(OP(=O)(O)O)CC2)n2cncc21]

2-(7-methyl-6-phenyl-5H-pyrrolo[1,2-
c]imidazol-5-yl)-1-(1-(oxetan-3-
yl)piperidin-4-yl)ethan-1-ol

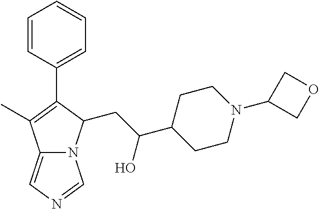 [SMILES: CC1=C(c2ccccc2)C(CC(O)C2CCN(C3COC3)CC2)n2cncc21]

2-(7-methyl-6-phenyl-5H-pyrrolo[1,2-
c]imidazol-5-yl)-1-(1-oxaspiro[3.5]nonan-
7-yl)ethan-1-ol

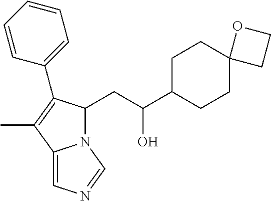 [SMILES: CC1=C(c2ccccc2)C(CC(O)C2CCC3(CCO3)CC2)n2cncc21]

1-(1-(azetidin-3-yl)piperidin-4-yl)-2-(7-
methyl-6-phenyl-5H-pyrrolo[1,2-
c]imidazol-5-yl)ethan-1-ol

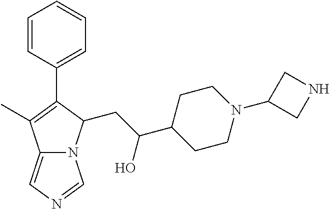 [SMILES: CC1=C(c2ccccc2)C(CC(O)C2CCN(C3CNC3)CC2)n2cncc21]

2-(7-methyl-6-phenyl-5H-pyrrolo[1,2-
c]imidazol-5-yl)-1-(1-oxaspiro[4.5]decan-
8-yl)ethan-1-ol

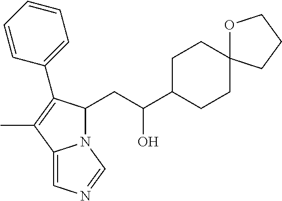 [SMILES: CC1=C(c2ccccc2)C(CC(O)C2CCC3(CCCO3)CC2)n2cncc21]

2-(7-methyl-6-phenyl-5H-pyrrolo[1,2-
c]imidazol-5-yl)-1-(1-(pyrimidin-5-
yl)piperidin-4-yl)ethan-1-ol

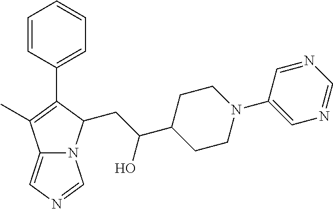 [SMILES: CC1=C(c2ccccc2)C(CC(O)C2CCN(c3cncnc3)CC2)n2cncc21]

N-(4-(1-hydroxy-2-(7-methyl-6-phenyl-5H-
pyrrolo[1,2-c]imidazol-5-
yl)ethyl)cyclohexyl)benzamide

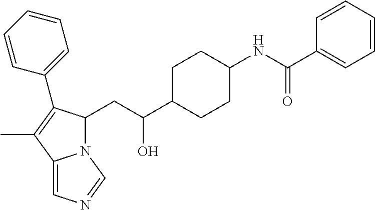 [SMILES: CC1=C(c2ccccc2)C(CC(O)C2CCC(NC(=O)c3ccccc3)CC2)n2cncc21]

2-(7-methyl-6-phenyl-5H-pyrrolo[1,2-
c]imidazol-5-yl)-1-(1-(pyridin-4-
yl)piperidin-4-yl)ethan-1-ol

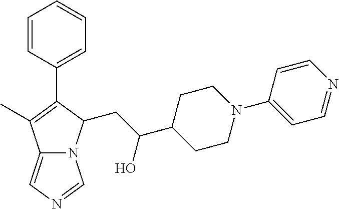 [SMILES: CC1=C(c2ccccc2)C(CC(O)C2CCN(c3ccncc3)CC2)n2cncc21]

N-(4-(1-hydroxy-2-(7-methyl-6-phenyl-5H-
pyrrolo[1,2-c]imidazol-5-
yl)ethyl)cyclohexyl)benzamide

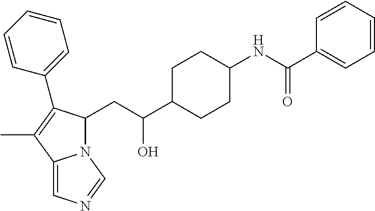 [SMILES: CC1=C(c2ccccc2)C(CC(O)C2CCC(NC(=O)c3ccccc3)CC2)n2cncc21]

2-(7-methyl-6-phenyl-5H-pyrrolo[1,2-
c]imidazol-5-yl)-1-(1-(oxazol-4-
yl)piperidin-4-yl)ethan-1-ol

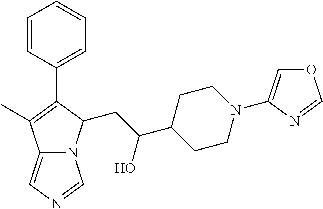 [SMILES: CC1=C(c2ccccc2)C(CC(O)C2CCN(c3cocn3)CC2)n2cncc21]

benzyl 4-(1-hydroxy-2-(7-methyl-6-phenyl-
5H-pyrrolo[1,2-c]imidazol-5-
yl)ethyl)cyclohexane-1-carboxylate

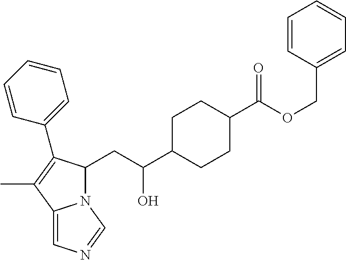 [SMILES: CC1=C(c2ccccc2)C(CC(O)C2CCC(C(=O)OCc3ccccc3)CC2)n2cncc21]

1-(1-(1H-imidazol-4-yl)piperidin-4-yl)-2-
(7-methyl-6-phenyl-5H-pyrrolo[1,2-
c]imidazol-5-yl)ethan-1-ol

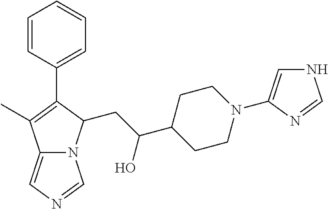 [SMILES: CC1=C(c2ccccc2)C(CC(O)C2CCN(c3c[nH]cn3)CC2)n2cncc21]

pyridin-4-ylmethyl 4-(1-hydroxy-2-(7-
methyl-6-phenyl-5H-pyrrolo[1,2-
c]imidazol-5-yl)ethyl)cyclohexane-1-
carboxylate

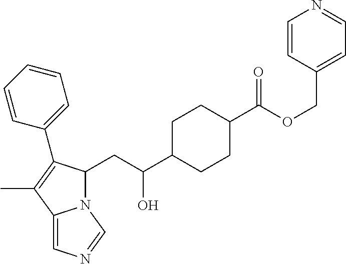 [SMILES: CC1=C(c2ccccc2)C(CC(O)C2CCC(C(=O)OCc3ccncc3)CC2)n2cncc21]

1-(1-(1H-pyrazol-3-yl)piperidin-4-yl)-2-(7-
methyl-6-phenyl-5H-pyrrolo[1,2-
c]imidazol-5-yl)ethan-1-ol

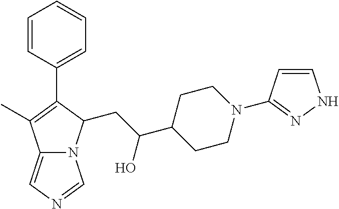 [SMILES: CC1=C(c2ccccc2)C(CC(O)C2CCN(c3cc[nH]n3)CC2)n2cncc21]

1-(4-(1-hydroxy-2-(7-methyl-6-phenyl-5H-
pyrrolo[1,2-c]imidazol-5-
yl)ethyl)cyclohexyl)cyclopropan-1-ol

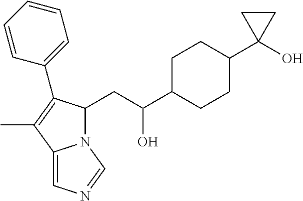 [SMILES: CC1=C(c2ccccc2)C(CC(O)C2CCC(C3(O)CC3)CC2)n2cncc21]

1-(1-(2-aminophenyl)piperidin-4-yl)-2-(7-
methyl-6-phenyl-5H-pyrrolo[1,2-
c]imidazol-5-yl)ethan-1-ol

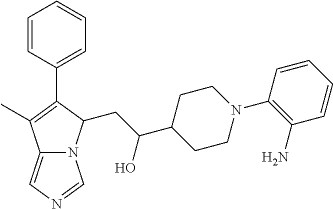 [SMILES: CC1=C(c2ccccc2)C(CC(O)C2CCN(c3ccccc3N)CC2)n2cncc21]

3-(1-hydroxy-2-(7-methyl-6-phenyl-5H-
pyrrolo[1,2-c]imidazol-5-
yl)ethyl)cyclobutan-1-ol

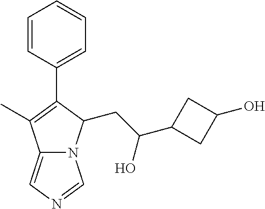 [SMILES: CC1=C(c2ccccc2)C(CC(O)C2CC(O)C2)n2cncc21]

1-(3-methoxycyclobutyl)-2-(7-methyl-6-
phenyl-5H-pyrrolo[1,2-c]imidazol-5-
yl)ethan-1-ol

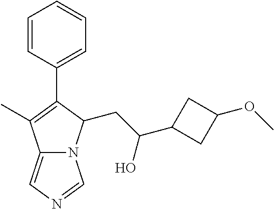

3-(1-hydroxy-2-(7-methyl-6-phenyl-5H-
pyrrolo[1,2-c]imidazol-5-
yl)ethyl)cyclobutane-1-carboxylic acid

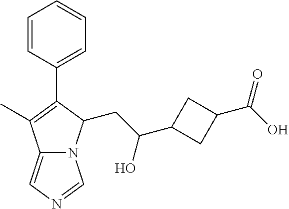 [SMILES: CC1=C(c2ccccc2)C(CC(O)C2CC(C(=O)O)C2)n2cncc21]

methyl 3-(1-hydroxy-2-(7-methyl-6-
phenyl-5H-pyrrolo[1,2-c]imidazol-5-
yl)ethyl)cyclobutane-1-carboxylate

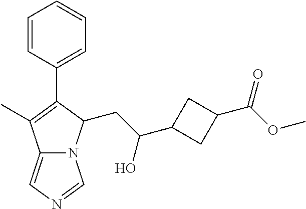

3-(1-hydroxy-2-(7-methyl-6-phenyl-5H-
pyrrolo[1,2-c]imidazol-5-
yl)ethyl)cyclobutane-1-carboxamide

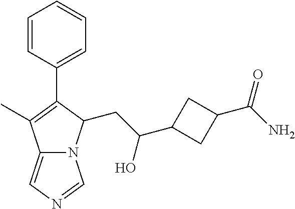 [SMILES: CC1=C(c2ccccc2)C(CC(O)C2CC(C(N)=O)C2)n2cncc21]

3-(1-hydroxy-2-(7-methyl-6-phenyl-5H-
pyrrolo[1,2-c]imidazol-5-yl)ethyl)-N-
methylcyclobutane-1-carboxamide

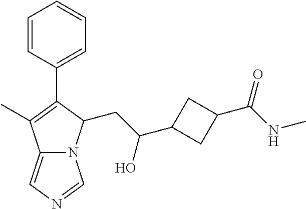

### Biological Assays

In-vitro human indoleamine 2,3-dioxygenase 1 (IDO1) enzyme assay: In the in-vitro human indoleamine 2,3-dioxygenase 1 (hIDO1) enzyme assay for screening inhibitor compounds, hIDO1 with an N-terminal histidine tag expressed and purified from E. coli (BPS Bioscience, San Diego, Calif., USA) was used. All other materials were procured from Sigma-Aldrich, St. Louis, Mo., USA.

The assay method for monitoring the conversion of L-tryptophan to N-formylkynurenine by hlDO1 was carried out as follows. hlDO1 (50 ng) was incubated with tryptophan (80 μM) in the presence of ascorbic acid (10 mM), methylene blue (10 μM), catalase (100 μg/ml) and 0.01% Tween-20 in sodium phosphate buffer (50 mM; pH 6.5) at 37° C. for 60 min. The reaction was terminated with 200 mM piperidine (PIP) and further incubated at about 65° C. for about 20 minutes to convert N-formylkynurenine (NFK) to NFK-PIP. The reaction mixture was then incubated at room temperature for about 1 hour. The fluorescence intensity was read in a fluorescence microplate reader at an excitation wavelength of 400 nm and emission wavelength of 500 nm. Percent inhibition at each concentration of test compounds was determined by estimating the decrease in NFK-PIP. Data were analyzed using nonlinear regression to generate IC₅₀ values using Graph Pad Prism® 6.

The % inhibition values for hIDO1 enzyme at 10.0 μM concentration of the compounds of present invention are as follows (A: ≥50%, B: ≤50%):

| Example | % inhibition |
|---|---|
| No | hIDO1 |
| 1 | B |
| 2 | B |
| 3 | B |
| 4 | B |
| 5 | B |
| 6 | B |
| 7 | B |
| 8 | B |
| 9 | B |
| 10 | B |
| 11 | A |
| 12 | A |
| 13 | B |
| 14 | B |
| 15 | A |
| 16 | A |
| 17 | A |
| 18 | A |
| 19 | A |
| 20 | A |
| 21 | B |
| 22 | A |
| 23 | A |
| 24 | A |
| 25 | A |
| 26 | A |
| 27 | A |
| 28 | A |
| 29 | A |
| 30 | A |
| 31 | A |
| 32 | B |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | B |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | B |
| 87 | A |
| 88 | A |
| 89 | A |
| 90 | A |
| 91 | A |
| 92 | B |
| 93 | B |
| 94 | A |
| 95 | A |
| 96 | A |
| 97 | B |
| 98 | A |
| 99 | A |
| 100 | A |
| 101 | A |
| 102 | B |
| 103 | B |
| 104 | B |
| 105 | A |
| 106 | B |
| 107 | A |
| 108 | A |
| 109 | A |
| 110 | A |
| 111 | A |
| 112 | A |
| 113 | A |
| 114 | A |
| 115 | B |
| 116 | B |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | B |
| 131 | A |
| 132 | A |
| 133 | A |
| 134 | A |
| 135 | B |
| 136 | A |
| 137 | A |
| 138 | A |
| 139 | A |
| 140 | B |
| 141 | B |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | B |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | B |
| 173 | A |
| 174 | A |
| 175 | B |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |

The % inhibition values for hIDO1 enzyme at 1.0 μM concentration of the compounds of resent invention are as follows (A: ≥50%, B: ≤50):

| Example | % inhibition |
|---|---|
| No | hIDO1 |
| 15 | B |
| 16 | B |
| 17 | A |
| 18 | A |
| 19 | A |
| 20 | A |
| 21 | B |
| 22 | B |
| 23 | B |
| 24 | B |
| 25 | A |
| 26 | B |
| 27 | B |
| 28 | A |
| 29 | A |
| 30 | A |
| 31 | B |
| 32 | B |
| 33 | B |
| 34 | A |
| 35 | B |
| 36 | A |
| 37 | A |
| 38 | A |
| 39 | A |
| 40 | A |
| 41 | A |
| 42 | A |
| 43 | B |
| 44 | A |
| 45 | B |
| 46 | B |
| 47 | A |
| 48 | A |
| 49 | B |
| 50 | B |
| 51 | A |
| 52 | B |
| 53 | A |
| 54 | A |
| 55 | A |
| 56 | A |
| 57 | A |
| 58 | A |
| 59 | B |
| 60 | B |
| 61 | B |
| 62 | A |
| 63 | A |
| 64 | A |
| 65 | B |
| 66 | A |
| 67 | A |
| 68 | A |
| 69 | A |
| 70 | A |
| 71 | A |
| 72 | A |
| 73 | B |
| 74 | B |
| 75 | A |
| 76 | A |
| 77 | A |
| 78 | A |
| 79 | A |
| 80 | A |
| 81 | A |
| 82 | B |
| 83 | A |
| 84 | A |
| 85 | A |
| 86 | B |
| 87 | B |
| 89 | A |
| 90 | B |
| 91 | A |
| 92 | B |
| 93 | B |
| 94 | B |
| 95 | B |
| 96 | B |
| 97 | B |
| 98 | B |
| 99 | B |
| 100 | B |
| 101 | B |
| 102 | B |
| 103 | B |
| 104 | B |
| 105 | B |
| 106 | B |
| 107 | B |
| 108 | A |
| 109 | B |
| 110 | B |
| 111 | A |
| 112 | A |
| 113 | B |
| 114 | B |
| 115 | B |
| 116 | B |
| 117 | A |
| 118 | A |
| 119 | A |
| 120 | A |
| 121 | B |
| 122 | A |
| 123 | B |
| 124 | B |
| 125 | A |
| 126 | A |
| 127 | A |
| 128 | A |
| 129 | A |
| 130 | B |
| 131 | A |
| 132 | A |
| 133 | B |
| 134 | B |
| 135 | B |
| 136 | A |
| 137 | A |
| 138 | A |
| 139 | A |
| 140 | B |
| 141 | B |
| 142 | B |
| 143 | B |
| 144 | A |
| 145 | A |
| 146 | B |
| 147 | B |
| 148 | A |
| 149 | A |
| 150 | A |
| 151 | B |
| 152 | B |
| 153 | B |
| 154 | A |
| 155 | B |
| 156 | A |
| 157 | A |
| 158 | A |
| 159 | B |
| 160 | B |
| 161 | A |
| 162 | B |
| 163 | A |
| 164 | B |
| 165 | B |
| 166 | B |
| 167 | A |
| 168 | A |
| 169 | B |
| 170 | B |
| 171 | A |
| 172 | B |
| 173 | B |
| 174 | B |
| 175 | B |
| 176 | A |
| 177 | B |
| 178 | A |
| 179 | A |
| 180 | A |
| 181 | A |
| 182 | A |
| 183 | A |
| 184 | A |
| 185 | A |
| 186 | A |
| 187 | A |
| 188 | B |
| 189 | A |

The IC₅₀ values for hIDO1 enzyme of the compounds of present invention are as follows:

| Example | hIDO1- |
|---|---|
| No | IC₅₀ (μM) |
| 11 | >0.5 |
| 12 | >0.5 |
| 17 | <0.5 |
| 18 | <0.5 |
| 19 | <0.5 |
| 20 | >0.5 |
| 25 | <0.5 |
| 28 | <0.5 |
| 29 | <0.5 |
| 30 | >0.5 |
| 31 | >0.5 |
| 37 | <0.5 |
| 38 | <0.5 |
| 39 | <0.5 |
| 41 | <0.5 |
| 42 | <0.5 |
| 48 | >0.5 |
| 51 | >0.5 |
| 53 | <0.5 |
| 55 | <0.5 |
| 56 | <0.5 |
| 57 | <0.5 |
| 58 | <0.5 |
| 62 | <0.5 |
| 64 | <0.5 |
| 67 | >0.5 |
| 69 | >0.5 |
| 71 | <0.5 |
| 75 | <0.5 |
| 76 | <0.5 |
| 77 | <0.5 |
| 79 | >0.5 |
| 81 | <0.5 |
| 88 | >0.5 |
| 89 | <0.5 |
| 91 | <0.5 |
| 111 | >0.5 |
| 112 | <0.5 |
| 117 | >0.5 |
| 119 | <0.5 |
| 120 | <0.5 |
| 122 | <0.5 |
| 126 | <0.5 |
| 127 | >0.5 |
| 128 | <0.5 |
| 129 | >0.5 |
| 131 | <0.5 |
| 132 | <0.5 |
| 137 | <0.5 |
| 139 | <0.5 |
| 144 | <0.5 |
| 145 | <0.5 |
| 148 | <0.5 |
| 149 | <0.5 |
| 150 | <0.5 |
| 152 | >0.5 |
| 154 | <0.5 |
| 156 | <0.5 |
| 157 | >0.5 |
| 158 | <0.5 |
| 161 | >0.5 |
| 163 | <0.5 |
| 167 | <0.5 |
| 168 | >0.5 |
| 170 | >0.5 |
| 176 | <0.5 |
| 178 | <0.5 |
| 179 | <0.5 |
| 180 | <0.5 |
| 181 | <0.5 |
| 182 | <0.5 |
| 183 | <0.5 |
| 184 | <0.5 |
| 185 | <0.5 |
| 186 | <0.5 |
| 187 | >0.5 |
| 189 | <0.5 |

### In-Vitro Human Tryptophan 2,3-Dioxygenase (TDO) Enzyme Assay:

In the in-vitro human tryptophan 2,3-dioxygenase (hTDO) enzyme assay for screening inhibitor compounds, hTDO with an N-terminal histidine tag expressed and purified from E. coli (BPS Bioscience, San Diego, Calif., USA) was used. All other materials were procured from Sigma-Aldrich, St. Louis, Mo., USA.

The assay monitoring method for the conversion of L-tryptophan to N-formylkynurenine by hTDO was carried out as follows. hTDO (125 ng) was incubated in the presence of 200 μM L-tryptophan, 100 mM sodium phosphate buffer (pH 7.0), 0.01% Tween-20 and 100 μM ascorbic acid at about 37° C. for about 60 minutes. The reaction was terminated with 200 mM piperidine (PIP) and further incubated at about 65° C. for about 20 minutes to convert N-formylkynurenine (NFK) to NFK-PIP. The reaction mixture was then incubated at room temperature for about 75 minutes. The fluorescence intensity was read in a fluorescence microplate reader at an excitation wavelength of 400 nm and emission wavelength of 500 nm. Percent inhibition at each concentration of test compounds was determined by estimating the decrease in NFK-PIP. Data were analyzed using nonlinear regression to generate IC₅₀ values using Graph Pad Prism® 6.

The % inhibition values for hTDO enzyme at 1.0 μM concentration of the compounds of present invention are as follows (A: ≥50%, B: ≤50%):

| Example | % inhibition |
|---|---|
| No | hIDO1 |
| 17 | A |
| 18 | A |
| 19 | A |
| 20 | A |
| 23 | A |
| 24 | A |
| 25 | A |
| 26 | A |
| 27 | A |
| 28 | A |
| 29 | A |
| 30 | A |
| 31 | B |
| 34 | A |
| 35 | A |
| 36 | A |
| 37 | A |
| 38 | A |
| 39 | A |
| 40 | A |
| 41 | A |
| 42 | A |
| 43 | B |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | B |
| 57 | B |
| 58 | B |
| 59 | B |
| 60 | B |
| 61 | B |
| 62 | A |
| 63 | A |
| 64 | B |
| 65 | B |
| 66 | A |
| 67 | A |
| 68 | A |
| 69 | B |
| 70 | A |
| 71 | A |
| 73 | B |
| 74 | B |
| 75 | A |
| 76 | B |
| 77 | A |
| 78 | A |
| 79 | A |
| 80 | A |
| 81 | A |
| 82 | B |
| 83 | A |
| 84 | A |
| 85 | B |
| 86 | A |
| 87 | A |
| 88 | A |
| 89 | A |
| 90 | A |
| 91 | A |
| 92 | B |
| 93 | B |
| 94 | A |
| 95 | B |
| 96 | B |
| 97 | A |
| 98 | A |
| 99 | A |
| 100 | A |
| 101 | A |
| 102 | B |
| 103 | B |
| 104 | B |
| 105 | B |
| 106 | B |
| 107 | A |
| 108 | A |
| 109 | A |
| 110 | A |
| 111 | A |
| 112 | A |
| 113 | A |
| 114 | A |
| 115 | B |
| 116 | B |
| 117 | A |
| 118 | A |
| 119 | A |
| 120 | A |
| 121 | B |
| 122 | A |
| 123 | A |
| 124 | B |
| 125 | B |
| 126 | A |
| 127 | A |
| 128 | A |
| 129 | A |
| 130 | B |
| 131 | A |
| 132 | A |
| 133 | A |
| 134 | A |
| 135 | A |
| 136 | A |
| 137 | A |
| 138 | A |
| 139 | A |
| 140 | B |
| 141 | B |
| 142 | A |
| 143 | A |
| 144 | B |
| 145 | A |
| 146 | B |
| 147 | B |
| 148 | B |
| 149 | B |
| 150 | A |
| 151 | B |
| 152 | B |
| 153 | B |
| 154 | A |
| 155 | B |
| 156 | B |
| 157 | B |
| 158 | A |
| 159 | B |
| 160 | B |
| 161 | B |
| 162 | B |
| 163 | A |
| 164 | B |
| 165 | B |
| 166 | B |
| 167 | B |
| 168 | A |
| 170 | A |
| 173 | B |
| 174 | B |
| 175 | B |
| 176 | A |
| 177 | A |
| 178 | B |
| 179 | B |
| 180 | A |
| 181 | B |
| 182 | B |
| 183 | B |
| 184 | A |
| 185 | A |
| 186 | B |
| 187 | B |
| 188 | B |
| 189 | A |

The % inhibition values for hTDO enzyme at 10.0 μM concentration of the compounds of resent invention are as follows (A: ≥50%, B: ≤550):

| Example | % inhibition |
|---|---|
| No | hIDO1 |
| 1 | A |
| 2 | A |
| 3 | A |
| 4 | A |
| 5 | B |
| 6 | B |
| 7 | A |
| 8 | A |
| 9 | A |
| 10 | A |
| 11 | A |
| 12 | A |
| 13 | B |
| 14 | B |
| 15 | A |
| 16 | A |
| 17 | A |
| 18 | A |
| 19 | A |
| 20 | A |
| 21 | B |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 36 | A |
| 71 | A |
| 72 | A |
| 78 | A |
| 88 | A |
| 89 | A |
| 91 | A |
| 168 | A |
| 169 | A |
| 170 | A |
| 171 | A |
| 172 | B |

Many of these compounds have shown good (<0.5 μM) IC₅₀ values against hTDO-enzyme assay.

### In-Vitro Human Indoleamine 2, 3-Dioxygenase 1 (IDO1) HEK293 Cell Based Assay:

Human indoleamine 2, 3-dioxygenase 1 (hIDO1) cell based assay for screening inhibitor compounds used a stable recombinant HEK293 cell line, human IDO1-HEK293, expressing tetracycline-inducible human indoleamine 2, 3-dioxygenase (Genbank accession number NM_002164) procured from BPS Bioscience, San Diego, Calif., USA. All other materials were procured from Sigma-Aldrich, St. Louis, Mo., USA.

Human IDO1-HEK293 cells were seeded at 25,000 cells, with MEM media containing 10% FBS, in a tissue culture-treated 96-well plate followed by incubation at about 37° C. in a CO₂ incubator overnight. The next day medium was replaced with different concentrations of reference or test compounds in growth medium (100 μl) and 100 μl of growth medium containing 0.2 μg/ml of doxycycline and 200 μg/ml L-Tryptophan to induce IDO1 expression followed by incubation at about 37° C. in a CO₂ incubator around 24 hours. 140 μL of medium was then transferred to a fresh 96 well plate followed by addition of 10 μL of 6.1 N trichloroacetic acid to each well and incubated at about 50° C. for about 30 minutes followed by centrifugation at 2500×g for about 10 minutes. 100 μL of clear supernatant was transferred to a transparent 96-well plate and mixed with 100 μL of freshly prepared 2% 4-(Dimethylamino) benzaldehyde in glacial acetic acid. The plate was incubated at room temperature for about 10 minutes and absorbance measured at 480 nm using a micro plate reader. Data were analyzed using nonlinear regression to generate IC₅₀ values using Graph Pad Prism® 6.

Many of the present invention compounds that showed activity in the enzyme based biochemical hIDO1 assay were also active in the hIDO-HEK293 cell line based assay.

Although the invention herein has been described with reference to particular embodiments, it is to be understood that these embodiments are merely illustrative of the principles and applications of the present invention. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. It is therefore to be understood that numerous modifications may be made to the illustrative embodiments and that other arrangements may be devised without departing from the spirit and scope of the present invention as described above. All the publications and patent applications cited in this application are herein incorporated by reference to the same extent as if each individual publication or patent application was specifically and individually indicated to be incorporated herein by reference.

1. A compounds of the Formula (I): 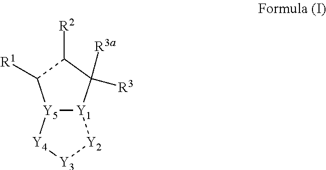
wherein,
each R¹, R² & R³ is selected independently from a radical 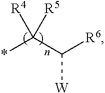
hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
and R¹ and R² are combined together with their adjacent carbon atom to form 5-8 membered substituted or unsubstituted monocyclic or 10-12 membered substituted or unsubstituted bicyclic cycloalkyl or heterocycloalkyl ring;
R^{3a} is selected from hydrogen, halogen, substituted or unsubstituted alkyl, substituted or unsubstituted cycloalkyl, substituted or unsubstituted aryl or substituted or unsubstituted heteroaryl;
R^{A}, R^{B} and R^{C} are independently selected from hydrogen, substituted or unsubstituted alkyl, substituted or unsubstituted arylalkyl, substituted or unsubstituted aryl, substituted or unsubstituted cycloalkyl, substituted or unsubstituted cycloalkylalkyl, substituted or unsubstituted heterocycloalkyl, substituted or unsubstituted heteroaryl, substituted or unsubstituted heteroarylalkyl;
R⁴ and R⁵ are independently selected from hydrogen, substituted or unsubstituted alkyl or substituted or unsubstituted aryl;
R⁶ is selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
W is selected from oxo (C═O), thio (C═S), OR^{A}, SR^{A}, NR^{A}R^{B} or halogen;
n is an integer 1-6;
Y₁, Y₂, Y₃, Y₄ and Y₅ are independently selected from CR^{D}R^{E}, N or NR^{D};
each R^{D} & R^{E} is independently selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
--- bond is a single or double bond; including pharmaceutically acceptable salts, pharmaceutically acceptable solvates, pharmaceutically acceptable hydrates, tautomers, stereoisomers, ester prodrugs, or combination thereof.

2. The compounds of claim 1 with the general Formula (IB): 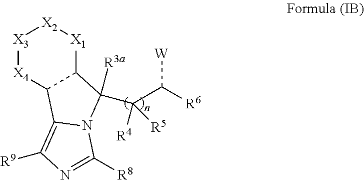
wherein,
X₁, X₂, X₃ and X₄ are independently selected from (CR^{D}R^{E})ₚ, O, S, NR^{D}, SO or SO₂ p can be an integer 0-3;
each R^{D} & R^{E} is independently selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
R⁸ and R⁹ are independently selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
R^{3a} is selected from hydrogen, halogen, substituted or unsubstituted alkyl, substituted or unsubstituted cycloalkyl, substituted or unsubstituted aryl or substituted or unsubstituted heteroaryl;
R⁴ & R⁵ is independently selected from hydrogen, substituted or unsubstituted alkyl or substituted or unsubstituted aryl;
R⁶ is selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
each R^{A}, R^{B} and R^{C} is independently selected from hydrogen, substituted or unsubstituted alkyl, haloalkyl, substituted or unsubstituted arylalkyl, substituted or unsubstituted aryl, substituted or unsubstituted cycloalkyl, substituted or unsubstituted cycloalkylalkyl, substituted or unsubstituted heterocycloalkyl, substituted or unsubstituted heteroaryl, substituted or unsubstituted heteroarylalkyl;
W is selected from oxo (C═O), thio (C═S), OR^{A}, SR^{A}, NR^{A}R^{B} or halogen;
n is an integer 1-6;
--- bond is a single or double bond; including pharmaceutically acceptable salts, pharmaceutically acceptable solvates, pharmaceutically acceptable hydrates, tautomers, stereoisomers, ester prodrugs, or combination thereof.

3. The compounds of claim 1 with the Formula (IC): 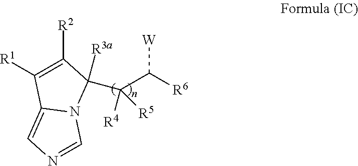
wherein,
each R¹ and R² are selected independently from a radical 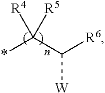
hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
each R^{A}, R^{B} and R^{C} is independently selected from hydrogen, substituted or unsubstituted alkyl, haloalkyl, substituted or unsubstituted arylalkyl, substituted or unsubstituted aryl, substituted or unsubstituted cycloalkyl, substituted or unsubstituted cycloalkylalkyl, substituted or unsubstituted heterocycloalkyl, substituted or unsubstituted heteroaryl, substituted or unsubstituted heteroarylalkyl;
R^{3a} is selected from hydrogen, halogen, substituted or unsubstituted alkyl, substituted or unsubstituted cycloalkyl, substituted or unsubstituted aryl or substituted or unsubstituted heteroaryl;
R⁴ & R⁵ are independently selected from hydrogen, substituted or unsubstituted alkyl or substituted or unsubstituted aryl;
R⁶ is selected from hydrogen, halogen, nitro, cyano, substituted or unsubstituted alkyl, substituted or unsubstituted alkenyl, substituted or unsubstituted alkynyl, substituted or unsubstituted aryl, substituted or unsubstituted arylalkyl, substituted or unsubstituted aryloxy, substituted or unsubstituted heteroaryl, substituted or unsubstituted heteroarylalkyl, substituted or unsubstituted heteroaryloxy, substituted or unsubstituted cycloalkyl, substituted or unsubstituted cycloalkenyl, substituted or unsubstituted cycloalkylalkyl, substituted or unsubstituted heterocycloalkyl, substituted or unsubstituted heterocycloalkylalkyl, substituted or unsubstituted spiroalkyl, —OR^{A}, —R^{A}OR^{B}, —SR^{A}, —C(O)OR^{A}, —R^{A}C(O)OR^{B}, —C(O)NR^{A}R^{B}, —C(O)R^{A}, —C(S)R^{A}, —OC(O)R^{A}, —OC(O)OR^{A}, —OC(O)NR^{A}R^{B}, —OR^{A}C(O)NR^{B}R^{C}, —NR^{A}R^{B}, —N(R^{A})C(O)R^{B}, —N(R^{A})C(S)R^{B}, —NR^{A}SOR^{B}, —NR^{A}SO₂R^{B}, —N(R^{A})C(O)OR^{B}, —N(R^{A})C(O)NR^{B}R^{C}, —N(R^{A})C(S)NR^{B}R^{C}, —S(O)R^{A}, —S(O)₂R^{A}, —S(O)OR^{A}, —S(O)₂OR^{A}, —S(O)NR^{A}R^{B}, or —S(O)₂NR^{A}R^{B};
W is selected from oxo (C═O), thio (C═S), OR^{A}, SR^{A}, NR^{A}R^{B} or halogen;
n is an integer 1-6;
--- bond is a single or double bond; including pharmaceutically acceptable salts, pharmaceutically acceptable solvates, pharmaceutically acceptable hydrates, tautomers, stereoisomers, ester prodrugs, or combination thereof.

4. A compound of claim 1 selected from the group consisting of:
1-Phenyl-2-(6,7,8,9-tetrahydro-5H-imidazo[5,1-a]isoindol-5-yl)-ethanone;
1-Phenyl-2-(6,7,8,9-tetrahydro-5H-imidazo[5,1-a]isoindol-5-yl)-ethanol;
1-(3-Chloro-phenyl)-2-(6,7,8,9-tetrahydro-5H-imidazo[5,1-a]isoindol-5-yl)-ethanone;
1-(3-Chloro-phenyl)-2-(6,7,8,9-tetrahydro-5H-imidazo[5,1-a]isoindol-5-yl)-ethanol;
1-Phenyl-2-[6-(toluene-4-sulfonyl)-4,6,7,8-tetrahydro-5H-2,6,8a-triaza-cyclopenta [a] inden-8-yl]-ethanone;
1-Phenyl-2-[6-(toluene-4-sulfonyl)-4,6,7,8-tetrahydro-5H-2,6,8a-triaza-cyclopenta [a] inden-8-yl]-ethanol;
1-(3-Benzyloxy-phenyl)-2-(6,7,8,9-tetrahydro-5H-imidazo[5,1-a]isoindol-5-yl)-ethanone;
1-(3-Benzyloxy-phenyl)-2-(6,7,8,9-tetrahydro-5H-imidazo[5,1-a]isoindol-5-yl)-ethanol;
1-(2,5-Difluoro-phenyl)-2-(6,7,8,9-tetrahydro-5H-imidazo[5,1-a]isoindol-5-yl)-ethanone;
1-(2,5-Difluoro-phenyl)-2-(6,7,8,9-tetrahydro-5H-imidazo[5,1-a]isoindol-5-yl)-ethanol;
2-(7-Methyl-6-phenyl-5H-pyrrolo[1,2-c]imidazol-5-yl)-1-phenyl-ethanone;
2-(7-Methyl-6-phenyl-5H-pyrrolo[1,2-c]imidazol-5-yl)-1-phenyl-ethanol;
1-Phenyl-2-(7-phenyl-5H-pyrrolo[1,2-c]imidazol-5-yl)-ethanone;
1-Phenyl-2-(7-phenyl-5H-pyrrolo[1,2-c]imidazol-5-yl)-ethanol;
2-[6-(3-Fluoro-phenyl)-7-methyl-5H-pyrrolo[1,2-c]imidazol-5-yl]-1-phenyl-ethanone;
2-[6-(3-Fluoro-phenyl)-7-methyl-5H-pyrrolo[1,2-c]imidazol-5-yl]-1-phenyl-ethanol;
1-(3-Chloro-phenyl)-2-(7-methyl-6-phenyl-5H-pyrrolo[1,2-c]imidazol-5-yl)-ethanone;
4-[2-(7-Methyl-6-phenyl-5H-pyrrolo[1,2-c]imidazol-5-yl)-acetyl]-benzoic acid methyl ester;
1-(3-Chloro-phenyl)-2-(7-methyl-6-phenyl-5H-pyrrolo[1,2-c]imidazol-5-yl)-ethanol;
1-(3-Fluoro-phenyl)-2-(7-methyl-6-phenyl-5H-pyrrolo[1,2-c]imidazol-5-yl)-ethanone;
2-(6-Methyl-7-phenyl-5H-pyrrolo[1,2-c]imidazol-5-yl)-1-phenyl-ethanone;
(7-Methyl-6-phenyl-5H-pyrrolo[1,2-c]imidazol-5-yl)-acetic acid ethyl ester;
3-[2-(7-Methyl-6-phenyl-5H-pyrrolo[1,2-c]imidazol-5-yl)-acetyl]-benzoic acid methyl ester;
4-[2-(7-Methyl-6-phenyl-5H-pyrrolo[1,2-c]imidazol-5-yl)-acetyl]-benzoic acid;
2-(7-Methyl-6-phenyl-5H-pyrrolo[1,2-c]imidazol-5-yl)-1-naphthalen-2-yl-ethanone;
1-Cyclopropyl-2-(7-methyl-6-phenyl-5H-pyrrolo[1,2-c]imidazol-5-yl)-ethanone;
2-(7-Methyl-6-phenyl-5H-pyrrolo[1,2-c]imidazol-5-yl)-1-p-tolyl-ethanone;
1-Benzo[1,3]dioxol-5-yl-2-(7-methyl-6-phenyl-5H-pyrrolo[1,2-c]imidazol-5-yl)-ethanone;
2-(7-Methyl-6-phenyl-5H-pyrrolo[1,2-c]imidazol-5-yl)-1-(3-trifluoromethyl-phenyl)-ethanone;
2-(7-Methyl-6-phenyl-5H-pyrrolo[1,2-c]imidazol-5-yl)-1-phenyl-ethanone (Isomer-I);
2-(7-Methyl-6-phenyl-5H-pyrrolo[1,2-c]imidazol-5-yl)-1-phenyl-ethanone (Isomer-II);
1-(2-methoxyphenyl)-2-(7-methyl-6-phenyl-5H-pyrrolo[1,2-c]imidazol-5-yl)ethan-1-one;
1-(3-Methoxy-phenyl)-2-(7-methyl-6-phenyl-5H-pyrrolo[1,2-c]imidazol-5-yl)-ethanone;
1-(4-fluorophenyl)-2-(7-methyl-6-phenyl-5H-pyrrolo[1,2-c]imidazol-5-yl)ethan-1-one;
1-(4-fluorophenyl)-2-(7-methyl-6-phenyl-5H-pyrrolo[1,2-c]imidazol-5-yl)ethan-1-ol;
1-(2,5-difluorophenyl)-2-(7-methyl-6-phenyl-5H-pyrrolo[1,2-c]imidazol-5-yl)ethan-1-one;
1-(4-methoxyphenyl)-2-(7-methyl-6-phenyl-5H-pyrrolo[1,2-c]imidazol-5-yl)ethan-1-one;
1-(4-chlorophenyl)-2-(7-methyl-6-phenyl-5H-pyrrolo[1,2-c]imidazol-5-yl)ethan-1-one;
1-(3-chloro-4-fluorophenyl)-2-(7-methyl-6-phenyl-5H-pyrrolo[1,2-c]imidazol-5-yl)ethan-1-one;
2-(7-methyl-6-phenyl-5H-pyrrolo[1,2-c]imidazol-5-yl)-1-(naphthalen-1-yl)ethan-1-one;
1-([1,1′-biphenyl]-4-yl)-2-(7-methyl-6-phenyl-5H-pyrrolo[1,2-c]imidazol-5-yl)ethan-1-one (Isomer-I);
1-([1,1′-biphenyl]-4-yl)-2-(7-methyl-6-phenyl-5H-pyrrolo[1,2-c]imidazol-5-yl)ethan-1-one (Isomer-I);
1-([1,1′-biphenyl]-4-yl)-2-(7-methyl-6-phenyl-5H-pyrrolo[1,2-c]imidazol-5-yl)ethan-1-one;
1-([1,1′-biphenyl]-4-yl)-2-(7-methyl-6-phenyl-5H-pyrrolo[1,2-c]imidazol-5-yl)ethan-1-ol;
2-(7-methyl-6-phenyl-5H-pyrrolo[1,2-c]imidazol-5-yl)-1-(thiophen-3-yl)ethan-1-one;
1-(4-(dimethylamino)phenyl)-2-(7-methyl-6-phenyl-5H-pyrrolo[1,2-c]imidazol-5-yl)ethan-1-one;
2-(7-methyl-6-phenyl-5H-pyrrolo[1,2-c]imidazol-5-yl)-1-(5,6,7,8-tetrahydronaphthalen-2-yl)ethan-1-one;
2-(7-methyl-6-phenyl-5H-pyrrolo[1,2-c]imidazol-5-yl)-1-(4-phenoxyphenyl)ethan-1-one;
2-(7-methyl-6-phenyl-5H-pyrrolo[1,2-c]imidazol-5-yl)-1-(4-morpholinophenyl)ethan-1-one;
2-(7-methyl-6-phenyl-5H-pyrrolo[1,2-c]imidazol-5-yl)-1-(4-(piperidin-1-yl)phenyl)ethan-1-one;
1-(4-bromophenyl)-2-(7-methyl-6-phenyl-5H-pyrrolo[1,2-c]imidazol-5-yl)ethan-1-one;
1-(4-bromophenyl)-2-(7-methyl-6-phenyl-5H-pyrrolo[1,2-c]imidazol-5-yl)ethan-1-ol;
2-(7-methyl-6-phenyl-5H-pyrrolo[1,2-c]imidazol-5-yl)-1-(4-(pyridin-4-yl)phenyl)ethan-1-one;
2-(7-methyl-6-phenyl-5H-pyrrolo[1,2-c]imidazol-5-yl)-1-(4-(pyrimidin-5-yl)phenyl)ethan-1-one;
2-(7-methyl-6-phenyl-5H-pyrrolo[1,2-c]imidazol-5-yl)-1-(4-(pyridin-3-yl)phenyl)ethan-1-one;
2-(7-methyl-6-phenyl-5H-pyrrolo[1,2-c]imidazol-5-yl)-1-(4-(thiophen-3-yl)phenyl)ethan-1-one;
1-(4′-hydroxy-[1,1′-biphenyl]-4-yl)-2-(7-methyl-6-phenyl-5H-pyrrolo[1,2-c]imidazol-5-yl)ethan-1-one;
1-(2′-fluoro-[1,1′-biphenyl]-4-yl)-2-(7-methyl-6-phenyl-5H-pyrrolo[1,2-c]imidazol-5-yl)ethan-1-one;
2-(7-methyl-6-phenyl-5H-pyrrolo[1,2-c]imidazol-5-yl)-1-(4-(pyridin-4-yl)phenyl)ethan-1-ol;
2-(7-methyl-6-phenyl-5H-pyrrolo[1,2-c]imidazol-5-yl)-1-(4-(thiophen-3-yl)phenyl)ethan-1-ol;
2-(7-methyl-6-phenyl-5H-pyrrolo[1,2-c]imidazol-5-yl)-1-(4′-(trifluoromethyl)-[1,1′-biphenyl]-4-yl)ethan-1-one;
2-(7-methyl-6-phenyl-5H-pyrrolo[1,2-c]imidazol-5-yl)-1-(4′-methyl-[1,1′-biphenyl]-4-yl)ethan-1-one;
1-(4′-fluoro-[1,1′-biphenyl]-4-yl)-2-(7-methyl-6-phenyl-5H-pyrrolo[1,2-c]imidazol-5-yl)ethan-1-one;
2-(7-methyl-6-phenyl-5H-pyrrolo[1,2-c]imidazol-5-yl)-1-(4-(5-methylthiophen-2-yl)phenyl)ethan-1-one;
1-([1,1′:4′,1″-terphenyl]-4-yl)-2-(7-methyl-6-phenyl-5H-pyrrolo[1,2-c]imidazol-5-yl)ethan-1-one;
2-(7-methyl-6-phenyl-5H-pyrrolo[1,2-c]imidazol-5-yl)-1-(4′-(methylsulfonyl)-[1,1′-biphenyl]-4-yl)ethan-1-one;
1-(4-(1H-imidazol-5-yl)phenyl)-2-(7-methyl-6-phenyl-5H-pyrrolo[1,2-c]imidazol-5-yl)ethan-1-one;
1-(3-Bromo-phenyl)-2-(7-methyl-6-phenyl-5H-pyrrolo[1,2-c]imidazol-5-yl)-ethanone;
1-Biphenyl-3-yl-2-(7-methyl-6-phenyl-5H-pyrrolo[1,2-c]imidazol-5-yl)-ethanone;
1-(2′-Fluoro-biphenyl-3-yl)-2-(7-methyl-6-phenyl-5H-pyrrolo[1,2-c]imidazol-5-yl)-ethanone;
4-[2-(7-Methyl-6-phenyl-5H-pyrrolo[1,2-c]imidazol-5-yl)-acetyl]-benzoic acid methyl ester (Isomer-I);
4-[2-(7-Methyl-6-phenyl-5H-pyrrolo[1,2-c]imidazol-5-yl)-acetyl]-benzoic acid methyl ester (Isomer-II);
1-(4-Cyclohexylphenyl)-2-(7-methyl-6-phenyl-5H-pyrrolo[1,2-c]imidazol-5-yl)ethan-1-one;
1-(4-Cyclohexylphenyl)-2-(7-methyl-6-phenyl-5H-pyrrolo[1,2-c]imidazol-5-yl)ethan-1-ol;
1-(4-(benzyloxy)phenyl)-2-(7-methyl-6-phenyl-5H-pyrrolo[1,2-c]imidazol-5-yl)ethan-1-one;
1-(4-(2-fluorophenoxy) phenyl)-2-(7-methyl-6-phenyl-5H-pyrrolo[1,2-c]imidazol-5-yl)ethan-1-one;
N-methyl-4-(2-(7-methyl-6-phenyl-5H-pyrrolo[1,2-c]imidazol-5-yl)acetyl)benzamide;
N-methyl-4-(2-(7-methyl-6-phenyl-5H-pyrrolo[1,2-c]imidazol-5-yl)acetyl)benzamide;
4-(2-(7-methyl-6-phenyl-5H-pyrrolo[1,2-c]imidazol-5-yl)acetyl)-N-(pyridin-3-yl)benzamide;
N-(4-chlorophenyl)-4-(2-(7-methyl-6-phenyl-5H-pyrrolo[1,2-c]imidazol-5-yl)acetyl)benzamide;
N-(2,4-difluorophenyl)-4-(2-(7-methyl-6-phenyl-5H-pyrrolo[1,2-c]imidazol-5-yl)acetyl)benzamide;
N-(4-chlorophenyl)-4-(1-hydroxy-2-(7-methyl-6-phenyl-5H-pyrrolo[1,2-c]imidazol-5-yl)ethyl)benzamide;
N-isobutyl-4-(2-(7-methyl-6-phenyl-5H-pyrrolo[1,2-c]imidazol-5-yl)acetyl)benzamide;
N-(2,4-dimethylphenyl)-4-(2-(7-methyl-6-phenyl-5H-pyrrolo[1,2-c]imidazol-5-yl)acetyl)benzamide;
4-(2-(7-methyl-6-phenyl-5H-pyrrolo[1,2-c]imidazol-5-yl)acetyl)-N-phenylbenzamide;
1-Cyclohexyl-2-(7-methyl-6-(p-tolyl)-5H-pyrrolo[1,2-c]-imidazol-5-yl)ethan-1-one;
1-Cyclohexyl-2-(7-methyl-6-(p-tolyl)-5H-pyrrolo[1,2-c]imidazol-5-yl)ethan-1-ol;
1-Cyclohexyl-2-(7-methyl-6-phenyl-5H-pyrrolo[1,2-c]imidazol-5-yl)-ethanone;
1-Cyclohexyl-2-(7-methyl-6-phenyl-5H-pyrrolo[1,2-c]imidazol-5-yl)-ethan-1-ol;
1-Cyclohexyl-2-(7-methyl-6-phenyl-5H-pyrrolo[1,2-c]imidazol-5-yl)ethan-1-ol (Isomer-I);
1-Cyclohexyl-2-(7-methyl-6-phenyl-5H-pyrrolo[1,2-c]imidazol-5-yl)ethan-1-ol (Isomer-II);
1-Cyclohexyl-2-(7-methyl-6-phenyl-5H-pyrrolo[1,2-c]imidazol-5-yl)ethan-1-ol (Isomer-III);
1-Cyclohexyl-2-(7-methyl-6-phenyl-5H-pyrrolo[1,2-c]imidazol-5-yl)ethan-1-ol (Isomer-IV);
Methyl 4-(2-(7-methyl-6-phenyl-5H-pyrrolo[1,2-c]imidazol-5-yl)acetyl)cyclohexane-1-carboxylate;
2-(6-(3-Chlorophenyl)-7-methyl-5H-pyrrolo[1,2-c]imidazol-5-yl)-1-cyclohexylethan-1-one;
2-(6-(3-Chlorophenyl)-7-methyl-5H-pyrrolo[1,2-c]imidazol-5-yl)-1-cyclohexylethan-1-ol;
2-(6-(2-fluorophenyl)-7-methyl-5H-pyrrolo[1,2-c]imidazol-5-yl)-1-(4-hydroxycyclohexyl)ethan-1-one (Isomer-I);
2-(6-(2-fluorophenyl)-7-methyl-5H-pyrrolo[1,2-c]imidazol-5-yl)-1-(4-hydroxycyclohexyl)ethan-1-one (Isomer-II);
1-(4-Hydroxycyclohexyl)-2-(7-methyl-6-phenyl-5H-pyrrolo[1,2-c]imidazol-5-yl)ethan-1-one;
2-(7-methyl-6-phenyl-5H-pyrrolo[1,2-c]imidazol-5-yl)-1-(tetrahydro-2H-pyran-4-yl)ethan-1-one;
1-Cyclohexyl-2-(6-(2-fluorophenyl)-7-methyl-5H-pyrrolo[1,2-c]imidazol-5-yl)ethan-1-one;
1-Cyclohexyl-2-(6,7-diphenyl-5H-pyrrolo[1,2-c]imidazol-5-yl)ethan-1-one;
1-Cyclohexyl-2-(6,7-diphenyl-5H-pyrrolo[1,2-c]imidazol-5-yl)ethan-1-ol;
1-cyclohexyl-2-(6-(4-methoxyphenyl)-7-methyl-5H-pyrrolo[1,2-c]imidazol-5-yl)ethan-1-one;
1-Cyclohexyl-2-(7-methyl-6-(o-tolyl)-5H-pyrrolo[1,2-c]imidazol-5-yl)ethan-1-ol;
1-Cyclohexyl-2-(7-isopropyl-6-phenyl-5H-pyrrolo[1,2-c]imidazol-5-yl)ethan-1-one;
1-(Adamantan-1-yl)-2-(7-methyl-6-phenyl-5H-pyrrolo[1,2-c]imidazol-5-yl)ethan-1-one;
1-(Adamantan-1-yl)-2-(7-methyl-6-phenyl-5H-pyrrolo[1,2-c]imidazol-5-yl)ethan-1-ol;
4-(2-(7-methyl-6-phenyl-5H-pyrrolo[1,2-c]imidazol-5-yl)acetyl)-N-phenylcyclohexane-1-carboxamide;
N-(2,4-difluorophenyl)-4-(2-(7-methyl-6-phenyl-5H-pyrrolo[1,2-c]imidazol-5-yl)acetyl)cyclohexane-1-carboxamide;
4-(1-hydroxy-2-(7-methyl-6-phenyl-5H-pyrrolo[1,2-c]imidazol-5-yl)ethyl)cyclohexan-1-ol;
4-(1-hydroxy-2-(7-methyl-6-phenyl-5H-pyrrolo[1,2-c]imidazol-5-yl)ethyl)-N-phenylcyclohexane-1-carboxamide;
2-(7-Methyl-6-phenyl-5H-pyrrolo[1,2-c]imidazol-5-yl)-1-((S)-1-tosylpyrrolidin-2-yl)ethan-1-one;
(1R)-2-(7-Methyl-6-phenyl-5H-pyrrolo[1,2-c]imidazol-5-yl)-1-((R)-1-tosylpyrrolidin-2-yl)ethan-1-ol;
1-((S)-1-benzoylpyrrolidin-2-yl)-2-(7-methyl-6-phenyl-5H-pyrrolo[1,2-c]imidazol-5-yl)ethan-1-one;
((2R)-2-((1R)-1-hydroxy-2-(7-methyl-6-phenyl-5H-pyrrolo[1,2-c]imidazol-5-yl)ethyl)pyrrolidin-1-yl)(phenyl)methanone;
4-(2-(7-methyl-6-phenyl-5H-pyrrolo[1,2-c]imidazol-5-yl)acetyl)-N-(pyridin-4-yl)cyclohexane-1-carboxamide;
N-(4-chloro-2-methylphenyl)-4-(2-(7-methyl-6-phenyl-5H-pyrrolo[1,2-c]imidazol-5-yl)acetyl)cyclohexane-1-carboxamide;
4-(1-hydroxy-2-(7-methyl-6-phenyl-5H-pyrrolo[1,2-c]imidazol-5-yl)ethyl)-N-(pyridin-4-yl)cyclohexane-1-carboxamide;
N-(4-chloro-2-methylphenyl)-4-(1-hydroxy-2-(7-methyl-6-phenyl-5H-pyrrolo[1,2-c]imidazol-5-yl)ethyl)cyclohexane-1-carboxamide;
4-(1-hydroxy-2-(7-methyl-6-phenyl-5H-pyrrolo[1,2-c]imidazol-5-yl)ethyl)cyclohexan-1-ol (Isomer-I);
4-(1-hydroxy-2-(7-methyl-6-phenyl-5H-pyrrolo[1,2-c]imidazol-5-yl)ethyl)cyclohexan-1-ol (Isomer-II);
N-cyclohexyl-4-(2-(7-methyl-6-phenyl-5H-pyrrolo[1,2-c]imidazol-5-yl)acetyl)cyclohexane-1-carboxamide;
1-(4-(2-(7-methyl-6-phenyl-5H-pyrrolo[1,2-c]imidazol-5-yl)acetyl)cyclohexane-1-carbonyl)piperidin-4-one;
(4-(1-hydroxy-2-(7-methyl-6-phenyl-5H-pyrrolo[1,2-c]imidazol-5-yl)ethyl)cyclohexyl)(4-hydroxypiperidin-1-yl)methanone;
N-cyclohexyl-4-(1-hydroxy-2-(7-methyl-6-phenyl-5H-pyrrolo[1,2-c]imidazol-5-yl)ethyl)cyclohexane-1-carboxamide;
4-(1-Hydroxy-2-(7-methyl-6-phenyl-5H-pyrrolo[1,2-c]imidazol-5-yl)ethyl)cyclohexan-1-ol;
4-(1-hydroxy-2-(7-methyl-6-phenyl-5H-pyrrolo[1,2-c]imidazol-5-yl)ethyl)-N-(pyridin-3-yl)cyclohexane-1-carboxamide;
4-(1-hydroxy-2-(7-methyl-6-phenyl-5H-pyrrolo[1,2-c]imidazol-5-yl)ethyl)-N-(4-hydroxycyclohexyl)cyclohexane-1-carboxamide (Compound 129);
(1 s,4s)-4-(2-(7-methyl-6-phenyl-5H-pyrrolo[1,2-c]imidazol-5-yl)acetyl)cyclohexane-1-carboxylic acid (Compound 130);
Methyl (1R,4s)-4-((1 S)-1-hydroxy-2-(7-methyl-6-phenyl-5H-pyrrolo[1,2-c]imidazol-5-yl)ethyl)cyclohexane-1-carboxylate;
4-(1-hydroxy-2-(7-methyl-6-phenyl-5H-pyrrolo[1,2-c]imidazol-5-yl)ethyl)-N-(pyridazin-3-yl)cyclohexane-1-carboxamide;
4-(1-hydroxy-2-(7-methyl-6-phenyl-5H-pyrrolo[1,2-c]imidazol-5-yl)ethyl)cyclohexane-1-carboxylic acid;
4-(1-hydroxy-2-(7-methyl-6-phenyl-5H-pyrrolo[1,2-c]imidazol-5-yl)ethyl)cyclohexan-1-ol (mixture of cis or trans);
N-methyl-4-(2-(7-methyl-6-phenyl-5H-pyrrolo[1,2-c]imidazol-5-yl)acetyl)cyclohexane-1-carboxamide;
2-(7-methyl-6-phenyl-5H-pyrrolo[1,2-c]imidazol-5-yl)-1-(4-phenylcyclohexyl)ethan-1-one;
4-[1-Hydroxy-2-(7-methyl-6-phenyl-5H-pyrrolo[1,2-c]imidazol-5-yl)-ethyl]-piperidine-1-carboxylic acid benzyl ester;
2-(2-Fluorophenyl)-1-(4-(2-(7-methyl-6-phenyl-5H-pyrrolo[1,2-c]imidazol-5-yl)acetyl)piperidin-1-yl)ethan-1-one;
2-(2-Fluorophenyl)-1-(4-(1-hydroxy-2-(7-methyl-6-phenyl-5H-pyrrolo[1,2-c]imidazol-5-yl)ethyl)piperidin-1-yl)ethan-1-one;
1-(1-Benzylpiperidin-4-yl)-2-(7-methyl-6-phenyl-5H-pyrrolo[1,2-c]imidazol-5-yl)ethan-1-one;
1-(1-Benzylpiperidin-4-yl)-2-(7-methyl-6-phenyl-5H-pyrrolo[1,2-c]imidazol-5-yl)ethan-1-ol;
1-(1-Benzoylpiperidin-4-yl)-2-(7-methyl-6-phenyl-5H-pyrrolo[1,2-c]imidazol-5-yl)ethan-1-one;
2-(7-Methyl-6-phenyl-5H-pyrrolo[1,2-c]imidazol-5-yl)-1-(1-(phenylsulfonyl)piperidin-4-yl)ethan-1-one;
(4-(1-Hydroxy-2-(7-methyl-6-phenyl-5H-pyrrolo[1,2-c]imidazol-5-yl)ethyl)piperidin-1-yl)(phenyl)methanone;
2-(7-Methyl-6-phenyl-5H-pyrrolo[1,2-c]imidazol-5-yl)-1-(1-(phenylsulfonyl)piperidin-4-yl)ethan-1-ol;
Ethyl 4-(2-(7-methyl-6-phenyl-5H-pyrrolo[1,2-c]imidazol-5-yl)acetyl)piperidine-1-carboxylate;
1-(1-(Cyclohexanecarbonyl)piperidin-4-yl)-2-(7-methyl-6-phenyl-5H-pyrrolo[1,2-c]imidazol-5-yl)ethan-1-one;
Ethyl 4-(1-hydroxy-2-(7-methyl-6-phenyl-5H-pyrrolo[1,2-c]imidazol-5-yl)ethyl)piperidine-1-carboxylate;
Cyclohexyl(4-(1-hydroxy-2-(7-methyl-6-phenyl-5H-pyrrolo[1,2-c]imidazol-5-yl)ethyl)piperidin-1-yl)methanone;
(4-(1-hydroxy-2-(7-methyl-6-phenyl-5H-pyrrolo[1,2-c]imidazol-5-yl)ethyl)piperidin-1-yl)(phenyl)methanone;
(4-(1-hydroxy-2-(7-methyl-6-phenyl-5H-pyrrolo[1,2-c]imidazol-5-yl)ethyl)piperidin-1-yl)(phenyl)methanone;
{4-[1-Hydroxy-2-(7-methyl-6-phenyl-5H-pyrrolo[1,2-c]imidazol-5-yl)-ethyl]-piperidin-1-yl}-pyridin-2-yl-methanone;
Ethyl 4-(1-hydroxy-2-(7-methyl-6-phenyl-5H-pyrrolo[1,2-c]imidazol-5-yl)ethyl)piperidine-1-carboxylate;
Ethyl 4-(1-hydroxy-2-(7-methyl-6-phenyl-5H-pyrrolo[1,2-c]imidazol-5-yl)ethyl)piperidine-1-carboxylate;
Cyclohexyl(4-(1-hydroxy-2-(7-methyl-6-phenyl-5H-pyrrolo[1,2-c]imidazol-5-yl)ethyl)piperidin-1-yl)methanone;
Cyclohexyl(4-(1-hydroxy-2-(7-methyl-6-phenyl-5H-pyrrolo[1,2-c]imidazol-5-yl)ethyl)piperidin-1-yl)methanone;
{4-[1-Hydroxy-2-(7-methyl-6-phenyl-5H-pyrrolo[1,2-c]imidazol-5-yl)-ethyl]-piperidin-1-yl}-pyridin-3-yl-methanone;
4-[1-Hydroxy-2-(7-methyl-6-phenyl-5H-pyrrolo[1,2-c]imidazol-5-yl)-ethyl]-piperidine-1-carboxylic acid phenyl amide;
1-(1-Benzoyl-azetidin-3-yl)-2-(7-methyl-6-phenyl-5H-pyrrolo[1,2-c]imidazol-5-yl)-ethanone;
{4-[1-Hydroxy-2-(7-methyl-6-phenyl-5H-pyrrolo[1,2-c]imidazol-5-yl)-ethyl]-piperidin-1-yl}-pyridin-2-yl-methanone (Isomer-I);
{4-[1-Hydroxy-2-(7-methyl-6-phenyl-5H-pyrrolo[1,2-c]imidazol-5-yl)-ethyl]-piperidin-1-yl}-pyridin-2-yl-methanone (Isomer-II);
{4-[1-Hydroxy-2-(7-methyl-6-phenyl-5H-pyrrolo[1,2-c]imidazol-5-yl)-ethyl]-piperidin-1-yl}-pyridin-4-yl-methanone;
1-(1-Cyclohexanesulfonyl-piperidin-4-yl)-2-(7-methyl-6-phenyl-5H-pyrrolo[1,2-c]imidazol-5-yl)-ethanol;
{4-[1-Hydroxy-2-(7-methyl-6-phenyl-5H-pyrrolo[1,2-c]imidazol-5-yl)-ethyl]-piperidin-1-yl}-(2-methyl-pyridin-4-yl)-methanone;
1-(1-Methanesulfonyl-piperidin-4-yl)-2-(7-methyl-6-phenyl-5H-pyrrolo[1,2-c]imidazol-5-yl)-ethanol;
2-{4-[1-Hydroxy-2-(7-methyl-6-phenyl-5H-pyrrolo[1,2-c]imidazol-5-yl)-ethyl]-piperidine-1-carbonyl}-benzoic acid;
4-[1-Hydroxy-2-(7-methyl-6-phenyl-5H-pyrrolo[1,2-c]imidazol-5-yl)-ethyl]-piperidine-1-carboxylic acid pyridin-2-ylamide;
5-(2-Cyclohexyl-2-fluoro-ethyl)-7-methyl-6-phenyl-5H-pyrrolo[1,2-c]imidazole;
1-Phenyl-2-(6-phenyl-5H-pyrrolo[1,2-c]imidazol-5-yl)-ethanone;
1-(7-Methyl-6-phenyl-5H-pyrrolo[1,2-c]imidazole-5-yl)-hexan-2-ol;
1-(7-Methyl-6-phenyl-5H-pyrrolo[1,2-c]imidazol-5-yl)-decan-2-ol;
2-(7-Methyl-6-phenyl-5H-pyrrolo[1,2-c]imidazol-5-yl)-N-propylacetamide;
Ethyl 5-(7-methyl-6-phenyl-5H-pyrrolo[1,2-c]imidazol-5-yl)-4-oxopentanoate;
4-Hydroxy-1-(4-hydroxypiperidin-1-yl)-5-(7-methyl-6-phenyl-5H-pyrrolo[1,2-c]imidazol-5-yl)pentan-1-one;
1-(4-Hydroxypiperidin-1-yl)-2-(7-methyl-6-phenyl-5H-pyrrolo[1,2-c]imidazol-5-yl)ethan-1-one;
2-(2-fluorophenyl)-N-(4-(2-(7-methyl-6-phenyl-5H-pyrrolo[1,2-c]imidazol-5-yl)acetyl)phenyl)acetamide;
4-[2-(7-Methyl-6-phenyl-5H-pyrrolo[1,2-c]imidazol-5-yl)-acetyl]-cyclohexanone oxime;
4-[1-Hydroxy-2-(7-methyl-6-phenyl-5H-pyrrolo[1,2-c]imidazol-5-yl)-ethyl]-cyclohexanone oxime;
N-cyclohexyl-4-(1-hydroxy-2-(7-methyl-6-phenyl-5H-pyrrolo[1,2-c]imidazol-5-yl)ethyl)piperidine-1-carboxamide;
1-(4-(1-hydroxy-2-(7-methyl-6-phenyl-5H-pyrrolo[1,2-c]imidazol-5-yl)ethyl)piperidin-1-yl)-2-phenylethan-1-one;
N-(3-chlorophenyl)-4-(1-hydroxy-2-(7-methyl-6-phenyl-5H-pyrrolo[1,2-c]imidazol-5-yl)ethyl)piperidine-1-carboxamide;
N-(3-chloro-4-fluorophenyl)-4-(1-hydroxy-2-(7-methyl-6-phenyl-5H-pyrrolo[1,2-c]imidazol-5-yl)ethyl)piperidine-1-carboxamide;
4-(1-hydroxy-2-(7-methyl-6-phenyl-5H-pyrrolo[1,2-c]imidazol-5-yl)ethyl)-N-(6-methylpyridin-2-yl)piperidine-1-carboxamide;
1-(4,4-difluorocyclohexyl)-2-(7-methyl-6-phenyl-5H-pyrrolo[1,2-c]imidazol-5-yl)ethan-1-ol;
tert-butyl (1R,4s)-4-((1S)-1-hydroxy-2-(7-methyl-6-phenyl-5H-pyrrolo[1,2-c]imidazol-5-yl)ethyl)cyclohexane-1-carboxylate;
2-(7-Methyl-6-phenyl-5H-pyrrolo[1,2-c]imidazol-5-yl)-1-(4-phenyl-cyclohexyl)-ethanol;
1-(4-(1-hydroxy-2-(7-methyl-6-phenyl-5H-pyrrolo[1,2-c]imidazol-5-yl)ethyl)piperidin-1-yl)-2-methylpropan-1-one;
2-(7-methyl-6-phenyl-5H-pyrrolo[1,2-c]imidazol-5-yl)-1-(1-(pyridin-2-yl)piperidin-4-yl)ethan-1-ol;
Ethyl (1R,4s)-4-((1S)-1-hydroxy-2-(7-methyl-6-phenyl-5H-pyrrolo[1,2-c]imidazol-5-yl)ethyl)cyclohexane-1-carboxylate;
N-(2-(4-(1-hydroxy-2-(7-methyl-6-phenyl-5H-pyrrolo[1,2-c]imidazol-5-yl)ethyl)piperidin-1-yl)ethyl)methanesulfonamide;
2-(7-methyl-6-phenyl-5H-pyrrolo[1,2-c]imidazol-5-yl)-1-(1-phenylpiperidin-4-yl)ethan-1-ol;
1-(1-isobutylpiperidin-4-yl)-2-(7-methyl-6-phenyl-5H-pyrrolo[1,2-c]imidazol-5-yl)ethan-1-ol;
(4-(1-hydroxy-2-(7-methyl-6-phenyl-5H-pyrrolo[1,2-c]imidazol-5-yl)ethyl)piperidin-1-yl)(4-hydroxyphenyl)methanone;
(2-fluorophenyl)(4-(1-hydroxy-2-(7-methyl-6-phenyl-5H-pyrrolo[1,2-c]imidazol-5-yl)ethyl)piperidin-1-yl)methanone;
(4-(1-hydroxy-2-(7-methyl-6-phenyl-5H-pyrrolo[1,2-c]imidazol-5-yl)ethyl)piperidin-1-yl)(piperidin-4-yl)methanone;
azetidin-3-yl(4-(1-hydroxy-2-(7-methyl-6-phenyl-5H-pyrrolo[1,2-c]imidazol-5-yl)ethyl)piperidin-1-yl)methanone;
(4-(1-hydroxy-2-(7-methyl-6-phenyl-5H-pyrrolo[1,2-c]imidazol-5-yl)ethyl)piperidin-1-yl)(2-hydroxyphenyl)methanone;
2-(7-methyl-6-phenyl-5H-pyrrolo[1,2-c]imidazol-5-yl)-1-(1-(oxetan-3-yl)piperidin-4-yl)ethan-1-ol;
1-(1-(azetidin-3-yl)piperidin-4-yl)-2-(7-methyl-6-phenyl-5H-pyrrolo[1,2-c]imidazol-5-yl)ethan-1-ol;
2-(7-methyl-6-phenyl-5H-pyrrolo[1,2-c]imidazol-5-yl)-1-(1-(pyrimidin-5-yl)piperidin-4-yl)ethan-1-ol;
2-(7-methyl-6-phenyl-5H-pyrrolo[1,2-c]imidazol-5-yl)-1-(1-(pyridin-4-yl)piperidin-4-yl)ethan-1-ol;
2-(7-methyl-6-phenyl-5H-pyrrolo[1,2-c]imidazol-5-yl)-1-(1-(oxazol-4-yl)piperidin-4-yl)ethan-1-ol;
1-(1-(1H-imidazol-4-yl)piperidin-4-yl)-2-(7-methyl-6-phenyl-5H-pyrrolo[1,2-c]imidazol-5-yl)ethan-1-ol;
1-(1-(1H-pyrazol-3-yl)piperidin-4-yl)-2-(7-methyl-6-phenyl-5H-pyrrolo[(7-m 1,2-c]imidazol-5-yl)ethan-1-ol;
1-(1-(2-aminophenyl)piperidin-4-yl)-2-(7-methyl-6-phenyl-5H-pyrrolo[1,2-c]imidazol-5-yl)ethan-1-ol;
methyl 4-(4-(1-hydroxy-2-(7-methyl-6-phenyl-5H-pyrrolo[1,2-c]imidazol-5-yl)ethyl)piperidin-1-yl)benzoate;
methyl 3-(4-(1-hydroxy-2-(7-methyl-6-phenyl-5H-pyrrolo[1,2-c]imidazol-5-yl)ethyl)piperidin-1-yl)benzoate;
4-(1-amino-2-(7-methyl-6-phenyl-5H-pyrrolo[1,2-c]imidazol-5-yl)ethyl)cyclohexan-1-ol;
4-(1-hydroxy-2-(7-methyl-6-phenyl-5H-pyrrolo[1,2-c]imidazol-5-yl)ethyl)cyclohexane-1-sulfonamide;
4-(1-hydroxy-2-(7-methyl-6-phenyl-5H-pyrrolo[1,2-c]imidazol-5-yl)ethyl)-1-(hydroxymethyl)cyclohexan-1-ol;
4-(1-hydroxy-2-(7-methyl-6-phenyl-5H-pyrrolo[1,2-c]imidazol-5-yl)ethyl)cyclohexyl acetate;
4-(1-hydroxy-2-(7-methyl-6-phenyl-5H-pyrrolo[1,2-c]imidazol-5-yl)ethyl)cyclohexyl benzoate;
4-(1-hydroxy-2-(7-methyl-6-phenyl-5H-pyrrolo[1,2-c]imidazol-5-yl)ethyl)cyclohexyl dihydrogen phosphate;
2-(7-methyl-6-phenyl-5H-pyrrolo[1,2-c]imidazol-5-yl)-1-(1-oxaspiro[3.5]nonan-7-yl)ethan-1-ol;
2-(7-methyl-6-phenyl-5H-pyrrolo[1,2-c]imidazol-5-yl)-1-(1-oxaspiro[4.5]decan-8-yl)ethan-1-ol;
N-(4-(1-hydroxy-2-(7-methyl-6-phenyl-5H-pyrrolo[1,2-c]imidazol-5-yl)ethyl)cyclohexyl)benzamide;
N-(4-(1-hydroxy-2-(7-methyl-6-phenyl-5H-pyrrolo[1,2-c]imidazol-5-yl)ethyl)cyclohexyl)benzamide;
Benzyl 4-(1-hydroxy-2-(7-methyl-6-phenyl-5H-pyrrolo[1,2-c]imidazol-5-yl)ethyl)cyclohexane-1-carboxylate;
pyridin-4-ylmethyl 4-(1-hydroxy-2-(7-methyl-6-phenyl-5H-pyrrolo[1,2-c]imidazol-5-yl)ethyl)cyclohexane-1-carboxylate;
1-(4-(1-hydroxy-2-(7-methyl-6-phenyl-5H-pyrrolo[1,2-c]imidazol-5-yl)ethyl)cyclohexyl)cyclopropan-1-ol;
3-(1-hydroxy-2-(7-methyl-6-phenyl-5H-pyrrolo[1,2-c]imidazol-5-yl)ethyl)cyclobutan-1-ol;
1-(3-methoxycyclobutyl)-2-(7-methyl-6-phenyl-5H-pyrrolo[1,2-c]imidazol-5-yl)ethan-1-ol;
3-(1-hydroxy-2-(7-methyl-6-phenyl-5H-pyrrolo[1,2-c]imidazol-5-yl)ethyl)cyclobutane-1-carboxylic acid;
methyl 3-(1-hydroxy-2-(7-methyl-6-phenyl-5H-pyrrolo[1,2-c]imidazol-5-yl)ethyl)cyclobutane-1-carboxylate;
3-(1-hydroxy-2-(7-methyl-6-phenyl-5H-pyrrolo[1,2-c]imidazol-5-yl)ethyl)cyclobutane-1-carb oxamide;
3-(1-hydroxy-2-(7-methyl-6-phenyl-5H-pyrrolo[1,2-c]imidazol-5-yl)ethyl)-N-methylcyclobutane-1-carboxamide; including pharmaceutically acceptable salts, pharmaceutically acceptable solvates, pharmaceutically acceptable hydrates, tautomers, stereoisomers, ester prodrugs, or combination thereof.

5. A pharmaceutical composition comprising a compound according to claim 1 and at least one pharmaceutically acceptable excipient.

6. The pharmaceutical composition according to claim 5, wherein the pharmaceutically acceptable excipient is a carrier or diluent.

7. A method for preventing, ameliorating or treating a indoleamine 2,3-dioxygenase (IDO) and/or tryptophan 2,3-dioxygenase (TDO) mediated disease, disorder or syndrome in a subject in need thereof comprising administering to the subject a therapeutically effective amount of a compound according to claim 1.

8. The method according to claim 7, wherein the a indoleamine 2,3-dioxygenase (IDO) and/or tryptophan 2,3-dioxygenase (TDO) mediated disease, disorder or syndrome is cancer, an inflammatory condition, an infectious disease, Chagas disease, a central nervous system disease or disorder, depression, psychosis, psychiatric disorders, bipolar disorders, a neurodegenerative disorder, trauma, age-related cataracts, organ transplant rejection, viral infection, anti-retroviral therapy, treating or preventing HIV/AIDS, chronic HBV, malaria, schizophrenia, HCV, inflammation-associated arthritis or autoimmune arthritis, allergic airways disease, joint inflammation, multiple sclerosis, Parkinson's disease (PD), Alzheimer's disease, stroke, amyotrophic lateral sclerosis, dementia, cognitive disorders, psychotic disorders/cognitive disorder/dementia associated with various neurodegenerative diseases, allergic encephalomyelitis, Huntington's disease, anxiety, insomnia, atherosclerosis, coronary artery disease, kidney disease, sepsis-induced hypotension, Psychiatric disorders and pain, chronic pain, General anaesthesia, Cataracts, Endometriosis, Contraception and abortion, coronary heart disease, chronic renal failure, or post anaesthesia cognitive dysfunction.

9. A method of treating cancer in a subject in need thereof comprising administering to the subject a therapeutically effective amount of a compound according to claim 1.

10. A method according to claim 9, wherein the cancer is selected from a solid or liquid tumour including cancer of the eye, brain (such as gliomas, glioblastomas, medullablastomas, craniopharyngioma, ependymoma, and astrocytoma), colon, parathyroid gland, gall bladder, head and neck, breast, bone, hypopharyngeal gland, lung, bronchus, liver, skin (melanomas), ureter, urethra, urothelium, testicles, vaginal, anus, mouth, lip, throat, oral cavity, nasal cavity, Gastro-intestinal, Gastric stomach, Gastro-intestinal stromal cells, small intestine, laryngeal gland, ovary, thyroid, bile duct, cervix, heart, spinal cord, kidney, oesophagus, nasopharyngeal gland, pituitary gland, salivary gland, prostate, penile tissue, pancreas, adrenal glands; an epithelial and squamous cell cancers of various tissue types, an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukemia (T-PLL), large granular lymphocytic leukemia, adult T-cell leukemia/lymphoma (ATLL), juvenile myelomonocytic leukaemia, Hodgkin's lymphoma, classical Hodgkin's lymphoma, non-Hodgkin's lymphoma, mantle cell lymphoma, follicular lymphoma, primary effusion lymphoma, AIDS-related lymphoma, diffuse B cell lymphoma, Burkitt lymphoma, and cutaneous T-cell lymphoma), Barret's adenocarcinoma, cervical cancer, esophageal cancer, ovarian cancer, colo-rectal cancer, prostate cancer, hematologic cancers, cancer of Billary Tract, blood cancer, large intestinal colon carcinoma, histiocytic lymphoma, lung adenocarcinoma, astrocytoma, meningioma, medulloblastoma and peripheral neuroectodermal tumors, diffuse large B-cell lymphoma (DLBCL), gall bladder carcinoma, bronchial carcinoma, small cell lung carcinoma, non-small cell lung carcinoma (NSCLC), multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma, metastatic carcinomas or plasmocytoma.

11. A method for preventing, ameliorating or treating an indoleamine 2,3-dioxygenase (IDO) and/or tryptophan 2,3-dioxygenase (TDO) mediated disease in a subject in need thereof comprising administering to the subject a therapeutically effective amount of a pharmaceutical composition according to claim 5.

12. The method according to claim 11, wherein the indoleamine 2,3-dioxygenase (IDO) and/or tryptophan 2,3-dioxygenase (TDO) mediated disease is cancer, an inflammatory condition, an infectious disease, Chagas disease, a central nervous system disease or disorder, depression, psychosis, psychiatric disorders, bipolar disorders, a neurodegenerative disorder, trauma, age-related cataracts, organ transplant rejection, viral infection, anti-retroviral therapy, treating or preventing HIV/AIDS, chronic HBV, malaria, schizophrenia, HCV, inflammation-associated arthritis or autoimmune arthritis, allergic airways disease, joint inflammation, multiple sclerosis, Parkinson's disease (PD), Alzheimer's disease, stroke, amyotrophic lateral sclerosis, dementia, cognitive disorders, psychotic disorders/cognitive disorder/dementia associated with various neurodegenerative diseases, allergic encephalomyelitis, Huntington's disease, anxiety, insomnia, atherosclerosis, coronary artery disease, kidney disease, sepsis-induced hypotension, Psychiatric disorders and pain, chronic pain, General anaesthesia, Cataracts, Endometriosis, Contraception and abortion, coronary heart disease, chronic renal failure, or post anaesthesia cognitive dysfunction.
